(19) United States
(12) Patent Application Publication
(10) Pub. No.: US 2013/0196903 A1
(43) Pub. Date: Aug. 1, 2013

(54) Multimeric Inhibitors of Viral Fusion and Uses Thereof

(71) Applicant: Antonello Pessi, Rome (IT)

(72) Inventor: Antonello Pessi, Rome (IT)

(73) Assignee: JV BIO SRL, Naplea (IT)

(21) Appl. No.: 13/816,163

(22) PCT Filed: Aug. 11, 2011
(86) PCT No.: PCT/EP2011/063888

§ 371 Date: Apr. 18, 2013

Related U.S. Application Data
(60) Provisional application No. 61/372,632, filed on Aug. 11, 2010.

(51) Int. Cl. A61K 47/48

(52) U.S. Cl. CPC A61K 47/48123
USPC 514/3.8; 530/323; 514/3.7

(57) ABSTRACT

The present invention relates to novel multimeric inhibitors of viral entry into cells and their use for the prophylaxis and treatment of viral infections.

### TECHNICAL FIELD OF THE INVENTION

The present invention relates to novel (multimeric) inhibitors of viral entry into cells and their use for the prophylaxis and treatment of viral infections.

### BACKGROUND OF THE INVENTION

“Enveloped viruses”, such as orthomyxoviruses, paramyxoviruses, retroviruses, flaviviruses, rhabdoviruses and alphaviruses, are surrounded by a lipid bilayer originating from the host plasma membrane (11). This envelope contains glycoproteins that mediate receptor binding and fusion between viral and host cell membranes. Cholesterol and sphingolipids such as sphingomyelin are often enriched in these viral lipid bilayers, particularly in lipid-rich rafts in their plasma membrane (3, 7). A number of transmembrane proteins and receptors, including CD4 which is the primary receptor for HIV envelope gp120, are particularly enriched in lipid rafts.

To accomplish the mixing of cellular and viral contents, fusion proteins like gp41 of HIV must undergo a complex series of conformational changes, triggered by the initial binding of the virus to the target cell (2). For gp41, the fusion-active conformation contains two heptad repeat regimes: HRN (proximal to the N terminus) and HRC (proximal to the C terminus). The hydrophobic fusion peptide region inserts into the host cell membrane, whereas the HRN region of gp41 forms a trimeric coiled coil structure. HRC regions then fold back within the hydrophobic grooves of the HRN coiled coil, forming a hairpin structure containing a thermodynamically stable six-helix bundle that draws the viral and cellular membranes together for fusion (2). It has been shown that interference with HRN-HRC interaction inhibits viral entry and hence viral replication (WO 2005/067960 A1).

A number of HRN-binding peptides have been described in the prior art, like C34 and T20, and the latter has been approved for the treatment of HIV with the name enfuvirtide (9). For other viruses however, the HRN-binding peptides corresponding to C34/T20 are of relatively low potency. It has been shown that the efficacy of peptide fusion inhibitors depends on two variables: the strength of interaction of the peptide with the target fusion protein; and the time window before the transient fusion intermediate collapses to the post-fusion structure—i.e., the kinetics of fusion (16, 19). For viruses with fast fusion kinetics, the bulk concentration of the inhibitor required to achieve adequate concentration at the site where fusion occurs can be very high, translating in low antiviral potency.

The enveloped virus designated as respiratory syncytial virus (RSV) is the most important cause of viral lower respiratory tract illness (LRTI) in infants and children (2). In the United States, it is estimated that 70,000-126,000 infants are hospitalized annually with RSV pneumonia or bronchiolitis and that the rate of hospitalization for bronchiolitis has increased since 1980 (4). Although it is traditionally regarded as a pediatric pathogen, RSV also causes life-threatening pulmonary disease in bone marrow transplant recipients and older persons. The enveloped viruses designated as human parainfluenza viruses types 1, 2, and 3 (HPIV1, HPIV2 and HPIV3, respectively) are also important respiratory pathogens in infancy and early childhood: ˜25% of individuals in this age group will develop clinically significant HPIV disease (3). In a recent study involving young children with illnesses associated with a positive viral culture, HPIVs were recovered from 18% of outpatients with upper respiratory tract illness (URTI), 22% with LRTI, and 64% with croup (3). HPIV1 and HPIV2 are the principal causes of croup, which occurs primarily in children 6-48 months of age. HPIV3 causes bronchiolitis and pneumonia predominantly in children <12 months of age. Collectively, HPIVs are second only to RSV as important causes of viral LRTI in young children (1). Like RSV, HPIV3 can cause severe LRTI in immuno-compromised patients. By the time they are 2 years of age, almost all children will have been infected with RSV, and ˜50% will have been infected twice. HPIV3 also infects children early in life: ˜60% and ˜80% will have been infected before the ages of 2 and 4 years, respectively. Infection with HPIV1 and HPIV2 occurs when children are slightly older, but, by 5 years of age, most children have been infected with these viruses at least once. RSV epidemics occur during the winter and early spring in temperate climates and during the rainy season in some, but not all, tropical climates. Currently, in the United States, HPIV1 epidemics occur in the fall of odd-numbered years, HPIV2 epidemics occur biennially or annually in the fall, and HPIV3 epidemics occur annually in the spring and summer.

Both RSV and HPIVs but also many other enveloped viruses especially of the paramyxovirus Paramyxoviridae family of the Mononegavirales order can re-infect individuals throughout life, many of which will cause upper respiratory tract infections (URTI). Primary infection with RSV or HPIV3 does not always elicit immune responses that will protect the lower respiratory tract, because RSV- and HPIV3-associated LRTI can occur in young children experiencing second infections.

Licensed vaccines for RSV and HPIVs and enveloped viruses are not currently available. Furthermore, it is frequently observed that primary infection with an enveloped virus such as a paramyxovirus generates merely a suboptimal immune response especially in young infants.

A recently described method teaches how to selectively enrich peptide fusion inhibitors into lipid rafts where fusion occurs, thus increasing their antiviral potency: conjugation to the peptide of a cholesterol group (4). An increase in antiviral potency by addition of a cholesterol group has been reported for the HIV-inhibitory peptide C34 (4), and for peptides derived from the fusion protein of human parainfluenza virus type 3 (HPIV3) (17), which are inhibitors of both HPIV3 and henipavirus infection (13, 14). Conjugation of cholesterol therefore represents a method to augment the antiviral potency of peptide fusion inhibitors, and its mechanism of action is to increase the rate of inhibitor association with the fusion intermediates.

An alternative way to improve potency is to decrease the rate of inhibitor dissociation from the fusion intermediate. This has been explored for HIV, where a number of C34 variants have been engineered to interact more strongly with the HRN of gp41. For example, since C34 shows essentially no helical structure prior to folding onto HRN to form the 6-helix bundle, analogs have been designed with enhanced helical structure in solution, which reduces the entropy penalty for binding to HRN, and translates into greater strength of association (1, 5, 10, 12). However, although these peptides form complexes with HRN with much increased stability with respect to C34, none is significantly more potent than C34 (1).

In a further approach, on the basis of the three-dimensional structure of the HIV-1 gp41, fusogenic core conformation an anti-HIV peptide termed “sifuvirtide” or “Fusonex” was designed which blocks the six-helix bundle formation between C34-biotin and a counterpart NHR peptide N36. The fusion inhibition concentration thereof is lower than that of T20 (21, EP-A-1 421 946).

Summarizing the above, therapeutic substances for many enveloped viruses are currently not available. Also when available, therapeutic substances against enveloped viruses still exhibit severe side-effects. This is frequently the case as these pharmaceuticals are applied in substantial doses which are required to interfere with the pathogenic event of viral entry into a cell.

Thus, there is a need to develop more effective virus inhibitors that can thus be administered in smaller doses and that preferably are effective against multiple different members of the enveloped virus family, offering a universal therapeutic and prophylactic approach effective against multiple different diseases.

### BRIEF SUMMARY OF THE INVENTION

The present inventors have identified novel and improved (multimeric) inhibitors of viral fusion, which are surprisingly effective against enveloped viruses.

In a first aspect, the present invention relates to a multimeric inhibitor of viral fusion comprising:
(i) at least two polypeptides capable of inhibiting fusion of at least one enveloped virus with a cellular membrane, and
(ii) a membrane integrating lipid selected from the group consisting of cholesterol, a sphingolipid, a glycolipid, a glycerophospholipid and membrane integrating derivatives thereof, which is attached to said polypeptides;

or a pharmaceutically acceptable salt thereof.

In a preferred embodiment of the first aspect at least one of said polypeptides is preferably selected from the group consisting of
a) a polypeptide comprising an amino sequence WX₁EWX₂REINX₃YX₄SLIX₅SLIEEX₆QX₇QQX₈KNEX₉X₁₀LX₁₁X₁₂L (SEQ ID NO: 188), wherein
   X₁ is selected from M, Nle (norleucine), Q and N, preferably from N or Q, most preferably N;
   X₂ is selected from D and E, preferably E;
   X₃ is selected from N and K, preferably K;
   X₄ is selected from T and I, preferably T;
   X₅ is selected from H and Y, preferably Y;
   X₆ is selected from S, A, L and Abu (2-aminobutyric acid), preferably S or L, more preferably A;
   X₇ is selected from N and K, preferably N;
   X₈ is selected from E, e (D-glutamic acid), D, d (D-aspartic acid), preferably E or D, more preferably D;
   X₉ is selected from K, k (D-lysine), R, r (D-arginine), preferably K or R, more preferably K;
   X₁₀ may not be present or is selected from E, D, A, preferably E or D, more preferably D;
   X₁₁ may not be present or is selected from L, K, R, preferably L or K, more preferably L; and
   X₁₂ may not be present or is selected from E, e (D-glutamic acid), A, preferably E or e, more preferably E;
b) a polypeptide comprising an amino acid sequence having at least 75% identity to WNEWEREINKYTSLIYSLIEEAQNQQDKNEKDLLEL (SEQ ID NO: 192); or
c) a polypeptide comprising the amino acid sequence SWETWEREIENYTRQIYRILEESQEQQDRNERDLLE (SEQ ID NO: 189),

In a second aspect, the present invention relates to a pharmaceutical composition comprising the multimeric inhibitor according to the first aspect of the present invention or a pharmaceutically acceptable salt thereof and a pharmaceutically acceptable excipient.

In a third aspect, the present invention relates to a multimeric inhibitor according to the first aspect of the present invention or a pharmaceutically acceptable salt thereof for the treatment or prevention of infection(s) by (an) enveloped virus(es).

In a fourth aspect, the present invention relates to a method for making a broad-spectrum multimeric inhibitor of viral fusion effective against at least two, preferably three or four, different enveloped viruses, wherein the method comprises the steps of:
(i) generating at least two polypeptides each comprising a peptide, wherein at least one of said peptides is a hybrid peptide which is capable of inhibiting fusion of at least two, preferably three or four, different enveloped viruses by binding to a HR1 domain or HR2 domain of a Type I viral fusogenic protein of said enveloped viruses selected from the group consisting of HR domains with an amino acid sequence according to SEQ ID NO: 1 to SEQ ID NO: 17, SEQ ID NO: 105, and SEQ ID NO: 144 to SEQ ID NO: 151, and wherein said hybrid peptide comprises amino acids from HR domains of a Type I viral fusogenic protein of at least two different enveloped viruses; and
(ii) covalently linking a membrane integrating lipid selected from the group consisting of cholesterol, a sphingolipid, a glycolipid, a glycerophospholipid and membrane integrating derivatives thereof to the C-terminal or N-terminal region of said polypeptides.

In a fifth aspect, the present invention relates to a method for making a broad-spectrum multimeric inhibitor of viral fusion effective against at least two, preferably three or four, different enveloped viruses, wherein the method comprises the steps of:
(i) generating at least two polypeptides each comprising a peptide, wherein at least one of said polypeptides is preferably defined as in the first aspect and/or wherein at least one of said peptides is a hybrid peptide which is capable of inhibiting fusion of at least two, preferably three or four, different enveloped viruses by binding to a beta-sheet domain of a Type II viral fusogenic protein of said enveloped viruses selected from the group consisting of Dengue virus, West Nile virus, Yellow fever virus, and Japanese encephalitis virus, and wherein said hybrid peptide comprises amino acids from membrane-proximal regions (MPRs) of a Type II viral fusogenic protein of at least two different enveloped viruses selected from the group consisting of MPRs with an amino acid sequence according to SEQ ID NO: 137 to SEQ ID NO: 143; and
(ii) covalently linking a membrane integrating lipid selected from the group consisting of cholesterol, a sphingolipid, a glycolipid, a glycerophospholipid and membrane integrating derivatives thereof to the C-terminal region of said polypeptides.

In a sixth aspect, the present invention relates to a method for making a broad-spectrum multimeric inhibitor of viral fusion effective against at least two, preferably three or four, different enveloped viruses, wherein the method comprises the steps of:
(i) generating at least two polypeptides each comprising a peptide, wherein at least one of said polypeptides is preferably defined as in the first aspect and/or wherein at least one of said peptides is a hybrid peptide which is capable of inhibiting fusion of at least two, preferably three or four, different enveloped viruses by binding to a HR domain of a Type III viral fusogenic protein of said enveloped viruses selected from the group consisting of Herpes simplex virus (HSV), Human herpesvirus 6A; Human herpesvirus 6B, and Cytomegalovirus, and wherein said hybrid peptide comprises amino acids from HR domains of a Type III viral fusogenic protein of at least two different enveloped viruses selected from the group consisting of HR domains with an amino acid sequence according to SEQ ID NO: 129 to SEQ ID NO: 136; and
(ii) covalently linking a membrane integrating lipid selected from the group consisting of cholesterol, a sphingolipid, a glycolipid, a glycerophospholipid and membrane integrating derivatives thereof to the C-terminal or N-terminal region of said polypeptides.

In a seventh aspect, the present invention relates to a method for making a broad-spectrum multimeric inhibitor of viral fusion effective against at least two, preferably three or four, different enveloped viruses, wherein the method comprises the steps of:
(i) generating at least two polypeptides each comprising a peptide, wherein at least one of said polypeptides is preferably defined as in the first aspect and/or wherein at least one of said peptides is a hybrid peptide which is capable of inhibiting fusion of at least two, preferably three or four, different enveloped viruses by binding to a HR1 domain or HR2 domain of a Type I viral fusogenic protein of said enveloped viruses selected from the group consisting of Influenza virus, Parainfluenza virus, Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Rabies virus, Junin virus, Machupo virus, Guanarito virus, and Lassa virus, and wherein said hybrid peptide comprises amino acids from HR domains of a Type I viral fusogenic protein of at least two different enveloped viruses selected from the group consisting of HR domains with an amino acid sequence according to SEQ ID NO: 18 to SEQ ID NO: 34, SEQ ID NO: 50 to SEQ ID NO: 54, SEQ ID NO: 83 to SEQ ID NO: 99, SEQ ID NO: 102 to SEQ ID NO: 104 and SEQ ID NO: 120 to SEQ ID NO: 128; and
(ii) covalently linking a membrane integrating lipid selected from the group consisting of cholesterol, a sphingolipid, a glycolipid, a glycerophospholipid and membrane integrating derivatives thereof to the C-terminal or N-terminal region of said polypeptides.

In an eighth aspect, the present invention relates to a monomeric inhibitor of viral fusion comprising:
(i) one polypeptide capable of inhibiting fusion of at least one enveloped virus with a cellular membrane, wherein said polypeptide is selected from the group consisting of
   a) a polypeptide comprising an amino sequence WX₁EWX₂REINX₃YX₄SLIX₅SLIEEX₆QX₇QQX₈KNEX₉X₁₀LX₁₁X₁₂L (SEQ ID NO: 188), wherein
      X₁ is selected from M, Nle (norleucine), Q and N, preferably from N or Q, most preferably N;
      X₂ is selected from D and E, preferably E;
      X₃ is selected from N and K, preferably K;
      X₄ is selected from T and I, preferably T;
      X₅ is selected from H and Y, preferably Y;
      X₆ is selected from S, A, L and Abu (2-aminobutyric acid), preferably S or L, more preferably A;
      X₇ is selected from N and K, preferably N;
      X₈ is selected from E, e (D-glutamic acid), D, d (D-aspartic acid), preferably E or D, more preferably D;
      X₉ is selected from K, k (D-lysine), R, r (D-arginine), preferably K or R, more preferably K;
      X₁₀ may not be present or is selected from E, D, A, preferably E or D, more preferably D;
      X₁₁ may not be present or is selected from L, K, R, preferably L or K, more preferably L; and
      X₁₂ may not be present or is selected from E, e (D-glutamic acid), A, preferably E or e, more preferably E;
   b) a polypeptide comprising an amino acid sequence having at least 75% identity to WNEWEREINKYTSLIYSLIEEAQNQQDKNEKDLLEL (SEQ ID NO: 192); or
   c) a polypeptide comprising the amino acid sequence SWETWEREIENYTRQIYRILEESQEQQDRNERDLLE (SEQ ID NO: 189);
(ii) a membrane integrating lipid selected from the group consisting of cholesterol, a sphingolipid, a glycolipid, a glycerophospholipid and membrane integrating derivatives thereof, which is attached to said polypeptides;

or a pharmaceutically acceptable salt thereof.

In a ninth aspect, the present invention relates to a pharmaceutical composition comprising the inhibitor according to the eighth aspect of the present invention or a pharmaceutically acceptable salt thereof and a pharmaceutically acceptable excipient.

In a tenth aspect, the present invention relates to an inhibitor according to the eighth aspect of the present invention or a pharmaceutically acceptable salt thereof for the treatment or prevention of infection(s) by (an) enveloped virus(es), preferably Human immunodeficiency virus (HIV).

In an eleventh aspect, the present invention relates for making a monomeric inhibitor of viral fusion effective against at least one enveloped virus, wherein the method comprises the steps of:
(i) generating a polypeptide defined as in the eighth aspect which is capable of inhibiting fusion of an enveloped virus, preferably Human immunodeficiency virus (HIV), and
(ii) covalently linking a membrane integrating lipid selected from the group consisting of cholesterol, a sphingolipid, a glycolipid, a glycerophospholipid and membrane integrating derivatives thereof to the C-terminal or N-terminal region of said polypeptides.

This summary of the invention does not necessarily describe all features of the invention.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland) and as described in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, edited by Susan Budavari et al., CRC Press, 1996, and the United States Pharmacopeia-25/National Formulary-20, published by the United States Pharmcopeial Convention, Inc., Rockville Md., 2001. The inhibitor molecules of the invention comprise amino acids which are designated following the standard one- or three-letter code according to WIPO standard ST.25 unless otherwise indicated. If not indicated otherwise, the one- or three letter code is directed at the naturally occurring L-amino acids. At one or more positions of the amino acid sequence of said one or more polypeptides the inhibitor molecules of the invention may comprise the D-form of an amino acid which is indicated by lower case letters. (e.g. e: D-glutamic acid, r: D-arginine, k: D-lysine) Furthermore, at one ore more positions of the amino acid sequence of said one or more polypeptides the inhibitor molecules may comprise modified and/or unusual amino acids like norleucine (Nle) or 2-aminobutyric acid (Abu).

Throughout this specification and the claims which follow, unless the context requires otherwise, the word “comprise”, and variations such as “comprises” and “comprising”, will be understood to imply the inclusion of a stated feature, integer or step or group of features, integers or steps but not the exclusion of any other feature, integer or step or group of integers or steps. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

As used in this specification and the appended claims, the singular forms “a”, “an”, and “the” include plural referents, unless the content clearly dictates otherwise.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

The inventors of the present invention have identified novel multimeric inhibitors of viral fusion comprising membrane integrating lipid-conjugated polypeptides with improved antiviral potency. For example, single polypeptides capable of inhibiting fusion of an enveloped virus with the cellular membrane could be rendered more effective when comprised as polypeptide multimers, e.g. dimers, trimers, or tetramers, in the multimeric inhibitor of viral fusion of the present invention and when attached to a membrane integrating lipid, e.g. cholesterol. This permits the application of reduced amounts of therapeutic and prophylactic inhibitory polypeptides to achieve the same health benefit at a low dose than that is achieved by respective non-modified inhibitory polypeptides of the state of the art at a respectively larger dose.

In detail, the inventors of the present invention surprisingly found that a multimeric inhibitor comprising at least two polypeptides capable of inhibiting fusion of at least one enveloped virus with the cellular membrane and a membrane integrating lipid selected from the group consisting of cholesterol, a sphingolipid, a glycolipid, a glycerophospholipid and membrane integrating derivatives thereof which is attached to said polypeptides is very effective in inhibiting viral fusion.

Thus, in a first aspect, the present invention relates to a (broad-spectrum) multimeric inhibitor of viral fusion comprising, essentially consisting of, or consisting of:
(i) at least two polypeptides, preferably at least 3, 4, 5, or 6 polypeptides, capable of inhibiting fusion of at least one enveloped virus, preferably at least 2, 3, or 4 (different) enveloped viruses, with a cellular membrane, and
(ii) a membrane integrating lipid selected from the group consisting of cholesterol, a sphingolipid, a glycolipid, a glycerophospholipid and membrane integrating derivatives thereof, which is attached to said polypeptides;

or a pharmaceutically acceptable salt thereof.

In a preferred embodiment of the first aspect at least one of said polypeptides is preferably selected from the group consisting of
a) a polypeptide comprising an amino sequence WX₁EWX₂REINX₃YX₄SLIX₅SLIEEX₆QX₇QQX₈KNEX₉X₁₀LX₁₁X₁₂L (SEQ ID NO: 188), wherein
   X₁ is selected from M, Nle (norleucine), Q and N, preferably from N or Q, most preferably N;
   X₂ is selected from D and E, preferably E;
   X₃ is selected from N and K, preferably K;
   X₄ is selected from T and I, preferably T;
   X₅ is selected from H and Y, preferably Y;
   X₆ is selected from S, A, L and Abu (2-aminobutyric acid), preferably S or L, more preferably A;
   X₇ is selected from N and K, preferably N;
   X₈ is selected from E, e (D-glutamic acid), D, d (D-aspartic acid), preferably E or D, more preferably D;
   X₉ is selected from K, k (D-lysine), R, r (D-arginine), preferably K or R, more preferably K;
   X₁₀ may not be present or is selected from E, D, A, preferably E or D, more preferably D;
   X₁₁ may not be present or is selected from L, K, R, preferably L or K, more preferably L; and
   X₁₂ may not be present or is selected from E, e (D-glutamic acid), A, preferably E or e, more preferably E;
b) a polypeptide comprising an amino acid sequence having at least 75% identity to WNEWEREINKYTSLIYSLIEEAQNQQDKNEKDLLEL (SEQ ID NO: 192); or
c) a polypeptide comprising the amino acid sequence SWETWEREIENYTRQIYRILEESQEQQDRNERDLLE (SEQ ID NO: 189)

A preferred sequence of at least one of said at least two polypeptides comprises, essentially consists of, or consists of WNEWEREINKYTSLIYSLIEEAQNQQDKNEKDLLEL (SEQ ID NO: 192) or an amino acid sequence having at least 75% identity to this sequence.

Said at least 2, 3, or 4 enveloped viruses may be different enveloped viruses, e.g. different types of enveloped viruses.

As used herein, the terms “protein”, “polypeptide” or “peptide” may refer to both naturally occurring proteins, polypeptides, or peptides and synthesized (synthetic) proteins, polypeptides, or peptides that may include naturally or non-naturally occurring amino acids. Proteins, polypeptides, or peptides can be also chemically modified by modifying a side chain or a free amino or carboxy-terminus of a natural or non-naturally occurring amino acid. This chemical modification includes the addition of further chemical moieties as well as the modification of functional groups in side chains of the amino acids, such as a glycosylation.

The term “hybrid peptide”, as used herein, refers in preferred embodiments to a peptide which comprises amino acids from homologous regions of at least two (different), preferably three or four, enveloped viruses, e.g. enveloped viruses from different types of viruses and/or an amino acid sequence comprising an amino sequence WX₁EWX₂REINX₃YX₄SLIX₅SLIEEX₆QX₇QQX₈KNEX₉X₁₀LX₁₁X₁₂L (SEQ ID NO: 188), a polypeptide comprising an amino acid sequence having at least 75% identity to WNEWEREINKYTSLIYSLIEEAQNQQDKNEKDLLEL (SEQ ID NO: 192); or an amino sequence SWETWEREIENYTRQIYRILEESQEQQDRNERDLLE (SEQ ID NO: 189); wherein X₁ to X₁₂ are defined as described above. Said amino acids may be from HR domains, preferably HR1 domains or HR2 domains, of at least two (different), preferably three or four, enveloped viruses, e.g. enveloped viruses from different types of viruses such as two different viruses of the paramyxoviridae family, or may be from membrane-proximal regions (MRPs) of at least two (different), preferably three or four, enveloped viruses, e.g. enveloped viruses from different types of viruses such as two different viruses of the flaviviridae family. The inventors of the present invention have found that in this way improved multimeric inhibitors can be produced with broader specificity and/or improved viral fusion inhibitory function. Homologous regions can be identified bioinformatically by sequence alignments. The term “hybrid polypeptide”, as used herein, refers in preferred embodiments to a polypeptide comprising, essentially consisting of, or consisting of a hybrid peptide as defined above.

The degree of sequence similarity or identity over the entire length of a polypeptide comprised in the inhibitor of the present invention is obtained by using the best sequence alignment, wherein the best sequence alignment is obtainable with art known tools, e.g. Align, using standard settings, preferably EMBOSS::needle, Matrix: Blosum62, Gap Open 10.0, Gap Extend 0.5, with the amino acid sequences set forth in the present application. It is preferred that alignment will be over the entire length of the two proteins and, thus, that the alignment score will be determined on this basis. It is, however, possible that the polypeptide comprised in the inhibitor of the present invention may comprise C-terminal/N-terminal or internal deletions or additions, e.g. through N- or C-terminal fusions of the sequences. In this case only the best aligned region may be used for the assessment of the similarity and identity, respectively.

In the context of the present invention, the term “attached” means that the membrane integrating lipid is chemically associated with the at least two polypeptides. In a preferred embodiment of the multimeric inhibitor of the present invention, the membrane integrating lipid is linked, more preferably covalently linked with the at least two polypeptides, e.g. directly or optionally via a linker and/or linker amino acids. Said linker may connect the membrane integrating lipid, preferably cholesterol or a derivative thereof, with said at least two polypeptides.

In the context of the eighth aspect of the present invention, the term “attached” means that the membrane integrating lipid is chemically associated with the polypeptide. In a preferred embodiment of the monomeric inhibitor of the eighth aspect of the present invention, the membrane integrating lipid is linked, more preferably covalently linked with the polypeptide, e.g. directly or optionally via a linker and/or linker amino acids. Said linker may connect the membrane integrating lipid, preferably cholesterol or a derivative thereof, with said polypeptide.

The term “membrane integrating lipid”, as used herein, can be any lipid as specified, e.g. cholesterol, a sphingolipid, a glycolipid, a glycerophospholipid or a membrane integrating derivatives thereof, as long as it has the capability to insert into a cell membrane or an equivalent artificial lipid bilayer. Preferably, a “membrane integrating lipid” and membrane integrating derivatives thereof are capable of forming rafts as described in Xu, J. Biol. Chem. 276, (2001) 33540-33546 and Wang, Biochemistry 43, (2004) 1010-8. In a preferred embodiment of the inhibitor of the present invention, the membrane integrating lipid is a glycolipid selected from the group consisting of a ganglioside, a cerebroside, a globoside and a sulfatide. The ganglioside may be, for example, selected from the group consisting of GD1a, GD1b, GM1, GD3, GM2, GM3, GQ1a and GQ1b. If the membrane integrating lipid is a sphingolipid then in a preferred embodiment it is a sphingomyelin or ceramide. In a preferred embodiment, the membrane integrating lipid is a sphingolipid having a structure according to formula IX:

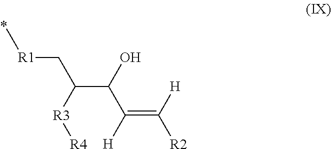

wherein

* denotes where the lipid is attached to said polypeptides (optionally via a linker) and wherein R1 through R4 are selected from the following list:

| R1 | R2 | R3 | R4 |
|---|---|---|---|
| COCH₂CH₂COO | (CH₂)₁₂CH₃ | NHCO | (CH₂)₁₄CH₃ |
| COCH₂O | (CH₂)₁₂CH₃ | NHCO | (CH₂)₁₄CH₃ |
| COCH₂CH₂COO | (CH₂)₁₂CH₃ | NHCO | (CH₂)₁₄CH₃ |
| COCH₂CH₂COO | (CH₂)₁₂CH₃ | NHCO | (CH₂)₁₈CH₃ |
| COCH₂CH₂COO | (CH₂)₁₂CH₃ | NHCO | (CH₂)₇CHCH(CH₂)₅CH₃ |
| COCH₂CH₂COO | (CH₂)₁₇CH3 | NHCO | (CH₂)₂₈CH₃ |
| COCH₂CH₂CONH | (CH₂)₁₂CH₃ | NHCO | (CH₂)₁₄CH₃ |
| COCH₂CH₂COO | (CH₂)₁₂CH₃ | NH | (CH₂)₁₅CH₃ |
| COCH₂CH₂COO | (CH₂)₁₂CH₃ | NHSO₂ | (CH₂)₁₄CH₃ |
| COCH₂CH₂COO | (CH₂)₁₂CH₃ | NHCONH | (CH₂)₁₇CH₃ |
| COCH₂CH₂COO | (CH₂)₁₇CH₃ | OCO | (CH₂)₂₈CH₃ |
| COCH₂CH₂COO | (CH₂)₁₂CH₃ | NHCONH | (CH₂)₁₅CH₃ |

If the membrane integrating lipid is a glycerophospholipid then in a preferred embodiment it is selected from the group of glycerophospholipids consisting of phosphatidylcholine, phosphatidylethanolamine and phosphatidylserine. Cholesterol is capable of inserting into the cell membrane. This property appears to be at least in part responsible for the significant inhibition of viral entry observed by the present inventors. Accordingly, the present invention also comprises membrane integrating derivatives of cholesterol. Such derivatives are structurally related to cholesterol in that they have the same steroid basic structure, i.e. (8R,9S,10R,13S,14S)-10,13-dimethyl-1,2,6,7,8,9,11,12,14,15,16,17-dodecahydrocyclopenta[a]phenanthren and have a comparable ability to insert into a lipid bilayer with the lipid composition as human cells as cholesterol. Preferred integrating derivatives of cholesterol include ergosterol, 7-dihydrocholosterol and stigmasterol. The ability to insert into a lipid bilayer can be tested by art known methods using, e.g. fluorescently labelled cholesterol and structural derivatives thereof on an artificial lipid bilayer. In a preferred embodiment, an integrating derivative of a membrane integrating lipid useful in the invention has the ability to integrate into a lipid raft comprised in a cell membrane. A membrane integrating lipid that can integrate into a lipid raft can generally also form one. Thus, if a lipid can integrate into and, thus, form a lipid raft can be tested, for example, as described in Xu, J. Biol. Chem. 276, (2001) 33540-33546, Wang, Biochemistry 43, (2004) 1010-8.

The term “enveloped virus” refers to a virus having an outer lipoprotein bilayer acquired by budding through a host membrane, e.g. host cell membrane. The structure underlying the envelope may be based on helical or icosahedral symmetry and may be formed before or as the virus leaves the cell. In the majority of cases, enveloped viruses use cellular membranes as sites allowing them to direct assembly. The formation of the particle inside the cell, maturation and release are in many cases a continuous process. The site of assembly varies for different viruses. Not all enveloped viruses bud from the cell (surface) membrane, many viruses use cytoplasmic membranes such as the golgi complex, others such as herpesviruses which replicate in the nucleus may utilize the nuclear membrane. In these cases, the virus is usually extruded into some form of vacuole, in which it is transported to the cell surface and subsequently released.

The term “(host) cell”, as used herein, refers to any cell which may be entered/infected by an enveloped virus, for example, a vertebrate cell such as an avian cell or a mammalian cell, e.g. an animal or a human cell.

The terms “cellular membrane” or “membrane of a cell”, as used herein, refer to any biological membrane which is part of a cell and which may be overcome/passed through by an enveloped virus during viral entry/cell infection (e.g. via membrane fusion or endocytosis). Both terms are interchangeable used herein. For example, said terms refer to a membrane which separates the interior of a cell from the outside environment (e.g. a cell/plasma membrane), or to a membrane which separates the interior of a component/organelle (e.g. endosome such as late endosome) of a cell from the cytosol (endosomal membrane such as late endosomal membrane). The cell membrane (also called the plasma membrane or plasmalemma), for example, means a biological membrane which separates the interior of a cell from the outside environment. The cell membrane surrounds all cells and it is selectively-permeable, controlling the movement of substances in and out of cells. It contains a wide variety of biological molecules, primarily proteins and lipids, e.g. cholesterol, which are involved in a variety of cellular processes such as cell adhesion, ion channel conductance and cell signalling. The plasma membrane also serves as the attachment point for the intracellular cytoskeleton.

The term “fusion of an enveloped virus with a cellular membrane”, as used herein, refers to a process which occurs if an enveloped virus traverses/passes through a membrane of a cell during virus entry/cell infection. The entry of enveloped viruses into target cells occurs via fusion of the viral membrane with a cellular membrane. Viral entry may be achieved by means of a membrane fusion reaction, occurring either directly at the cell surface following particle binding, or in low-pH endosomes after endocytosis of bound virions. In the case of viral entry through membrane fusion, for example, viral receptors attach to the receptors on the surface of the cell and secondary receptors may be present to initiate the puncture of the cell membrane or fusion with the host cell, followed by the unfolding of the viral envelope. In essence, the envelope of the virus blends with the cell membrane, releasing its contents into the cell. Examples include the human immunodeficiency virus (HIV), the Herpes simplex virus (HSV), or the Parainfluenza virus, e.g. HPIV 1, 2, 3, or 4. In the case of viral entry via endocytosis, for example, the virus enters the cell by receptor-mediated endocytosis, then moves from endocytic vesicles to early endosomes and finally to late endosomes where the virus fuses with the endosomal membrane to release viral genes. Examples include the Hepatitis C virus or the Influenza virus.

The term “pharmaceutically acceptable”, as used herein, means approved by a regulatory agency of the Federal or a state government or listed in the U.S. Pharmacopeia or other generally recognized pharmacopeia for use in animals, and more particularly in humans.

The term “pharmaceutically acceptable salt” refers to a salt of the inhibitor of viral fusion of the present invention. Suitable pharmaceutically acceptable salts of the inhibitor of viral fusion of the present invention include acid addition salts which may, for example, be formed by mixing a solution of the inhibitor of viral fusion of the present invention with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the inhibitor of viral fusion of the invention carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (see, for example, Berge, S. M., et al, “Pharmaceutical Salts”, Journal of Pharmaceutical Science, 1977, 66, 1-19). Certain inhibitors of viral fusion of the present invention contain both basic and acidic functionalities, e.g. Glu, Asp, Gln, Asn, Lys, or Arg that allow the compounds to be converted into either base or acid addition salts.

Said polypeptides comprised in the inhibitor of the present invention are capable of inhibiting fusion of at least one enveloped virus, preferably at least 2, 3, or 4 enveloped viruses, with a cellular membrane (e.g. cell/plasma membrane or endosomal membrane). The inhibition of fusion of an enveloped virus with the cellular membrane may occur, for example, by binding to (i) the (lipid) membrane of a cell, (ii) the (lipid) membrane of an enveloped virus, (iii) a protein associated with the (lipid) membrane of an enveloped virus, and/or (iv) a protein associated with the (lipid) membrane of a cell.

Preferably, the at least one enveloped virus, preferably at least 2, 3, or 4 enveloped viruses, is (are) (individually) selected from the group (of virus families) consisting of orthomyxoviridae, paramyxoviridae, filoviridae, retroviridae, coronaviridae, bornaviridae, togaviridae, arenaviridae, herpesviridae, hepadnaviridae, flaviviridae, rhabdoviridae. More preferably, the at least one enveloped virus, preferably at least 2, 3, or 4 enveloped viruses, is (are) (individually) selected from the group (of virus genera) consisting of orthomyxovirus, paramyxovirus, filovirus, retrovirus, coronavirus, bornavirus, togavirus, arenavirus, herpesvirus, hepadnavirus, flavivirus, rhabdovirus. Most preferably, the at least one enveloped virus, preferably at least 2, 3, or 4 enveloped viruses, is (are) (individually) selected from the group (of viruses) consisting of Influenza virus, Parainfluenza virus, Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Herpes simplex virus (HSV), Human herpesvirus (HHV) 6A, Human herpesvirus (HHV) 6B, Cytomegalovirus, Varicella-zoster virus, Chikunguya virus, Hepatitis C virus (HCV), Rabies virus, Dengue virus (DV), West Nile virus, Junin virus, Machupo virus, Guanarito virus, Japanese encephalitis virus, Yellow fever virus, and Lassa virus.

The membrane-integrating lipid attached to said polypeptides will in preferred embodiments allow the polypeptides to bind to a plasma membrane via lipid rafts and/or to be internalized into a cell preferably via lipid rafts. Many enveloped viruses enter cells via lipid rafts such as the influenza virus so that it is advantageous if said polypeptides exhibit their inhibitory ability not only on the cell surface but also intracellularly. Internalization can be studies by several approaches such as those described in Dyer & Benjamins, J. Neurosci. (1988) 883-891, D. C. Blakey1 et al., J. Cell Biochem. Biophys. 24-25 (1994) 175-183, Coffey et al., J. Pharmacol. Exp. Ther. 310 (2004) 896-904. The average skilled person is also well capable of testing, without undue burden, if said polypeptides bind to a (lipid) membrane of a cell (e.g. cell/plasma membrane or endosomal membrane) or an enveloped virus (e.g. membrane of a Influenza virus, Parainfluenza virus, Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Herpes simplex virus (HSV), Human herpesvirus (HHV) 6A, Human herpesvirus (HHV) 6B, Cytomegalovirus, Varicella-zoster virus, Chikunguya virus, Hepatitis C virus (HCV), Rabies virus, Dengue virus (DV), West Nile virus, Junin virus, Machupo virus, Guanarito virus, Japanese encephalitis virus, Yellow fever virus, or Lassa virus). For such analysis various tools such as fluorescence-based methods (e.g. colocalization studies, quenching etc.), electron microscopy studies and the like are readily available and suitable.

The person skilled in the art can easily determine whether said polypeptide(s) comprised in the inhibitor of the present invention are capable of inhibiting fusion of at least one enveloped virus with a cellular membrane (e.g. cell/plasma membrane or endosomal membrane), for example, via the binding mechanisms mentioned above, by (i) producing a recombinant enveloped virus capable of expressing a detectable marker protein, e.g. a green fluorescent protein (GFP), an enhanced green fluorescent protein (EGFP), or a blue fluorescent protein (BFP) within a cell, preferably a mammalian cell, e.g. a human cell, (ii) infecting a cell, preferably a mammalian cell, e.g. a human cell, with said recombinant enveloped virus, (iii) incubating said cell in the presence of (a) test polypeptide/polypeptides and in the absence of (a) test polypeptide/polypeptides (control), and (iv) assessing whether the marker protein, e.g. GFP, can be detected within said cell (e.g. within the cytosol or a component such as an endosome of said cell), for example, by fluorescence microscopy. Thus, if the polypeptide(s) is (are) capable of inhibiting fusion of at least one enveloped virus with a cellular membrane (e.g. cell/plasma membrane or endosomal membrane), no or only a few GFP molecules can be detected within said cell (e.g. within the cytosol or a component such as an endosome of said cell) contrary to the control experiment, wherein a cell is incubated with the enveloped virus alone.

Alternatively, the person skilled in the art can easily determine whether said polypeptide(s) comprised in the inhibitor of the present invention are capable of inhibiting fusion of at least one enveloped virus with a cellular membrane (e.g. cell/plasma membrane or endosomal membrane) by (i) labelling an enveloped virus with fluorescent lipophilic dyes, e.g. by incubating the enveloped virus with 1,1′-dioctadecyl-3,3,3′,3′-tetramethylindodicarbocyanine (DiD) (Molecular probes), (ii) infecting a cell, preferably a mammalian cell, e.g. a human cell, with the fluorescent lipophilic dye-labelled virus, e.g. DiD-labelled virus, (iii) incubating said cell in the presence of (a) test polypeptide/polypeptides and in the absence of (a) test polypeptide/polypeptides (control), (iv) exciting the fluorescent lipophilic dye-labelled virus, e.g. DiD-labelled virus with a laser, e.g. a 633 nm helium-neon laser (Melles-Griot), and (v) assessing whether the lipophilic dye-labelled virus, e.g. DiD-labelled virus, can be detected within said cell (e.g. within the cytosol or a component such as an endosome of said cell) incubated in the presence and absence of (a) test polypeptide/polypeptides, e.g. by obtaining fluorescence images from said cell. The fluorescent lipophilic dye DiD spontaneously partitions into the viral membrane. The dye-labelled viruses are still infectious and dye-labelling does not affect the viral infectivity. The surface density of the DiD-Dye is sufficiently high so that dye-labelled viruses can be clearly detected. See for example Lakadamyali et al., “Visualizing infection of individual influenza viruses”, 2003, PNAS, Vol. 100, No. 16, pages 9280-9285). Thus, if the polypeptide(s) is (are) capable of inhibiting fusion of at least one enveloped virus with a cellular membrane (e.g. cell/plasma membrane or endosomal membrane), no or only a weak fluorescence signal can be detected within said cell (e.g. within the cytosol or a component such as an endosome of said cell) contrary to the control experiment, wherein a cell is incubated with the fluorescent lipophilic dye labelled virus, e.g. DiD-labelled virus, alone. The skilled person knows about the different living cycle of the enveloped viruses referred to herein. For example, the skilled person knows that, for example, the fusion process of a paramyxovirus occurs at the surface of the cell/plasma membrane of a cell at neutral pH and that, thus, the success of the inhibition of viral fusion after polypeptide administration may be controlled, for example, by verifying the presence of said virus in the cytosol of said cell or that, for example, the fusion process of an influenza virus occurs at the surface of the endosomal membrane of a cell in the presence of an acidic pH and that, thus, the success of the inhibition of viral fusion after polypeptide administration may be controlled, for example, by verifying the presence of said virus in the endosome of said cell.

It is preferred that said at least two polypeptides, preferably at least 3, 4, 5, or 6 polypeptides, comprised in the multimeric inhibitor of the present invention are capable of inhibiting fusion of at least one enveloped virus, preferably at least 2, 3 or 4 enveloped viruses, (e.g. Influenza virus, Parainfluenza virus, Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Herpes simplex virus (HSV), Human herpesvirus (HHV) 6A, Human herpesvirus (HHV) 6B, Cytomegalovirus, Varicella-zoster virus, Chikunguya virus, Hepatitis C virus (HCV), Rabies virus, Dengue virus (DV), West Nile virus, Junin virus, Machupo virus, Guanarito virus, Japanese encephalitis virus, Yellow fever virus, or Lassa virus) by binding to a viral coat protein of at least one enveloped virus, preferably at least 2, 3, or 4 (different) enveloped viruses.

Preferably, the a monomeric inhibitor of viral fusion of the eighth aspect bind to
(i) a viral coat protein of at least one enveloped virus, or
(ii) a protein which is associated with the cellular membrane and which mediates the entry of an enveloped virus into a cell.

It is preferred that said polypeptide comprised in the monomeric inhibitor of the eighth aspect of the present invention is capable of inhibiting fusion of at least one enveloped virus, preferably the Human immunodeficiency virus (HIV).

The term “viral coat protein” of an enveloped virus, as used herein, refers to any protein which is present in the outer layer of an enveloped virus. The viral coat protein may be a protein which is directly or indirectly involved in the fusion process of an enveloped virus. A viral coat protein which is directly involved in viral fusion is, for example, a protein which immediately mediates viral entry, e.g. a fusogenic protein. Such a protein is, for example, a protein which undergoes conformational changes triggered by the initial binding of a virus to a target cell or to a target cell component/organelle and which fusion-active conformation finally leads to the fusion of the membrane of said cell (e.g. cell/plasma membrane) with the viral membrane. A viral coat protein which is indirectly involved in viral fusion is, for example, a protein which binds to another protein forming a protein complex (e.g. protein dimer, trimer, or multimer) or to a complex of proteins directly involved in the viral fusion process, e.g. a fusogenic protein, a complex of fusogenic proteins, or a protein, such as protein M2 of the influenza A virus, which creates the physico-chemical conditions required for the fusion protein to function, for example, by modifying the pH of the organelle (in the example the endosome) where fusion occurs.

Preferably, the viral coat protein is a viral fusogenic protein, more preferably a Type I, II, or III viral fusogenic protein.

The term “viral fusogenic protein” of an enveloped virus, as used herein, means a viral protein that mediates penetration into a (host) cell. As mentioned above, the fusogenic proteins also called penetrenes of enveloped viruses can be divided on the basis of common structural motifs into at least three classes, namely in Type I, II, or III viral fusogenic proteins. Viruses of the family Orthomyxoviridae, Paramyxoviridae, Filoviridae, Retroviridae, Coronaviridae, Arenaviridae, and Rhabdoviridae, for example, encode class I penetrenes which are also known as Type I viral fusion proteins or α-penetrenes. Type I viral fusogenic proteins are well known in the art and comprise one or more of the following: a “fusion peptide” which is a cluster of hydrophobic and aromatic amino acids located at or near the amino terminus, an amino terminal helix (N-helix, HR1), a carboxyl terminal helix (C-helix, HR2), usually an aromatic amino acid (aa) rich pre-membrane domain and a carboxyl terminal anchor. Viruses of the family Togaviridae and Flaviviridae, for example, encode class II penetrenes which are also known as Type II viral fusion proteins or β-penetrenes. Type II viral fusogenic proteins are also well known in the art and comprise one or more of the following: domain I, domain II, domain III (all three domains comprised mostly of anti-parallel β sheets), a membrane proximal α-helical stem domain (also known as membrane-proximal region (MRP)) and a carboxyl terminal anchor. The fusion loops of Type II viral fusogenic proteins are internal and located in domain II. Viruses of the family Herpesviridae, for example, encode class III penetrenes which are also known as Type III viral fusion proteins or γ-penetrenes. Type III viral fusogenic proteins are also well known in the art and comprise one or more of the following: an internal fusion domain comprised of beta sheets, other beta sheet domains, an extended alpha helical domain, a membrane proximal stem domain and a carboxyl terminal anchor (see for Type I, II, and III viral fusogenic proteins Table 1 below and, for example, Garry et al., “Proteomics computational analyses suggest that baculovirus GP64 superfamily proteins are class III penetrenes”, Virology Journal 2008, 5:28 and see also: Harrison S., “Viral membrane fusion”, Nat Struct Mol Biol. 2008 July; 15(7): 690-698).

Table 1 summarizes the fusogenic proteins present in enveloped viruses referred to herein. In addition, Table 1 gives information about the sites of viral fusion.

TABLE 1

| | | | Type of | |
|---|---|---|---|---|
| | | | Fusogenic | |
| Virus | Family | Genus | protein | Fusion occurs |
| Influenza A, B | Orthomyxoviridae | Infuenzavirus A, B | Type I | Endosome |
| Parainfluenza 1, 3 | Paramyxoviridae | Paramyxovirus | Type I | Plasma membrane |
| Parainfluenza 2, 4 | Paramyxoviridae | Rubulavirus | Type I | Plasma membrane |
| Sendai | Paramyxoviridae | Paramyxovirus | Type I | Plasma membrane |
| Measles | Paramyxoviridae | Morbillivirus | Type I | Plasma membrane |
| Newcastle | Paramyxoviridae | Rubulavirus | Type I | Plasma membrane |
| Disease Virus | | | | |
| Mumps | Paramyxoviridae | Rubulavirus | Type I | Plasma membrane |
| Respiratory | Paramyxoviridae | Pneumovirus | Type I | Plasma membrane |
| syncytial virus | | | | |
| Human | Paramyxoviridae | Pneumovirus | Type I | Plasma membrane |
| Metapneumovirus | | | | |
| Hendra | Paramyxoviridae | Henipavirus | Type I | Plasma membrane |
| Nipah | Paramyxoviridae | Henipavirus | Type I | Plasma membrane |
| Ebola | Filoviridae | Filovirus | Type I | Endosome |
| Marburg | Filoviridae | Filovirus | Type I | Endosome |
| HIV | Retroviridae | Lentivirus | Type I | Plasma membrane |
| SARS | Coronaviridae | Coronavirus | Type I | Plasma membrane |
| Junin | Arenaviridae | Arenavirus | Type I | Endosome |
| Machupo | Arenaviridae | Arenavirus | Type I | Endosome |
| Guanarito | Arenaviridae | Arenavirus | Type I | Endosome |
| Lassa | Arenaviridae | Arenavirus | Type I | Endosome |
| Rabies | Rhabdoviridae | Lyssavirus | Type I | Endosome |
| Chikunguya | Togaviridae | Alphavirus | Type II | Endosome |
| Hepatitis C | Flaviviridae | Flavivirus | Type II | Endosome |
| Dengue | Flaviviridae | Flavivirus | Type II | Endosome |
| West Nile | Flaviviridae | Flavivirus | Type II | Endosome |
| Yellow fever | Flaviviridae | Flavivirus | Type II | Endosome |
| Japanese | Flaviviridae | Flavivirus | Type II | Endosome |
| encephalitis | | | | |
| Herpes simplex | Herpesviridae | Simplexvirus | Type III | Plasma membrane |
| (HSV) | | | | |
| Human | Herpesviridae | Roseolovirus | Type III | Plasma membrane |
| Herpesvirus | | | | |
| (HHV) 6A, 6B | | | | |
| Cytomegalovirus | Herpesviridae | Cytomegalovirus | Type III | Plasma membrane |
| Varicella zoster | Herpesviridae | Varicellovirus | Type III | Plasma membrane |

Most preferably, the fusogenic protein is a protein or peptide selected from the group consisting of HIV gp41 (Type I fusogenic protein, e.g. accession number AAA19156.1), HIV gp120, influenza hemagglutinin (Type I fusogenic protein, e.g. accession number AAA43099.1 or CAA40728.1), protein F of paramyxoviruses (Type I fusogenic protein, e.g. accession number AAV54052.1), protein GP2 of filoviruses (Type I fusogenic protein, e.g. accession number Q89853.1 or AAV48577.1), protein E of flaviviruses (Type II of fusogenic protein, e.g. accession number AAR87742.1), protein E1 of alphaviruses (Type II of fusogenic protein), protein S of coronaviruses (Type I of fusogenic protein, e.g. accession number AAP33697.1 or BAC81404.1), protein gH of herpesviruses (Type III fusogenic protein), protein gB of herpesviruses (Type III fusogenic proteins), and protein G2 of arenaviruses (Type I fusogenic protein, e.g. accession number BAA00964.2 or P03540). HIV gp41 and HIV gp120 are part of the same protein gp160, while gp120 is the receptor-binding subunit, gp41 the fusogenic subunit. Gp41 is a Type I fusogenic protein.

It is also preferred that said at least two polypeptides, preferably at least 3, 4, 5, or 6 polypeptides, comprised in the multimeric inhibitor of the present invention, or the monomeric inhibitor of the eighth aspect of the invention are/is capable of inhibiting fusion of at least one enveloped virus by binding to a protein which is associated with the cellular membrane, preferably cell/plasma membrane or endosomal membrane, and which mediates the entry of an enveloped virus into a cell. More preferably, said protein is associated with the cellular membrane (e.g. cell/plasma membrane or endosomal membrane) and mediates the entry of an enveloped virus selected from the group consisting of Influenza virus, Parainfluenza virus, Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Herpes simplex virus (HSV), Human herpesvirus (HHV) 6A, Human herpesvirus (HHV) 6B, Cytomegalovirus, Varicella-zoster virus, Chikunguya virus, Hepatitis C virus (HCV), Rabies virus, Dengue virus (DV), West Nile virus, Junin virus, Machupo virus, Guanarito virus, Japanese encephalitis virus, Yellow fever virus, and Lassa virus into a cell. Most preferably, the protein which is associated with the cellular membrane (e.g. cell/plasma membrane or endosomal membrane) and which mediates the entry of an enveloped virus into a cell is selected from the group consisting of CD4, CCR5, CXCR4, integrins like integrin alpha-4 beta-7, glycoproteins containing sialic acid as terminal group, human angiotensin-converting enzyme 2 (ACE2), herpesvirus entry mediator (HVEM), nectin-1, proteins containing 3-O sulfated heparan sulfate, the C-type lectins DC-SIGN and DC-SIGNR, the L-Type lectin L-SIGN, nicotinic acetylcholine receptor (nAChR), neuronal cell adhesion molecule (NCAM), p75 neurotrophin receptor (p75NTR), insulin-degrading enzyme (IDE), Ephrin B2, Ephrin B3, CD81, and scavanger receptor B1 (SR-B1).

It is within the skill of the artisan to experimentally determine if said at least two polypeptides, preferably at least 3, 4, 5, or 6 polypeptides, comprised in the multimeric inhibitor of the present invention, or the polypeptide of the monomeric inhibitor of the eighth aspect of the invention, bind(s) to the aforementioned polypeptides or proteins (e.g. viral fusogenic proteins or proteins associated with the cellular membrane mediating the entry of an enveloped virus into a cell), for example, by using a pull down assay. Also other binding assays well known in the art and suitable to determine binding affinities between binding partners can be used such as e.g. ELISA-based assays, fluorescence resonance energy transfer (FRET)-based assays, co-immunoprecipitation assays and plasmon-resonance assays. The binding can be detected by fluorescence means, e.g. using a fluorescently labelled secondary antibody, or enzymatically as is well known in the art. Also radioactive assays may be used to assess binding. In order to further determine whether said binding results in the inhibition of fusion of at least one enveloped virus with the membrane of a cell (e.g. cell/plasma membrane or endosomal membrane), the above mentioned assays, e.g. tracking of single lipophilic dye-labelled viruses in living cells by using fluorescence microscopy in the absence and presence of a polypeptide/polypeptides comprised in the inhibitor of the present invention, may be used.

The at least two polypeptides as set out above, preferably the at least 3, 4, 5, or 6 polypeptides, comprised in the multimeric inhibitor of the present invention may be may be identical or different. Preferably, the multimeric inhibitor of the present invention comprises at least 2, 3, 4, 5, or 6 identical polypeptides.

The inventors of the present invention have identified novel multimeric inhibitors which are effective in the inhibition of viral fusion and which are based on fusogenic proteins, particularly Type I, II and III fusogenic proteins, of enveloped viruses. These inhibitors are, for example, based on peptides that bind to domains of Type I, II, or III fusogenic proteins which are known to facilitate fusion with the cellular membrane such as plasma/cell membrane, e.g. by interacting with the respective cellular receptors.

Accordingly, in a preferred embodiment of the inhibitor of the present invention, said at least two polypeptides, preferably at least 3, 4, 5, or 6 polypeptides, each comprise, essentially consist of, or consist of a (hybrid) peptide, wherein at least one of said peptides, preferably at least 2, 3, 4, 5, or 6 of said peptides, (e.g. each peptide comprised in said polypeptides) is (are) capable of inhibiting fusion of at least one enveloped virus by binding to a domain of a Type I, II, or III fusogenic protein which facilitates fusion with the cellular membrane, preferably plasma/cell membrane or endosomal membrane, and/or wherein at least one of said peptides is selected from the group consisting of WX₁EWX₂REINX₃YX₄SLIX₅SLIEEX₆QX₇QQX₈KNEX₉X₁₀LX₁₁X₁₂L (SEQ ID NO: 188), a polypeptide comprising an amino acid sequence having at least 75% identity to WNEWEREINKYTSLIYSLIEEAQNQQDKNEKDLLEL (SEQ ID NO: 192) or SWETWEREIENYTRQIYRILEESQEQQDRNERDLLE (SEQ ID NO: 189), wherein X₁ to X₁₂ are defined as described above.

In a further preferred embodiment of the multimeric inhibitor of the present invention, said at least two polypeptides, preferably at least 3, 4, 5, or 6 polypeptides, each comprise, essentially consist of, or consist of a (hybrid) peptide, wherein at least one of said peptides, preferably at least 2, 3, 4, 5, or 6 of said peptides, (e.g. each peptide comprised in said polypeptides) is (are) capable of inhibiting fusion of at least one enveloped virus, preferably at least 2, 3, or 4 (different) enveloped viruses, by binding to
(i) a heptad repeat (HR) domain of a Type I or III viral fusogenic protein of at least one enveloped virus, preferably at least 2, 3, or 4 (different) enveloped viruses, and/or
(ii) a beta-sheet domain of a Type II viral fusogenic protein of at least one enveloped virus, preferably a beta-sheet domain comprised in domain II of a Type II viral fusogenic protein of at least one enveloped virus, preferably at least 2, 3, or 4 (different) enveloped viruses;
   and/or wherein at least one of said peptides is selected from the group consisting of WX₁EWX₂REINX₃YX₄SLIX₅SLIEEX₆QX₇QQX₈KNEX₉X₁₀LX₁₁X₁₂L (SEQ ID NO: 188), a polypeptide comprising an amino acid sequence having at least 75% identity to WNEWEREINKYTSLIYSLIEEAQNQQDKNEKDLLEL (SEQ ID NO: 192) or SWETWEREIENYTRQIYRILEESQEQQDRNERDLLE (SEQ ID NO: 189), wherein X₁ to X₁₂ are defined as described above.

In a more preferred embodiment of the multimeric inhibitor of the present invention, said at least two polypeptides, preferably at least 3, 4, 5, or 6 polypeptides, each comprise, essentially consist of, or consist of a (hybrid) peptide, wherein at least one of said peptides, preferably at least 2, 3, 4, 5, or 6 of said peptides, (e.g. each peptide comprised in said polypeptides) is (are) capable of inhibiting fusion of at least one enveloped virus, preferably at least 2, 3, or 4 (different) enveloped viruses, by binding to
(i) a heptad repeat 1 (HR1) domain or heptad repeat 2 (HR2) domain of a Type I viral fusogenic protein of at least one enveloped virus, preferably at least 2, 3, or 4 (different) enveloped viruses, and/or
(ii) a beta-sheet domain comprised in domain II of a Type II viral fusogenic protein of at least one enveloped virus, preferably at least 2, 3, or 4 (different) enveloped viruses; and/or wherein at least one of said peptides is selected from the group consisting of WX₁EWX₂REINX₃YX₄SLIX₅SLIEEX₆QX₇QQX₈KNEX₉X₁₀LX₁₁X₁₂L (SEQ ID NO: 188), a polypeptide comprising an amino acid sequence having at least 75% identity to WNEWEREINKYTSLIYSLIEEAQNQQDKNEKDLLEL (SEQ ID NO: 192) or SWETWEREIENYTRQIYRILEESQEQQDRNERDLLE (SEQ ID NO: 189), wherein X₁ to X₁₂ are defined as described above.

The term “heptad repeat (HR)”, as used herein, refers to a sequence of amino acids in alpha-helical structure with periodicity of 7, ‘abcdef’ where a hydrophobic amino acid is present at the ‘a’ and positions. Depending on the nature of the amino acids in position ‘a’ and ‘d’, heptad repeats form assemblies of 2, 3, four or six chains, called coiled-coils (dimeric coiled coil, trimeric coiled coil, tetrameric coiled coil, hexameric coiled coil, also known as six-helix bundle). The term “HR1”, as used herein, refers to the amino terminal heptad repeat, i.e. repeat proximal to the N-terminus (N-helix, HRN), while the term “HR2”, as used herein, refers to the carboxyl terminal repeat, i.e. repeat proximal to the C-terminus (C-helix, HRC). The HR1 domains of type I fusogenic proteins, for example, typically form a homo-trimeric coiled coil (three HR1). In gp41 of HIV, the HR2 domain binds to the HR1 domain forming a hexameric coiled coil (3 HR1-3HR2). A HR can be identified in the primary sequence of a protein by computer programs like LearnCoil [Singh, M., B. Berger, et al. (1999). LearnCoil-VMF: computational evidence for coiled-coil-like motifs in many viral membrane-fusion proteins. Journal of Molecular Biology 290(5): 1031-1041].

The term “beta-sheet domain”, as used herein, refers to a sequence of amino acids comprised in domains I, II, and III of Type II fusogenic proteins. Generally, beta-sheets consist of beta strands connected laterally by at least two or three backbone hydrogen bonds, forming a generally twisted, pleated sheet. Domains I, II, and III are composed almost entirely of beta-sheets (β-sheets). For example, in a full-length molecule, said three domains are organized/oriented as follows: Domain I is a β-barrel that contains the N-terminus and two long insertions that connect adjacent β-strands and together form the elongated domain II. The first of these insertions contains the highly conserved fusion peptide loop at its tip, connecting the c and d β-strands of domain II (termed the cd-loop) and containing 4 conserved disulfide bonds including several that are located at the base of the fusion loop. The second insertion contains the ij loop at its tip, adjacent to the fusion loop, and one conserved disulfide bond at its base. A hinge region is located between domains I and II. On the other side of domain I, a short linker region connects domain I to domain III, a β-barrel with an immunoglobulin-like fold stabilized by three conserved disulfide bonds (see for example Kielian M. “Class II virus membrane fusion proteins”, Virology Journal 2006, 344, 38-47). A beta-sheet domain can also be identified in the primary sequence of a protein by computer programs known to the person skilled in the art.

Preferably,
(i) the HR1 domain of a Type I viral fusogenic protein is selected from the group consisting of HR1 domains with an amino acid sequence according to SEQ ID NO: 1 to SEQ ID NO: 17, SEQ ID NO: 105, and SEQ ID NO: 144 to SEQ ID NO: 150, or
(ii) the HR2 domain of a Type I viral fusogenic protein with an amino acid sequence according to SEQ ID NO: 151.

Thus, in a preferred embodiment, the multimeric inhibitor comprises
(i) at least two polypeptides, preferably at least 3, 4, 5, or 6 polypeptides, each comprise, essentially consist of, or consist of a (hybrid) peptide, wherein at least one of said peptides, preferably at least 2, 3, 4, 5, or 6 of said peptides, (e.g. each peptide comprised in said polypeptides) is (are) capable of inhibiting fusion of at least one enveloped virus by binding to
   (ia) a HR1 domain of a Type I viral fusogenic protein of at least one enveloped virus selected from the group consisting of HR1 domains with an amino acid sequence according to SEQ ID NO: 1 to SEQ ID NO: 17, SEQ ID NO: 105, and SEQ ID NO: 144 to SEQ ID NO: 150, and/or
   (ib) a HR2 domain of a Type I viral fusogenic protein of one (an) enveloped virus with an amino acid sequence according to SEQ ID NO: 151, and
(ii) a membrane integrating lipid selected from the group consisting of cholesterol, a sphingolipid, a glycolipid, a glycerophospholipid and membrane integrating derivatives thereof, which is attached to the C-terminal region of each polypeptide;

or a pharmaceutically acceptable salt thereof.

The peptides as set out above comprised in the at least two polypeptides mentioned above, preferably at least 2, 3, 4, 5, or 6 polypeptides, comprised in the multimeric inhibitor of the present invention may be may be identical or different. Preferably, the peptides as set out above comprised in the at least two polypeptides mentioned above, preferably at least 2, 3, 4, 5, or 6 polypeptides, comprised in the multimeric inhibitor of the present invention are identical. Thus, for example, the multimeric inhibitor of the present invention may comprise (i) 2 polypeptides each comprising an identical peptide binding to a domain selected from the group consisting of a HR domain, such as HR1 or HR2 domain, and a beta-sheet domain, (ii) 3 polypeptides each comprising an identical peptide binding to a domain selected from the group consisting of a HR domain, such as HR1 or HR2 domain, and a beta-sheet domain, (iii) 4 polypeptides each comprising an identical peptide binding to a domain selected from the group consisting of a HR domain, such as HR1 or HR2 domain, and a beta-sheet domain, (iv) 5 polypeptides each comprising an identical peptide binding to a domain selected from the group consisting of a HR domain, such as HR1 or HR2 domain, and a beta-sheet domain, or (v) 6 polypeptides each comprising an identical peptide binding to a domain selected from the group consisting of a HR domain, such as HR1 or HR2 domain, and a beta-sheet domain.

In another preferred embodiment of the multimeric inhibitor of the present invention, at least one of said (hybrid) peptides, preferably at least 2, 3, 4, 5, or 6 of said peptides, that are comprised in said at least two polypeptides, preferably at least 3, 4, 5, or 6 polypeptides, (e.g. each peptide comprised in said polypeptides)
(i) has (have) a length of at least ten contiguous amino acids and is (are) from a HR domain of a Type I or III viral fusogenic protein of at least one enveloped virus, preferably at least 2, 3, or 4 (different) enveloped viruses, or
(ii) has (have) a length of at least ten contiguous amino acids and is (are) from a membrane-proximal region (MPR) of a Type II, viral fusogenic protein of at least one enveloped virus, preferably at least 2, 3, or 4 (different) enveloped viruses.

The term “membrane-proximal region (MPR)”, as used herein, refers to a region comprised in viral fusogenic proteins, preferably Type II viral fusogenic proteins, which is adjacent, i.e. N-terminal to or C-terminal to, preferably N-terminal to, the transmembrane region of the viral fusogenic proteins, preferably Type II viral fusogenic proteins. The length of the membrane proximal region is preferably less than 150 consecutive amino acids, more preferably less than 100 consecutive amino acids and most preferably less than 75 consecutive amino acids.

In more preferred embodiment of the multimeric inhibitor of the present invention, at least one of said (hybrid) peptides, preferably at least 2, 3, 4, 5, or 6 of said peptides, that are comprised in said at least two polypeptides, preferably at least 3, 4, 5, or 6 polypeptides (e.g. each peptide comprised in said polypeptides)
(i) has (have) a length of at least ten contiguous amino acids and is (are) from a HR1 domain or HR2 domain of a Type I viral fusogenic protein of at least one enveloped virus, preferably at least 2, 3, or 4 (different) enveloped viruses.

Said at least 2, 3, or 4 enveloped viruses may be different enveloped viruses, e.g. different types of enveloped viruses.

Thus, it is preferred that at least one of said (hybrid) peptides, preferably at least 2, 3, 4, 5, or 6 of said peptides, having a length of at least ten contiguous amino acids that are comprised in said at least two polypeptides, preferably at least 3, 4, 5, or 6 polypeptides, (e.g. each peptide comprised in said polypeptides)
(i) is (are) from a HR2 domain of a Type I viral fusogenic protein of at least one enveloped virus and bind(s) to the HR1 domain of a Type I viral fusogenic protein of at least one enveloped virus, which is preferably selected from the group consisting of HR1 domains with an amino acid sequence according to SEQ ID NO: 1 to SEQ ID NO: 17, SEQ ID NO: 105, and SEQ ID NO: 144 to SEQ ID NO: 150,
(ii) is (are) from a HR1 domain of a Type I viral fusogenic protein of one (an) enveloped virus and bind(s) to the HR2 domain of a Type I viral fusogenic protein of one (an) enveloped virus which has preferably an amino acid sequence according to SEQ ID NO: 151,
(iii) is (are) from a membrane-proximal region (MRP) of a Type II viral fusogenic protein of at least one enveloped virus and bind(s) to the beta-sheet domain, preferably the beta-sheet domain comprised in domain II, of a Type II viral fusogenic protein of at least one enveloped virus, or
(iv) is (are) from a HR domain of a Type III viral fusogenic protein of one (an) enveloped virus and bind(s) to a HR domain which is also comprised in said Type III viral fusogenic protein.

Preferably, the above mentioned HR domain, preferably HR1 domain or HR2 domain, and/or membrane-proximal region (MPR) may be a naturally occurring or synthetic (synthesized) HR domain, preferably HR1 domain or HR2 domain, and/or membrane-proximal region (MPR).

A HR domain, particularly a HR1 or HR2 domain, or a beta-sheet domain binding peptide or peptides that is (are) part of the polypeptides comprised in the multimeric inhibitors of viral fusion of the present invention can be identified with art known high throughput assay system, preferably phage display, wherein bacteria are transformed by phage each expressing a different peptide in fusion to a phage capsid protein. These fusion proteins will “display” the respective peptide on the surface of the bacterial cell, which then can be tested for interaction with the HR domain, particularly HR1 or HR2 domain, or the beta-sheet domain of interest, e.g. with one of the HR1 domains having a sequence according to SEQ ID NO: 1 to SEQ ID NO: 17, SEQ ID NO: 105, SEQ ID NO: 144 to SEQ ID NO: 150, or with the HR2 domain having a sequence according to SEQ ID NO: 151.

Alternatively, a HR domain, particularly a HR1 or HR2 domain, or a beta-sheet domain binding peptide or peptides may be designed using a rational peptide design approach. In such an approach a preferred starting point is the HR2 domain of a Type I viral fusogenic protein of an enveloped virus from which the respective HR1 domain originates. This HR2 domain is mutated at one or multiple positions (e.g. 1, 2, 3, 4, 5, 6, 7, 8, 9, or 10 positions by (a) amino acid substitution(s), deletion(s), and/or insertion(s)/addition(s)) and then assayed for binding activity to the HR1 domain. Another preferred starting point is the HR1 domain of a Type I viral fusogenic protein of an enveloped virus from which the respective HR2 domain originates. This HR1 domain is also mutated at one or multiple positions (e.g. 1, 2, 3, 4, 5, 6, 7, 8, 9, or 10 positions by (a) amino acid substitution(s), deletion(s), and/or insertion(s)/addition(s)) and then assayed for binding to the HR2 domain. A further preferred starting point is the membrane-proximal region of a Type II viral fusogenic protein of an enveloped virus from which the beta-sheet domain originates. This membrane-proximal region is also mutated at one or multiple positions (e.g. 1, 2, 3, 4, 5, 6, 7, 8, 9, or 10 positions by (a) amino acid substitution(s), deletion(s), and/or insertion(s)/addition(s)) and then assayed for binding to the beta-sheet domain. The modifications may then allow the production of peptides capable of inhibiting the fusion of more than one enveloped virus, preferably at least 2, 3, or 4 (different) enveloped viruses.

Suitable assays include any art known protein-protein interaction assay including without limitation in vitro interaction assays, preferably GST or antibody-pull-down assays wherein one of the two binding partners is expressed as a GST fusion protein or as a fusion with an antibody tag and the other binding partner is labelled, and in vivo interaction assays, preferable two-hybrid assays, as well as functional assays, wherein the respective derivative is tested for its ability to inhibit replication or entry of the respective virus. Such assays have been described in the art and have also been employed in the present invention to assess the suitability of a particular HR2 domain derivative to be used in the inhibition of viral fusion.

The above mentioned peptides that are comprised in the polypeptides which are part of the multimeric inhibitor of the present invention may be identical or may be different. In preferred embodiments, the above mentioned peptides that are comprised in the polypeptides which are part of the multimeric inhibitor of the present invention are identical (are the same).

The above mentioned multimeric inhibitors are very effective in the inhibition of viral fusion. Their improved effectiveness is based on their decreased “off-rate” and increased “on-rate”. The decreased “off-rate” of the multimeric inhibitor (e.g. HR2 as HR1-binding peptides or HR1 as HR2-binding peptides)/fusogenic protein or fusogenic domain/region (e.g. HR1 or HR2) complex (e.g. HR2-HR1 complex or HR1-HR2 complex) is achieved by exploiting the avidity of the interacting protein domains (e.g. HR2 as HR1-binding peptides or HR1 as HR2-binding peptides). “Avidity”, or “functional affinity”, as used herein, is a term commonly applied to describe the strength of the interaction of multivalent molecules, typically the interaction of antigens with multiple epitopes with antibodies with more than one paratope. Individually, each binding interaction may be readily broken, however, when more than one binding interaction is present at the same time, transient unbinding of a single site does not allow the molecule to diffuse away, and binding of that site is likely to be reinstated. The overall effect is synergistic, since avidity is a multiple of the affinities of the individual interactants, not a simple sum of their affinities. Therefore by incorporating more than one copy of a peptide (e.g. HR1-binding peptide or HR2-binding peptide) into a single inhibitory molecule, the inventors of the present invention have increased the avidity of the multimeric fusion inhibitor and hence have increased its antiviral potency. The increased “on-rate” of the multimeric inhibitor is additionally achieved by introducing in the molecule a suitably positioned cholesterol group, which concentrates it in lipid rafts.

It is a surprising finding of the inventors that the inhibitor of the present invention which comprises a membrane integrating lipid has the surprising property of not only significantly enhancing the inhibitory activity of the polypeptide(s) but also to extend the inhibitory activity of the polypeptide inhibitors to enveloped viruses that are not inhibited when the membrane integrating lipid is absent.

Thus, it is preferred that the multimeric inhibitor of the invention inhibits the fusion of at least 2, 3, or 4 (different) enveloped viruses selected from the group consisting of Influenza virus, Parainfluenza virus, Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Herpes simplex virus (HSV), Human herpesvirus (HHV) 6A, Human herpesvirus (HHV) 6B, Cytomegalovirus, Varicella-zoster virus, Chikunguya virus, Hepatitis C virus (HCV), Rabies virus, Dengue virus (DV), West Nile virus, Junin virus, Machupo virus, Guanarito virus, Japanese encephalitis virus, Yellow fever virus, and Lassa virus. It is more preferred that the multimeric inhibitor of the invention inhibits the fusion of at least 2, 3 or 4 (different) enveloped viruses selected from the group of Influenza virus, Parainfluenza virus, Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Rabies virus, Junin virus, Machupo virus, Guanarito virus, and Lassa virus. It is also more preferred that the multimeric inhibitor of the invention inhibits the fusion of at least 2, 3, or 4 (different) enveloped viruses selected from the group of Chikunguya virus, Hepatitis C virus (HCV), Dengue virus (DV), West Nile virus, Japanese encephalitis virus, and Yellow fever virus. It is further more preferred that the multimeric inhibitor of the invention inhibits the fusion of at least 2, 3, or 4 (different) enveloped viruses selected from the group of Herpes simplex virus (HSV), Human herpesvirus (HHV) 6A, Human herpesvirus (HHV) 6B, Cytomegalovirus, and Varicella-zoster virus. In a most preferred embodiment, the multimeric inhibitor of the invention is capable of interfering with the viral fusion with the (host) cell of at least the viruses human parainfluenza virus 3 (HPIV3), Nipah virus (NiV), Respiratory syncytical virus (RSV) and/or Simian parainfluenza virus 5 (SV5).

FIGS. 2-5 show preferred peptide sequences that may be comprised in a broad-spectrum antiviral agent of the invention. Accordingly, in one embodiment of the multimeric inhibitor of the invention, at least one of said peptides, preferably at least 2, 3, 4, 5, or 6 peptides, that are comprised in said at least two polypeptides, preferably at least 3, 4, 5, or 6 polypeptides, (e.g. each peptide comprised in said polypeptides) has (have) at least ten contiguous amino acids of SEQ ID NO: 99 or of a derivative thereof, wherein the derivative consists of the following amino acids:

amino acid 1 is selected from Val, Leu, and Tyr;

amino acid 2 is selected from Ala, Ser, Asp, Tyr, and Phe;

amino acid 3 is selected from Leu, Ile, Pro, and Thr;

amino acid 4 is selected from Asp, Leu, and Phe;

amino acid 5 is selected from Pro, Val, and Lys;

amino acid 6 is selected from Ile, Leu, Phe, Val, and Ala;

amino acid 7 is selected from Asp and Glu;

amino acid 8 is selected from Ile and Phe;

amino acid 9 is selected from Ser and Asp;

amino acid 10 is selected from Ile, Gln, Ala, and Ser;

amino acid 11 is selected from Glu, Asn, Ser, Gln, and Val;

amino acid 12 is selected from Leu, Ile, and Asn;

amino acid 13 is selected from Asn, Ala, and Ser;

amino acid 14 is selected from Lys, Ala, Gln, and Ser;

amino acid 15 is selected from Ala, Val, Met, and Ile;

amino acid 16 is selected from Lys and Asn;

amino acid 17 is selected from Ser, Lys, Glu, and Gln;

amino acid 18 is selected from Asp, Ser, and Lys;

amino acid 19 is selected from Leu and Ile;

amino acid 20 is selected from Glu, Ser, Asn, and Gln;

amino acid 21 is selected from Glu, Asp, and Gln;

amino acid 22 is selected from Ser, Ala, and Ile;

amino acid 23 is selected from Lys and Leu;

amino acid 24 is selected from Glu, Gln, Ala, and Asp;

amino acid 25 is selected from Trp, His, Phe, and Tyr;

amino acid 26 is selected from Ile and Leu;

amino acid 27 is selected from Arg, Ala, and Lys;

amino acid 28 is selected from Arg, Gln, Lys, and Glu;

amino acid 29 is selected from Ser, Ala, and Ile;

amino acid 30 is selected from Asn, Asp, and Gln;

amino acid 31 is selected from Gly, Thr, Glu, Lys, Arg, and Gln;

amino acid 32 is selected from Lys, Tyr, Leu, and Ile;

amino acid 33 is Leu;

amino acid 34 is selected from Asp, Ser, and His;

amino acid 35 is selected from Ser, Ala, Asn, and Thr;

amino acid 36 is selected from Ile and Val; and

wherein the derivative may optionally comprise the three additional amino acids Pro, Ser, and Asp between amino acid 6 and amino acid 7.

Also preferred is a multimeric inhibitor of the invention wherein the derivative of SEQ ID NO: 99 mentioned above consists of the following amino acids: Amino acid 1 can be Val or Tyr; Amino acid 2 can be Ala, Ser, Asp, Tyr, and Phe; Amino acid 3 is Leu; Amino acid 4 can be Asp or Leu; Amino acid 5 can be Pro, Val, or Lys; Amino acid 6 can be Ile or Phe; Amino acid 7 is Asp; Amino acid 8 can be Ile or Phe; Amino acid 9 can be Ser or Asp; Amino acid 10 can be Ile, Ala, or Ser; Amino acid 11 can be Glu or Gln; Amino acid 12 is Leu; Amino acid 13 can be Asn or Ser; Amino acid 14 can be Lys, Gln, or Ser; Amino acid 15 can be Ala or Val; Amino acid 16 can be Lys or Asn; Amino acid 17 can be Ser, Lys, Glu, or Gln; Amino acid 18 can be Asp, Ser, or Lys; Amino acid 19 is Leu; Amino acid 20 can be Glu or Asn; Amino acid 21 can be Glu or Gln; Amino acid 22 can be Ser or Ala; Amino acid 23 can be Lys or Leu; Amino acid 24 can be Glu, Gln, or Ala; Amino acid 25 can be Trp, or His; Amino acid 26 is Ile; Amino acid 27 is Arg or Ala; Amino acid 28 can be Arg, Gln, or Glu; Amino acid 29 can be Ser or Ala; Amino acid 30 can be Asn or Asp; Amino acid 31 can be Gly, Thr, Glu, or Lys; Amino acid 32 can be Lys, Tyr, or Ile; Amino acid 33 is Leu; Amino acid 34 can be Asp, Ser, or His; Amino acid 35 can be Ser, Ala, or Asn and wherein Amino acid 36 is Ile.

Also preferred is an inhibitor of the invention wherein the derivative of SEQ ID NO: 99 mentioned above consists of the following amino acids: Amino acid 1 can be Val or Tyr; Amino acid 2 can be Ala, Ser, Asp, Tyr, and Phe; Amino acid 3 is Leu; Amino acid 4 can be Asp or Leu; Amino acid 5 can be Pro, Val, or Lys; Amino acid 6 can be Ile or Phe; Amino acid 7 is Asp; Amino acid 8 can be Ile or Phe; Amino acid 9 can be Ser or Asp; Amino acid 10 can be Ile, Ala, or Ser; Amino acid 11 can be Glu or Gln; Amino acid 12 is Leu; Amino acid 13 can be Asn or Ser; Amino acid 14 can be Lys, Gln, or Ser; Amino acid 15 can be Ala, Val, or Ile; Amino acid 16 can be Lys or Asn; Amino acid 17 can be Ser, Lys, Glu, or Gln; Amino acid 18 can be Asp, Ser, or Lys; Amino acid 19 is Leu; Amino acid 20 can be Glu or Asn; Amino acid 21 can be Glu or Gln; Amino acid 22 can be Ser, Ala, or Ile; Amino acid 23 can be Lys or Leu; Amino acid 24 can be Glu, Gln, or Ala; Amino acid 25 can be Trp, or His; Amino acid 26 is Ile; Amino acid 27 is Arg or Ala; Amino acid 28 can be Arg, Gln, or Glu; Amino acid 29 can be Ser, Ala, or Ile; Amino acid 30 can be Asn or Asp; Amino acid 31 can be Gly, Thr, Glu, or Lys; Amino acid 32 can be Lys, Tyr, or Ile; Amino acid 33 is Leu; Amino acid 34 can be Asp, Ser, or His; Amino acid 35 can be Ser, Ala, or Asn and wherein Amino acid 36 is Ile.

Also preferred is a multimeric inhibitor of the invention wherein the derivative of SEQ ID NO: 99 mentioned above consists of the following amino acids: Amino acid 1 can be Val or Tyr; Amino acid 2 can be Ala, Ser, Asp, Tyr, and Phe; Amino acid 3 is Leu; Amino acid 4 can be Asp or Leu; Amino acid 5 can be Pro, Val, or Lys; Amino acid 6 can be Ile or Phe; Amino acid 7 is Asp; Amino acid 8 is Ile; Amino acid 9 can be Ser or Asp; Amino acid 10 can be Ile, Ala, or Ser; Amino acid 11 can be Glu, Gln, or Val; Amino acid 12 is Leu; Amino acid 13 can be Asn or Ser; Amino acid 14 can be Lys, Gln, or Ser; Amino acid 15 can be Ala, Val, or Ile; Amino acid 16 can be Lys or Asn; Amino acid 17 can be Ser, Lys, Glu, or Gln; Amino acid 18 can be Asp, Ser, or Lys; Amino acid 19 is Leu; Amino acid 20 can be Glu or Asn; Amino acid 21 can be Glu or Gln; Amino acid 22 can be Ser, Ala, or Ile; Amino acid 23 can be Lys or Leu; Amino acid 24 can be Glu, Gln, or Ala; Amino acid 25 can be Trp, or His; Amino acid 26 is Ile; Amino acid 27 is Arg or Ala; Amino acid 28 can be Arg, Gln, or Glu; Amino acid 29 can be Ser, Ala, or Ile; Amino acid 30 can be Asn or Asp; Amino acid 31 can be Gly, Thr, Glu, or Lys; Amino acid 32 can be Lys, Tyr, or Ile; Amino acid 33 is Leu; Amino acid 34 can be Asp, Ser, or His; Amino acid 35 can be Ser, Ala, or Asn; and wherein Amino acid 36 is Ile.

The inventors of the present invention have found that especially potent multimeric inhibitors that have a broad antiviral specificity are producible when attaching a membrane integrating lipid as described herein to the polypeptides of the multimeric inhibitor of the invention, wherein the polypeptides comprise (a) hybrid peptide(s) comprising amino acids corresponding to the fusogenic domains/regions (e.g. HR2 domains) of at least two different enveloped viruses. Particularly, the inventors substituted two amino acids (QK) of a wild-type HPIV3 peptide according to SEQ ID NO: 98 with two amino acids (KI) from Hendravirus or Nipah virus and produced peptides effectively inhibiting the viral fusion of at least two enveloped viruses when coupled to a membrane integrating lipid. Thus, in a preferred embodiment of the above outlined multimeric inhibitors, amino acid 31 of the derivative is Lys and amino acid 32 of the derivative is Ile. In addition, the inventors of the present invention have found that when amino acids of SEQ ID NO: 99 are substituted, improved inhibitors can also be produced with broader specificity and/or improved viral fusion inhibitory function. SEQ ID NOs: 106, 107, 108, 109, 110, 111, 112, 113, 114, 115, 116, 117, 118 and 119 specify preferred amino acid substitutions and preferred conserved amino acids as derived from multiple sequence alignments as shown e.g. in FIGS. 2-5.

In a preferred embodiment of the inhibitor of the present invention, at least one of said peptides, preferably at least 2, 3, 4, 5, or 6 peptides, that are comprised in said at least two polypeptides, preferably at least 3, 4, 5, or 6 polypeptides, (e.g. each peptide comprised in said polypeptides) has (have) the amino sequence WX₁EWX₂REINX₃YX₄SLIX₅SLIEEX₆QX₇QQX₈KNEX₉X₁₀LX₁₁X₁₂L (SEQ ID NO: 188), wherein X₁ to X₁₂ are defined as set out above which is preferably capable of inhibiting fusion of at least one enveloped virus, preferably HIV, with the cellular membrane (e.g. cell/plasma membrane or endosomal membrane). The inhibition of fusion of an enveloped virus with a cellular membrane of a cell (e.g. cell/plasma membrane or endosomal membrane) may occur, for example, by binding to (i) the (lipid) membrane of a cell, (ii) the (lipid) membrane of an enveloped virus, (iii) a protein associated with the (lipid) membrane of an enveloped virus, and/or (iv) a protein associated with the (lipid) membrane of a cell.

In a preferred embodiment of the inhibitor of the present invention, at least one of said peptides, preferably at least 2, 3, 4, 5, or 6 peptides, that are comprised in said at least two polypeptides, preferably at least 3, 4, 5, or 6 polypeptides, (e.g. each peptide comprised in said polypeptides) has (have) the amino acid sequence WNEWEREINKYTSLIYSLIEEAQNQQDKNEKDLLEL (SEQ ID NO: 192) or a sequence having at least 75%, preferably 85%, more preferably 90%, even more preferably 95% and most preferably 98% or 99%, i.e. 75, 76, 77, 78, 79, 80, 81, 82, 83, 84, 85, 86, 87, 88, 89, 90, 91, 92, 93, 94, 95, 96, 97, 98, or 99%, sequence identity thereto, which is preferably capable of inhibiting fusion of at least one enveloped virus, preferably HIV, with the cellular membrane (e.g. cell/plasma membrane or endosomal membrane). The inhibition of fusion of an enveloped virus with a cellular membrane of a cell (e.g. cell/plasma membrane or endosomal membrane) may occur, for example, by binding to (i) the (lipid) membrane of a cell, (ii) the (lipid) membrane of an enveloped virus, (iii) a protein associated with the (lipid) membrane of an enveloped virus, and/or (iv) a protein associated with the (lipid) membrane of a cell.

Thus, in a preferred embodiment of the multimeric inhibitor of the present invention, at least one of said peptides, preferably at least 2, 3, 4, 5, or 6 peptides, that are comprised in said at least two polypeptides, preferably at least 3, 4, 5, or 6 polypeptides, (e.g. each peptide comprised in said polypeptides) has (have) at least ten contiguous amino acids of an amino acid sequence selected from the group consisting of SEQ ID NOs: 106, 107, 108, 109, 110, 111, 112, 113, 114, 115, 116, 117, 118, and 119; wherein each X recited in the sequences specified by said SEQ ID NOs is individually selected from any amino acid with the proviso that said amino acid sequence has at least 85%, preferably 90%, more preferably 95%, and most preferably 98% or 99%, i.e. 85, 86, 87, 88, 89, 90, 91, 92, 93, 94, 95, 96, 97, 98, or 99%, sequence identity with SEQ ID NO: 99.

In a more preferred embodiment of the multimeric inhibitor of the present invention, at least one of said peptides, preferably at least 2, 3 4, 5, or 6 peptides, that are comprised in said at least two polypeptides, preferably at least 3, 4, 5, or 6, (e.g. each peptide comprised in said polypeptides) has (have) an amino acid sequence selected from the group consisting of SEQ ID NOs: 106, 107, 108, 109, 110, 111, 112, 113, 114, 115, 116, 117, 118, and 119; wherein each X recited in the sequences specified by said SEQ ID NOs is individually selected from any amino acid with the proviso that the peptide has at least 85%, preferably 90%, more preferably 95%, and most preferably 98% or 99%, i.e. 85, 86, 87, 88, 89, 90, 91, 92, 93, 94, 95, 96, 97, 98, or 99%, sequence identity with SEQ ID NO: 99.

In another more preferred embodiment of the multimeric inhibitor of the present invention, at least one of said peptides, preferably at least 2, 3, 4, 5, or 6 peptides, that are comprised in said at least two polypeptides, preferably at least 3, 4, 5, or 6 polypeptides, (e.g. each peptide comprised in said polypeptides) has (have) the amino acid sequence V₁XXDXXDISXXL₁₂XXXK₁₆XXLXXS₂₂XXXI₂₆XXS₂₉XKILXXI₃₆ (SEQ ID NO: 110) or a derivative thereof, wherein the derivative comprises at least one of the following amino acids substitutions:

V₁ may be substituted with L, A or I;

L₁₂ may be substituted with I or V;

K₁₆ may be substituted with N or H;

S₂₂ may be substituted with A;

I₂₆ may be substituted with L or V;

S₂₉ may be substituted with A; and/or

I₃₆ may be substituted with V or L;

wherein each X is individually selected from any amino acid with the proviso that the peptide has at least 85%, preferably 90%, more preferably 95%, and most preferably 98% or 99%, i.e. 85, 86, 87, 88, 89, 90, 91, 92, 93, 94, 95, 96, 97, 98, or 99%, sequence identity with SEQ ID NO: 99.

In a most preferred embodiment of the multimeric inhibitor of the present invention, at least one of said peptides, preferably at least 2, 3, 4, 5, or 6 peptides, that are comprised in said at least two polypeptides, preferably at least 3, 4, 5, or 6 polypeptides, (e.g. each peptide comprised in said polypeptides) has (have) at least ten contiguous amino acids from a HR2 domain of a Type I viral fusogenic protein of at least one enveloped virus, wherein the amino acid sequence of said domain is (individually) selected from the group consisting of SEQ ID NO: 18 to SEQ ID NO: 104, SEQ ID NO: 120 to SEQ ID NO: 127, and a sequence having at least 85%, preferably 90%, more preferably 95%, and most preferably 98% or 99%, i.e. 85, 86, 87, 88, 89, 90, 91, 92, 93, 94, 95, 96, 97, 98, or 99%, sequence identity thereto.

In another most preferred embodiment of the multimeric inhibitor of the present invention, at least one of said peptides, preferably at least 2, 3, 4, 5, or 6 peptides, that are comprised in said at least two polypeptides, preferably at least 3, 4, 5, or 6 polypeptides, (e.g. each peptide comprised in said polypeptides) has (have) an amino acid sequence (individually) selected from the group consisting of SEQ ID NO: 18 to SEQ ID NO: 104, SEQ ID NO: 120 to SEQ ID NO: 127 and a sequence having at least 85%, preferably 90%, more preferably 95%, and most preferably 98% or 99%, i.e. 85, 86, 87, 88, 89, 90, 91, 92, 93, 94, 95, 96, 97, 98, or 99%, sequence identity thereto.

In a further preferred embodiment of the multimeric inhibitor of the present invention, at least one of said peptides, preferably at least 2, 3, 4, 5, or 6 peptides, that are comprised in said at least two polypeptides, preferably at least 3, 4, 5, or 6 polypeptides, (e.g. each peptide comprised in said polypeptides) has (have) at least ten contiguous amino acids from a HR1 domain of a Type I viral fusogenic protein of an enveloped virus, wherein the amino acid sequence of said domain is (individually) selected from the group consisting of SEQ ID NO: 128 and a sequence having at least 85%, preferably 90%, more preferably 95%, and most preferably 98% or 99%, i.e. 85, 86, 87, 88, 89, 90, 91, 92, 93, 94, 95, 96, 97, 98, or 99%, sequence identity thereto.

In a more preferred embodiment of the multimeric inhibitor of the present invention, at least one of said peptides, preferably at least 2, 3, 4, 5, or 6 peptides, that are comprised in said at least two polypeptides, preferably at least 3, 4, 5, or 6 polypeptides, (e.g. each peptide comprised in said polypeptides) has (have) an amino acid sequence (individually) selected from the group consisting of SEQ ID NO: 128 and a sequence having at least 85%, preferably 90%, more preferably 95%, and most preferably 98% or 99%, i.e. 85, 86, 87, 88, 89, 90, 91, 92, 93, 94, 95, 96, 97, 98, or 99%, sequence identity thereto.

In a further preferred embodiment of the multimeric inhibitor of the present invention, at least one of said peptides, preferably at least 2, 3, 4, 5, or 6 peptides, that are comprised in said at least two polypeptides, preferably at least 3, 4, 5, or 6 polypeptides, (e.g. each peptide comprised in said polypeptides) has (have) at least ten contiguous amino acids from a HR domain of a Type III viral fusogenic protein of an enveloped virus, wherein the amino acid sequence of said domain is (individually) selected from the group consisting of SEQ ID NO: 129 to SEQ ID NO: 136 and a sequence having at least 85%, preferably 90%, more preferably 95%, and most preferably 98% or 99%, i.e. 85, 86, 87, 88, 89, 90, 91, 92, 93, 94, 95, 96, 97, 98, or 99%, sequence identity thereto.

In a more preferred embodiment of the multimeric inhibitor of the present invention, at least one of said peptides, preferably at least 2, 3, 4, 5, or 6 peptides, that are comprised in said at least two polypeptides, preferably at least 3, 4, 5, or 6 polypeptides, (e.g. each peptide comprised in said polypeptides) has (have) an amino acid sequence (individually) selected from the group consisting of SEQ ID NO: 129 to SEQ ID NO: 136 and a sequence having at least 85%, preferably 90%, more preferably 95%, and most preferably 98% or 99%, i.e. 85, 86, 87, 88, 89, 90, 91, 92, 93, 94, 95, 96, 97, 98, or 99%, sequence identity thereto.

It is also a preferred embodiment of the multimeric inhibitor of the present invention that at least one of said peptides, preferably at least 2, 3, 4, 5, or 6 peptides, that are comprised in said at least two polypeptides, preferably at least 3, 4, 5, or 6 polypeptides, (e.g. each peptide comprised in said polypeptides) has (have) at least ten contiguous amino acids from a membrane-proximal region (MPR) of a Type II viral fusogenic protein of an enveloped virus of SEQ ID NO: 137 or of a derivative thereof, wherein the derivative consists of the following amino acids:

amino acid 1 is Ala,

amino acid 2 is Trp;

amino acid 3 is Asp;

amino acid 4 is Phe;

amino acid 5 is selected from Gly and Ser;

amino acid 6 is Ser;

amino acid 7 is selected from Ile, Leu, Val, and Ala;

amino acid 8 is Gly;

amino acid 9 is Gly;

amino acid 10 is selected from Val, Leu, and Phe;

amino acid 11 is selected from Phe and Leu;

amino acid 12 is selected from Thr and Asn;

amino acid 13 is Ser;

amino acid 14 is selected from Val, Ile, and Leu;

amino acid 15 is Gly;

amino acid 16 is Lys;

amino acid 17 is selected from Leu, Ala, Met, and Gly;

amino acid 18 is selected from Ile, Leu, and Val;

amino acid 19 is His;

amino acid 20 is selected from Gln and Thr;

amino acid 21 is selected from Ile and Val; and

amino acid 22 is Phe.

In a more preferred embodiment of the multimeric inhibitor of the present invention, at least one of said peptides, preferably at least 2, 3, 4, 5, or 6 peptides, that are comprised in said at least two polypeptides, preferably at least 3, 4, 5, or 6 polypeptides, (e.g. each peptide comprised in said polypeptides) has (have) at least ten contiguous amino acids from a membrane-proximal region (MPR) of a Type II viral fusogenic protein of an enveloped virus, wherein the amino acid sequence of said domain is (individually) selected from the group consisting of SEQ ID NO: 137 to SEQ ID NO: 143 and a sequence having at least 85%, preferably 90%, more preferably 95%, and most preferably 98% or 99%, i.e. 85, 86, 87, 88, 89, 90, 91, 92, 93, 94, 95, 96, 97, 98, or 99%, sequence identity thereto.

In a most preferred embodiment of the multimeric inhibitor of any of the present invention, at least one of said peptides, preferably at least 2, 3, 4, 5, or 6 peptides, that are comprised in said at least two polypeptides, preferably at least 3, 4, 5, or 6 polypeptides, (e.g. each peptide comprised in said polypeptides) has (have) an amino acid sequence (individually) selected from the group consisting of SEQ ID NO: 137 to SEQ ID NO: 143 and a sequence having at least 85%, preferably 90%, more preferably 95%, and most preferably 98% or 99%, i.e. 85, 86, 87, 88, 89, 90, 91, 92, 93, 94, 95, 96, 97, 98, or 99%, sequence identity thereto.

In a preferred embodiment of the monomeric inhibitor of the eighth aspect of the present invention, said one polypeptide comprising an amino sequence WX₁EWX₂REINX₃YX₄SLIX₅SLIEEX₆QX₇QQX₈KNEX₉X₁₀LX₁₁X₁₂L (SEQ ID NO: 188), wherein X₁ to X₁₂ are defined as set out above, is preferably capable of inhibiting fusion of at least one enveloped virus, preferably HIV, with the cellular membrane (e.g. cell/plasma membrane or endosomal membrane).

In a preferred embodiment of the monomeric inhibitor of the eighth aspect of the present invention, said one polypeptide comprising an amino sequence WNEWEREINKYTSLIYSLIEEAQNQQDKNEKDLLEL (SEQ ID NO: 192) or a sequence having at least 75%, preferably 85%, more preferably 90%, even more preferably 95% and most preferably 98% or 99%, i.e. 75, 76, 77, 78, 79, 80, 81, 82, 83, 84, 85, 86, 87, 88, 89, 90, 91, 92, 93, 94, 95, 96, 97, 98, or 99%, sequence identity thereto, is preferably capable of inhibiting fusion of at least one enveloped virus, preferably HIV, with the cellular membrane (e.g. cell/plasma membrane or endosomal membrane).

In a preferred embodiment of the monomeric inhibitor of the eighth aspect of the present invention, said one polypeptide comprising the amino sequence SWETWEREIENYTRQIYRILEESQEQQDRNERDLLE (SEQ ID NO: 189) is preferably capable of inhibiting fusion of at least one enveloped virus, preferably HIV, with the cellular membrane (e.g. cell/plasma membrane or endosomal membrane).

All naturally occurring or synthetic HR domains e.g. HR1 or HR2 domains, or membrane-proximal regions (MRP) (peptides) which may be comprised in the improved multimeric inhibitors of the present invention are outlined in Table 2 below as SEQ ID NO: 18 to SEQ ID NO: 104 and SEQ ID NO: 106 to 143. In detail, preferred (inhibitory) peptides from HR2 (Type I viral fusogenic protein) are outlined in Table 2 below as SEQ ID NO: 18 to SEQ ID NO: 104 and SEQ ID NO: 106 to SEQ ID NO: 127. Particularly (inhibitory) HR2 hybrid peptides are outlined in Table 2 below as SEQ ID NO: 35 to SEQ ID NO: 49, SEQ ID NO: 55 to SEQ ID NO: 82 and SEQ ID NO: 100 to SEQ ID NO: 101. A preferred (inhibitory) peptide from HR1 (Type I viral fusogenic protein) is outlined in Table 2 below as SEQ ID NO: 128. Further, preferred (inhibitory) peptides from HR (Type III viral fusogenic protein) are outlined in Table 2 below as SEQ ID NO: 129 to SEQ ID NO: 136 and preferred (inhibitory) peptides from MPR (Type II viral fusogenic protein) are outlined in Table 2 below as SEQ ID NO: 137 to SEQ ID NO: 143.

TABLE 2

| | | SEQ |
|---|---|---|
| Name of target | | ID |
| virus | Amino Acid Sequence | NO: |
| Human | IDISIELNKAKSDLEESKEWIRRSNQKLDSIGNWH | 18 |
| parainfluenza | | |
| virus | | |
| 3 (HPIV3) | | |
| Human | VDISLNLASATNFLEESKIELMKAKAIISAVGGWH | 19 |
| parainfluenza | | |
| virus | | |
| 1 (HPIV 1) | | |
| Sendai virus | IDISLNLADATNFLQDSKAELEKARKILSEVGRWY | 20 |
| Sendai virus | VDISLNLADATNFLQDSKAELEKARKILSEVGRWY | 21 |
| Mumps virus | ISTELSKVNASLQNAVKYIKESNHQLQSV | 22 |
| Newcastle | ISTELGNVNNSISNALDKLEESNSKLDKV | 23 |
| disease | | |
| virus | | |
| Measles virus | VGTNLGNAIAKLEDAKELLESSDQILRSM | 24 |
| Measles virus | VGTSLGSAIAKLEDAKELLESSDQILRSM | 25 |
| Newcastle | ISTELGNVNNSISNALNKLEESNRKLDKV | 26 |
| disease | | |
| virus | | |
| Respiratory | FDASISQVNEKINQSLAFIRKSDELLHNVNAGKSTT | 27 |
| syncytical | | |
| virus (RSV) | | |
| RSV | FDASISQVNEKINQSLAFIRRSDELLHNVNTGKS | 28 |
| Hendra virus | KVDISSQISSMNQSLQQSKDYIKEAQKILDTVN | 29 |
| (HeV) | | |
| Nipah virus | KVDISSQISSMNQSLQQSKDYIKEAQRLLDTVN | 30 |
| (NiV) | | |
| Ebola virus | IEPHDWTKNITDKIDQIIHDFVDK | 31 |
| (EBOV) | | |
| EBOV | IEPHDWTKNITDKINQIIHDFID | 32 |
| EBOV | IEPHDWTKNITDEINQIKHDFID | 33 |
| Marburg virus | IGIEDLSKNISEQIDQIKKDEQKEGT | 34 |
| HPIV3/HeV | VALDPIDISIVLNKAKSDLEESKEWIRRSNKILDSI | 35 |
| HPIV3/HeV | VALDPIDISIVLNKAKSDLEESKEWIRRSNRLLDSI | 36 |
| HPIV3/HeV | VALDPIDISIVLNKAKSDLEESKEWIRESNKILDSI | 37 |
| HPIV3/HeV | VALDPIDISIVLNKAKSDLEESKEWIRESNRLLDSI | 38 |
| HPIV3/HeV | IDISIVLNKAKSDLEESKEWIRRSNGKLDSI | 39 |
| HPIV3/HeV | VALDPIDISEVLNKAKSDLEESKEWIRRSNGKLDSI | 40 |
| HPIV3/HeV | VALDPIDISIVLNKMKSDLEESKEWIRRSNGKLDSI | 41 |
| HPIV3/HeV | VALDPIDISIVLNKIKSDLEESKEWIRRSNGKLDSI | 42 |
| HPIV3/HeV | VALDPIDISIVLNKAKSDLEESKEWIRRSNGILDSI | 43 |
| HPIV3/HeV | VALDPIDISIVLNKAKSDLEESKEWIRESNGKLDSI | 44 |
| HPIV3/HeV | VALDPIDISIVLNKAKSDLEESKEWIRKSNGKLDSI | 45 |
| HPIV3/HeV | VALDPIDISIVLNKAKSELEESKEWIRRSNGKLDSI | 46 |
| HPIV3/HeV | VALDPIDISIVLNKAKSXLEESKEWIRRSNGKLDSI | 47 |
| HPIV3/HeV | VALDPIDISIVLNKAKSDLEESKEWIRRSNKILESI | 48 |
| HPIV3/HeV | VALDPIDISIVLNKAKSDLEESKEWIRRSNGKLDSIGSGSGC | 49 |
| EBOV | IEPHDWTKNITEKIDQIIHDFVDK | 50 |
| EBOV | IEPHDWTKNITDKIDEIIHDFVDK | 51 |
| EBOV | IEPHDWTKNITDKIEQIIKDFVDK | 52 |
| EBOV | IEPHDWTKNITDKIDQIIHDFVDKGSGSGC | 53 |
| RSV | SDEFDASISQVNEKINQSLAFIRKSDELLHNV | 54 |
| HPIV3/RSV | SALDPIDISIELNKAKSDLEESKEWIRRSNGK | 55 |
| HPIV3/RSV | VDLDPIDISIELNKAKSDLEESKEWIRRSNGK | 56 |
| HPIV3/RSV | VAEDPIDISIELNKAKSDLEESKEWIRRSNGK | 57 |
| HPIV3/RSV | VALFPIDISIELNKAKSDLEESKEWIRRSNGK | 58 |
| HPIV3/RSV | VALDDIDISIELNKAKSDLEESKEWIRRSNGK | 59 |
| HPIV3/RSV | VALDPADISIELNKAKSDLEESKEWIRRSNGK | 60 |
| HPIV3/RSV | VALDPISISIELNKAKSDLEESKEWIRRSNGK | 61 |
| HPIV3/RSV | VALDPIDISQELNKAKSDLEESKEWIRRSNGK | 62 |
| HPIV3/RSV | VALDPIDISIVLNKAKSDLEESKEWIRRSNGK | 63 |
| HPIV3/RSV | VALDPIDISIENNKAKSDLEESKEWIRRSNGK | 64 |
| HPIV3/RSV | VALDPIDISIELEKAKSDLEESKEWIRRSNGK | 65 |
| HPIV3/RSV | VALDPIDISIELNKIKSDLEESKEWIRRSNGK | 66 |
| HPIV3/RSV | VALDPIDISIELNKANSDLEESKEWIRRSNGK | 67 |
| HPIV3/RSV | VALDPIDISIELNKAKQDLEESKEWIRRSNGK | 68 |
| HPIV3/RSV | VALDPIDISIELNKAKSSLEESKEWIRRSNGK | 69 |
| HPIV3/RSV | VALDPIDISIELNKAKSDLAESKEWIRRSNGK | 70 |
| HPIV3/RSV | VALDPIDISIELNKAKSDLEFSKEWIRRSNGK | 71 |
| HPIV3/RSV | VALDPIDISIELNKAKSDLEEIKEWIRRSNGK | 72 |
| HPIV3/RSV | VALDPIDISIELNKAKSDLEESREWIRRSNGK | 73 |
| HPIV3/RSV | VALDPIDISIELNKAKSDLEESKKWIRRSNGK | 74 |
| HPIV3/RSV | VALDPIDISIELNKAKSDLEESKESIRRSNGK | 75 |
| HPIV3/RSV | VALDPIDISIELNKAKSDLEESKEWDRRSNGK | 76 |
| HPIV3/RSV | VALDPIDISIELNKAKSDLEESKEWIERSNGK | 77 |
| HPIV3/RSV | VALDPIDISIELNKAKSDLEESKEWIRLSNGK | 78 |
| HPIV3/RSV | VALDPIDISIELNKAKSDLEESKEWIRRLNGK | 79 |
| HPIV3/RSV | VALDPIDISIELNKAKSDLEESKEWIRRSHGK | 80 |
| HPIV3/RSV | VALDPIDISIELNKAKSDLEESKEWIRRSNNK | 81 |
| HPIV3/RSV | VALDPIDISIELNKAKSDLEESKEWIRRSNGV | 82 |
| Influenza A virus | GTYDHDVYRDEALNNRFQIKGVELKSGYKDW | 83 |
| Influenza A virus | GTFNAGEFSLPTFDSLNITAASLNDDGL | 84 |
| Influenza A virus | GTYDHTEYAEESKLKRQEIDGIKLKSED | 85 |
| Influenza A virus | GTYDHKEFEEESKINRQEIEGVKLDSSG | 86 |
| Influenza A virus | NTYDHSTYREEAMQNRVKIDPVKLSSGY | 87 |
| Influenza A virus | NTYDHSQYREEALLNRLNINSVKLSSGY | 88 |
| Influenza A virus | GTYDHDVYRDEALNNRFQIKGVELKSGY | 89 |
| Influenza A virus | GTYDHDIYRDEAINNRFQIQGVKLIQGY | 90 |
| Influenza A virus | GTYDYPKYEEESKLNRNEIKGVKLSSMG | 91 |
| Influenza A virus | GTYDYPQYSEEARLNREEISGVKLESMG | 92 |
| Influenza A virus | GTYDYPKYSEESKLNREEIDGVKLESMG | 93 |
| Influenza A virus | GTYDHDVYRDEALNNRFQIKGVELKSG | 94 |
| Influenza A virus | GTYDHDVYRDEALNNRFQIKG | 95 |
| RSV | SDEFDASISQVNEKINQSLAFIRKSDELLHNVNAGK | 96 |
| RSV | YDPLVFPSDEFDASISQVNEKINQSLAFIRKSDELLHNV | 97 |
| HPIV3 | VALDPIDISIELNKAKSDLEESKEWIRRSNQKLDSI | 98 |
| HPIV3 | VALDPIDISIELNKAKSDLEESKEWIRRSNGKLDSI | 99 |
| HPIV3/HeV | VALDPIDISIVLNKIKSDLEESKEWIRRSNKILDSI | 100 |
| HPIV3/HeV | VALDPIDISIVLNKIKSDLEESKEWIRRSNKILDSIGSGSGC | 101 |
| SV5 | LSIDPLDISQNLAAVNKSLSDALQHLAQSDTYLSAI | 102 |
| HeV | VYTDKVDISSQISSMNQSLQQSKDYIKEAQKILDTV | 103 |
| NiV | VFTDKVDISSQISSMNQSLQQSKDYIKEAQRLLDTV | 104 |
| Artificial sequence | XXXXXXXXXXXXXXXXXXXXXXXXXXXXXXKIXXXX | 106 |
| Artificial sequence | XXXXXXXXXXVXXXXXXXXXXXXXXXXXXXKIXXXX | 107 |
| Artificial sequence | XXXXXXXXXXVXXXIXXXXXXXXXXXXXXXKIXXXX | 108 |
| Artificial sequence | XXXDXXDISXVXXXXXXXLXXXXXXXXXXXKILXXX | 109 |
| Artificial sequence | VXXDXXDISXXLXXXKXXLXXSXXXIXXSXKILXXI | 110 |
| Artificial sequence | VXXDXXDISXVLXXIKXXLXXSXXXIXXSXKILXXI | 111 |
| Artificial sequence | VXXDXXDISXVLXXIKXXLXXSXXXIXXSXKILXXIGSGSGC | 112 |
| Artificial sequence | XXXXXXXXXXXXXXXXXXXXXXXXXXXXXXGKXXXX | 113 |
| Artificial sequence | XXXXXXXXXXVXXXXXXXXXXXXXXXXXXXGKXXXX | 114 |
| Artificial sequence | XXXXXXXXXXVXXXIXXXXXXXXXXXXXXXGKXXXX | 115 |
| Artificial sequence | XXXDXXDISXVXXXXXXXLXXXXXXXXXXXGKLXXX | 116 |
| Artificial sequence | VXXDXXDISXXLXXXKXXLXXSXXXIXXSXGKLXXI | 117 |
| Artificial sequence | VXXDXXDISXVLXXIKXXLXXSXXXIXXSXGKLXXI | 118 |
| Artificial sequence | VXXDXXDISXVLXXAKXXLXXSXXXIXXSXGKLXXIGSGSGC | 119 |
| HIV | WMEWDREINNYTSLIHSLIEESQNQQEKNEQELL | 120 |
| Measles Virus | PISLERLDVGTNLGNAIAKLEDAKELLESSDQILR | 121 |
| SARS Virus | GDISGINASVVNIQKEIDRLNEVAKNL | 122 |
| Junin Virus | SYLNISDFRNDWILESDFLISEMLSKEYSD | 123 |
| Machupo Virus | SYLNISEFRNDWILESDHLISEMLSKEYAE | 124 |
| Guanarito Virus | SYLNESDFRNEWILESDHLISEMLSKEYQD | 125 |
| Lassa Virus | SYLNETHFSDDIEQQADNMITEMLQKEYME | 126 |
| hMPV | FNVALDQVFENIENSQALVDQSNRILSSAEKG | 127 |
| hMPV | AKTIRLESEVTAIKNALKKTNEAVSTLGNGVRVLATAVRELKDFVSKN | 128 |
| HHV 6A, 6B | SPDELSRANVFDLENILREYNSYKSALYTIEAKIAT | 129 |
| HHV 6A, 6B | INTTESLTNYEKRVTRFYEPP | 130 |
| HHV 6A, 6B | ATFVDETLNDVDEVEALLLKFNNLGI | 131 |
| Cytomegalovirus | NVFDLEEIMREFNSYKQRVKYVEDKVVDP | 132 |
| Cytomegalovirus | NQVDLTETLERYQQRLNTYAL | 133 |
| HSV-1 | DYTEVQRRNQLHDLRFADIDTVI | 134 |
| HSV-1 | ARLQLLEARLQHLVAEILEREQ | 135 |
| HSV-1 | SDVAAATNADLRTALARADHQKTLF | 136 |
| DV1 | AWDFGSIGGVFTSVGKLIHQIF | 137 |
| DV2 | AWDFGSLGGVFTSIGKALHQVF | 138 |
| DV3 | AWDFGSVGGVLNSLGKMVHQIF | 139 |
| DV4 | AWDFGSVGGVFTSVGKAVHQVF | 140 |
| West Nile Virus | AWDFGSVGGVFTSVGKAVHQVF | 141 |
| Yellow Fever | AWDFSSAGGFFTSVGKGIHTVF | 142 |
| Virus | | |
| Japanese | AWDFGSIGGVFNSIGKAVHQVF | 143 |
| Encephalitis Virus | | |

The peptides as set out above comprised in the at least two polypeptides mentioned above, preferably at least 2, 3, 4, 5, or 6 polypeptides, comprised in the multimeric inhibitor of the present invention may be may be identical or different. Preferably, the peptides as set out above comprised in the at least two polypeptides mentioned above, preferably at least 2, 3, 4, 5, or 6 polypeptides, comprised in the multimeric inhibitor of the present invention are identical. Thus, for example, in a preferred embodiment, the multimeric inhibitor of the present invention comprises (i) 2 polypeptides each comprising, essentially consisting of, or consisting of an identical peptide having an amino acid sequence selected from the group consisting of SEQ ID NO: 18 to SEQ ID NO: 104 and SEQ ID NO: 106 to SEQ ID NO: 143, (ii) 3 polypeptides each comprising, essentially consisting of, or consisting of an identical peptide having an amino acid sequence selected from the group consisting of SEQ ID NO: 18 to SEQ ID NO: 104 and SEQ ID NO: 106 to SEQ ID NO: 143, (iii) 4 polypeptides each comprising, essentially consisting of, or consisting of an identical peptide having an amino acid sequence selected from the group consisting of SEQ ID NO: 18 to SEQ ID NO: 104 and SEQ ID NO: 106 to SEQ ID NO: 143, (iv) 5 polypeptides each comprising, essentially consisting of, or consisting of an identical peptide having an amino acid sequence selected from the group consisting of SEQ ID NO: 18 to SEQ ID NO: 104 and SEQ ID NO: 106 to SEQ ID NO: 143, or (v) 6 polypeptides each comprising, essentially consisting of, or consisting of an identical peptide having an amino acid sequence selected from the group consisting of SEQ ID NO: 18 to SEQ ID NO: 104 and SEQ ID NO: 106 to SEQ ID NO: 143.

As mentioned above, in a preferred embodiment of the multimeric inhibitor of the present invention, at least one of said peptides, preferably at least 2, 3, 4, 5, or 6 of said peptides, outlined above that are comprised in said at least two polypeptides, preferably at least 3, 4, 5, or 6 polypeptides, (e.g. each peptide comprised in said polypeptides) has (have) at least 85%, preferably 90%, more preferably 95%, and most preferably 98% or 99%, i.e. 85, 86, 87, 88, 89, 90, 91, 92, 93, 94, 95, 96, 97, 98, or 99%, sequence identity to an amino acid sequence selected from the group consisting of SEQ ID NO: 18 to SEQ ID NO: 104 and SEQ ID NO: 120 to SEQ ID NO: 143.

The peptide having at least 85%, preferably 90%, more preferably 95%, and most preferably 98% or 99%, i.e. 85, 86, 87, 88, 89, 90, 91, 92, 93, 94, 95, 96, 97, 98, or 99%, sequence identity to an amino acid sequence selected from the group consisting of SEQ ID NO: 18 to SEQ ID NO: 104 and SEQ ID NO: 120 to SEQ ID NO: 143 may be designated as “peptide derivate”. Said “peptide derivate” may alternatively be designated as “domain binding derivate”, e.g. HR binding derivate such as HR1 or HR2 binding derivate or beta-sheet binding derivate (see also functional assays below). The peptide having an amino acid sequence selected from the group consisting of SEQ ID NO: 18 to SEQ ID NO: 104 and SEQ ID NO: 120 to SEQ ID NO: 143 may be designated as “reference (wild-type) peptide”.

Preferably, the sequence identity is over a continuous stretch of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 125, 150, 175, 200 or more amino acids, more preferably, over the whole length of the respective reference (wild-type) peptide. For example, a peptide having a sequence according to SEQ ID NO: 99 and carrying two amino acid substitutions exhibits a sequence identity of about 94% over the whole length of the respective reference (wild-type) peptide having a sequence according to SEQ ID NO: 99.

As used herein, the term “identity” or “identical” in the context of peptide, peptide or protein sequences refers to the number of residues in the two sequences (a reference sequence as indicated herein as SEQ ID NO and a second sequence, i.e. the sequence in question) that are identical when aligned, for example, over the entire length of the reference sequence for maximum correspondence as is well known in the art. Specifically, the percent sequence identity of the two sequences, whether nucleic acid or amino acid sequences, is the number of exact matches (nucleotides or amino acids, respectively) between the reference sequence and the aligned second sequence divided by the length of the reference sequence and multiplied by 100. Alignment tools that can be used to align two sequences are well known to the person skilled in the art and can, for example, be obtained on the World Wide Web, e.g., ClustalW (www.ebi.ac.uk/clustalw) or Align (http://www.ebi.ac.uk/emboss/align/index.html). The alignments between two sequences may be carried out using standard settings, for Align EMBOSS::needle with the parameters set preferably to: Matrix: Blosum62 for protein sequences and “DNAfull” for nucleic acid sequences; Gap Open=10.0; and Gap Extend=0.5. Those skilled in the art understand that it may be necessary to introduce gaps in either sequence to produce a satisfactory alignment.

In an alternative preferred embodiment of multimeric inhibitor of the present invention, at least one of said peptides, preferably at least 2, 3, 4, 5, or 6 of said peptides, outlined above that are comprised in said at least two polypeptides, preferably at least 3, 4, 5, or 6 polypeptides, (e.g. each peptide comprised in said polypeptides) has (have) an amino acid sequence according to SEQ ID NO: 18 to SEQ ID NO: 104 or SEQ ID NO: 120 to SEQ ID NO: 143 or is (are) a (domain binding) peptide derivative thereof, which comprise(s) 1, 2, 3, 4, 5, or 6, preferably 1, 2, or 3, amino acid changes (e.g. (a) amino acid substitution(s), deletion(s), and/or addition(s)/insertion(s)) with respect to the amino acid sequence according to SEQ ID NO: 18 to SEQ ID NO: 104 or SEQ ID NO: 120 to SEQ ID NO: 143. Said “peptide derivate” may also be designated as “domain binding derivate”, e.g. HR such as HR1 or HR2 binding derivate or beta-sheet binding derivate (see also functional assays below).

It should be noted that the sequences of the peptide derivates (the peptides in question) aforementioned above that vary from the respective reference (wild-type) sequence (e.g. SEQ ID NO: 18 to SEQ ID NO: 104 or SEQ ID NO: 120 to SEQ ID NO: 143) are only regarded as sequences within the context of the present invention, if the modifications with respect to the amino acid sequence on which they are based (wild-type sequence) do not negatively affect the ability of the multimeric inhibitor wherein they are comprised to inhibit the fusion of at least one enveloped virus, for example, by still binding to a viral fusogenic protein, preferably to a domain, e.g. HR domain such as HR1 or HR2 domain, or beta-sheet domain, comprised therein.

The domain binding activity, e.g. HR domain such as HR1 or HR2 domain or beta-sheet domain binding activity, of the peptide derivate (the peptide in question) is not substantially altered, if the binding is at least 50%, preferably at least 60%, preferably at least 70%, preferably at least 80%, preferably at least 90% or at least 100% or more of the binding observed for respective reference (wild-type) peptide, e.g. HR domain such as HR1 or HR2 domain or beta-sheet domain binding peptide, (e.g. peptide having a sequence according to SEQ ID NO: 18 to SEQ ID NO: 104 or SEQ ID NO: 120 to SEQ ID NO: 143). For example, (i) the HR1 domain binding activity is not substantially altered, if the binding is at least 50%, preferably at least 60%, preferably at least 70%, preferably at least 80%, preferably at least 90% or at least 100% or more of the binding observed for the respective reference HR2 domain peptide on which the HR2 domain peptide derivate is based, (ii) the HR2 domain binding activity is not substantially altered, if the binding is at least 50%, preferably at least 60%, preferably at least 70%, preferably at least 80%, preferably at least 90% or at least 100% or more of the binding observed for the respective reference HR1 domain peptide on which the HR1 domain peptide derivate is based, or (iii) the beta-sheet domain binding activity is not substantially altered, if the binding is at least 50%, preferably at least 60%, preferably at least 70%, preferably at least 80%, preferably at least 90% or at least 100% or more of the binding observed for the respective reference membrane-proximal region peptide on which the membrane-proximal region peptide derivate is based.

The domain binding activity may be assayed as set out above using in vitro and in vivo binding assays as well as functional assays. For example, a respective reference HR2 domain peptide having a sequence according to SEQ ID NO: 18 to SEQ ID NO: 104 or SEQ ID NO: 120 to SEQ ID NO: 127 (see Table 2 above) (positive control) may be tested together with a HR2 domain peptide derivate thereof for binding to a HR1 domain having a sequence according to SEQ ID NO: 1 to SEQ ID NO: 17, SEQ ID NO: 105, or SEQ ID NO: 144 to 150. If the respective reference HR2 domain hybrid peptide having a sequence according to SEQ ID NO: 35 to SEQ ID NO: 49, SEQ ID NO: 55 to SEQ ID NO: 82 or SEQ ID NO: 100 to SEQ ID NO: 101, wherein the hybrid peptide comprises sequence elements of HR2 domains of multiple different viruses (positive control), is tested together with the HR2 domain hybrid peptide derivate thereof, the HR1 domain used in in vitro or in vivo binding assays may be from any of the viruses from that the HR2 domain was taken from. In some of above cases the HR2 peptide is similar or identically present in two viruses. In those cases the HR1 domain may be taken from one of said viruses for a functional assay testing the HR1 binding activity of the HR2 domain peptide derivate (the peptide in question).

Further, for example, a respective reference HR1 domain peptide having a sequence according to SEQ ID NO: 128 (see Table 2 above) (positive control) may be tested together with a HR1 domain peptide derivate thereof for binding to a HR2 domain having a sequence according to SEQ ID NO: 151. Furthermore, for example, a respective reference MPR peptide having a sequence according to SEQ ID NO: 137 to SEQ ID NO: 143 (see Table 2 above) (positive control) may be tested together with a MPR peptide derivate thereof for binding to a beta-sheet domain, preferably a beta-sheet domain comprised in domain II, of a Type II fusogenic protein, or a respective reference HR domain peptide having a sequence according to SEQ ID NO: 129 to SEQ ID NO: 136 (see Table 2 above) (positive control) may be tested together with a HR peptide derivate thereof for binding to a domain of a Type III fusogenic protein.

It is preferred, that at least one of said peptides, preferably at least 2, 3, 4, 5, or 6 of said peptides, outlined above that are comprised in said at least two polypeptides, preferably at least 3, 4, 5, or 6 polypeptides, (e.g. each peptide comprised in said polypeptides) has (have) a length of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, or 200 contiguous amino acids and/or has (have) a length of not more than 45, 50, 60, 70, 80, 90, 100, 110, 120, 130, 140, 150, 160, 170, 180, 190, or 200 contiguous amino acids. It is further preferred that at least one of said peptides, preferably at least 2, 3, 4, 5, or 6 of said peptides, outlined above that are comprised in said at least two polypeptides, preferably at least 3, 4, 5, or 6 polypeptides, (e.g. each peptide comprised in said polypeptides) has (have) a length of between 8 and 200 contiguous amino acids, or between 10 and 200 contiguous amino acids, more preferably between 15 and 150 contiguous amino acids, or between 20 and 100 contiguous amino acids, and most preferably between 20 and 75 contiguous amino acids, or between 20 and 50 contiguous amino acids, i.e. 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, or 200 amino acids.

Considering the above, it is preferred that at least one of said peptides, preferably at least 2, 3, 4, 5, or 6 of said peptides, outlined above that are comprised in said at least two polypeptides, preferably at least 3, 4, 5, or 6 polypeptides, (e.g. each peptide comprised in said polypeptides)
(i) has (have) a length of at least 14, at least 15, at least 16, at least 17, at least 18, at least 19, at least 20, at least 21, at least 22, or at least 23 contiguous amino acids of above peptides according to SEQ ID NO: 18 to 94 and 96-104,
(ii) has (have) a length of at least 14, at least 15, at least 16, at least 17, at least 18, at least 19, at least 20, at least 21, at least 22, or at least 23 contiguous amino acids of above peptides according to SEQ ID NO: 18 to 94 and 96-104 and comprises one amino acid change with respect to the amino acid sequences according to SEQ ID NO: 18 to 94 and 96-104,
(iii) has (have) a length of at least 14, at least 15, at least 16, at least 17, at least 18, at least 19, at least 20, at least 21, at least 22, or at least 23 contiguous amino acids of above peptides according to SEQ ID NO: 18 to 94 and 96-104 and comprises two amino acid changes with respect to the amino acid sequences according to SEQ ID NO: 18 to 94 and 96-104, or
(iv) has (have) a length of at least 14, at least 15, at least 16, at least 17, at least 18, at least 19, at least 20, at least 21, at least 22, or at least 23 contiguous amino acids of above peptides according to SEQ ID NO: 18 to 94 and 96-104 and comprises three amino acid changes with respect to the amino acid sequences according to SEQ ID NO: 18 to 94 and 96-104.

It is preferred that at least one of said peptides, preferably at least 2, 3, 4, 5, or 6 of said peptides, outlined above that are comprised in said at least two polypeptides, preferably at least 3, 4, 5, or 6 polypeptides, (e.g. each peptide comprised in said polypeptides)
(i) has (have) a length of at least 24 contiguous amino acids of above peptides according to SEQ ID NO: 18 to 31 and 34 to 94 and 96-104,
(ii) has (have) a length of at least 24 contiguous amino acids of above peptides according to SEQ ID NO: 18 to 31 and 34 to 94 and 96-104 and comprises one amino acid change with respect to the amino acid sequences according to SEQ ID NO: 18 to 31 and 34 to 94 and 96-104,
(iii) has (have) a length of at least 24 contiguous amino acids of above peptides according to SEQ ID NO: 18 to 31 and 34 to 94 and 96-104 and comprises two amino acid changes with respect to the amino acid sequences according to SEQ ID NO: 18 to 31 and 34 to 94 and 96-104, or
(iv) has (have) a length of at least 24 contiguous amino acids of above peptides according to SEQ ID NO: 18 to 31 and 34 to 94 and 96-104 and comprises three amino acid changes with respect to the amino acid sequences according to SEQ ID NO: 18 to 31 and 34 to 94 and 96-104.

It is preferred that at least one of said peptides, preferably at least 2, 3, 4, 5, or 6 of said peptides, outlined above that are comprised in said at least two polypeptides, preferably at least 3, 4, 5, or 6 polypeptides, (e.g. each peptide comprised in said polypeptides)
(i) has (have) a length of at least 25 or at least 26 contiguous amino acids of above peptides according to SEQ ID NO: 18 to 30 and 35 to 94 and 96-104,
(ii) has (have) a length of at least 25 or at least 26 contiguous amino acids of above peptides according to SEQ ID NO: 18 to 30 and 32 to 94 and 96-104 and comprises one amino acid change with respect to the amino acid sequences according to SEQ ID NO: 18 to 30, 34 to 49 and 53 to 94 and 96-104,
(iii) has (have) a length of at least 25 or at least 26 contiguous amino acids of above peptides according to SEQ ID NO: 18 to 30, 34 to 49 and 53 to 94 and 96-104 and comprises two amino acid changes with respect to the amino acid sequences according to SEQ ID NO: 18 to 30, 34 to 49 and 53 to 94 and 96-104, or
(iv) has (have) a length of at least 25 or at least 26 contiguous amino acids of above peptides according to SEQ ID NO: 18 to 30, 34 to 49 and 53 to 94 and 96-104 and comprises three amino acid changes with respect to the amino acid sequences according to SEQ ID NO: 18 to 30, 34 to 49 and 53 to 94 and 96-104.

It is preferred that at least one of said peptides, preferably at least 2, 3, 4, 5, or 6 of said peptides, outlined above that are comprised in said at least two polypeptides, preferably at least 3, 4, 5, or 6 polypeptides, (e.g. each peptide comprised in said polypeptides)
(i) has (have) a length of at least 27, at least 28 or at least 29 contiguous amino acids of above peptides according to SEQ ID NO: 18 to 30, 35 to 49 and 53 to 83 and 96-104,
(ii) has (have) a length of at least 27, at least 28 or at least 29 contiguous amino acids of above peptides according to SEQ ID NO: 18 to 30, 35 to 49 and 53 to 83 and 96-104 and comprises one amino acid change with respect to the amino acid sequences according to SEQ ID NO: 18 to 30, 35 to 49 and 53 to 83 and 96-104,
(iii) has (have) a length of at least 27, at least 28 or at least 29 contiguous amino acids of above peptides according to SEQ ID NO: 18 to 30, 35 to 49 and 53 to 83 and 96-104 and comprises two amino acid changes with respect to the amino acid sequences according to SEQ ID NO: 18 to 30, 35 to 49 and 53 to 83 and 96-104, or
(iv) has (have) a length of at least 27, at least 28 or at least 29 contiguous amino acids of above peptides according to SEQ ID NO: 18 to 30, 35 to 49 and 53 to 83 and 96-104 and comprises three amino acid changes with respect to the amino acid sequences according to SEQ ID NO: 18 to 30, 35 to 49 and 53 to 83 and 96-104.

It is preferred that at least one of said peptides, preferably at least 2, 3, 4, 5, or 6 said peptides, outlined above that are comprised in said at least two polypeptides, preferably at least 3, 4, 5, or 6 polypeptides, (e.g. each peptide comprised in said polypeptides)
(i) has (have) a length of at least 30 contiguous amino acids of above peptides according to SEQ ID NO: 18 to 21, 27 to 30, 35 to 49 and 53 to 83 and 96-104,
(ii) has (have) a length of at least 30 contiguous amino acids of above peptides according to SEQ ID NO: 18 to 21, 27 to 30, 35 to 49 and 53 to 83 and 96-104 and comprises one amino acid change with respect to the amino acid sequences according to SEQ ID NO: 18 to 21, 27 to 30, 35 to 49 and 53 to 83 and 96-104,
(iii) has (have) a length of at least 30 contiguous amino acids of above peptides according to SEQ ID NO: 18 to 21, 27 to 30, 35 to 49 and 53 to 83 and 96-104 and comprises two amino acid changes with respect to the amino acid sequences according to SEQ ID NO: 18 to 21, 27 to 30, 35 to 49 and 53 to 83 and 96-104, or
(iv) has (have) a length of at least 30 contiguous amino acids of above peptides according to 18 to 21, 27 to 30, 35 to 49 and 53 to 83 and 96-104 and comprises three amino acid changes with respect to the amino acid sequences according to 18 to 21, 27 to 30, 35 to 49 and 53 to 83 and 96-104.

It is preferred that at least one of said peptides, preferably at least 2, 3, 4, 5, or 6 of said peptides, outlined above that are comprised in said at least two polypeptides, preferably at least 3, 4, 5, or 6 polypeptides, (e.g. each peptide comprised in said polypeptides)
(i) has (have) a length of at least 31 contiguous amino acids of above peptides according to SEQ ID NO: 18 to 21, 27 to 30, 35 to 49 and 54 to 82 and 96-104,
(ii) has (have) a length of at least 31 contiguous amino acids of above peptides according to SEQ ID NO: 18 to 21, 27 to 30, 35 to 49 and 54 to 82 and 96-104 and comprises one amino acid change with respect to the amino acid sequences according to SEQ ID NO: 18 to 21, 27 to 30, 35 to 49 and 54 to 82 and 96-104,
(iii) has (have) a length of at least 31 contiguous amino acids of above peptides according to SEQ ID NO: 18 to 21, 27 to 30, 35 to 49 and 54 to 82 and 96-104 and comprises two amino acid changes with respect to the amino acid sequences according to SEQ ID NO: 18 to 21, 27 to 30, 35 to 49 and 54 to 82 and 96-104, or
(iv) has (have) a length of at least 31 contiguous amino acids of above peptides according to 18 to 21, 27 to 30, 35 to 49 and 54 to 82 and 96-104 and comprises three amino acid changes with respect to the amino acid sequences according to 18 to 21, 27 to 30, 35 to 49 and 54 to 82 and 96-104.

It is preferred that at least one of said peptides, preferably at least 2, 3, 4, 5, or 6 of said peptides, outlined above that are comprised in said at least two polypeptides, preferably at least 3, 4, 5, or 6 polypeptides, (e.g. each peptide comprised in said polypeptides)
(i) has (have) a length of at least 32 contiguous amino acids of above peptides according to SEQ ID NO: 18 to 21, 27 to 30, 35 to 38, 40 to 49 and 54 to 82 and 96-104,
(ii) has (have) a length of at least 31 contiguous amino acids of above peptides according to SEQ ID NO: 18 to 21, 27 to 30, 35 to 38, 40 to 49 and 54 to 82 and 96-104 and comprises one amino acid change with respect to the amino acid sequences according to SEQ ID NO: 18 to 21, 27 to 30, 35 to 38, 40 to 49 and 54 to 82 and 96-104,
(iii) has (have) a length of at least 31 contiguous amino acids of above peptides according to SEQ ID NO: 18 to 21, 27 to 30, 35 to 38, 40 to 49 and 54 to 82 and 96-104 and comprises two amino acid changes with respect to the amino acid sequences according to SEQ ID NO: 18 to 21, 27 to 30, 35 to 38, 40 to 49 and 54 to 82 and 96-104, or
(iv) has (have) a length of at least 31 contiguous amino acids of above peptides according to 18 to 21, 27 to 30, 35 to 38, 40 to 49 and 54 to 82 and 96-104 and comprises three amino acid changes with respect to the amino acid sequences according to 18 to 21, 27 to 30, 35 to 38, 40 to 49 and 54 to 82 and 96-104.

It is preferred that at least one of said peptides, preferably at least 2, 3, 4, 5, or 6 of said peptides, outlined above that are comprised in said at least two polypeptides, preferably at least 3, 4, 5, or 6 polypeptides, (e.g. each peptide comprised in said polypeptides)
(i) has (have) a length of at least 33 contiguous amino acids of above peptides according to SEQ ID NO: 18 to 21, 27 to 30, 35 to 38, and 40 to 49 and 96-104,
(ii) has (have) a length of at least 32 contiguous amino acids of above peptides according to SEQ ID NO: 18 to 21, 27 to 30, 35 to 38, and 40 to 49 and 96-104 and comprises one amino acid change with respect to the amino acid sequences according to SEQ ID NO: 18 to 21, 27 to 30, 35 to 38, and 40 to 49 and 96-104,
(iii) has (have) a length of at least 31 contiguous amino acids of above peptides according to SEQ ID NO: 18 to 21, 27 to 30, 35 to 38, and 40 to 49 and 96-104 and comprises two amino acid changes with respect to the amino acid sequences according to SEQ ID NO: 18 to 21, 27 to 30, 35 to 38, and 40 to 49 and 96-104, or
(iv) has (have) a length of at least 31 contiguous amino acids of above peptides according to 18 to 21, 27 to 30, 35 to 38, and 40 to 49 and 96-104 and comprises three amino acid changes with respect to the amino acid sequences according to 18 to 21, 27 to 30, 35 to 38, and 40 to 49 and 96-104.

It is preferred that at least one of said peptides, preferably at least 2, 3, 4, 5, or 6 of said peptides, outlined above that are comprised in said at least two polypeptides, preferably at least 3, 4, 5, or 6 polypeptides, (e.g. each peptide comprised in said polypeptides)
(i) has (have) a length of at least 27 contiguous amino acids of above peptides according to SEQ ID NO: 120 to SEQ ID NO: 127 or SEQ ID NO: 128, or
(ii) has (have) a length of at least 27 contiguous amino acids of above peptides according to SEQ ID NO: 120 to SEQ ID NO: 127 or SEQ ID NO: 128 and comprises one, two, or three amino acid change(s) with respect to the amino acid sequences according to SEQ ID NO: 120 to SEQ ID NO: 127 or SEQ ID NO: 128.

It is preferred that at least one of said peptides, preferably at least 2, 3, 4, 5, or 6 of said peptides, outlined above that are comprised in said at least two polypeptides, preferably at least 3, 4, 5, or 6 polypeptides, (e.g. each peptide comprised in said polypeptides)
(i) has (have) a length of at least 21 contiguous amino acids of above peptides according to SEQ ID NO: 129 to SEQ ID NO: 136, or
(ii) has (have) a length of at least 21 contiguous amino acids of above peptides according to SEQ ID NO: 129 to SEQ ID NO: 136 and comprises one, two, or three amino acid change(s) with respect to the amino acid sequences according to SEQ ID NO: 129 to SEQ ID NO: 136.

It is preferred that at least one of said peptides, preferably at least 2, 3, 4, 5, or 6 of said peptides, outlined above that are comprised in said at least two polypeptides, preferably at least 3, 4, 5, or 6 polypeptides, (e.g. each peptide comprised in said polypeptides)
(i) has (have) a length of at least 22 contiguous amino acids of above peptides according to SEQ ID NO: 137 to SEQ ID NO: 143, or
(ii) has (have) a length of at least 22 contiguous amino acids of above peptides according to SEQ ID NO: 137 to SEQ ID NO: 143 and comprises one, two, or three amino acid change(s) with respect to the amino acid sequences according to SEQ ID NO: 137 to SEQ ID NO: 143.

It should be noted that the aforementioned peptides that may be comprised in the polypeptides of the multimeric inhibitor of the present invention outlined above may have a different length or an identical length, e.g. a length of at least 10, 15, 20, 25, 30, 35, 40, 45, 50, 60, 70, 80, 90, 100, 125, 150, 175, or 200 contiguous amino acids and/or not more than 50, 60, 70, 80, 90, 100, 125, 175, or 200 contiguous amino acids. It is preferred that the aforementioned peptides that are comprised in the polypeptides of the multimeric inhibitor of the present invention outlined above have an identical length. Thus, in particular preferred embodiments, the multimeric inhibitor of the present invention comprises 2, 3, or 4 polypeptides each comprising, essentially consisting of, or consisting of an identical peptide. Thus, for example, in a preferred embodiment the multimeric inhibitor of the present invention comprises (i) 2 polypeptides each comprising, essentially consisting of, or consisting of an identical peptide having a length of at least 10 contiguous amino acids and/or not more than 150 contiguous amino acids of SEQ ID NO: 18 to SEQ ID NO: 104 or SEQ ID NO: 106 to SEQ ID NO: 143, (ii) 3 polypeptides each comprising, essentially consisting of, or consisting of an identical peptide having a length of at least 10 contiguous amino acids and/or not more than 150 contiguous amino acids of SEQ ID NO: 18 to SEQ ID NO: 104 or SEQ ID NO: 106 to SEQ ID NO: 143, or (iii) 4 polypeptides each comprising, essentially consisting of, or consisting of an identical peptide having a length of at least 10 contiguous amino acids and/or not more than 150 contiguous amino acids of SEQ ID NO: 18 to SEQ ID NO: 104 or SEQ ID NO: 106 to SEQ ID NO: 143, (iv) 5 polypeptides each comprising, essentially consisting of, or consisting of an identical peptide having a length of at least 10 contiguous amino acids and/or not more than 150 contiguous amino acids of SEQ ID NO: 18 to SEQ ID NO: 104 or SEQ ID NO: 106 to SEQ ID NO: 143, or (v) 6 polypeptides each comprising, essentially consisting of, or consisting of an identical peptide having a length of at least 10 contiguous amino acids and/or not more than 150 contiguous amino acids of SEQ ID NO: 18 to SEQ ID NO: 104 or SEQ ID NO: 106 to SEQ ID NO: 143.

The term “antibody or fragment thereof”, as used herein, refers to immunoglobulin molecules and immunologically active portions of immunoglobulin molecules, i.e. molecules that contain an antigen binding site that specifically binds an antigen. Also comprised are immunoglobulin-like proteins that are selected through techniques including, for example, phage display to specifically bind to a target molecule or target protein. The immunoglobulin molecules can be of any type (e.g., IgG, IgE, IgM, IgD, IgA and IgY), class (e.g., IgG1, IgG2, IgG3, IgG4, IgA1 and IgA2) or subclass of immunoglobulin molecule. The “antibodies and fragments thereof” include, but are not limited to, polyclonal, monoclonal, monovalent, bispecific, heteroconjugate, multispecific, human, humanized (in particular CDR-grafted), deimmunized, or chimeric antibodies, single chain antibodies (e.g. scFv), Fab fragments, F(ab′)₂ fragments, fragments produced by a Fab expression library, diabodies or tetrabodies (Holliger P. et al., 1993), nanobodies, anti-idiotypic (anti-Id) antibodies, and epitope-binding fragments of any of the above.

In some embodiments, the antibody fragments are mammalian, preferably human antigen-binding antibody fragments and include, but are not limited to, Fab, Fab′ and F(ab′)₂, Fd, single-chain Fvs (scFv), single-chain antibodies, disulfide-linked Fvs (dsFv) and fragments comprising either a VL or VH domain. Antigen-binding antibody fragments, including single-chain antibodies, may comprise the variable domain(s) alone or in combination with the entirety or a portion of the following: hinge region, CL, CH1, CH2, and CH3 domains. The antigen-binding fragments may also comprise any combination of variable domain(s) with a hinge region, CL, CH1, CH2, and CH3 domains.

Antibodies usable in the invention may be from any animal origin including birds and mammals. Preferably, the antibodies are human, simian (e.g. chimpanzee, bonobo, macaque), rodent (e.g. mouse and rat), donkey, sheep rabbit, goat, guinea pig, camel, horse, or chicken. It is particularly preferred that the antibodies are of human or murine origin. As used herein, “human antibodies” include antibodies having the amino acid sequence of a human immunoglobulin and include antibodies isolated from human immunoglobulin libraries or from animals transgenic for one or more human immunoglobulin and that do not express endogenous immunoglobulins, as described for example in U.S. Pat. No. 5,939,598 by Kucherlapati & Jakobovits.

In the context of this invention, the unique part of an antigen recognized by an antibody or fragment thereof is called an “epitope”. The different regions that an antibody comprises are well known in the art and are described e.g. in Janeway C A, Jr et al. (2001), Immunobiology, 5th ed., Garland Publishing.

As used herein, an antibody or antibody fragment is considered to “specifically bind” to a second compound (e.g. an antigen, such as a target protein), if it has a dissociation constant K_{D} to said second compound of 100 μM or less, preferably 50 μM or less, preferably 30 μM or less, preferably 20 μM or less, preferably 10 μM or less, preferably 5 μM or less, more preferably 1 μM or less, more preferably 900 nM or less, more preferably 800 nM or less, more preferably 700 nM or less, more preferably 600 nM or less, more preferably 500 nM or less, more preferably 400 nM or less, more preferably 300 nM or less, more preferably 200 nM or less, even more preferably 100 nM or less, even more preferably 90 nM or less, even more preferably 80 nM or less, even more preferably 70 nM or less, even more preferably 60 nM or less, even more preferably 50 nM or less, even more preferably 40 nM or less, even more preferably 30 nM or less, even more preferably 20 nM or less, and even more preferably 10 nM or less.

The term “CDR”, as used herein in the context of an antibody or a fragment thereof, refers to any of the antibodies complementarity determining regions. In the variable (V) domain of an antibody there are three CDRs (CDR1, CDR2 and CDR3). Since antibodies are typically composed of two polypeptide chains, there is a frequency of about six CDRs for each antigen receptor that can come into contact with the antigen (each heavy and light chain contains three CDRs). Among these, CDR3 shows the greatest variability. CDR domains have been extensively studied and, thus, the average skilled person is well capable of identifying CDR regions, i.e. CDR1, CDR2 and CDR3 within a polypeptide sequence of a VL and VH domain of an antigen receptor.

In one preferred method, the CDR1, CDR2 and CDR3 regions of the VL domain are determined as follows: CDR1 of the VL domain: The first amino acid of CDR1 is located at approx. residue 23 or 24 of the VL domain. The residue before the first amino acid of the CDR1 is a conserved Cys residue. The residues following the last amino acid of the CDR1 region is a conserved Trp residue followed typically by Tyr-Gln, but also, Leu-Gln, Phe-Gln or Tyr-Leu. The length of the CDR1 of the VL domain is between 10 and 17 residues. CDR2 of the VL domain: CDR2 is generally located 16 residues after the end of CDR1. The residues before the first amino acid of CDR2 are generally Ile-Tyr, but also, Val-Tyr, Ile-Lys, Ile-Phe or similar. The length of the CDR2 region is generally 7 residues. CDR3 of the VL domain: CDR3 region of the VL domain starts 33 residues after the end of the CDR2 region. The preceeding residue before the first amino acid of CDR3 is always Cys. CDR3 is followed by the amino acids Phe-Gly-XXX-Gly. The length of the CDR3 region is typically between 7 to 11 residues.

In one preferred method, the CDR1, CDR2 and CDR3 regions of the VH domain are determined as follows: CDR1 of the VH domain: The first amino acid of CDR1 is located at approx. residue 26 of the VH domain (always 4 or 5 residues after a Cys). The amino acid after the CDR1 will be a Trp (Typically Trp-Val, but also, Trp-Ile or Trp-Ala). The length of the CDR1 of the VH domain is between 10 to 12 residues. CDR2 of the VH domain: The CDR2 domain starts at residue 15 after the end of the CDR1 of the VH domain. The CDR2 domain is preceded typically by the amino acids Leu-Glu-Trp-Ile-Gly or a variation thereof. The CDR2 domain will be followed by the three amino acids (Lys/Arg)-(Leu/Ile/Val/Phe/Thr/Ala)-(Thr/Ser/Ile/Ala) and comprises a total of about 16 to 19 residues. CDR3 of the VH domain: The first amino acid of the CDR3 of the VH domain will be located 33 residues after the end of the CDR2 of the VH domain and will start always 3 amino acids after a conserved Cys residue (the preceding sequence is typically Cys-Ala-Arg). The residues following the CDR3 will be Trp-Gly-XXX-Gly. The CDR3 of the VH domain will typically have a length of between 3 to 25 residues.

The following Table 3 provides an overview over the preferred antibodies referred to herein:

TABLE 3

| Antibody | Target specifically bound by the Antibody |
|---|---|
| MAB CR6261 | Hemagglutinin of influenza A virus |
| MAB D5 | gp41 of HIV |
| MAB 2F5 | gp41 of HIV |
| MAB 4E10 | gp41 of HIV |
| MAB VRC01 | gp120 of HIV |
| MAB VRC02 | gp120 of HIV |
| MAB PALIVIZUMAB | Protein F of respiratory syncytial virus |
| MAB MOTAVIZUMAB | Protein F of respiratory syncytial virus |

The following Table 4 provides an overview over the preferred amino acid sequences referred to herein:

TABLE 4

| SEQ | |
|---|---|
| ID NO: | Description |
| 152 | FAB D5 LIGHT CHAIN |
| 153 | FAB D5 HEAVY CHAIN |
| 154 | LIGHT CHAIN MUTANT A (THR20CYS) OF FAB |
| | D5 FOR LIPID CONJUGATION |
| 155 | LIGHT CHAIN MUTANT B (THR22CYS) OF FAB |
| | D5 FOR LIPID CONJUGATION |
| 156 | FAB 2F5 LIGHT CHAIN |
| 157 | FAB 2F5 HEAVY CHAIN |
| 158 | LIGHT CHAIN MUTANT A (THR20CYS) OF |
| | FAB 2F5 FOR LIPID CONJUGATION |
| 159 | LIGHT CHAIN MUTANT B (THR22CYS) OF |
| | FAB 2F5 FOR LIPID CONJUGATION |
| 160 | HEAVY CHAIN CDR3 DOUBLE MUTANT OF FAB 2F5 |
| 161 | FAB 4E10 LIGHT CHAIN |
| 162 | FAB 4E10 HEAVY CHAIN |
| 163 | LIGHT CHAIN MUTANT A (THR20CYS) OF |
| | FAB 4E10 FOR LIPID CONJUGATION |
| 164 | LIGHT CHAIN MUTANT B (SER22CYS) OF |
| | FAB 4E10 FOR LIPID CONJUGATION |
| 165 | FAB VRC01 LIGHT CHAIN |
| 166 | FAB VRC01 HEAVY CHAIN |
| 167 | LIGHT CHAIN MUTANT A (ILE20CYS) OF |
| | FAB VRC01 FOR LIPID CONJUGATION |
| 168 | LIGHT CHAIN MUTANT B (SER22CYS) OF |
| | FAB VRC01 FOR LIPID CONJUGATION |
| 169 | FAB VRC02 VL |
| 170 | FAB VRC02 VH |
| 171 | VL MUTANT A (ILE20CYS) OF FAB VRC02 |
| | FOR LIPID CONJUGATION |
| 172 | VL MUTANT B (SER22CYS) OF FAB VRC02 |
| | FOR LIPID CONJUGATION |
| 173 | FAB CR6261 LIGHT CHAIN |
| 174 | FAB CR6261 HEAVY CHAIN |
| 175 | LIGHT CHAIN MUTANT A (THR19CYS) OF |
| | FAB CR6261 FOR LIPID CONJUGATION |
| 176 | LIGHT CHAIN MUTANT B (SER21CYS) OF |
| | FAB CR6261 FOL LIPID CONJUGATION |
| 177 | FAB PALIVIZUMAB LIGHT CHAIN |
| 178 | FAB PALIVIZUMAB HEAVY CHAIN |
| 179 | LIGHT CHAIN MUTANT A (THR20CYS) OF |
| | FAB PALIVIZUMAB FOR LIPIDCONJUGATION |
| 180 | LIGHT CHAIN MUTANT B (THR22CYS) OF |
| | FAB PALIVIZUMAB FOR LIPIDCONJUGATION |
| 181 | FAB MOTAVIZUMAB LIGHT CHAIN |
| 182 | FAB MOTAVIZUMAB HEAVY CHAIN |
| 183 | LIGHT CHAIN MUTANT A (THR20CYS) OF |
| | FAB MOTAVIZUMAB FOR LIPIDCONJUGATION |
| 184 | LIGHT CHAIN MUTANT B (THR22CYS) OF |
| | FAB MOTAVIZUMAB FOR LIPIDCONJUGATION |
| 185 | MAB 2F5 HEAVY CHAIN CDR3 |
| 186 | MAB 4E10 HEAVY CHAIN CDR3 |

The inventors of the present invention have identified novel multimeric inhibitors of viral fusion comprising membrane integrating lipid-conjugated antibodies or fragments thereof with improved potency. For example, single antibodies or fragments thereof capable of inhibiting fusion of an enveloped virus with the cellular membrane could be rendered more effective when comprised as multimers, e.g. dimers, trimers, or tetramers, in the multimeric inhibitor of viral fusion of the present invention and when attached to a membrane integrating lipid, e.g. cholesterol. Without being bound by theory it is assumed that antibodies that are modified by attaching them to a membrane integrating lipid exhibit an improved partition ratio between antibodies in the extracellular medium and antibodies bound to a lipid membrane such as the membrane of a cell or an enveloped virus particle, for example. As an example, the multimeric inhibitors of viral fusion comprising membrane integrating lipid-conjugated antibodies or fragments thereof preferably localize to the plasma membrane especially to lipid-raft microdomains of the plasma membrane, where they can block viral entry much more effectively. This permits the application of reduced amounts of therapeutic and prophylactic antibodies to achieve the same health benefit at a low dose than that is achieved by a respective non-modified antibody of the state of the art at a respectively larger dose.

It is preferred that at least one of said polypeptides comprised in the multimeric inhibitor of the present invention is an antibody or a fragment thereof. It is more preferred that at least 2, 3, 4, 5, or 6 of said polypeptides comprised in the multimeric inhibitor of the present invention are antibodies or fragments thereof. It is most preferred that all of said polypeptides, e.g. 2, 3, 4, 5 or 6 polypeptides, comprised in the multimeric inhibitor of the present invention are antibodies or fragments thereof.

The antibodies and fragments thereof can be modified to enhance stability and to enhance antigen binding. Factors effecting stability include exposure of hydrophobic residues that are hidden at the interface of a whole Ig molecule at the constant domain interface; hydrophobic region exposure on the Fv surface leading to intermolecular interaction; and hydrophilic residues in the interior of the Fv beta sheet or at the normal interface between VH and VL (Chowdhury et al., Engineering scFvs for Improved Stability, p. 237-254 in Recombinant Antibodies for Cancer Therapy Methods and Protocols, (Eds. Welschof and Krauss) Humana Press, Totowa, N.J., 2003.). Stability can be enhanced by substituting problematic residues impacting on stability. Such modifications can be achieved by e.g. effecting up to one, two, three, four, five, six, seven, eight, nine or up to ten single amino acid substitutions, deletions, modifications and/or insertions, preferably up to three and most preferably a single substitution, deletion, modification and/or insertion in a polypeptide chain of the antibody or fragment thereof of the invention. Techniques for enhancing single chain antibody stability taking into account problematic residues are well known in art. (Chowdhury et al., Engineering scFvs for Improved Stability, p. 237-254 in Recombinant Antibodies for Cancer Therapy Methods and Protocols, (Eds. Welschof and Krauss) Humana Press, Totowa, N.J., 2003.).

Furthermore, the antibody or fragment thereof, or the antibodies or fragments thereof comprised in the multimeric inhibitor of the present invention is (are) capable of inhibiting fusion of at least one enveloped virus, preferably at least 2, 3, or 4, enveloped viruses, with a cellular membrane (e.g. cell/plasma membrane or endosomal membrane). The inhibition of fusion of an enveloped virus with a cellular membrane of a cell (e.g. cell/plasma membrane or endosomal membrane) may occur, for example, by binding to (i) the (lipid) membrane of a cell, (ii) the (lipid) membrane of an enveloped virus, (iii) a protein associated with the (lipid) membrane of an enveloped virus, and/or (iv) a protein associated with the (lipid) membrane of a cell.

It is preferred that the at least one enveloped virus, preferably at least 2, 3, or 4 enveloped viruses, is (are) (individually) selected from the group consisting of orthomyxoviridae, paramyxoviridae, filoviridae, retroviridae, coronaviridae, bornaviridae, togaviridae, arenaviridae, herpesviridae, hepadnaviridae, flaviviridae, rhabdoviridae. More preferably, the at least one enveloped virus, preferably at least 2, 3, or 4 enveloped viruses, is (are) selected from the group (of virus genera) consisting of orthomyxovirus, paramyxovirus, filovirus, retrovirus, coronavirus, bornavirus, togavirus, arenavirus, herpesvirus, hepadnavirus, flavivirus, rhabdovirus. Most preferably, the at least one enveloped virus, preferably at least 2, 3, or 4 enveloped viruses, is (are) (individually) selected from the group consisting of Influenza virus, Parainfluenza virus, Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Herpes simplex virus (HSV), Human herpesvirus (HHV) 6A, Human herpesvirus (HHV) 6B, Cytomegalovirus, Varicella-zoster virus, Chikunguya virus, Hepatitis C virus (HCV), Rabies virus, Dengue virus (DV), West Nile virus, Junin virus, Machupo virus, Guanarito virus, Japanese encephalitis virus, Yellow fever virus, and Lassa virus.

The membrane-integrating lipid attached to the antibody or fragment thereof will in preferred embodiments allow the antibody or fragment thereof to bind to a plasma membrane via lipid rafts and/or to be internalized into a cell preferably via lipid rafts. Many enveloped viruses enter cells via lipid rafts such as the influenza virus so that it is advantageous if an antibody exhibits the ability of neutralizing such viruses not only on the cell surface but also intracellularly. Internalization can be studies by several approaches such as those described in Dyer & Benjamins, J. Neurosci. (1988) 883-891, D. C. Blakey1 et al., J. Cell Biochem. Biophys. 24-25 (1994) 175-183, Coffey et al., J. Pharmacol. Exp. Ther. 310 (2004) 896-904. The average skilled person is also well capable of testing, without undue burden, if an antibody or fragment thereof binds to a (lipid) membrane of a cell (e.g. cell/plasma membrane or endosomal membrane) or an enveloped virus (e.g. membrane of a Influenza virus, Parainfluenza virus, Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Herpes simplex virus (HSV), Human herpesvirus (HHV) 6A, Human herpesvirus (HHV) 6B, Cytomegalovirus, Varicella-zoster virus, Chikunguya virus, Hepatitis C virus (HCV), Rabies virus, Dengue virus (DV), West Nile virus, Junin virus, Machupo virus, Guanarito virus, Japanese encephalitis virus, Yellow fever virus, or Lassa virus). For such analysis various tools such as fluorescence-based methods (e.g. colocalization studies, quenching e.t.c), electron microscopy studies and the like are readily available and suitable.

The skilled person can also readily assess whether an antibody or a fragment thereof, or antibodies or fragments thereof comprised in the multimeric inhibitor of the present invention is (are) capable of inhibiting fusion of at least one enveloped virus with a cellular membrane (e.g. cell/plasma membrane or endosomal membrane), for example, via the binding mechanisms mentioned above, by (i) producing a recombinant enveloped virus capable of expressing a detectable marker protein, e.g. a green fluorescent protein (GFP), an enhanced green fluorescent protein (EGFP), or a blue fluorescent protein (BFP) within a cell, preferably a mammalian cell, e.g. a human cell, (ii) infecting a cell, preferably a mammalian cell, e.g. a human cell, with said recombinant enveloped virus, (iii) incubating said cell in the presence of a test antibody and in the absence of a test antibody (control), and (iv) assessing whether the marker protein, e.g. GFP, can be detected within said cell (e.g. within the cytosol or a component such as an endosome of said cell), for example, by fluorescence microscopy. Thus, if the antibody is capable of inhibiting fusion of at least one enveloped virus with a cellular membrane (e.g. cell/plasma membrane or endosomal membrane), no GFP can be detected within said cell (e.g. within the cytosol or a component such as an endosome of said cell) contrary to the control experiment, wherein a cell is incubated with the enveloped virus alone.

Alternatively, the skilled person can readily assess whether an antibody or a fragment thereof, or antibodies or fragments thereof comprised in the multimeric inhibitor of the present invention is (are) capable of inhibiting fusion of at least one enveloped virus with a cellular membrane (e.g. cell/plasma membrane or endosomal membrane) by (i) labelling an enveloped virus with fluorescent lipophilic dyes, e.g. by incubating the enveloped virus with 1,1′-dioctadecyl-3,3,3′,3′-tetramethylindodicarbocyanine (DiD) (Molecular probes), (ii) infecting a cell, preferably a mammalian cell, e.g. a human cell, with the fluorescent lipophilic dye-labelled virus, e.g. DiD-labelled virus, (iii) incubating said cell in the presence of a test antibody and in the absence of a test antibody (control), (iv) exciting the fluorescent lipophilic dye-labelled virus, e.g. DiD-labelled virus with a laser, e.g. a 633 nm helium-neon laser (Melles-Griot), and (v) assessing whether the lipophilic dye-labelled virus, e.g. DiD-labelled virus, can be detected within said cell (e.g. within the cytosol or a component such as an endosome of said cell) incubated in the presence and absence of a test antibody, e.g. by obtaining fluorescence images from said cell. The fluorescent lipophilic dye DiD spontaneously partitions into the viral membrane. The dye-labelled viruses are still infectious and dye-labelling does not affect the viral infectivity. The surface density of the DiD-Dye is sufficiently high so that dye-labelled viruses can be clearly detected. See for example Lakadamyali et al., “Visualizing infection of individual influenza viruses”, 2003, PNAS, Vol. 100, No. 16, pages 9280-9285). Thus, if the antibody is capable of inhibiting fusion of at least one enveloped virus with a cellular membrane (e.g. cell/plasma membrane or endosomal membrane), no fluorescence signal can be detected within said cell (e.g. within the cytosol or a component such as an endosome of said cell) contrary to the control experiment, wherein a cell is incubated with the fluorescent lipophilic dye labelled virus, e.g. DiD-labelled virus, alone. The skilled person knows about the different living cycle of the enveloped viruses referred to herein. For example, the skilled person knows that, for example, the fusion process of a paramyxovirus occurs at the surface of the cell/plasma membrane of a cell at neutral pH and that, thus, the success of the inhibition of viral fusion after antibody administration may be controlled, for example, by verifying the presence of said virus in the cytosol of said cell or that, for example, the fusion process of an influenza virus occurs at the surface of the endosomal membrane of a cell in the presence of an acidic pH and that, thus, the success of the inhibition of viral fusion after antibody administration may be controlled, for example, by verifying the presence of said virus in the endosome of said cell.

It is preferred that the antibody or fragment thereof, or the antibodies or fragments thereof comprised in the multimeric inhibitor of the present invention is (are) capable of inhibiting fusion of at least one enveloped virus by binding to a viral coat protein of at least one enveloped virus, preferably at least 2, 3, or 4 enveloped viruses, more preferably a viral fusogenic protein such as a Type I, II, or III viral fusogenic protein, and most preferably a protein or peptide selected from the group consisting of HIV gp41 (Type I fusogenic protein, e.g. accession number AAA19156.1), HIV gp120, influenza hemagglutinin (Type I fusogenic protein, e.g. accession number AAA43099.1 or CAA40728.1), protein F of paramyxoviruses (Type I fusogenic protein, e.g. accession number AAV54052.1), protein GP2 of filoviruses (Type I fusogenic protein, e.g. accession number Q89853.1 or AAV48577.1), protein E of flaviviruses (Type II of fusogenic protein, e.g. accession number AAR87742.1), protein E1 of alphaviruses (Type II of fusogenic protein), protein S of coronaviruses (Type I of fusogenic protein, e.g. accession number AAP33697.1 or BAC81404.1), protein gH of herpesviruses (Type III fusogenic protein), protein gB of herpesviruses (Type III fusogenic proteins), and protein G2 of arenaviruses (Type I fusogenic protein, e.g. accession number BAA00964.2 or P03540). HIV gp41 and HIV gp120 are part of the same protein gp160, while gp120 is the receptor-binding subunit, gp41 the fusogenic subunit. Gp41 is a Type I fusogenic protein.

It is also preferred that the antibody or fragment thereof, or the antibodies or fragments thereof comprised in the multimeric inhibitor of the present invention is (are) capable of inhibiting fusion of at least one, preferably one (an), enveloped virus by binding to a protein which is associated with the cellular membrane, preferably cell/plasma membrane or endosomal membrane, and which mediates the entry of an enveloped virus into a cell. More preferably, said protein is associated with the cellular membrane (e.g. cell/plasma membrane or endosomal membrane) and mediates the entry of an enveloped virus selected from the group consisting of Influenza virus, Parainfluenza virus, Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Herpes simplex virus (HSV), Human herpesvirus (HHV) 6A, Human herpesvirus (HHV) 6B, Cytomegalovirus, Varicella-zoster virus, Chikunguya virus, Hepatitis C virus (HCV), Rabies virus, Dengue virus (DV), West Nile virus, Junin virus, Machupo virus, Guanarito virus, Japanese encephalitis virus, Yellow fever virus, and Lassa virus into a cell. Most preferably, the protein which is associated with the cellular membrane (e.g. cell/plasma membrane or endosomal membrane) and which mediates the entry of an enveloped virus into a cell is selected from the group consisting of CD4, CCR5, CXCR4, integrins like integrin alpha-4 beta-7, glycoproteins containing sialic acid as terminal group, human angiotensin-converting enzyme 2 (ACE2), herpesvirus entry mediator (HVEM), nectin-1, proteins containing 3-0 sulfated heparan sulfate, the C-type lectins DC-SIGN and DC-SIGNR, the L-Type lectin L-SIGN, nicotinic acetylcholine receptor (nAChR), neuronal cell adhesion molecule (NCAM), p75 neurotrophin receptor (p75NTR), insulin-degrading enzyme (IDE), Ephrin B2, Ephrin B3, CD81, and scavanger receptor B1 (SR-B 1).

It is within the skill of the artisan to experimentally determine, if an antibody or fragment thereof, or antibodies or fragments thereof comprised in the multimeric inhibitor of the present invention bind(s) to an antigen such as one of the aforementioned polypeptides or proteins. For example, it is possible to analyze the interaction between the antibody or fragment thereof and the polypeptide or protein using a pull down assay. For example, the polypeptide or protein may be purified and immobilized on a solid phase such as beads. In one embodiment, the beads linked to the polypeptide may be contacted with the antibody or fragment thereof, washed and probed with a secondary antibody specific for an invariant part of the antibody or fragment thereof, available in the state of the art. Also other binding assays well known in the art and suitable to determine binding affinities between two binding partners can be used such as e.g. ELISA-based assays, fluorescence resonance energy transfer (FRET)-based assays, co-immunoprecipitation assays and plasmon-resonance assays. The binding can be detected by fluorescence means, e.g. using a fluorescently labelled secondary antibody, or enzymatically as is well known in the art. Also radioactive assays may be used to assess binding. Thus, any of the aforementioned exemplary methods can be used to determine if an antibody or fragment thereof comprised in the multimeric inhibitor of the invention binds to a specific polypeptide or protein and optionally also to determine with what dissociation constant K_{D} the antibody or fragment thereof binds the mentioned antigen. In order to further determine whether said binding results in the inhibition of fusion of an enveloped virus with a cellular membrane (e.g. cell/plasma membrane or endosomal membrane), the above mentioned assays, e.g. tracking of single lipophilic dye-labelled viruses in living cells by using fluorescence microscopy in the absence and presence of an antibody comprised in the multimeric inhibitor of the present invention, may be used.

It is further preferred that at least one of said polypeptides comprised in the multimeric inhibitor of viral fusion is an antibody or a fragment thereof, wherein the membrane integrating lipid is attached, preferably linked, more preferably covalently linked (optionally via a linker), to an amino acid comprised in a VL; VH; VL; VH1, CH2, or CH3 domain of said antibody or fragment thereof. It is more preferred that at least 2, 3, 4, 5, or 6 of said polypeptides comprised in the multimeric inhibitor of viral fusion are antibodies or fragments thereof, wherein the membrane integrating lipid is attached, preferably linked, more preferably covalently linked (optionally via a linker), to an amino acid comprised in a VL; VH; VL; VH1, CH2, or CH3 domain of said antibodies or fragments thereof. It is most preferred that all of said polypeptides, e.g. at least 2, 3, or 4 polypeptides, comprised in the multimeric inhibitor of the present invention are antibodies or fragments thereof, wherein the membrane integrating lipid is attached, preferably linked, more preferably covalently linked (optionally via a linker), to an amino acid comprised in a VL; VH; VL; VH1, CH2, or CH3 domain of said antibodies or fragments thereof.

Preferably, the amino acid is located:
N-terminal to the CDR-1 region of the VL domain of said antibody or fragment thereof,
(ii) N-terminal to the CDR-1 region of the VH domain of said antibody or fragment thereof,
(iii) within the CDR-3 region of the VL domain of said antibody or fragment thereof, or
(iv) within the CDR-3 region of the VH domain of said antibody or fragment thereof.

As described above the CDR-regions of antibodies are well characterized in the art and can be determined by the skilled person for any antibody or antibody-fragment. FIGS. 6 to 13 as shown below specify particularly preferred amino acids of the light and heavy chain of the Fab-fragment of an antibody that can be used to covalently attach the lipid (optionally via a linker).

Preferred locations of the amino acid are:

(i) at position 20 or 22 of the VL domain of said antibody or fragment thereof,

(ii) at position 19 or 21 of the VL domain of said antibody or fragment thereof,

(iii) at position 7 or 25 of the VH domain of said antibody or fragment thereof,

(iv) at position 197 of the CL domain of said antibody or fragment thereof,

(v) at position 125 of the CH1 domain of said antibody or fragment thereof,

(vi) at position 248 or 326 of the CH2 domain of said antibody or fragment thereof, or

(vii) at position 415 or 442 of the CH3 domain of said antibody or fragment thereof.

Most preferred positions are position 19, 20, 21 and 22 of the VL domain. As used herein “position” refers to the location of said amino acid within the heavy or light chain of the antibody or fragment thereof. The position specifies an amino acid which is located at the indicated number of amino acids downstream of the first N-terminal amino acid of the respective light or heavy chain of said antibody or fragment thereof. As mentioned above, several examples of preferred Fab-fragments and their respective preferred locations of the amino acid are provided in FIGS. 6 to 13 below. Using sequence alignments the average skilled artisan is well capable of determining these and the aforementioned amino acid positions within the VL or VH domain of any given antibody, preferably counted from the N-terminus of said VL or VH domain.

The above mentioned linker that may optionally be present in the multimeric inhibitor of the present invention connects the membrane integrating lipid, preferably cholesterol, with said antibody or fragment thereof, or antibodies or fragments thereof. The term “linker” is defined below. Preferred embodiments of the linker are also further described below. The term “covalently linked” refers to a covalent bond between an amino acid of the antibody or fragment thereof, or antibodies or fragments hereof and the membrane integrating lipid, e.g. cholesterol, or said linker as described in more detail below that may be placed between the antibody or fragment thereof, or antibodies or fragments thereof and said membrane integrating lipid. Preferably, the membrane integrating lipid, e.g. cholesterol, or linker is covalently linked to the antibody or fragment thereof or antibodies or fragments thereof via a bond selected from the group consisting of an amide bond, an ester bond, a thioether bond, a thioester bond, an aldehyde bond and an oxyme bond. In preferred embodiments of the multimeric inhibitor of the present invention, the membrane integrating lipid, e.g. cholesterol, is covalently linked via a free —OH, —NH₃, or —COOH group of the lipid, optionally via said linker, to the C-terminus of the light chain or heavy chain of said antibody or fragment thereof or antibodies or fragments thereof. Non-cleavable linker systems are preferred (see blow).

The antibodies or fragments thereof comprised in the multimeric inhibitor of the invention may be identical and the location of the amino acid for attachment, e.g. linkage, to the membrane integrating lipid may be identical or different, or the antibodies or fragments thereof comprised in the multimeric inhibitor of viral fusion may be different and the location of the amino acid for attachment, e.g. linkage, to the membrane integrating lipid may be identical or different. In preferred embodiments, the antibodies or fragments thereof comprised in the multimeric inhibitor of the present invention are identical and the location of the amino acid for attachment, e.g. linkage, to the membrane integrating lipid is identical.

Thus, considering the above, in a preferred embodiment, at least one polypeptide comprised in the multimeric inhibitor of viral fusion is an antibody or a fragment thereof comprising a heavy chain or light chain having an amino acid sequence selected from the group consisting of SEQ ID NO: 152 to SEQ ID NO: 186. In a more preferred embodiment, at least 2, 3, 4, 5, or 6 polypeptides comprised in the multimeric inhibitor of viral fusion are antibodies or fragments thereof comprising a heavy chain or light chain having an amino acid sequence (individually) selected from the group consisting of SEQ ID NO: 152 to SEQ ID NO: 186. In a most preferred embodiment, the multimeric inhibitor of the invention comprises 2, 3, or 4 antibodies or fragments thereof all comprising an identical heavy chain or light chain having an amino acid sequence selected from the group consisting of SEQ ID NO: 152 to SEQ ID NO: 186.

Preferably, the amino acid to which said membrane integrating lipid is attached, more preferably linked, most preferably covalently linked (optionally via a linker), to the antibody or fragment thereof, or antibodies or fragments thereof, is comprised in the light chain of said antibody or fragment thereof, or said antibodies or fragments thereof, and most preferably is comprised in a VL domain of said antibody or fragment thereof, or antibodies or fragments thereof.

Accordingly, it is further preferred that at least one of said polypeptides comprised in the multimeric inhibitor of viral fusion is an antibody or a fragment thereof comprising a heavy chain with a VH domain and a light chain with a VL domain, wherein the VH and VL domains respectively have an amino acid sequence selected from i) to xviii):

| | VH-domain | VL-domain |
|---|---|---|
| | (SEQ ID NO) | (SEQ ID NO): |
| i) | 153 | 154; |
| ii) | 153 | 155; |
| iii) | 157 | 158; |
| iv) | 157 | 159; |
| v) | 160 | 158; |
| vi) | 160 | 159; |
| vii) | 162 | 163; |
| viii) | 162 | 164; |
| ix) | 166 | 167; |
| x) | 166 | 168; |
| xi) | 170 | 171; |
| xii) | 170 | 172; |
| xiii) | 174 | 175; |
| xiv) | 174 | 176; |
| xv) | 178 | 179; |
| xvi) | 178 | 180; |
| xvii) | 182 | 183; |
| xviii) | 182 | 184; |

and wherein the light and heavy chain in total optionally comprise one, two or three single amino acid substitutions, deletions, modifications and/or insertions.

It is more preferred that at least 2, 3, 4, 5, or 6 of said polypeptides comprised in the multimeric inhibitor of viral fusion are antibodies or fragments thereof comprising a heavy chain with a VH domain and a light chain with a VL domain, wherein the VH and VL domains respectively have an amino acid sequence (individually) selected from i) to xviii):

| | VH-domain | VL-domain |
|---|---|---|
| | (SEQ ID NO) | (SEQ ID NO): |
| i) | 153 | 154; |
| ii) | 153 | 155; |
| iii) | 157 | 158; |
| iv) | 157 | 159; |
| v) | 160 | 158; |
| vi) | 160 | 159; |
| vii) | 162 | 163; |
| viii) | 162 | 164; |
| ix) | 166 | 167; |
| x) | 166 | 168; |
| xi) | 170 | 171; |
| xii) | 170 | 172; |
| xiii) | 174 | 175; |
| xiv) | 174 | 176; |
| xv) | 178 | 179; |
| xvi) | 178 | 180; |
| xvii) | 182 | 183; |
| xviii) | 182 | 184; |

and wherein the light and heavy chain in total optionally comprise one, two or three single amino acid substitutions, deletions, modifications and/or insertions.

In a most preferred embodiment, the multimeric inhibitor of the present invention comprises at least 2, 3, or 4 antibodies with identical VH and VL domains having an amino acid sequence of any of i) trough xviii) as mentioned above. For example, in a most preferred embodiment, the multimeric inhibitor of the present invention comprises two antibodies with identical VH and VL domains having an amino acid sequence selected from i) to xviii) as mentioned above, comprises three antibodies with identical VH and VL domains having an amino acid sequence selected from i) to xviii) as mentioned above, or comprises four antibodies with identical VH and VL domains having an amino acid sequence selected from i) to xviii) as mentioned above.

It is preferred that said membrane integrating lipid is attached, more preferably linked such as covalently linked (optionally via a linker), in the aforementioned embodiments (i), (iii), (v), (ix), (xi), (xv), and (xvii) to an amino acid, preferably cysteine, at position 20 of the respective VL domain of the antibody or fragment thereof, or antibodies or fragments thereof. It is preferred that said membrane integrating lipid is attached, more preferably linked such as covalently linked (optionally via a linker), in the aforementioned embodiments (ii), (iv), (vi), (viii), (x), (xii), (xvi), and (xviii) to an amino acid, preferably cysteine, at position 22 of the respective VL domain of the antibody or fragment thereof, or antibodies or fragments thereof. It is further preferred that said membrane integrating lipid is attached, more preferably linked such as covalently linked (optionally via a linker), in the aforementioned embodiment (xiii) to an amino acid, preferably cysteine, at position 19 of the VL domain of the antibody or fragment thereof, or antibodies or fragments thereof. It is further preferred that said membrane integrating lipid is attached, more preferably linked such as covalently linked (optionally via a linker), in the aforementioned embodiment (xiv) to an amino acid, preferably cysteine, at position 21 of the VL domain of the antibody or fragment thereof, or antibodies or fragments thereof.

Preferably, the aforementioned antibody/antibodies and fragment/fragments thereof is (are) also capable of specifically binding to a lipid membrane such as a lipid-raft microdomain in a plasma membrane via said membrane integrating lipid.

In a preferred embodiment of the multimeric inhibitor of the present invention, said inhibitor comprises at least one polypeptide which is an antibody selected from the group consisting of a polyclonal antibody, a monoclonal antibody, a chimeric antibody, a humanized antibody, a human antibody, a diabody, a tetrabody, a nanobody, a chimeric antibody, and a deimmunized antibody. In a more preferred embodiment of the multimeric inhibitor of the present invention, said inhibitor comprises at least 2, 3, 4, 5, or 6 polypeptides which are antibodies (individually) selected from the group consisting of a polyclonal antibody, a monoclonal antibody, a chimeric antibody, a humanized antibody, a human antibody, a diabody, a tetrabody, a nanobody, a chimeric antibody, and a deimmunized antibody. In another preferred embodiment of the multimeric inhibitor of the present invention, said inhibitor comprises at least one polypeptide which is an antibody fragment selected from the group consisting of Fab, F(ab′)₂, Fd, Fv, single-chain Fv, and disulfide-linked Fvs (dsFv). In a more preferred embodiment of the multimeric inhibitor of the present invention, said inhibitor comprises at least 2, 3, 4, 5, or 6 polypeptides which are antibody fragments (individually) selected from the group consisting of Fab, F(ab′)₂, Fd, Fv, single-chain Fv, and disulfide-linked Fvs (dsFv). Most preferably, the antibodies or fragments thereof comprised in the multimeric inhibitor of the present invention are identical. Thus, for example, in a preferred embodiment, the multimeric inhibitor of the present invention comprises at least 2, 3, or 4 identical monoclonal antibodies or polyclonal antibodies. The antibody or a fragment thereof is preferably capable of binding to a lipid membrane.

In a further preferred embodiment of the multimeric inhibitor of the present invention, said inhibitor comprises at least one polypeptide which is a monoclonal antibody or a fragment thereof selected from the group consisting of MAB F10, MAB CR6261, MAB D5, MAB 2F5, MAB 4E10, MAB VRC01, MAB VRC02, palivizumab, and motavizumab, wherein said monoclonal antibody optionally comprises one or two single amino acid substitutions, deletions, modifications and/or insertions. In a more preferred embodiment of the multimeric inhibitor of the present invention, said inhibitor comprises at least 2, 3, 4, 5, or 6 polypeptides which are monoclonal antibodies or fragments thereof (individually) selected from the group consisting of MAB F10, MAB CR6261, MAB D5, MAB 2F5, MAB 4E10, MAB VRC01, MAB VRC02, palivizumab, and motavizumab, wherein said monoclonal antibody optionally comprises one or two single amino acid substitutions, deletions, modifications and/or insertions. Most preferably, said monoclonal antibodies or fragments thereof comprised in the multimeric inhibitor of the present invention are identical. Thus, for example, in a preferred embodiment, the multimeric inhibitor of the present invention comprises at least 2, 3, or 4 identical MAB F10, MAB CR6261, MAB D5, MAB 2F5, MAB 4E10, MAB VRC01, MAB VRC02, palivizumab, or motavizumab monoclonal antibodies or fragments thereof.

As used throughout this application, the phrase “a single amino acid substitution, deletion, modification and/or insertion” of a protein or polypeptide generally refers to a modified version of the recited protein or polypeptide, e.g. one amino acid of the protein or polypeptide may be deleted, inserted, modified and/or substituted. If the polypeptide or protein comprises several single amino acid substitutions, deletions, modifications and/or insertions then the total number of such substitutions, deletions, modifications and/or insertions is indicated in each case. Said insertion is an insertion of the indicated number of single amino acids into the original polypeptide or protein. An amino acid of the protein or polypeptide may also be modified, e.g. chemically modified by the total number of modifications indicated. For example, the side chain or a free amino or carboxy-terminus of an amino acid of the protein or polypeptide may be modified by e.g. glycosylation, amidation, phosphorylation, ubiquitination, e.t.c. The chemical modification can also take place in vivo, e.g. in a host-cell, as is well known in the art. For examples, a suitable chemical modification motif, e.g. glycosylation sequence motif present in the amino acid sequence of the protein will cause the protein to be glycosylated. If the polypeptide or protein comprises one or more single amino acid substitutions, said substitutions may in each case independently be a conservative or a non-conservative substitution, preferably a conservative substitution. In a most preferred embodiment, all substitutions are of conservative nature as further defined below. In some embodiments, a substitution also includes the exchange of a naturally occurring amino acid with a not naturally occurring amino acid. A conservative substitution comprises the substitution of an amino acid with another amino acid having a chemical property similar to the amino acid that is substituted. Preferably, the conservative substitution is a substitution selected from the group consisting of:
(i) a substitution of a basic amino acid with another, different basic amino acid;
(ii) a substitution of an acidic amino acid with another, different acidic amino acid;
(iii) a substitution of an aromatic amino acid with another, different aromatic amino acid;
(iv) a substitution of a non-polar, aliphatic amino acid with another, different non-polar, aliphatic amino acid; and
(v) a substitution of a polar, uncharged amino acid with another, different polar, uncharged amino acid.

A basic amino acid is preferably selected from the group consisting of arginine, histidine, and lysine. An acidic amino acid is preferably aspartate or glutamate. An aromatic amino acid is preferably selected from the group consisting of phenylalanine, tyrosine and tryptophane. A non-polar, aliphatic amino acid is preferably selected from the group consisting of glycine, alanine, valine, leucine, methionine and isoleucine. A polar, uncharged amino acid is preferably selected from the group consisting of serine, threonine, cysteine, proline, asparagine and glutamine. In contrast to a conservative amino acid substitution, a non-conservative amino acid substitution is the exchange of one amino acid with any amino acid that does not fall under the above-outlined conservative substitutions (i) through (v).

If a protein or polypeptide comprises one or an indicated number of single amino acid deletions, then said amino acid(s) present in the reference polypeptide or protein sequence have been removed.

In yet another preferred embodiment, the antibody or fragment thereof is an antibody or fragment thereof with a CDR3 domain of the heavy chain which comprises or consists of the sequence:

| | |
|---|---|
| RRGPTTXXXXXXARGPVNAMDV | (SEQ ID NO: 185) |
| or | |
| EGTTGXXXXXXPIGAFAH; | (SEQ ID NO: 186) |

wherein X may be any amino acid and wherein the lipid is covalently bound to one of the amino acids designated as X; and

wherein said sequence according to SEQ ID NO: 185 or 186 optionally comprises one single amino acid substitution, deletion, modification and/or insertion.

Preferably, at least one antibody or fragment thereof with a CDR domain of the heavy chain which comprises or consists of the sequence according to SEQ ID NO: 185 or SEQ ID NO: 186 is comprised in the multimeric inhibitor of the present invention. More preferably, at least 2, 3, 4, 5, or 6 antibodies or fragments thereof with a CDR domain of the heavy chain which comprises or consists of the sequence according to SEQ ID NO: 185 or SEQ ID NO: 186 are comprised in the multimeric inhibitor of the present invention. Most preferably, the amino acid sequences of the antibodies or fragments thereof comprised in the multimeric inhibitor of the present invention are identical.

The following should be noted: It is preferred that at least one of said polypeptides comprised in the multimeric inhibitor of the present invention is an antibody or a fragment thereof as set out above. It is more preferred that at least 2, 3, 4, 5, or 6 of said polypeptides comprised in the multimeric inhibitor of the present invention are antibodies or fragments thereof as set out above. It is most preferred that all of said polypeptides, e.g. 2, 3, 4, 5 or 6 polypeptides, comprised in the multimeric inhibitor of the present invention are antibodies or fragments thereof as set out above. The above mentioned antibodies or fragments thereof that are comprised in the multimeric inhibitor of the present invention may be identical or different. Further, the above mentioned antibodies or fragments thereof may be combined with the above mentioned polypeptides which are no antibodies or fragments thereof, for example, polypeptides comprising or consisting of peptides from Type I, II, or III viral fusogenic proteins, e.g. peptides having an amino acid sequence according to SEQ ID NO: 18 to 104, or SEQ ID NO: 106 to SEQ ID NO: 143. It is most preferred that the multimeric inhibitor of the present invention comprises at least 2, 3, or 4 identical polypeptides which are antibodies or fragments thereof as set out above.

To exert its antiviral activity, e.g. by binding to HR1 or HR2 on the surface of the viral envelope, the inhibitor of the present invention has to be in a preferred orientation. This orientation is achieved, if the membrane integrating lipid, e.g. cholesterol, is attached at (e.g. linked such as covalently linked to) the C-terminal region or N-terminal region of the polypeptides as set out above comprised in the inhibitor of the present invention.

Thus, in a preferred embodiment of the inhibitor of the present invention, the membrane integrating lipid (optionally via a linker and/or linker amino acids) is attached, preferably linked, more preferably covalently linked, to
(i) the C-terminal region of at least one, preferably at least 2, 3, 4, 5, or 6, of said polypeptide(s), preferably at least 3, 4, 5, or 6 polypeptides, or
(ii) the N-terminal region of at least one, preferably at least 2, 3, 4, 5, or 6, of said polypeptide(s), preferably at least 3, 4, 5, or 6 polypeptides.

More preferably, the membrane integrating lipid (optionally via a linker and/or linker amino acids) is attached to
(i) the C-terminal region of any of said at least two polypeptides, preferably at least 3, 4, 5, or 6 polypeptides, or
(ii) the N-terminal region of any of said at least two polypeptides, preferably at least 3, 4, 5, or 6 polypeptides.

In a more preferred embodiment of the multimeric inhibitor of the present invention, the membrane integrating lipid (optionally via a linker and/or linker amino acids) is attached, preferably linked, more preferably covalently linked, to
(i) the C-terminal region of at least one, preferably at least 2, 3, 4, 5, or 6, of said at least two polypeptides, preferably at least 3, 4, 5, or 6 polypeptides, which comprise(s) a HR2 domain of a Type I viral fusogenic protein of at least one enveloped virus, preferably a HR2 domain according to SEQ ID NO: 18 to SEQ ID NO: 104, SEQ ID NO: 120 to SEQ ID NO: 127,
(ii) the N-terminal region of at least one, preferably at least 2, 3, 4, 5, or 6, of said at least two polypeptides, preferably at least 3, 4, 5, or 6 polypeptides, which comprise(s) a HR1 domain of a Type I viral fusogenic protein of at least one enveloped virus, preferably a HR1 domain according to SEQ ID NO: 128, or
(iii) the C-terminal region of at least one, preferably at least 2, 3, 4, 5, or 6, of said at least two polypeptides, preferably at least 3, 4, 5, or 6 polypeptides, which comprise(s) a membrane-proximal region (MPR) of a Type II viral fusogenic protein of at least one enveloped virus, preferably a membrane-proximal region (MRP) according to SEQ ID NO: 137 to SEQ ID NO: 143.

In a most preferred embodiment of the multimeric inhibitor of the present invention, the membrane integrating lipid (optionally via a linker/and or linker amino acids) is attached, preferably linked, more preferably covalently linked to
(i) the C-terminal region of said at least two polypeptides, preferably at least 3, 4, 5, or 6 polypeptides, each comprising a HR2 domain of a Type I viral fusogenic protein of at least one enveloped virus, preferably a HR2 domain according to SEQ ID NO: 18 to SEQ ID NO: 104, SEQ ID NO: 120 to SEQ ID NO: 127,
(ii) the N-terminal region of said at least two polypeptides, preferably at least 3, 4, 5, or 6 polypeptides, each comprising a HR1 domain of a Type I viral fusogenic protein of at least one enveloped virus, preferably a HR1 domain according to SEQ ID NO: 128, or
(iii) the C-terminal region of said at least two polypeptides, preferably at least 3, 4, 5, or 6 polypeptides, each comprising a membrane-proximal region (MPR) of a Type II viral fusogenic protein of at least one enveloped virus, preferably a membrane-proximal region (MRP) according to SEQ ID NO: 137 to SEQ ID NO: 143, preferably a membrane-proximal region (MRP) according to SEQ ID NO: 137 to SEQ ID NO: 143, or
(iv) the C-terminal region of at least one polypeptide (e.g. 1, 2, 3, 4, 5 or 6 polypeptides) comprising a HR2 domain of a Type I viral fusogenic protein of at least one enveloped virus, preferably a HR2 domain according to SEQ ID NO: 18 to SEQ ID NO: 104, SEQ ID NO: 120 to SEQ ID NO: 127, and the N-terminal region of at least one polypeptide (e.g. 1, 2, 3, 4, 5, or 6 polypeptides) comprising a HR1 domain of a Type I viral fusogenic protein of at least one enveloped virus, preferably a HR1 domain according to SEQ ID NO: 128.

It is possible that the inhibitor of the present invention comprises at least one polypeptide, preferably at least 2 or 3 polypeptides, as set out above, wherein the membrane integrating lipid (optionally via a linker/and or linker amino acids) is attached to the N-terminal region of said polypeptide(s) and at least one polypeptide, preferably at least 2 or 3 polypeptides, as set out above, wherein the membrane integrating lipid (optionally via a linker or linker amino acids) is attached to the C-terminal region of said polypeptide(s).

The term “C-terminal region” or N-terminal region” is used to refer to the 5 most C-terminally or N-terminally located amino acids, preferably the four most C-terminally or N-terminally located amino acids, preferably the three most C-terminally or N-terminally located amino acids, preferably the two most C-terminally or N-terminally located amino acids, or preferably the C-terminal amino acid or N-terminal amino acid. The C-terminal amino acid is generally that amino acid, which has a free carboxy group, while the N-terminal amino acid is generally that amino acid, which has a free amino group. Thus, in a preferred embodiment the membrane integrating lipid, e.g. cholesterol or a functional derivative thereof, is attached to the polypeptide(s) through the C-terminal amino acid of said polypeptide(s). In another preferred embodiment, the membrane integrating lipid, e.g. cholesterol or a functional derivative thereof, is attached to the polypeptides(s) trough the N-terminal amino acid of the said polypeptide(s). However, at least in some embodiments of the present invention as detailed further below, the free carboxy group and/or free amino group is modified, preferably to increase stability and/or biological half life, e.g. the half life in blood serum. It has been shown that the ability of synthetic peptides or synthetic protein fragments to survive the degradative action of aminopeptidases and serum proteolytic enzymes can be remarkably enhanced by modifications at their N-terminal amino group and/or C-terminal carboxy group. In such cases it is preferred that the attachment is through a side chain of the amino acid rather than through the carboxy group or amino group. In spite of such possible modification of the C-terminus and/or N-terminus the skilled person for the purpose of the determining the “C-terminal region” or the “C-terminal amino acid”, or the “N-terminal region” or the “N-terminal amino acid” is still capable of this determination by assessing the orientation of the peptide bonds between the amino acids preceding the modified C-terminal amino acid and/or N-terminal amino acid.

As indicated above, in a preferred embodiment of the inhibitor of the present invention, the C-terminal amino acid, the N-terminal amino acid, and/or one or more internal amino acids of at least one of said polypeptide(s) is (are) modified. In a more preferred embodiment of the inhibitor of the present invention,
(i) the C-terminal amino acid is modified by amidation,
(ii) the N-terminal amino acid comprises a chemical modification selected from the group consisting of one or more L-amino acids and/or D-amino acids, an acyl group, beta-alanine, 9H-fluoren-9-ylmethoxycarbonyl (Fmoc), Benzyloxy-carbonyl, and (t)ert-(B)ut(O)xy(c)arbonyl (Boc), and/or
(iii) at least two amino acids spaced by at least one amino acid apart are connected, preferably by an amide (lactam) bond, a disulfide bond, a thioether bond, or a hydrocarbon bridge between the amino acid side chains.

In another preferred embodiment the N-terminus is modified by reacting the free amino group with a mono or dicarboxylic organic acid, preferably acetic acid or succinic acid. As mentioned above, in a particularly preferred embodiment both the N-terminus and the C-terminus of the polypeptide(s) are modified. Preferred combinations are amidation at the C-terminus and acetylation or succinilation at the N-terminus. Accordingly, particularly preferred polypeptides comprised in the multimeric inhibitor of the present invention comprise an amino acid sequence as set out in SEQ ID NO: 18 to SEQ ID NO: 104 or SEQ ID NO: 106 to SEQ ID NO: 143 as defined above and comprise an acetylated or succinilated N-terminus and/or an amidated C-terminus.

The multimeric inhibitor of the present invention may comprise or consists of (i) at least two polypeptides as set out above, each preferably comprising, essentially consisting or consisting of a HR1 domain binding peptide as set out above, a HR2 domain binding peptide as set out above, or beta-sheet domain binding peptide as set out above, and a membrane integrating lipid selected from the group consisting of cholesterol, a sphingolipid, a glycolipid, a glycerophospholipid and membrane integrating derivatives thereof, (ii) at least two polypeptides as set out above, each preferably comprising, essentially consisting or consisting of a HR1 domain binding peptide as set out above, a HR2 domain binding peptide as set out above, or beta-sheet domain binding peptide as set out above, one or more linker amino acid(s), and a membrane integrating lipid selected from the group consisting of cholesterol, a sphingolipid, a glycolipid, a glycerophospholipid and membrane integrating derivatives thereof, (iii) at least two polypeptides as set out above, each preferably comprising, essentially consisting or consisting of a HR1 domain binding peptide as set out above, a HR2 domain binding peptide as set out above, or beta-sheet domain binding peptide as set out above, a linker, and a membrane integrating lipid selected from the group consisting of cholesterol, a sphingolipid, a glycolipid, a glycerophospholipid and membrane integrating derivatives thereof, or (iv) at least two polypeptides as set out above, each preferably comprising, essentially consisting of or consisting of a HR1 domain binding peptide as set out above, a HR2 domain binding peptide as set out above, or a beta-sheet domain binding peptide as set out above, one or more linker amino acid(s), a linker, and a membrane integrating lipid selected from the group consisting of cholesterol, a sphingolipid, a glycolipid, a glycerophospholipid and membrane integrating derivatives thereof, wherein said membrane integrated lipid is attached, preferably linked, more preferably covalently linked, to said polypeptides (e.g. over their C-terminal or N-terminal region) in one of the following ways: directly without an additional linker and/or one or more linking amino acid(s), via one or more linking amino acid(s), via a linker, or via a linker and one or more linking amino acid(s), respectively.

The term “covalently linked” refers to a covalent bond between an amino acid of the polypeptide(s) as set out above and the membrane integrating lipid, e.g. cholesterol, or said linker as described in more detail below that may be placed between the polypeptides as set out above and said membrane integrating lipid. Preferably, the membrane integrating lipid, e.g. cholesterol, or linker is covalently linked to the polypeptides as set out above via a bond selected from the group consisting of an amide bond, an ester bond, a thioether bond, a thioester bond, an aldehyde bond and an oxyme bond. In preferred embodiments of the inhibitor of the present invention, the membrane integrating lipid, e.g. cholesterol, is covalently linked via a free —OH, —NH₃, or —COOH group of the lipid, optionally via said linker, to the C-terminus or N-terminus of the polypeptide(s) as set out above. Non-cleavable linker systems are preferred. Examples of non-cleavable linker systems which can be used in this invention include the carbodiimide (EDC), the sulfhydryl-maleimide, and the periodate systems, which are all well known in the art. In the carbodiimide system, a water soluble carbodiimide reacts with carboxylic acid groups of the membrane integrating lipid, e.g. cholesterol, or of the antibody or fragment thereof, or antibodies or fragments thereof, resulting in the activation of this carboxyl group. The carboxyl group is subsequently coupled to an amino group present on the membrane integrating lipid, e.g. cholesterol, or antibody or fragment thereof, or antibodies or fragments thereof. The result of this reaction is a non-cleavable bond between the membrane integrating lipid, e.g. cholesterol, and the antibody or fragment thereof, or antibodies or fragments thereof. In the sulfhydryl-maleimide system, a sulfhydryl group is for example introduced onto an amine group of the antibody or fragment thereof, or antibodies or fragments thereof using a compound such as Traut's reagent. The membrane integrating lipid or linker including the membrane integrating lipid is then reacted with an NHS ester (such as gamma-maleimidobutyric acid NHS ester (GMBS)) to form a maleimide derivative that is reactive with sulfhydryl groups. The two activated compounds (e.g. antibody and lipid) are then reacted to form a covalent linkage that is non-cleavable. Periodate coupling requires the presence of oligosaccharide groups which can be present on the antibody or fragment thereof, or antibodies or fragments thereof. This allows forming active aldehyde groups from the carbohydrate groups that may be present on the antibody or fragment thereof or antibodies or fragments thereof. These groups can then be reacted with amino groups on the membrane integrating lipid or linker generating a stable conjugate. Alternatively, the periodate oxidized antibody can be reacted with a hydrazide derivative of a lipid or linker, which will also yield a stable conjugate.

It is further preferred that the membrane integrating lipid, preferably cholesterol, or the linker is attached, preferably linked, more preferably covalently linked, to a side chain of one of the amino acids in the C-terminal region or N-terminal region of the polypeptide(s) set out above, wherein preferred amino acids are naturally occurring or non-naturally occurring amino acids with chemical functionalities like —SH, —OH, —COOH—NH₂, —CH═O, —CR═O, —O—NH₂, —N═N═N, —C═C—, —NH—NH₂ groups, preferably Ser, Thr, Lys, Glu, Asp, or Cys. Preferably, said “C-terminal region” consists of the C-terminal 5 amino acids of the polypeptides of the inhibitor of the invention, or said “N-terminal region” consists of the N-terminal 5 amino acids of the polypeptides of the inhibitor of the invention.

As already mentioned above, the polypeptide(s) that form part of the inhibitor of the present invention (in addition to the peptides comprised therein) may optionally comprise one or more linker amino acid(s) at its C-terminus and/or N-terminus. Thus, in a preferred embodiment of the inhibitor of the present invention, at least one, preferably at least 2, 3, 4, 5, or 6, of said polypeptide(s), preferably at least 3, 4, 5, or 6 polypeptides, (e.g. any polypeptide comprised in the multimeric inhibitor) further comprises one or more linker amino acid(s) at its C-terminus and/or N-terminus. In such case the C-terminus or N-terminus to which the membrane integrating lipid, e.g. cholesterol or its derivative, will be linked will also comprise such linker amino acid(s). In that respect the term “linking” refers to a bond, preferably covalent bond, between a linker amino acid in the C-terminal region or N-terminal region of the polypeptide(s) and the membrane integrating lipid, e.g. cholesterol, or linker as described herein that is located between the membrane integrating lipid, e.g. cholesterol, and a linker amino acid in the C-terminal region or N-terminal region of the polypeptide(s).

The term “linker amino acids” is used herein to refer to small amino acids that preferably form unstructured domains, i.e. that do not adopt alpha-helical or beta-sheet structure, and are, thus, suitable to provide structural flexibility between the (peptides that are comprised in the) polypeptide(s) and the membrane integrating lipid, preferably cholesterol, comprised in the inhibitor of the present invention. Preferred examples of such amino acids comprise Cys, Ala, Gly, Ser and Pro. In a preferred embodiment of the inhibitor of the present invention the one or more linker amino acid(s) comprise(s) a cysteine at its (their) C-terminus and/or N-terminus. Thus, particular preferred linker amino acids are selected from the group consisting of (Gly)ₘ₊₁, (GlySerGly)ₘ, (Gly SerGlySerGly)ₘ, (GlyPro)ₘ, (Gly)ₘ₊₁Cys, (GlySerGly)ₘCys, (GlySerGlySerGly)ₘCys and (GlyPro)ₘCys, wherein m is an integer of 1 to 20, i.e. 1, 2, 3, 4, 5, 6, 7, 8, 9, 10, 11, 12, 13, 14, 15, 16, 17, 18, 19, or 20. Preferably the overall number of linker amino acids is below 50, preferably below 45, below 40, below 35, below 30, below 25, below 20, below 15, below 10, below 6, or below 5 linker amino acids, e.g. to avoid interference with the interaction with the HR domains such as HR1 or HR2 domains, or beta-sheet domains on the surface of the enveloped virus(s).

Particularly preferred polypeptides comprised in the multimeric inhibitor of the present invention comprise an amino acid sequence as set out in SEQ ID NO: 18 to SEQ ID NO: 104 or SEQ ID NO: 106 to SEQ ID NO: 143 as defined above and comprise one of the above indicated preferred linker amino acids, in particular GlySerGlySerGly, GlySerGlySerGlyCys, or GlySerGlyCys. Further particularly preferred polypeptides comprised in the multimeric inhibitor of the present invention comprise an amino acid sequence as set out in SEQ ID NO: 152 to SEQ ID NO: 186 as defined above and comprise one of the above indicated preferred linker amino acids, in particular Gly SerGlySerGly, GlySerGlySerGlyCys, or GlySerGlyCys.

Preferably, any polypeptide comprised in the multimeric inhibitor of the present invention comprises the same linker amino acids. Thus, as to the linking amino acids, the polypeptides comprised in the multimeric inhibitor of the present invention are preferably identical.

As mentioned above, said polypeptide(s) that is/are comprised in the inhibitor of the present invention are optionally attached to said membrane integrating lipid via a linker. It is preferred that the C-terminal region or N-terminal region of said polypeptide(s) that is/are comprised in the inhibitor of the present invention is attached to said membrane integrating lipid via a linker. It is more preferred that said polypeptide(s) that is/are comprised in the inhibitor of the present invention are covalently linked to said membrane integrating lipid via a linker, e.g. by their N-terminal or C-terminal region.

Thus, it is preferred that the multimeric inhibitor of viral fusion comprising, essentially consisting of, or consisting of:
(i) at least two polypeptides, preferably at least 3, 4, 5, or 6 polypeptides, capable of inhibiting fusion of at least one enveloped virus, preferably at least 2, 3, or 4 (different) enveloped viruses, with a cellular membrane, and
(ii) a membrane integrating lipid selected from the group consisting of cholesterol, a sphingolipid, a glycolipid, a glycerophospholipid and membrane integrating derivatives thereof, which is attached, preferably linked, more preferably covalently linked, to said polypeptide(s) optionally via a linker and/or via one or more linking amino acid(s),

or a pharmaceutically acceptable salt thereof.

The term “linker” preferably refers to an organic molecule that adopts a linear conformation. Typical linker may contain a polymeric spacer unit, preferably having between 1 to 30 repeats of a given monomer, and at one end of the spacer unit a moiety that allows linkage to an amino acid, preferably an amino acid containing a chemical functionality like —SH, —OH, —COOH, —NH₂, —HC═O, —RC═O, —O—NH₂, —N═N═N, —C═C—, —C═C, or —NH—NH₂, and at the other end a moiety allowing linkage to said membrane integrating lipid, e.g. cholesterol or a derivative thereof, preferably via the 3-oxygen moiety of the steroid structure.

In preferred embodiments of the present invention, said linker comprises, essentially consists, or consists of a moiety having a structure according to formula (I)

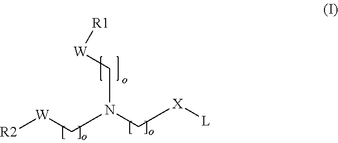

wherein each of R1 and R2 is independently selected from the group consisting of:

(i) R₃;

(ii) a structure according to formula (II):

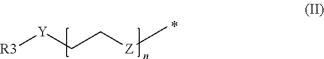

and

(iii) a structure according to formula (III):

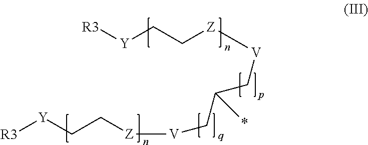

wherein

W is in each instance independently selected from —NH—C(O)—O—, —O—C(O)—NH—, —C(O)—O—, —O—C(O)—, —(CH₂)ₘ—, —NH—C(O)—, —C(O)—NH—, —NH—, and —C(X)— most preferably W is —C(O)—NH—;

V is in each instance independently selected from —(CH₂)ₘ—, —(CH₂)ₘ—C(X)—NH—, —NH—C(X)—(CH₂)ₘ—, —(CH₂)ₘ—NH—C(O)—O—, —O—C(O)—NH—(CH₂)ₘ—, —(CH₂)ₘ—C(O)—O—, —O—C(O)—(CH₂)ₘ—, —NH—C(X)—, —C(X)—NH—, —NH—C(O)—O—, —O—C(O)—NH—, —C(O)—O—, and —O—C(O)—; most preferably V is —CH₂CH₂—C(O)—NH—;

X is in each instance either O, S, or NH;

Y is in each instance independently selected from —C(O)CH₂—, —CH₂C(O)—, —NHCH₂—, —CH₂NH—, —NHC(O)—, —C(O)NH—, —NH—, —CH₂—, —CH₂C(O)NH— and —NHC(O)CH₂—; most preferably Y is —NHCH₂—;

Z is in each instance independently selected from —CH₂—, —NH—, —O—, —CH₂O—, —NHCH₂— and —OCH₂—; most preferably Z is —O—;

R₃ is in each case independently selected from any of said polypeptides, which may be the same or different;

m is in each instance independently selected from an integer of between 0 and 5, i.e. 0, 1, 2, 3, 4, or 5; preferably between 0 and 3, preferably m is the same in each instance;

n is in each instance independently selected from an integer of between 0 and 40, i.e. 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40; preferably between 3 and 10, preferably n is the same in each instance;

o is in each case independently selected from an integer of between 0 and 5, i.e. 0, 1, 2, 3, 4, or 5; preferably 2, preferably o is the same in each instance;

p is in each instance independently selected from an integer of between 0 and 5, i.e. 0, 1, 2, 3, 4, or 5; preferably between 0 and 3, preferably p is the same in each instance;

q is in each instance independently selected from an integer of between 0 and 5, i.e. 0, 1, 2, 3, 4, or 5; preferably between 0 and 3; preferably q is the same in each instance and/or preferably q≦p

L is said membrane integrating lipid; and

wherein * marks, where the structures (II-III) are linked to structure (I).

It is particularly preferred that the structure according to formula (III) has a structure according to formula (IV):

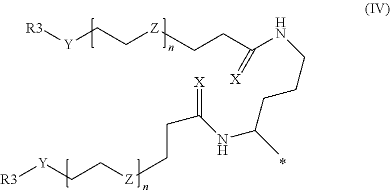

and, preferably o is 2.

More preferably, said moiety has a structure according to formula (V)

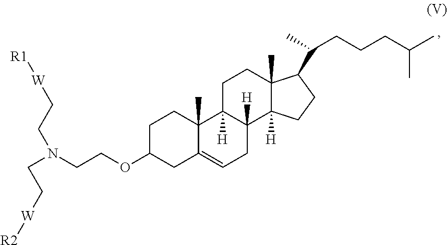

wherein

W is in each instance independently selected from —NH—C(O)—O—, —O—C(O)—NH—, —C(O)—O—, —O—C(O)—, (CH₂), —NH—C(O)—, —(O)C—NH—, and —NH—; and

m is an integer of between 0 and 3, i.e. 0, 1, 2, or 3; preferably 0.

Preferred examples of the linker of the multimeric inhibitor of viral fusion of the invention include linkers having a structure according to formulas (VI), (VII) and (VIII):

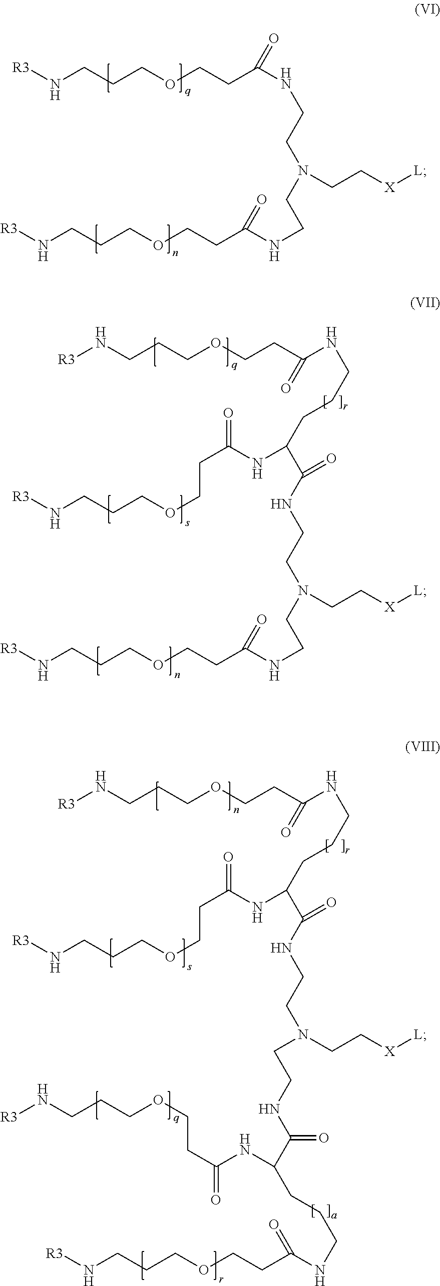

wherein

r and a are integers individually selected from 0 to 8, i.e. 0, 1, 2, 3, 4, 5, 6, 7, or 8; and

n, s, q and r are integers individually selected from 1 to 20, i.e. 1, 2, 3, 4, 5, 6, 7, 8, 9, 10, 11, 12, 13, 14, 15, 16, 17, 18, 19, or 20; preferably from 2 to 8.

As previously outlined, it is preferred that the C-terminal amino acid is Cys and that accordingly, in a preferred embodiment, the membrane integrating lipid, preferably cholesterol, or the linker is attached to the sulphur moiety of the amino acid linker or to the Cys residue that may naturally occur in the inhibitory polypeptide(s), e.g. within the HR domain such as HR1 or HR2 domain, or MPR. It is further preferred that the membrane integrating lipid or membrane integrating derivative thereof is attached via the linker to the polypeptides through the oxygen moiety at the 3 position of the cholesterol or derivative thereof.

In a preferred embodiment of the present invention, the linker comprises the following structure: NH₂—CH₂—CH₂—O—(CH₂—CH₂—O)ₙ—CH₂—CH₂—COOH, with n=1-35. Thus, a preferred linker has the structure [NH₂—CH₂—CH₂—O—(CH₂—CH₂—O)ₙ—CH₂—CH₂—CO]ₘCys wherein m is an integer of 1 to 20, i.e. 1, 2, 3, 4, 5, 6, 7, 8, 9, 10, 11, 12, 13, 14, 15, 16, 17, 18, 19, or 20, and n is an integer of 1 to 35, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35. Particularly preferred linker comprise a structure selected from the group consisting of Cys-(CH₂—CH₂—O)₄-cholesterol, Cys-(CH₂—CH₂—O)₂₄-cholesterol and NH—(CH₂—CH₂—O)₂₄—CO-Cys-(CH₂—CH₂—O)₄-cholesterol. An alternative nomenclature for these structures is C(PEG4-chol) (i.e. n=4), C(PEG24-chol) (i.e. n=24), and NH-PEG24-CO—C—(PEG4-chol) (i.e. n=4/24).

The more preferred multimeric inhibitors of the present invention are those, wherein the membrane integrating lipid, preferably cholesterol, is covalently linked through a linker as set out above in formulas (I) to (VIII) to the at least two polypeptides preferably comprising an amino sequence WX₁EWX₂REINX₃YX₄SLIX₅SLIEEX₆QX₇QQX₈KNEX₉X₁₀LX₁₁X₁₂L (SEQ ID NO: 188), a polypeptide comprising an amino acid sequence having at least 75% identity to WNEWEREINKYTSLIYSLIEEAQNQQDKNEKDLLEL (SEQ ID NO: 192) or SWETWEREIENYTRQIYRILEESQEQQDRNERDLLE (SEQ ID NO: 189) wherein X₁ to X₁₂ are defined as described above, preferably at least 3 or 4 polypeptides, and/or at least two polypeptides, preferably at least 3 or 4 polypeptides, comprising peptides having a length of at least 10 contiguous amino acids and/or not more than 150 contiguous amino acids of a sequence selected from the group consisting of SEQ ID NO: 1 to SEQ ID NO: 104, SEQ ID NO: 106 to SEQ ID NO: 143 and a sequence having at least 85% sequence identity thereto and further comprising C-terminal and/or N-terminal linker amino acids, preferably (Gly)ₘ₊₁, (GlySerGly)ₘ, (GlySerGlySerGly)ₘ, (GlyPro)ₘ, (Gly)ₘ₊₁Cys, (GlySerGly)ₘCys, (GlySerGlySerGly)ₘCys and (GlyPro)ₘCys, wherein m is an integer of 1 to 20, i.e. 1, 2, 3, 4, 5, 6, 7, 8, 9, 10, 11, 12, 13, 14, 15, 16, 17, 18, 19, or 20. The most preferred inhibitors of the present invention are those, wherein the membrane integrating lipid, preferably cholesterol, is linked through a linker as set out above in formulas (I) to (VIII) to the one ore more polypeptides, preferably at least 3 or 4 polypeptides, having a sequence selected from the group consisting of SEQ ID NO: 188, a sequence having at least 75% identity to SEQ ID NO: 192 and SEQ ID NO: 189 and/or SEQ ID NO: 1 to SEQ ID NO: 104, SEQ ID NO: 106 to SEQ ID NO: 143 and a sequence having at least 85% sequence identity thereto and further comprising C-terminal and/or N-terminal linker amino acids, preferably (Gly)ₘ₊₁, (GlySerGly)ₘ, (GlySerGlySerGly)ₘ, (GlyPro)ₘ, (Gly)ₘ₊₁Cys, (GlySerGly)ₘCys, (GlySerGlySerGly)ₘCys and (GlyPro)ₘCys, wherein m is an integer of 1 to 20, i.e. 1, 2, 3, 4, 5, 6, 7, 8, 9, 10, 11, 12, 13, 14, 15, 16, 17, 18, 19, or 20. Further most preferred multimeric inhibitors of the present invention are those, wherein the membrane integrating lipid, preferably cholesterol, is linked through a linker as set out above in formulas (I) to (VIII) to the at least two polypeptides preferably comprising peptides sequence selected from the group consisting of SEQ ID NO: 188 as defined above, a sequence having at least 75% identity to SEQ ID NO: 192 and SEQ ID NO: 189 and/or peptides having a sequence selected from the group consisting of SEQ ID NO: 152 to SEQ ID NO: 186 and further comprising C-terminal and/or N-terminal linker amino acids, preferably (Gly)ₘ₊₁, (GlySerGly)ₘ, (GlySerGlySerGly)ₘ, (GlyPro)ₘ, (Gly)ₘ₊₁Cys, (GlySerGly)ₘCys, (GlySerGlySerGly)ₘCys and (GlyPro)ₘCys, wherein m is an integer of 1 to 20, i.e. 1, 2, 3, 4, 5, 6, 7, 8, 9, 10, 11, 12, 13, 14, 15, 16, 17, 18, 19, or 20. Further most preferred inhibitors of the present invention are those, wherein the membrane integrating lipid, preferably cholesterol, is linked through a linker with a structure [NH₂—CH₂—CH₂—O—(CH₂—CH₂—O)ₙ—CH₂—CH₂—CO]ₘCys wherein m is an integer of 1 to 20, i.e. 1, 2, 3, 4, 5, 6, 7, 8, 9, 10, 11, 12, 13, 14, 15, 16, 17, 18, 19, or 20, and n is an integer of 1 to 35, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 to the at least two polypeptides comprising peptides having a sequence selected from the group consisting of SEQ ID NO: 188 as defined above, a sequence having at least 75% identity to SEQ ID NO: 192 and SEQ ID NO: 189 and/or comprising peptides having a sequence selected from the group consisting of SEQ ID NO: 152 to SEQ ID NO: 186. Particularly preferred inhibitors of the present invention are those which comprise a structure selected from the group consisting of Cys-(CH₂—CH₂—O)₄-cholesterol, Cys-(CH₂—CH₂—O)₂₄-cholesterol and NH—(CH₂—CH₂—O)₂₄—CO-Cys-(CH₂—CH₂—O)₄-cholesterol linked to the at least two polypeptides comprising peptides having a sequence selected from the group consisting of SEQ ID NO: 188 as defined above, a sequence having at least 75% identity to SEQ ID NO: 192 and SEQ ID NO: 189 and/or comprising peptides having a sequence selected from the group consisting of SEQ ID NO: 152 to SEQ ID NO: 186

The more preferred inhibitors of the eighth aspect of the present invention are those, wherein the membrane integrating lipid, preferably cholesterol, is covalently linked through a linker to the polypeptide comprising an amino sequence WX₁EWX₂REINX₃YX₄SLIX₅SLIEEX₆QX₇QQX₈KNEX₉X₁₀LX₁₁X₁₂L (SEQ ID NO: 188), a polypeptide comprising an amino acid sequence having at least 75% identity to WNEWEREINKYTSLIYSLIEEAQNQQDKNEKDLLEL (SEQ ID NO: 192) or SWETWEREIENYTRQIYRILEESQEQQDRNERDLLE (SEQ ID NO: 189) wherein X₁ to X₁₂ are defined as described above, and further comprising C-terminal and/or N-terminal linker amino acids, preferably (Gly)ₘ₊₁, (GlySerGly)ₘ, (GlySerGlySerGly)ₘ, (GlyPro)ₘ, (Gly)ₘ₊₁Cys, (GlySerGly)ₘCys, (GlySerGlySerGly)ₘCys and (GlyPro)ₘCys, wherein m is an integer of 1 to 20, i.e. 1, 2, 3, 4, 5, 6, 7, 8, 9, 10, 11, 12, 13, 14, 15, 16, 17, 18, 19, or 20.

In a second aspect, the present invention relates to a pharmaceutical composition comprising the multimeric inhibitor according to the first aspect of the present invention or a pharmaceutically acceptable salt thereof and a pharmaceutically acceptable excipient.

In a ninth aspect, the present invention relates to a pharmaceutical composition comprising the monomeric inhibitor according to the eighth aspect of the present invention or a pharmaceutically acceptable salt thereof and a pharmaceutically acceptable excipient.

For preparing pharmaceutical compositions comprising the inhibitors of the present invention, pharmaceutically acceptable excipient can be either solid or liquid. Solid form preparations include powders, tablets, pills, capsules, cachets, suppositories, and dispersible granules. A solid excipient can be one or more substances, which may also act as diluents, flavoring agents, binders, preservatives, tablet disintegrating agents, or an encapsulating material.

In powders, the excipient is preferably a finely divided solid, which is in a mixture with the finely divided inhibitor of the present invention. In tablets, the inhibitor of the present invention is mixed with the carrier having the necessary binding properties in suitable proportions and compacted in the shape and size desired.

The powders and tablets preferably contain from 5% to 80%, more preferably from 20% to 70% of the active compound. Suitable excipients are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration. As the polypeptide(s) comprised in the inhibitors of the present invention are prone to degradation by proteases it is preferred that any oral administration form retards the release of the inhibitors of the present invention to the lower intestinal tract, wherein protease activity is reduced.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Preferred administration forms are liquid form preparations include solutions, suspensions, and emulsions, for example, water or water/propylene glycol solutions. For parenteral injection, liquid preparations can be formulated in solution in, e.g. aqueous polyethylene glycol solution.

The pharmaceutical preparation is preferably in unit dosage form. In such form the preparation may be subdivided into unit doses containing appropriate quantities of the active component. The unit dosage form can be a packaged preparation, the package containing discrete quantities of preparation, such as packed tablets, capsules, and powders in vials or ampoules. Also, the unit dosage form can be a capsule, an injection vial, a tablet, a cachet, or a lozenge itself, or it can be the appropriate number of any of these in packaged form.

Certain amounts of the pharmaceutical composition according to the invention are preferred for treating or preventing a disease (e.g. infections by an enveloped virus), for example, between 5 and 400 mg more preferably between 10 and 375 mg and most preferably between 20 and 100 mg of an pharmaceutical composition comprising an multimeric inhibitor wherein at least one polypeptide is an antibody or a fragment thereof per m² body surface of the patient. It is, however, understood that depending on the severity of the disease, the type of the disease, as well as on the respective patient to be treated, e.g. the general health status of the patient, etc., different doses of the pharmaceutical composition according to the invention are required to elicit a therapeutic effect. The determination of the appropriate dose lies within the discretion of the attending physician.

In a third aspect, the present invention relates to a (broad-spectrum) multimeric inhibitor according to the first aspect of the present invention or a pharmaceutically acceptable salt thereof for the treatment or prevention of infection(s), preferably at least 2, 3, or 4 (different) infections, by (an) enveloped virus(es), preferably at least 2, 3, or 4 enveloped viruses. Preferably, the enveloped virus(es) is (are) (individually) selected from the group (of virus families) consisting of orthomyxoviridae, paramyxoviridae, filoviridae, retroviridae, coronaviridae, bornaviridae, togaviridae, arenaviridae, herpesviridae, hepadnaviridae, flaviviridae, and rhabdoviridae. More preferably, the enveloped virus(es) is (are) (individually) selected from the group (of virus genera) consisting of orthomyxovirus, paramyxovirus, filovirus, retrovirus, coronavirus, bornavirus, togavirus, arenavirus, herpesvirus, hepadnavirus, flavivirus, and lyssavirus. Most preferably, the enveloped virus(es) is (are) (individually) selected from the group (of viruses) consisting of Influenza virus, Parainfluenza virus, e.g. type 1 to 4 (HPIV1, HPIV2, HPIV3 or HPIV4), Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Herpes simplex virus (HSV), Human herpesvirus (HHV) 6A, Human herpesvirus (HHV) 6B, Cytomegalovirus, Varicella-zoster virus, Chikunguya virus, Hepatitis C virus (HCV), Rabies virus, Dengue virus (DV), West Nile virus, Junin virus, Machupo virus, Guanarito virus, Japanese encephalitis virus, Yellow fever virus, and Lassa virus.

In a further aspect, the present invention relates to the use of a (broad-spectrum) multimeric inhibitor according to the first aspect of the present invention, or a pharmaceutically acceptable salt thereof, or the use of a monomeric inhibitor according to the eighth aspect of the present invention, or a pharmaceutically acceptable salt thereof, for the production of a medicament for treating or preventing infection(s), preferably at least 2, 3, or 4 (different) infections, by (an) enveloped virus(es), preferably at least 2, 3, or 4 enveloped viruses. In a further aspect, the present invention relates to a (broad-spectrum) multimeric inhibitor according to the first aspect of the present invention, or a pharmaceutically acceptable salt thereof, or to a monomeric inhibitor according to the eighth aspect of the present invention, or a pharmaceutically acceptable salt thereof, for use in treating or preventing infection(s), preferably at least 2, 3, or 4 (different) infections, by (an) enveloped virus(es), preferably at least 2, 3, or 4 enveloped viruses. In a further aspect, the present invention relates to the use of a (broad-spectrum) multimeric inhibitor according to the first aspect of the present invention, or a pharmaceutically acceptable salt thereof, or the use of a monomeric inhibitor according to the eighth aspect of the present invention, or a pharmaceutically acceptable salt thereof, in a method of treating or preventing infection(s), preferably at least 2, 3, or 4 (different) infections, by (an) enveloped virus(es), preferably at least 2, 3, or 4 enveloped viruses. Preferably, the enveloped virus(es) is (are) (individually) selected from the group (of virus families) consisting of orthomyxoviridae, paramyxoviridae, filoviridae, retroviridae, coronaviridae, bornaviridae, togaviridae, arenaviridae, herpesviridae, hepadnaviridae, flaviviridae, and rhabdoviridae. More preferably, the enveloped virus(es) is (are) (individually) selected from the group (of virus genera) consisting of orthomyxovirus, paramyxovirus, filovirus, retrovirus, coronavirus, bornavirus, togavirus, arenavirus, herpesvirus, hepadnavirus, flavivirus, and lyssavirus. Most preferably, the enveloped virus(es) is (are) (individually) selected from the group (of viruses) consisting of Influenza virus, Parainfluenza virus, e.g. type 1 to 4 (HPIV1, HPIV2, HPIV3 or HPIV4), Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Herpes simplex virus (HSV), Human herpesvirus (HHV) 6A, Human herpesvirus (HHV) 6B, Cytomegalovirus, Varicella-zoster virus, Chikunguya virus, Hepatitis C virus (HCV), Rabies virus, Dengue virus (DV), West Nile virus, Junin virus, Machupo virus, Guanarito virus, Japanese encephalitis virus, Yellow fever virus, and Lassa virus.

Preferably, in case of the monomeric inhibitor according to the eighth aspect of the present invention, or a pharmaceutically acceptable salt thereof, the enveloped virus is Human immunodeficiency virus (HIV).

In a fourth aspect, the present invention relates to a method for making a broad-spectrum multimeric inhibitor of viral fusion effective against at least two, preferably three or four, different enveloped viruses, wherein the method comprises the steps of:
(i) generating at least two polypeptides each comprising, essentially consisting of, or consisting of a peptide as defined in the first aspect, and/or wherein at least one of said peptides is a hybrid peptide which is capable of inhibiting fusion of at least two, preferably three or four, different enveloped viruses by binding to a HR1 domain or HR2 domain of a Type I viral fusogenic protein of said enveloped viruses selected from the group consisting of HR domains with an amino acid sequence according to SEQ ID NO: 1 to SEQ ID NO: 17, SEQ ID NO: 105, and SEQ ID NO: 144 to SEQ ID NO: 151, and wherein said hybrid peptide comprises amino acids from HR domains of a Type I viral fusogenic protein of at least two different enveloped viruses; and
(ii) covalently linking a membrane integrating lipid selected from the group consisting of cholesterol, a sphingolipid, a glycolipid, a glycerophospholipid and membrane integrating derivatives thereof to the C-terminal or N-terminal region of said polypeptides.

Preferably, the present invention relates to a method for making a broad-spectrum multimeric inhibitor of viral fusion effective against at least two, preferably three or four, different enveloped viruses, wherein the method comprises the steps of:
(i) generating at least two polypeptides each comprising, essentially consisting of, or consisting of a peptide as defined in the first aspect, and/or wherein said peptides are hybrid peptides which are capable of inhibiting fusion of at least two, preferably three or four, different enveloped viruses by binding to a HR1 domain or HR2 domain of a Type I viral fusogenic protein of said enveloped viruses selected from the group consisting of HR domains with an amino acid sequence according to SEQ ID NO: 1 to SEQ ID NO: 17, SEQ ID NO: 105, and SEQ ID NO: 144 to SEQ ID NO: 151, and wherein said hybrid peptides comprise amino acids from HR domains of a Type I viral fusogenic protein of at least two different enveloped viruses; and
(ii) covalently linking a membrane integrating lipid selected from the group consisting of cholesterol, a sphingolipid, a glycolipid, a glycerophospholipid and membrane integrating derivatives thereof to the C-terminal or N-terminal region of said polypeptides.

It is preferred that the at least two, preferably three or four, different enveloped viruses are viruses of the family of paramyxoviridae and/or orthomyxoviridae. It is more preferred that the at least two, preferably three or four, different enveloped viruses are (individually) selected from the group consisting of Influenza virus, Parainfluenza virus, Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Rabies virus, Junin virus, Machupo virus, Guanarito virus, and Lassa virus.

In a fifth aspect, the present invention relates to a method for making a broad-spectrum multimeric inhibitor of viral fusion effective against at least two, preferably three or four, different enveloped viruses, wherein the method comprises the steps of:
(i) generating at least two polypeptides each comprising, essentially consisting of, or consisting of a peptide as defined in the first aspect, and/or wherein at least one of said peptides is a hybrid peptide which is capable of inhibiting fusion of at least two, preferably three or four, different enveloped viruses by binding to a beta-sheet domain of a Type II viral fusogenic protein of said enveloped viruses selected from the group consisting of Dengue virus, West Nile virus, Yellow fever virus, and Japanese encephalitis virus, and wherein said hybrid peptide comprises amino acids from membrane-proximal regions (MPRs) of a Type II viral fusogenic protein of at least two different enveloped viruses selected from the group consisting of MPRs with an amino acid sequence according to SEQ ID NO: 137 to SEQ ID NO: 143; and
(ii) covalently linking a membrane integrating lipid selected from the group consisting of cholesterol, a sphingolipid, a glycolipid, a glycerophospholipid and membrane integrating derivatives thereof to the C-terminal region of said polypeptides.

Preferably, the present invention relates to a method for making a broad-spectrum multimeric inhibitor of viral fusion effective against at least two, preferably three or four, different enveloped viruses, wherein the method comprises the steps of:
(i) generating at least two polypeptides each comprising, essentially consisting of, or consisting of a peptide as defined in the first aspect, and/or wherein said peptides are hybrid peptides which are capable of inhibiting fusion of at least two, preferably three or four, different enveloped viruses by binding to a beta-sheet domain of a Type II viral fusogenic protein of said enveloped viruses selected from the group consisting of Dengue virus, West Nile virus, Yellow fever virus, and Japanese encephalitis virus, and wherein said hybrid peptides comprise amino acids from membrane-proximal regions (MPRs) of a Type II viral fusogenic protein of at least two different enveloped viruses selected from the group consisting of MPRs with an amino acid sequence according to SEQ ID NO: 137 to SEQ ID NO: 143; and
(ii) covalently linking a membrane integrating lipid selected from the group consisting of cholesterol, a sphingolipid, a glycolipid, a glycerophospholipid and membrane integrating derivatives thereof to the C-terminal region of said polypeptides.

In a sixth aspect, the present invention relates to a method for making a broad-spectrum multimeric inhibitor of viral fusion effective against at least two, preferably three or four, different enveloped viruses, wherein the method comprises the steps of:
(i) generating at least two polypeptides each comprising, essentially consisting of, or consisting of a peptide as defined in the first aspect, and/or wherein at least one of said peptides is a hybrid peptide which is capable of inhibiting fusion of at least two, preferably three or four, different enveloped viruses by binding to a HR domain of a Type III viral fusogenic protein of said enveloped viruses selected from the group consisting of Herpes simplex virus (HSV), Human herpesvirus 6A; Human herpesvirus 6B, and Cytomegalovirus, and wherein said hybrid peptide comprises amino acids from HR domains of a Type III viral fusogenic protein of at least two different enveloped viruses selected from the group consisting of HR domains with an amino acid sequence according to SEQ ID NO: 129 to SEQ ID NO: 136; and
(ii) covalently linking a membrane integrating lipid selected from the group consisting of cholesterol, a sphingolipid, a glycolipid, a glycerophospholipid and membrane integrating derivatives thereof to the C-terminal or N-terminal region of said polypeptides.

Preferably, the present invention relates to a method for making a broad-spectrum multimeric inhibitor of viral fusion effective against at least two, preferably three or four, different enveloped viruses, wherein the method comprises the steps of:
(i) generating at least two polypeptides each comprising, essentially consisting of, or consisting of a peptide as defined in the first aspect, and/or wherein said peptides are hybrid peptides which are capable of inhibiting fusion of at least two, preferably three or four, different enveloped viruses by binding to a HR domain of a Type III viral fusogenic protein of said enveloped viruses selected from the group consisting of Herpes simplex virus (HSV), Human herpesvirus 6A; Human herpesvirus 6B, and Cytomegalovirus, and wherein said hybrid peptides comprise amino acids from HR domains of a Type III viral fusogenic protein of at least two different enveloped viruses selected from the group consisting of HR domains with an amino acid sequence according to SEQ ID NO: 129 to SEQ ID NO: 136; and
(ii) covalently linking a membrane integrating lipid selected from the group consisting of cholesterol, a sphingolipid, a glycolipid, a glycerophospholipid and membrane integrating derivatives thereof to the C-terminal or N-terminal region of said polypeptides.

In a seventh aspect, the present invention relates to a method for making a broad-spectrum multimeric inhibitor of viral fusion effective against at least two, preferably three or four, different enveloped viruses, wherein the method comprises the steps of:
(i) generating at least two polypeptides each comprising, essentially consisting of, or consisting of a peptide as defined in the first aspect, and/or wherein at least one of said peptides is a hybrid peptide which is capable of inhibiting fusion of at least two, preferably three or four, different enveloped viruses by binding to a HR1 domain or HR2 domain of a Type I viral fusogenic protein of said enveloped viruses selected from the group consisting of Influenza virus, Parainfluenza virus, Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Rabies virus, Junin virus, Machupo virus, Guanarito virus, and Lassa virus, and wherein said hybrid peptide comprises amino acids from HR domains of a Type I viral fusogenic protein of at least two different enveloped viruses selected from the group consisting of HR domains with an amino acid sequence according to SEQ ID NO: 18 to SEQ ID NO: 34, SEQ ID NO: 50 to SEQ ID NO: 54, SEQ ID NO: 83 to SEQ ID NO: 99, SEQ ID NO: 102 to SEQ ID NO: 104, and SEQ ID NO: 120 to SEQ ID NO: 128; and
(ii) covalently linking a membrane integrating lipid selected from the group consisting of cholesterol, a sphingolipid, a glycolipid, a glycerophospholipid and membrane integrating derivatives thereof to the C-terminal or N-terminal region of said polypeptides.

Preferably, the present invention relates to a method for making a broad-spectrum multimeric inhibitor of viral fusion effective against at least two, preferably three or four, different enveloped viruses, wherein the method comprises the steps of:
(i) generating at least two polypeptides each comprising, essentially consisting of, or consisting of a peptide as defined in the first aspect, and/or wherein said peptides are hybrid peptides which are capable of inhibiting fusion of at least two, preferably three or four, different enveloped viruses by binding to a HR1 domain or HR2 domain of a Type I viral fusogenic protein of said enveloped viruses selected from the group consisting of Influenza virus, Parainfluenza virus, Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Rabies virus, Junin virus, Machupo virus, Guanarito virus, and Lassa virus, and wherein said hybrid peptides comprise amino acids from HR domains of a Type I viral fusogenic protein of at least two different enveloped viruses selected from the group consisting of HR domains with an amino acid sequence according to SEQ ID NO: 18 to SEQ ID NO: 34, SEQ ID NO: 50 to SEQ ID NO: 54, SEQ ID NO: 83 to SEQ ID NO: 99, SEQ ID NO: 102 to SEQ ID NO: 104, and SEQ ID NO: 120 to SEQ ID NO: 128; and
(ii) covalently linking a membrane integrating lipid selected from the group consisting of cholesterol, a sphingolipid, a glycolipid, a glycerophospholipid and membrane integrating derivatives thereof to the C-terminal or N-terminal region of said polypeptides.

In an eleventh aspect, the present invention relates for making a monomeric inhibitor of viral fusion effective against at an enveloped virus, wherein the method comprises the steps of:
(i) generating a polypeptide comprising, essentially consisting of, or consisting of a peptide as defined in the eighth aspect which is capable of inhibiting fusion of an enveloped virus, preferably HIV, and
(ii) covalently linking a membrane integrating lipid selected from the group consisting of cholesterol, a sphingolipid, a glycolipid, a glycerophospholipid and membrane integrating derivatives thereof to the C-terminal or N-terminal region of said polypeptides.

Preferably each peptide comprised in the polypeptide(s) (e.g. 2, 3, 4, 5, or 6) generated in step (i) of the above mentioned methods is a peptide which is capable of inhibiting fusion of at least one enveloped virus, more preferably (i) by binding to a HR1 domain or HR2 domain of a Type I viral fusogenic protein of said at least one enveloped viruses selected from the group consisting of HR domains with an amino acid sequence according to SEQ ID NO: 1 to SEQ ID NO: 17, SEQ ID NO: 105, and SEQ ID NO: 144 to SEQ ID NO: 151, (ii) by binding to a beta-sheet domain of a Type II viral fusogenic protein of said at least one enveloped viruses selected from the group consisting of Chikunguya virus, Dengue virus, West Nile virus, Hepatitis C virus, Yellow fever virus, and Japanese encephalitis virus, (iii) by binding to a HR domain of a Type III viral fusogenic protein of said at least one enveloped viruses selected from the group consisting of Herpes simplex virus (HSV), Human herpesvirus 6A; Human herpesvirus 6B, Varicella-zoster virus and Cytomegalovirus, or (iv) by binding to a HR1 domain or HR2 domain of a Type I viral fusogenic protein of said at least one enveloped viruses selected from the group consisting of Influenza virus, Parainfluenza virus, Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Rabies virus, Junin virus, Machupo virus, Guanarito virus, and Lassa virus.

It is further preferred that at least two, preferably at least 3, 4, 5, or 6, more preferably at least 3, or 4, polypeptides each comprising a peptide, wherein at least one, preferably at least 2, 3, 4, 5, or 6, more preferably at least 2, 3, or 4, of said peptides (e.g. each peptide comprised in said polypeptide(s) is (are) (a) hybrid peptide(s) which is (are) capable of inhibiting fusion of at least two, preferably three or four, different enveloped viruses are generated in step (i) of the methods mentioned above. It is more preferred that at least two, preferably at least 3, 4, 5, or 6, more preferably at least 3, or 4, polypeptides each comprising a peptide, wherein said peptides are hybrid peptides which are capable of inhibiting fusion of at least two, preferably three or four, different enveloped viruses are generated in step (i) of the methods mentioned above. The hybrid peptides comprised in said polypeptides may be different or identical. In preferred embodiments of the above mentioned methods, polypeptides (e.g. 2, 3, 4, 5, or 6) are generated that each comprise, essentially consist of, or consist of identical hybrid peptides.

It is possible that the membrane integrating lipid is covalently linked to the N-terminal region of at least one polypeptide, preferably at least 2, or 3 polypeptides, as set out above, and covalently linked to the C-terminal region of at least one other polypeptide, preferably at least 2, or 3 other polypeptides, in step (ii) of the above mentioned methods. In this way, for example, a broad spectrum multimeric inhibitor that comprises at least one polypeptide (e.g. a HR1 binding domain), preferably at least 2 or 3 polypeptides, as set out above, wherein the membrane integrating lipid is covalently linked to the N-terminal region of said polypeptide(s) and at least one polypeptide (e.g. a HR2 binding domain), preferably at least 2, or 3 polypeptides, as set out above, wherein the membrane integrating lipid (optionally via a linker or linker amino acids) is covalently linked to the C-terminal region of said polypeptide(s) may be generated.

A further aspect of the invention is an inhibitor producible/obtainable according to the above mentioned methods of the invention.

HR2 domain hybrid peptides producible/obtainable (produced/obtained) by the method according to the fourth or seventh aspect of the present invention are outlined in Table 2 below as SEQ ID NO: 35 to SEQ ID NO: 49, SEQ ID NO: 55 to SEQ ID NO: 82 and SEQ ID NO: 100 to SEQ ID NO: 101.

As to the polypeptides comprising, or consisting of peptides which may be used as a starting bases for conducting the above mentioned method and as to the definition of specific terms mentioned in the method steps, e.g. as to the definition of the terms “polypeptides”, “peptides”, “hybrid peptide”, “HR domain”, “MPR”, “membrane integrating lipid”, etc., it is referred to the first to third aspect of the present invention.

A peptide (i) having a length of at least 10 contiguous amino acids of SEQ ID NO: 187 (VALDPIDISIVLNKAKSDLEESKEWIRRSNGKLDSI) or of a sequence having at least 85%, preferably 90%, more preferably 95%, and most preferably 98% or 99%, i.e. 85, 86, 87, 88, 89, 90, 91, 92, 93, 94, 95, 96, 97, 98, or 99%, sequence identity thereto, or (ii) having the sequence of SEQ ID NO: 187 or of a sequence having at least 85%, preferably 90%, more preferably 95%, and most preferably 98% or 99%, i.e. 85, 86, 87, 88, 89, 90, 91, 92, 93, 94, 95, 96, 97, 98, or 99%, sequence identity thereto can further be used in the context of the present invention. Said peptide may be comprised in the polypeptide(s) of the multimeric inhibitor of the present invention. It can also be used as a base material for the generation of broad spectrum multimeric inhibitors comprising hybrid peptides. It is from a HR2 domain and inhibits fusion of at least one enveloped virus by binding to a HR1 domain of a Type I viral fusogenic protein.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

The following Figures are merely illustrative of the present invention and should not be construed to limit the scope of the invention as indicated by the appended claims in any way.

### BRIEF DESCRIPTION OF THE DRAWINGS

FIG. 1: Generic structure for a fusion inhibitor with two identical peptide chains and a cholesterol group. Two of the possible connections between the PEG chains attached to cholesterol and the two peptide chains are shown, both featuring a thioether bond between the thiol group of a cysteine residue and a thiol-reactive moiety on the core structure.

FIGS. 2 to 5: Preferred peptide sequences that may be comprised in a broad-spectrum antiviral agent of the invention. “- - - ” may be any three amino acids, preferably PSD or no amino acids. Amino acids that can be used to substitute the respective amino acids of the reference sequence “VALDPIPSDDISIELNKAKSDLEESKEWIRRSNGKLDSI” or “VALDPIDISIELNK AKSDLEESKEWIRRSNGKLDSI (if “- - - ” means no amino acids at the indicated position) are indicated below the reference sequence. Thus, for example, in FIG. 2, the amino acid at position 1 may be any amino acid selected from the group consisting of Val, Leu and Tyr. As another example, in FIG. 2, the amino acid at position 2 may be any amino acid selected from the group consisting of Ala, Ser, Asp, Tyr and Phe.

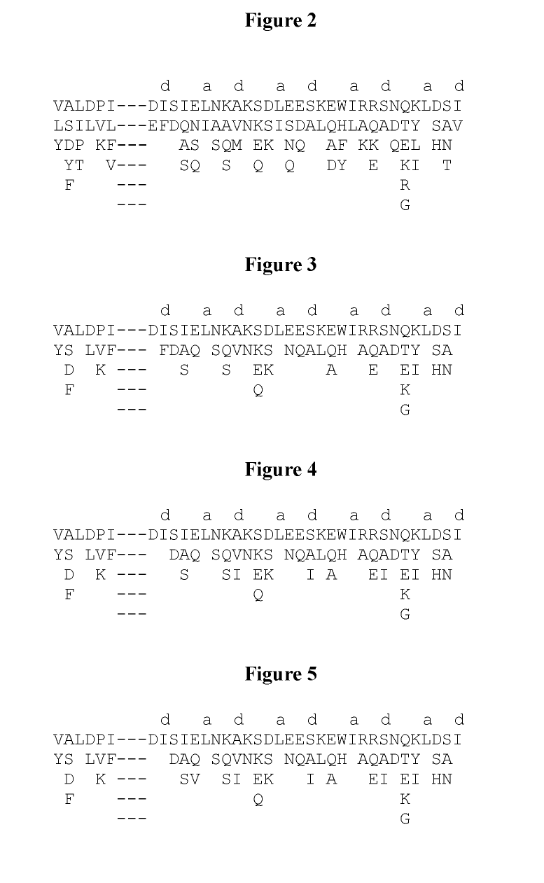

FIG. 6: Illustrates the preferred and optimized locations of cysteine amino acids in the Fab domain of MAB D5, that are ideally positioned for covalent linkage to a membrane integrating lipid or a linker including a membrane integrating lipid according to the invention. (*) marks introduced cysteine amino acids.

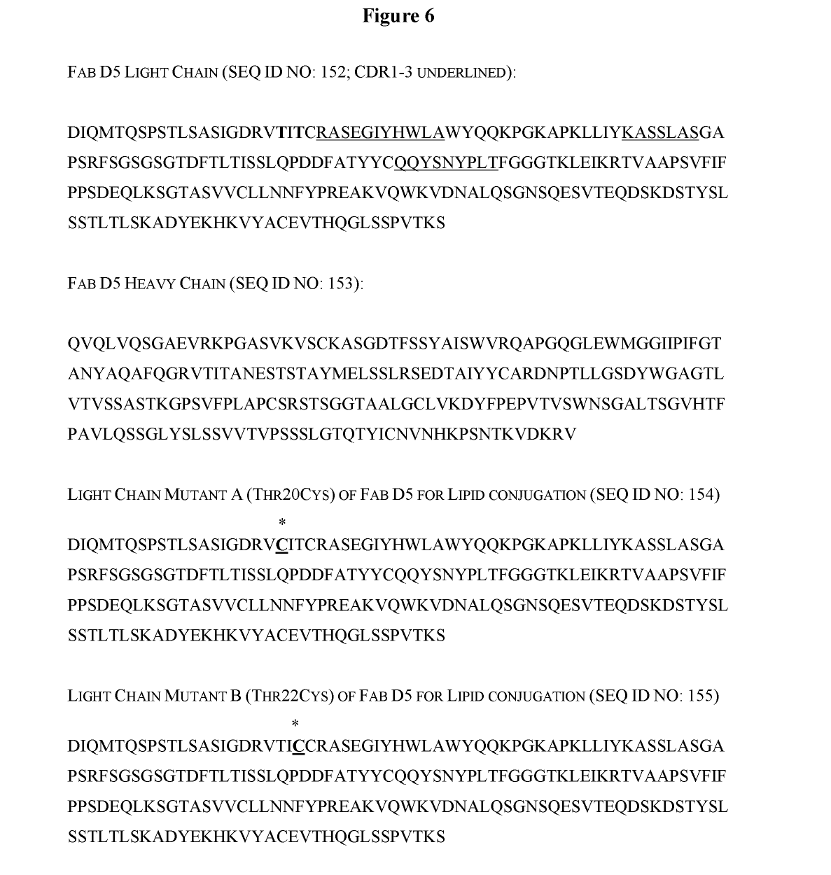

FIG. 7: Illustrates the preferred and optimized locations of cysteine amino acids in the Fab domain of MAB 2F5, that are ideally positioned for covalent linkage to a membrane integrating lipid or a linker including a membrane integrating lipid according to the invention. Also shown is a double mutant of Fab 2F5 with no antiviral activity. (*) marks introduced cysteine amino acids.

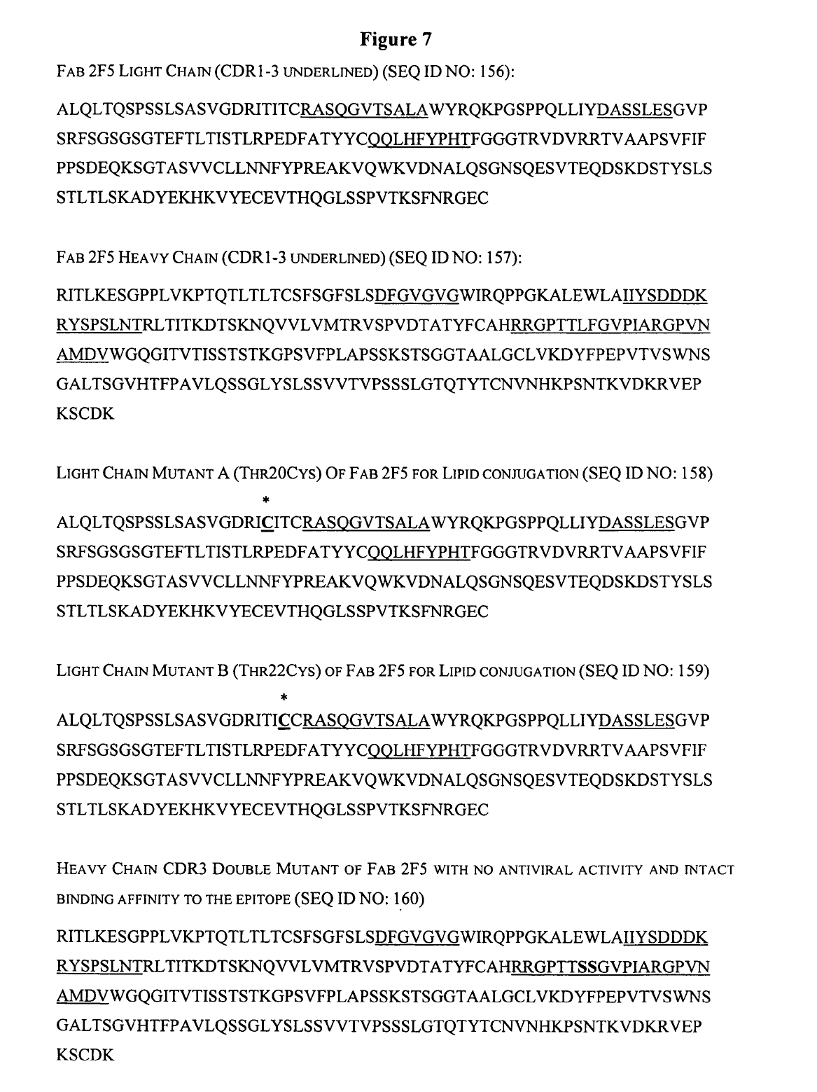

FIG. 8: Illustrates the preferred and optimized locations of cysteine amino acids in the Fab domain of MAB 4E10, that are ideally positioned for covalent linkage to a membrane integrating lipid or a linker including a membrane integrating lipid according to the invention. (*) marks introduced cysteine amino acids.

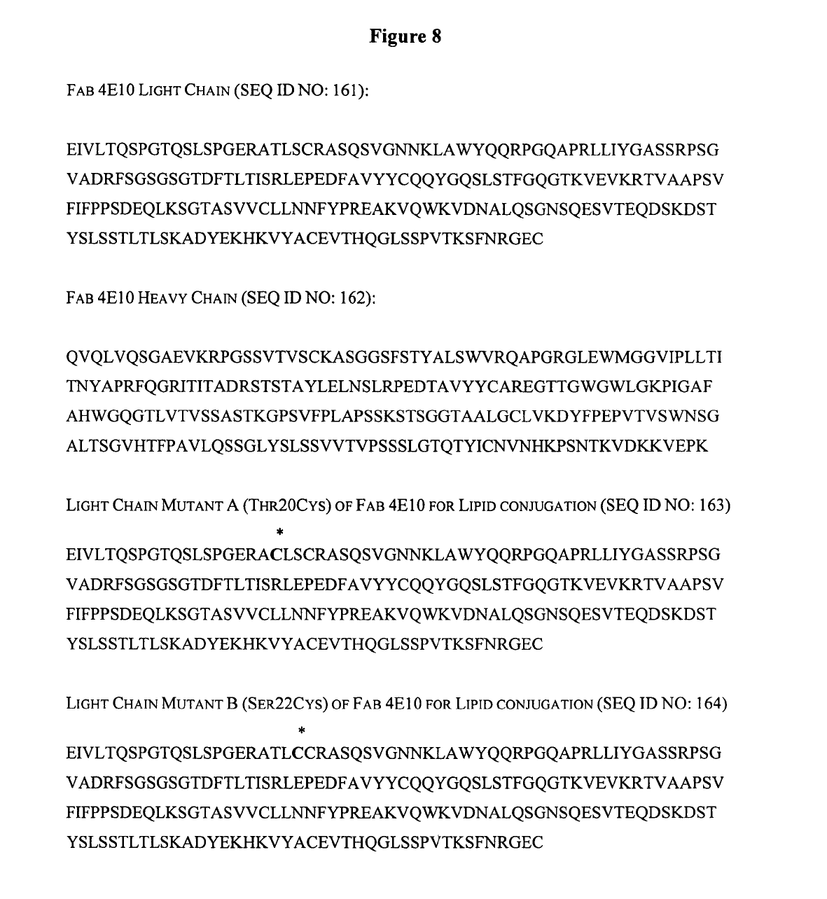

FIG. 9: Illustrates the preferred and optimized locations of cysteine amino acids in the Fab domain of MAB VRC01, that are ideally positioned for covalent linkage to a membrane integrating lipid or a linker including a membrane integrating lipid according to the invention. (*) marks introduced cysteine amino acids.

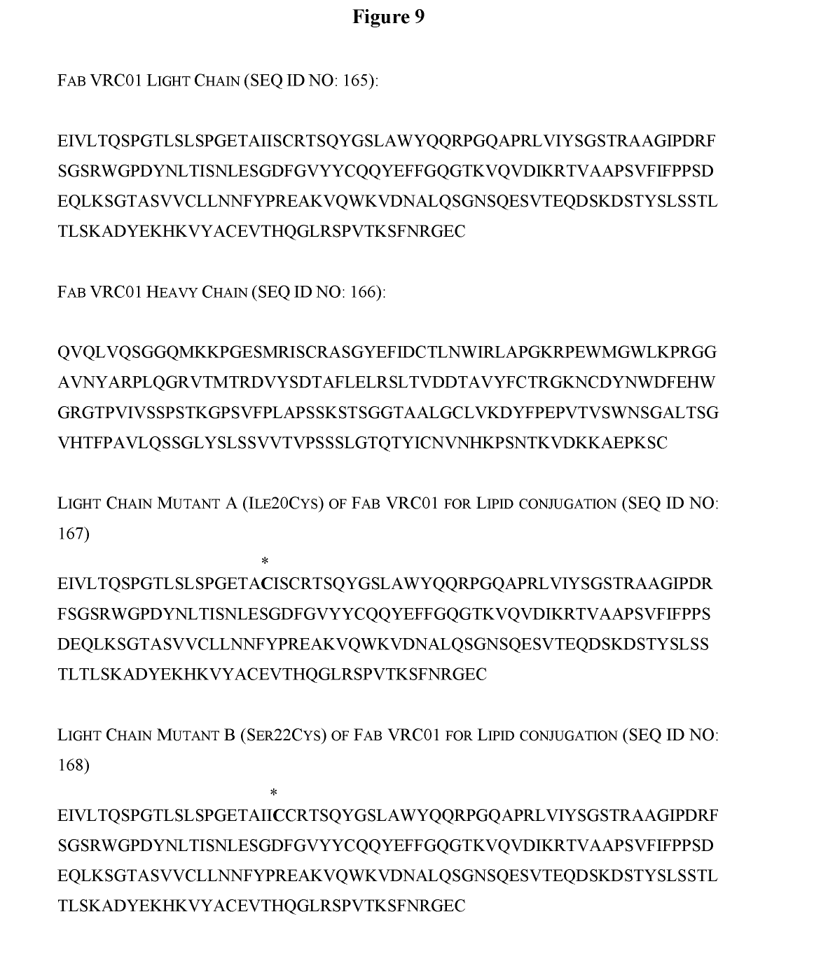

FIG. 10: Illustrates the preferred and optimized locations of cysteine amino acids in the Fab domain of MAB VRC02, that are ideally positioned for covalent linkage to a membrane integrating lipid or a linker including a membrane integrating lipid according to the invention. (*) marks introduced cysteine amino acids.

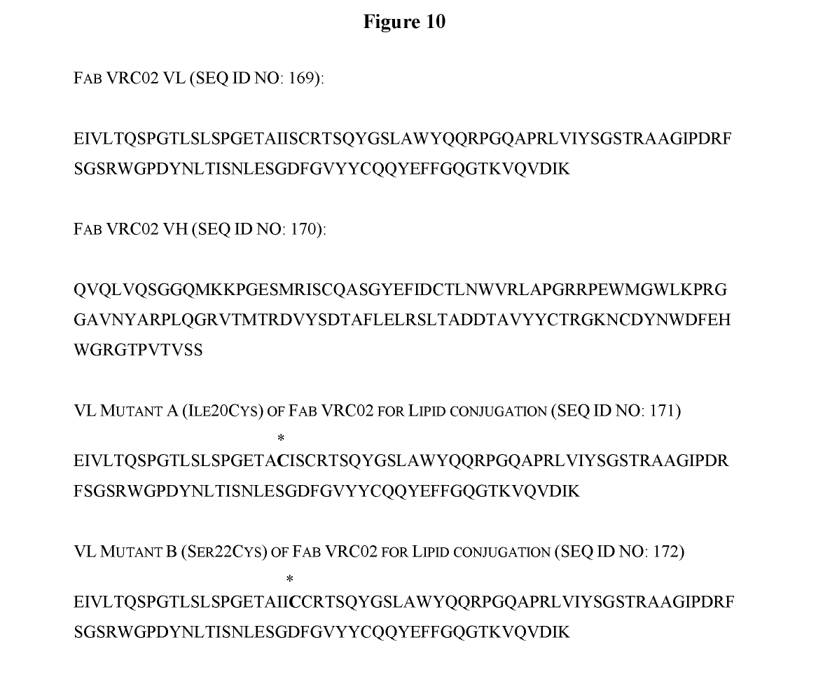

FIG. 11: Illustrates the preferred and optimized locations of cysteine amino acids in the Fab domain of mAb CR6261, that are ideally positioned for covalent linkage to a membrane integrating lipid or a linker including a membrane integrating lipid according to the invention. (*) marks introduced cysteine amino acids.

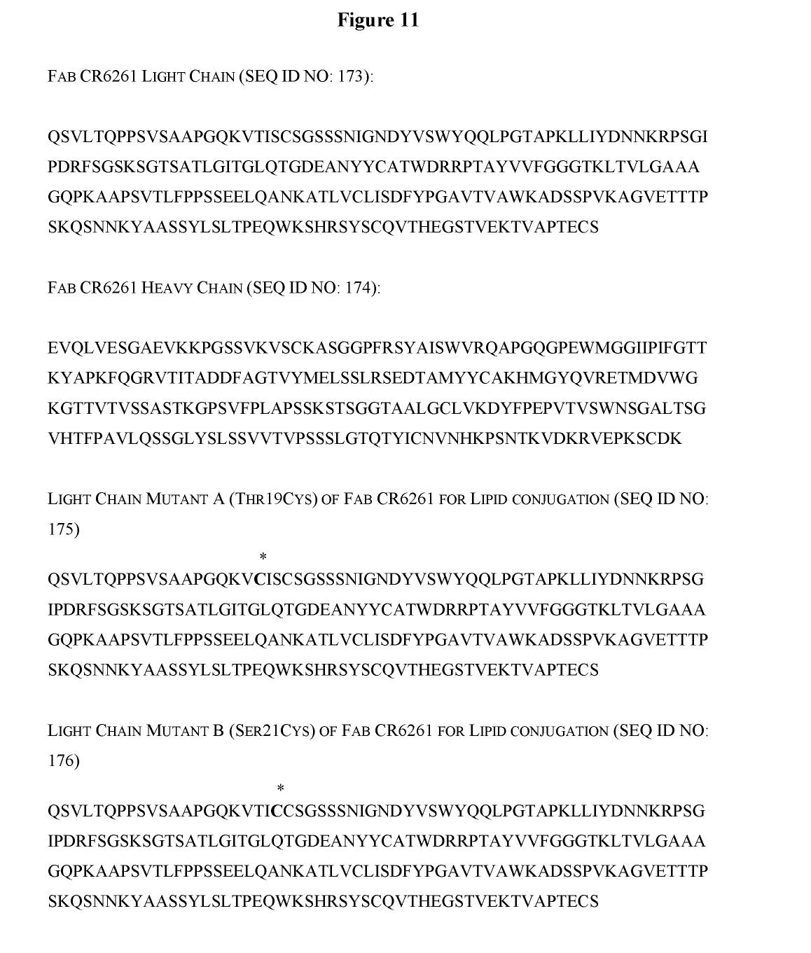

FIG. 12: Illustrates the preferred and optimized locations of cysteine amino acids in the Fab domain of Palivizumab, that are ideally positioned for covalent linkage to a membrane integrating lipid or a linker including a membrane integrating lipid according to the invention. (*) marks introduced cysteine amino acids.

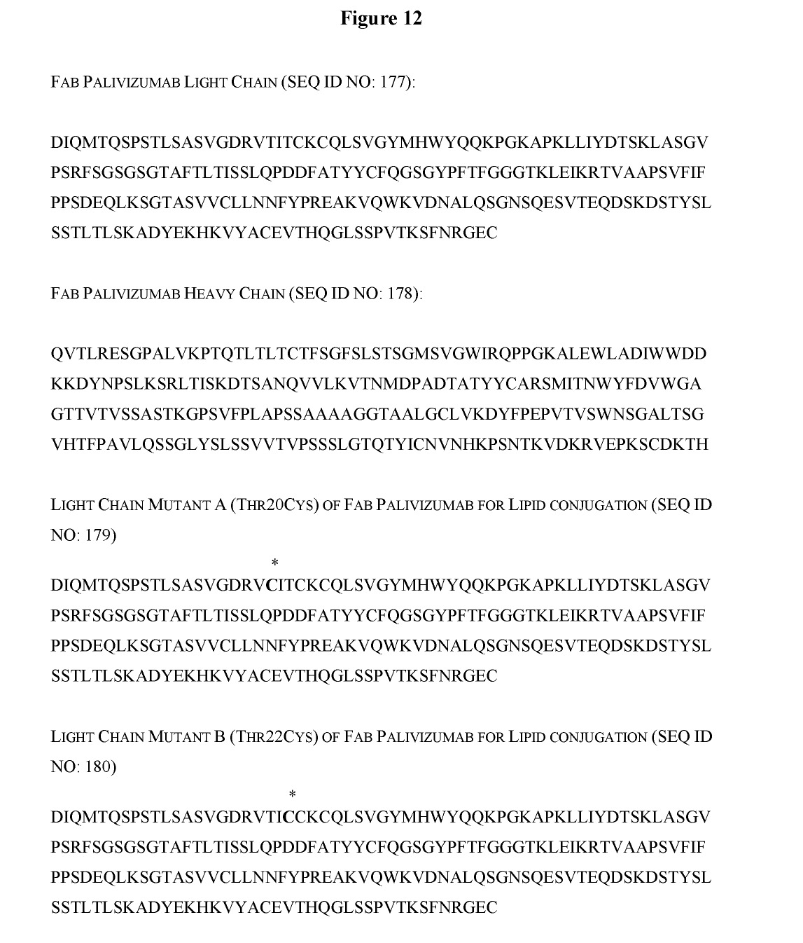

FIG. 13: Illustrates the preferred and optimized locations of cysteine amino acids in the Fab domain of Motavizumab, that are ideally positioned for covalent linkage to a membrane integrating lipid or a linker including a membrane integrating lipid according to the invention. (*) marks introduced cysteine amino acids.

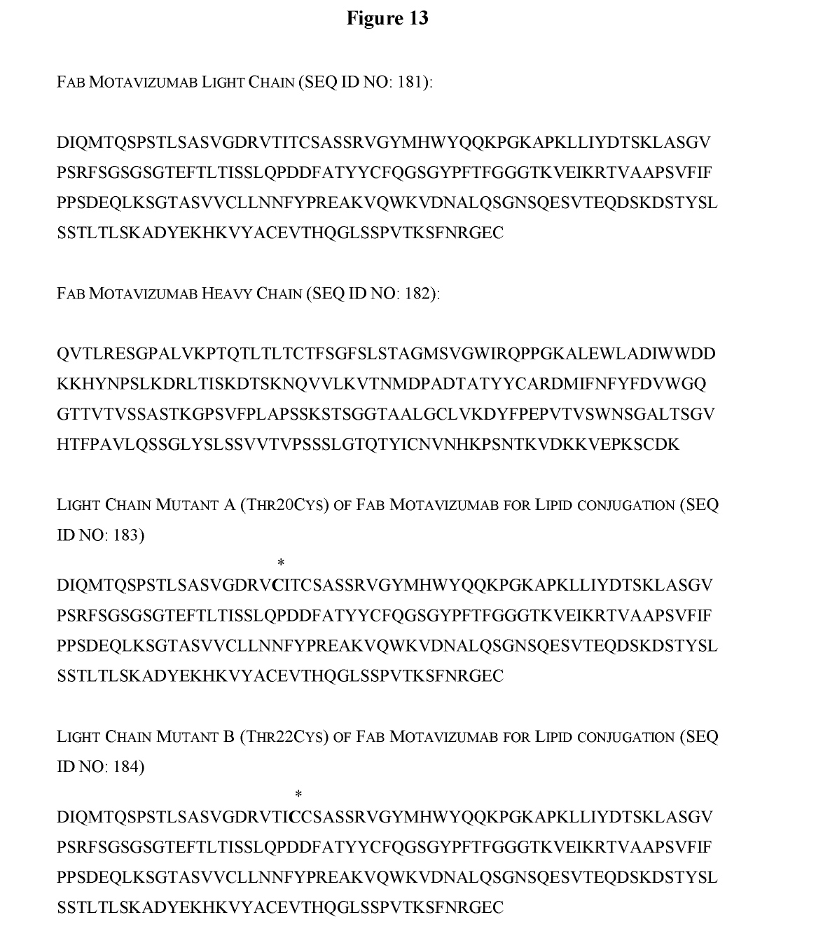

FIG. 14: Antiviral activity against HPIV3 of the dimeric Fusion Inhibitor [(Ac-VALDPIDISIVLNKAKSDLEESKEWIRRSNGKLDSIGSGSG-C(Mal-PEG₄)]₂-Chol and the corresponding monomeric fusion inhibitor Ac-VALDPIDISIVLNKAKSDLEESKEWIRRSNGKLDSIGSGSG-C(PEG₄-Chol), in a plaque reduction assay.

FIG. 15: Antiviral activity against Nipah virus (NiV) of the dimeric fusion inhibitor [(Ac-VALDPIDISIVLNKAKSDLEESKEWIRRSNGKLDSIGSGSG-C(Mal-PEG₄)]₂-Chol, the monomeric fusion inhibitor Ac-VALDPIDISIVLNKAKSDLEESKEWIRRSNGKLDSIGSGSG-C(PEG₄-Chol), and the control peptide lacking cholesterol Ac-VALDPIDISIVLNKAKSDLEESKEWIRRSNGKLDSIGSGSG-C(CH₂CONH₂), in a fusion inhibition assay.

FIG. 16: Antiviral activity of Cholesterol-derivatized inhibitors derived from the sequence of Human Parainfluenza Virus Type 3 (HPIV3) against Measles Virus (MV), Edmonton Strain. Shown is a comparison of the inhibition of MV fusion by the dimeric fusion inhibitor [(Ac-VALDPIDISIVLNKAKSDLEESKEWIRRSNGKLDSI-GSGSG-C(Mal-PEG₄)]₂-Chol (Δ), by the monomeric fusion inhibitor Ac-VALDPIDISIVLNKAKSDLEESKEWIRRSNGKLDSI-GSGSG-C(PEG₄-Chol) (X), by the dimeric fusion inhibitor with a benzyl group instead of cholesterol [(Ac-VALDPIDISIVLNKAKSDLEESKEWIRRSNGKLDSI-GSGSG-C(Mal-PEG₄)]₂-OBz (−), and by the control monomeric peptide without cholesterol Ac-VALDPIDISIVLNKAKSDLEESKEWIRRSNGKLDSI-GSGSG-C(CH₂CONH₂)(∘).

FIG. 17. Antiviral activity of Cholesterol-derivatized Inhibitors against Human Immunodeficiency Virus (HIV). Shown is the antiviral activity against a CCR5-dependent (R5-Bal) and a CXCR4-dependent (Lai/IIIB) strain of HIV-1 of the monomeric fusion inhibitor Ac-SWETWEREIENYTRQIYRILEESQEQQDRNERDLLEGSGC(PEG₄-Chol)-NH₂ (SEQ ID NO. 191) (black, ▴) and of the peptide inhibitor C34 lacking cholesterol (dark grey, ).

FIG. 18: Antiviral activity of Cholesterol-derivatized Inhibitors against Human Immunodeficiency Virus (HIV). Shown is the antiviral activity against a CCR5-dependent (R5-Bal) and a CXCR4-dependent (Lai/IIIB) strain of HIV-1 of the dimeric fusion inhibitor [(Ac-WMEWDREINNYTSLIHSLIEESQNQQEKNEQELL-GSG-C(MAL-PEG₄)]₂-Chol (grey, ), of the monomeric fusion inhibitor Ac-WMEWDREINNYTSLIHSLIEESQNQQEKNEQELL-GSG-C(PEG₄-Chol) (black, ▴), and of the monomeric peptide lacking cholesterol Ac-WMEWDREINNYTSLIHSLIEESQNQQEKNEQELL-GSG-C(CH₂CONH₂) (black, □).

FIG. 19: Peptide sequence of C34 (SEQ ID NO: 120) the generic amino acid sequence (SEQ ID NO: 188) comprised in a preferred polypeptide comprised in the inhibitor of the invention and sequences of preferred polypeptides.

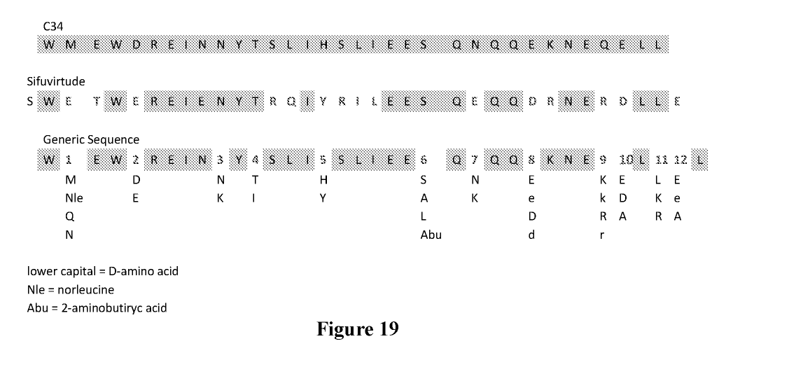

### EXAMPLES

### 1. Exemplary Monomeric Fusion Inhibitors

A derivative according to the present invention comprising only one polypeptide can be obtained by reaction of a suitable derivative of cholesterol derivatives bearing a bromoacetyl group, prepared as described in the example below, or by analogy, thereto, by using commercially available compounds or by well known methods from commercially available compounds. Derivatives of cholesterol are commercially available or can be made from commercially available materials by well known methods.

### 1.1. Example

### Synthesis of BrAc-PEG₄-Chol

### [Cholest-5-en-3-yl 1-bromo-2-oxo-6,9,12,15-tetraoxa-3-azaoctadecan-18-oate]

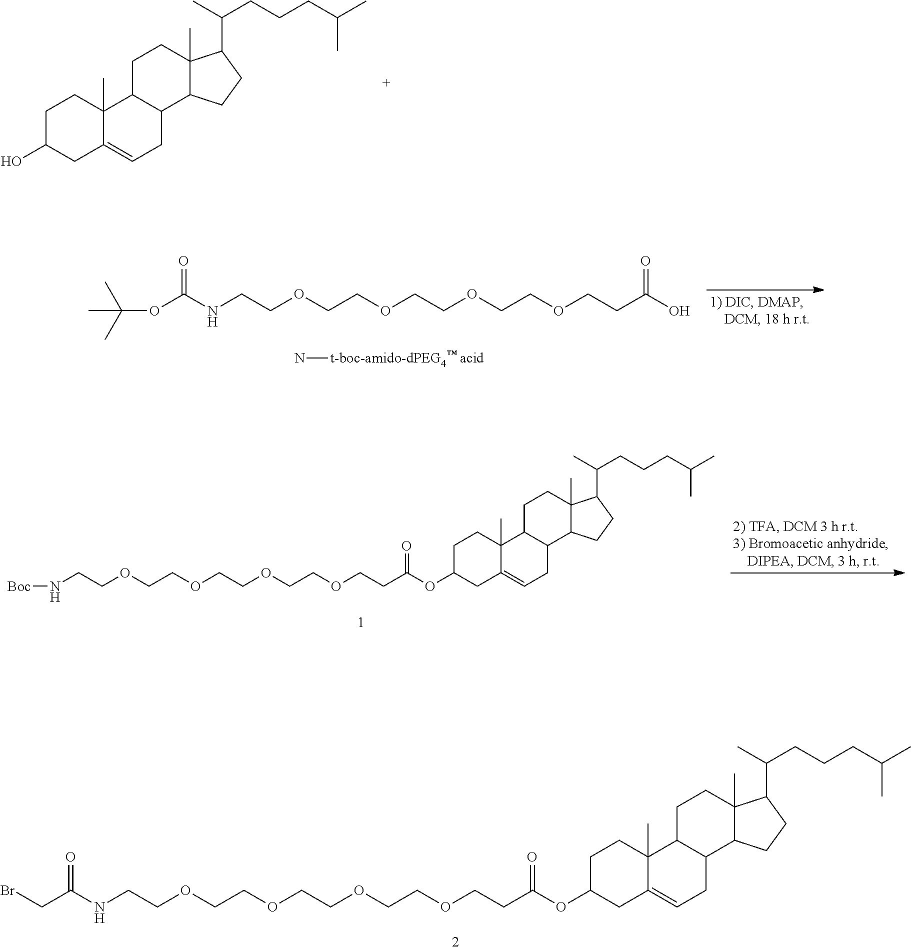

### 1. Cholest-5-en-3-yl 2,2-dimethyl-4-oxo-3,8,11,14,17-pentaoxa-5-azaicosan-20-oate (1)

N-t-boc-amido-dPEG₄™ acid (1 g, 2.7 mmol, Product No 10220, Quanta BioDesign, Ltd.) was added to a solution of cholesterol (0.99 g, 2.7 mmol) in 40 mL of CH₂Cl₂, followed by N,N′-diisopropylcarbodiimide (0.43 mL, 3.2 mmol) and 4-dimethylamino-pyridine (16 mg, 5%). The mixture was stirred at room temperature overnight and the solvent was evaporated under vacuo. The crude was dissolved in EtOAc, washed with HCl 1N, saturated NH₄Cl and brine, dried over Na₂SO₄, filtered and concentrated. The crude was purified by flash column chromatography (BIOTAGE) on silica gel with a gradient 25-50% EtOAc in petroleum ether to afford 1.48 g of desired compound as incolor oil (Yield 75%).

### 2. Cholest-5-en-3-yl 1-bromo-2-oxo-6,9,12,15-tetraoxa-3-azaoctadecan-18-oate (2)

Trifluoroacetic acid (2 mL, 26 mmol) was added to a solution of 1 (1.48 g, 2 mmol) in 10 ml of CH₂Cl₂ and the mixture was stirred at room temperature for 3 h. All the volatiles were removed under vacuo and the crude was lyophilized to obtain an incolor oil that was dissolved in 60 mL of CH₂Cl₂. Bromoacetic anhydride (0.62 g, 2.4 mmol) was added followed by N,N-diisopropylethylamine (0.65 mL, 3.7 mmol) and the mixture was stirred at room temperature for 3 h. The solvent was removed under vacuo and the crude purified by flash column chromatography on silica gel (BIOTAGE) with a gradient 50-90% of EtOAc in petroleum ether to obtain 1.1 g of desired compound as a colourless oil with a yield of 74% in two steps.

### 1.2. Synthesis of Ac-Trp-Gln-Glu-Trp-Glu-Arg-Glu-Ile-Asn-Lys-Tyr-Ile-Ser-Leu-Ile-Tyr-Ser-Leu-Ile-Glu-Glu-Ala-Gln-Asn-Gln-Gln-(D)Glu-Lys-Asn-Glu-(D)Lys-Ala-Leu-Leu-(D)Glu-Leu-Gly-Ser-Gly-Cys(PEG₄-Chol)-NH₂ (SEQ ID NO. 190)

### 1. Synthesis of Ac-Trp-Gln-Glu-Trp-Glu-Arg-Glu-Ile-Asn-Lys-Tyr-Ile-Ser-Leu-Ile-Tyr-Ser-Leu-Ile-Glu-Glu-Ala-Gln-Asn-Gln-Gln-(D)Glu-Lys-Asn-Glu-(D)Lys-Ala-Leu-Leu-(D)Glu-Leu-Gly-Ser-Gly-Cys-NH₂

The peptide was prepared by standard Solid-phase Peptide Synthesis, using Fmoc/t-Bu chemistry on a Pioneer Peptide Synthesizer (Applied Biosystems). To produce the peptide C-terminal amide, the peptide was synthesized on a Champion PEG-PS resin (Biosearch Technologies, Inc., Novato, Calif.) that had been previously derivatized with the Fmoc-Rink linker using DIPCDI/HOBt as activators. All the acylation reactions were performed for 60 min with 4-fold excess of activated amino acid over the resin free amino groups. Amino acids were activated with equimolar amounts of HBTU (2-(1H-benzotriazole-1-yl)-1,1,3,3-tetramethyluronium hexafluorophosphate) and a 2-fold molar excess of DIEA (N,N-diisopropyl-ethylamine). The side chain protecting groups were: tert-butyl for Asp, Glu, Ser; trityl for Asn and Cys; tert-butoxy-carbonyl for Lys, Trp; and 2,2,4,6,7-pentamethyldihydrobenzofuran-5-sulfonyl for Arg. At the end of the assembly, the dry peptide-resin was treated with 82.5% TFA, 5% phenol, 5% water, 5% thioanisole, 2.5% ethanedithiol for 1.5 h at room temperature. The resin was filtered and the solution was evaporated and the peptide pellet treated several times with diethylether to remove the organic scavengers. The final pellet was dried, resuspended in 1:1 (v/v) H₂O: acetonitrile and lyophilized.

PURIFICATION AND ANALYTICAL CHARACTERIZATION. The crude peptide was analyzed by liquid chromatography-mass spectrometry using a Waters-Micromass LCZ Platform with a Phenomenex, Jupiter C₄ column (150×4.6 mm, 5 μm) using as eluents (A) 0.1% TFA in water and (B) 0.1% TFA in acetonitrile, and the following linear gradient: 30% (B)-60% (B) in 20′-80% (B) in 3′-80% (B) for 3′, flow 1 ml/min. The crude peptide was dissolved at 1 mg/ml in 70% eluent A/30% eluent B.

The crude peptide was purified by reverse-phase HPLC with a XBridge C18 semi-preparative column (50×150 mm, 5 μm, 130 Å), using as eluents (A) 0.1% TFA in water and (B) 0.1% TFA in acetonitrile, and an isocratic step at 25% (B) for 5′ followed by the linear gradient: 25% (B)-40% (B) in 20′-80% (B) in 2′-80% (B) for 3′, flow 30 ml/min. The purified peptide was characterized by HPLC/MS on a Waters-Micromass LCZ platform as described above (theoretical M.W. 4825.4 Da, found 4824.3 Da).

### 2. Synthesis of Ac-Trp-Gln-Glu-Trp-Glu-Arg-Glu-Ile-Asn-Lys-Tyr-Ile-Ser-Leu-Ile-Tyr-Ser-Leu-Ile-Glu-Glu-Ala-Gln-Asn-Gln-Gln-(D)Glu-Lys-Asn-Glu-(D)Lys-Ala-Leu-Leu-(D)Glu-Leu-Gly-Ser-Gly-Cys(PEG₄-Chol)-NH₂

The peptide was prepared by conjugation between bromoacetyl-PEG₄-cholesterol (2) prepared in 1.1 above and the peptide Ac-Trp-Gln-Glu-Trp-Glu-Arg-Glu-Ile-Asn-Lys-Tyr-Ile-Ser-Leu-Ile-Tyr-Ser-Leu-Ile-Glu-Glu-Ala-Gln-Asn-Gln-Gln-(D)Glu-Lys-Asn-Glu-(D)Lys-Ala-Leu-Leu-(D)Glu-Leu-Gly-Ser-Gly-Cys-NH₂ prepared in 1 above. 2.26 mol of the peptide Ac-Trp-Gln-Glu-Trp-Glu-Arg-Glu-Ile-Asn-Lys-Tyr-Ile-Ser-Leu-Ile-Tyr-Ser-Leu-Ile-Glu-Glu-Ala-Gln-Asn-Gln-Gln-(D)Glu-Lys-Asn-Glu-(D)Lys-Ala-Leu-Leu-(D)Glu-Leu-Gly-Ser-Gly-Cys-NH₂ were dissolved in 600 μL of DMSO and 2.49 mol (1.1 eq) of (2) dissolved in 188 μL of THF, were added. Then 8 μL of DIEA (N,N-diisopropyl-ethylamine) were added to the mixture which was left stirring at room temperature. The reaction was monitored by liquid chromatography-mass spectrometry using a Waters-Micromass LCZ Platform with a Phenomenex, Jupiter C₄ column (150×4.6 mm, 5 μm) using a linear gradient of eluents (A) 0.1% TFA in water and (B) 0.1% TFA in acetonitrile, flow rate 1 ml/min. The reaction was complete after 3 h incubation.

PURIFICATION AND ANALYTICAL CHARACTERIZATION. The cholesterol-peptide product was purified by reverse-phase HPLC with semi-preparative Waters RCM Delta-Pak™ C₄ cartridges (25×200 mm, 15 μm), using as eluents (A) 0.1% TFA in water and (B) 0.1% TFA in acetonitrile, and an isocratic step at 45% (B) for 5′ followed by the linear gradient: 45% (B)-65% (B) in 30′-80% (B) in 2′-80% (B) for 3′, flow 30 ml/min. The purified peptide was characterized by HPLC/MS on a Waters-Micromass LCZ platform as described above (theoretical M.W. 5499.4 Da, found 5498.0.2 Da).

### 1.3. Synthesis of Ac-Ser-Trp-Glu-Thr-Trp-Glu-Arg-Glu-Ile-Glu-Asn-Tyr-Thr-Arg-Gln-Ile-Tyr-Arg-Ile-Leu-Glu-Glu-Ser-Gln-Glu-Gln-Gln-Asp-Arg-Asn-Glu-Arg-Asp-Leu-Leu-Glu-Gly-Ser-Gly-Cys(PEG₄-Chol)-NH₂ (SEQ ID NO. 191)

### 1. Synthesis of Ac-Ser-Trp-Glu-Thr-Trp-Glu-Arg-Glu-Ile-Glu-Asn-Tyr-Thr-Arg-Gln-Ile-Tyr-Arg-Ile-Leu-Glu-Glu-Ser-Gln-Glu-Gln-Gln-Asf-Arg-Asn-Glu-Arg-Asf-Leu-Leu-Glu-Gly-Ser-Gly-Cys-NH₂

The peptide was prepared by standard Solid-phase Peptide Synthesis, using Fmoc/t-Bu chemistry on a Pioneer Peptide Synthesizer (Applied Biosystems). To produce the peptide C-terminal amide, the peptide was synthesized on a Champion PEG-PS resin (Biosearch Technologies, Inc., Novato, Calif.) that had been previously derivatized with the Fmoc-Rink linker using DIPCDI/HOBt as activators. All the acylation reactions were performed for 60 min with 4-fold excess of activated amino acid over the resin free amino groups. Amino acids were activated with equimolar amounts of HBTU (2-(1H-benzotriazole-1-yl)-1,1,3,3-tetramethyluronium hexafluorophosphate) and a 2-fold molar excess of DIEA (N,N-diisopropyl-ethylamine). The side chain protecting groups were: tert-butyl for Asp, Glu, and Ser; trityl for Asn and Cys; tert-butoxy-carbonyl for Lys, Trp; and 2,2,4,6,7-pentamethyldihydrobenzofuran-5-sulfonyl for Arg. At the end of the assembly, the dry peptide-resin was treated with 82.5% TFA, 5% phenol, 5% water, 5% thioanisole, 2.5% ethanedithiol for 1.5 h at room temperature. The resin was filtered and the solution was evaporated and the peptide pellet treated several times with diethylether to remove the organic scavengers. The final pellet was dried, resuspended in 1:1 (v/v) H₂O: acetonitrile and lyophilized.

PURIFICATION AND ANALYTICAL CHARACTERIZATION. The crude peptide was analyzed by liquid chromatography-mass spectrometry using a Waters-Micromass LCZ Platform with a Phenomenex, Jupiter C₄ column (150×4.6 mm, 5 μm) using as eluents (A) 0.1% TFA in water and (B) 0.1% TFA in acetonitrile, and the following linear gradient: 30% (B)-60% (B) in 20′-80% (B) in 3′-80% (B) for 3′, flow 1 ml/min. The crude peptide was dissolved at 1 mg/ml in 70% eluent A/30% eluent B.

The crude peptide was purified by reverse-phase HPLC with a XBridge C18 semi-preparative column (50×150 mm, 5 μm, 130 Å), using as eluents (A) 0.1% TFA in water and (B) 0.1% TFA in acetonitrile, and an isocratic step at 25% (B) for 5′ followed by the linear gradient: 25% (B)-40% (B) in 20′-80% (B) in 2′-80% (B) for 3′, flow 30 ml/min. The purified peptide was characterized by HPLC/MS on a Waters-Micromass LCZ platform as described above (theoretical M.W. 5031.3 Da, found 5030.0 Da).

### 2. Synthesis of Ac-Ser-Trp-Glu-Thr-Trp-Glu-Arg-Glu-Ile-Glu-Asn-Tyr-Thr-Arg-Gln-Ile-Tyr-Arg-Ile-Leu-Glu-Glu-Ser-Gln-Glu-Gln-Gln-Asp-Arg-Asn-Glu-Arg-Asp-Leu-Leu-Glu-Gly-Ser-Gly-Cys(PEG₄-Chol)-NH₂

The peptide was prepared by conjugation between bromoacetyl-PEG₄-cholesterol (2) prepared in 1.1 above and the peptide Ac-Ser-Trp-Glu-Thr-Trp-Glu-Arg-Glu-Ile-Glu-Asn-Tyr-Thr-Arg-Gln-Ser-Gln-Glu-Gln-Gln-Asp-Arg-Asn-Glu-Arg-Asp-Leu-Leu-Glu-Gly-Ser-Gly-Cys-NH₂ prepared in 1 above. 2.26 mol of the peptide Ac-Ser-Trp-Glu-Thr-Trp-Glu-Arg-Glu-Ile-Glu-Asn-Tyr-Thr-Arg-Gln-Ile-Tyr-Arg-Ile-Leu-Glu-Glu-Ser-Gln-Glu-Gln-Gln-Asp-Arg-Asn-Glu-Arg-Asp-Leu-Leu-Glu-Gly-Ser-Gly-Cys-NH₂ were dissolved in 600 of DMSO and 2.49 mol (1.1 eq) of (2) dissolved in 188 μL of THF, were added. Then 8 μL of DIEA (N,N-diisopropyl-ethylamine) were added to the mixture which was left stirring at room temperature. The reaction was monitored by liquid chromatography-mass spectrometry using a Waters-Micromass LCZ Platform with a Phenomenex, Jupiter C₄ column (150×4.6 mm, 5 μm) using a linear gradient of eluents (A) 0.1% TFA in water and (B) 0.1% TFA in acetonitrile, flow rate 1 ml/min. The reaction was complete after 3 h incubation.

PURIFICATION AND ANALYTICAL CHARACTERIZATION. The cholesterol-peptide product was purified by reverse-phase HPLC with semi-preparative Waters RCM Delta-Pak™ C₄ cartridges (25×200 mm, 15 μm), using as eluents (A) 0.1% TFA in water and (B) 0.1% TFA in acetonitrile, and an isocratic step at 45% (B) for 5′ followed by the linear gradient: 45% (B)-65% (B) in 30′-80% (B) in 2′-80% (B) for 3′, flow 30 ml/min. The purified peptide was characterized by HPLC/MS on a Waters-Micromass LCZ platform as described (theoretical M.W. 5705.3 Da, found 5704.2 Da).

### 2. Exemplary Dimeric Fusion Inhibitors

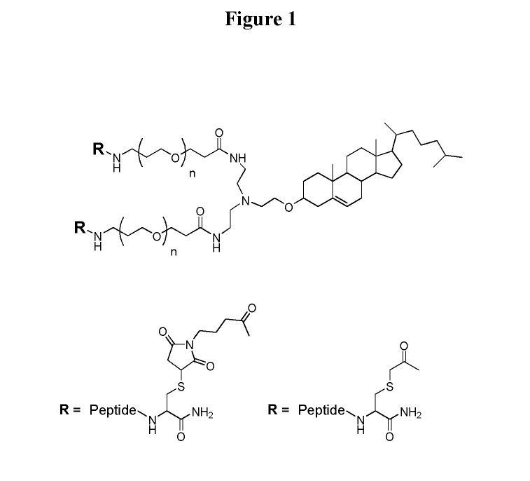

A generic structure for a fusion inhibitor with two identical peptide chains and a cholesterol group is shown in FIG. 1. It features a three-arm core, the first arm bearing a cholesterol group, and the other two bearing two identical peptide chains corresponding to the sequence of the fusion inhibitor. Each of the peptide-bearing arms can have a variable number of (CH₂—CH₂—O—) units (polyethylene glycol, PEG units) to modulate the distance between the peptide chains. The peptides can be attached to the core structure in a number of ways, known to those skilled in the art. In the example shown in FIG. 1, attachment is through a thioether bond between the thiol group of a cysteine residue on the peptide chain and a thiol-reactive moiety on the core structure; the thioether is formed by reaction with either a 4-maleimido-butyric acid (FIG. 1, bottom left) o with a bromoacetic acid (FIG. 1, bottom right).

### 2.1 Exemplary Dimeric Fusion Inhibitor of Human Immunodeficiency Virus (HIV)

The sequence of the HRN-binding peptide corresponds to the sequence of C34 (WMEWDREINNYTSLIHSLIEESQNQQEKNEQELL, SEQ ID NO: 120) with the addition of the C-terminal sequence Gly-Ser-Gly-Cys (4):

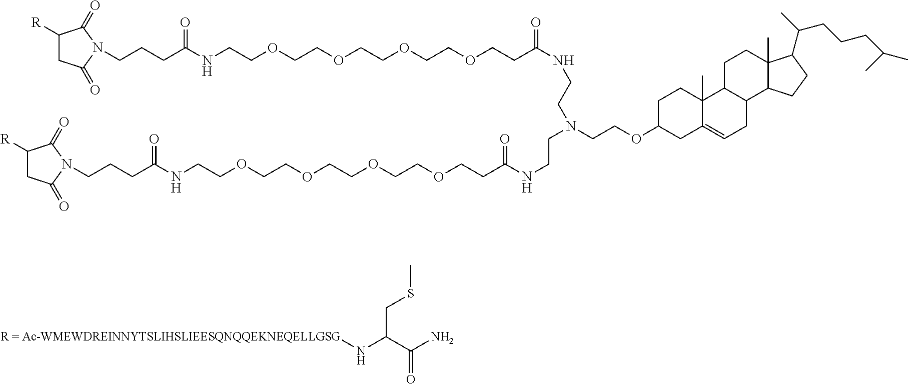

### 2.2 Exemplary Dimeric Fusion Inhibitor of Human Parainfluenza Virus Type 3 (HPIV3)

The sequence of the HRN-binding peptide corresponds to SEQ ID NO: 187 with the addition of the C-terminal sequence Gly-Ser-Gly-Ser-Gly-Cys:

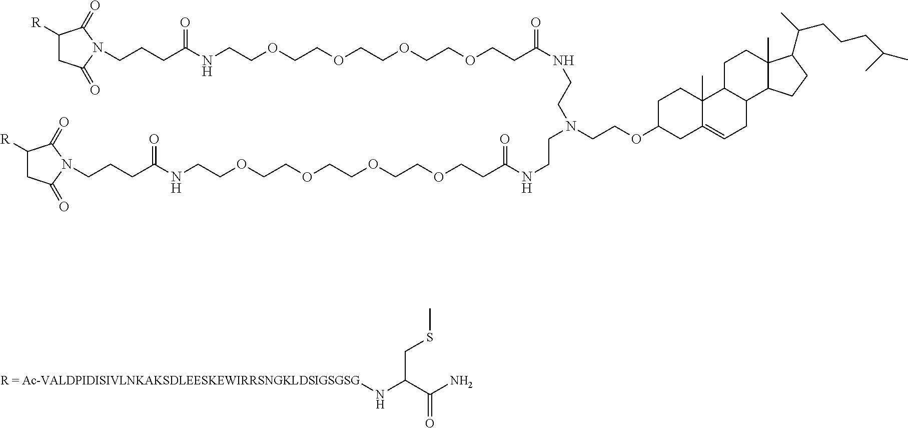

Similarly, a dimeric fusion inhibitor comprising HRN-binding peptides corresponding to SEQ ID NO: 99 with the addition of the C-terminal sequence Gly-Ser-Gly-Ser-Gly-Cys can be produced.

### 2.3 Exemplary Dimeric Fusion Inhibitor of Human Parainfluenza Virus Type 3/Hendra Virus (HPIV3/HeV)

The sequence of the HRN-binding peptide corresponds to SEQ ID NO: 100 with the addition of the C-terminal sequence Gly-Ser-Gly-Ser-Gly-Cys:

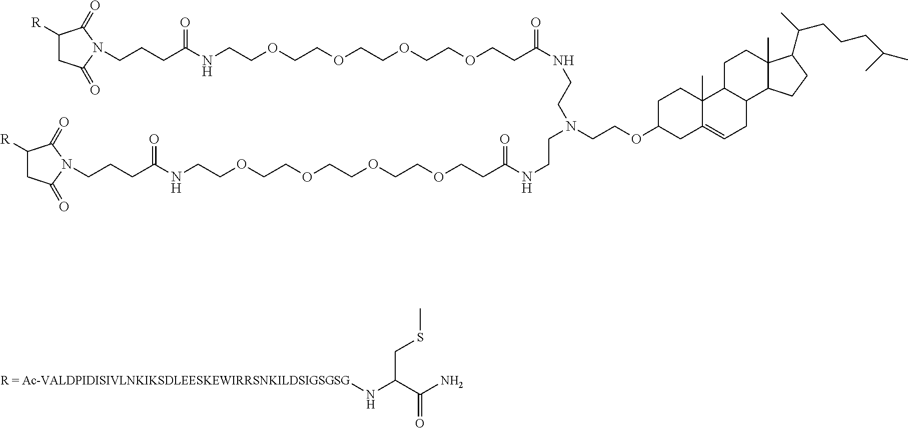

### 2.4 Exemplary Dimeric Fusion Inhibitor of Human Parainfluenza Virus Type 1 (HPIV1)

The sequence of the HRN-binding peptide corresponds to SEQ ID NO: 19, with the addition of the C-terminal sequence Gly-Ser-Gly-Ser-Gly-Cys:

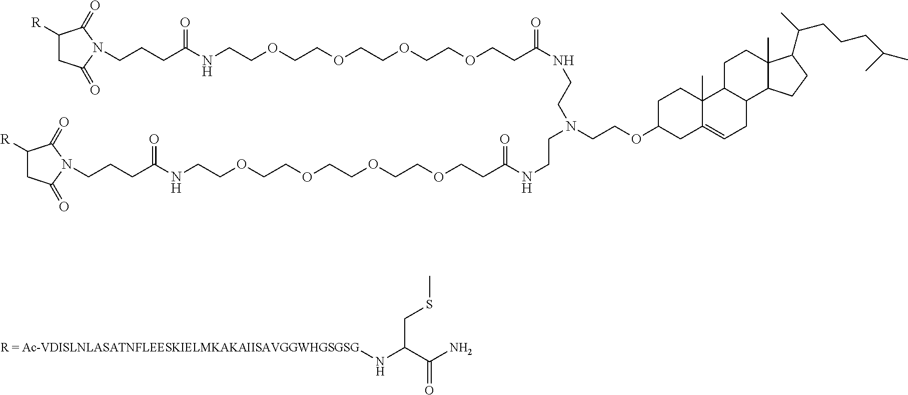

### 2.5 Exemplary Dimeric Fusion Inhibitor of Respiratory Syncytial Virus (RSV)

The sequence of the HRN-binding peptide corresponds to SEQ ID NO: 96, with the addition of the C-terminal sequence Gly-Ser-Gly-Ser-Gly-Cys:

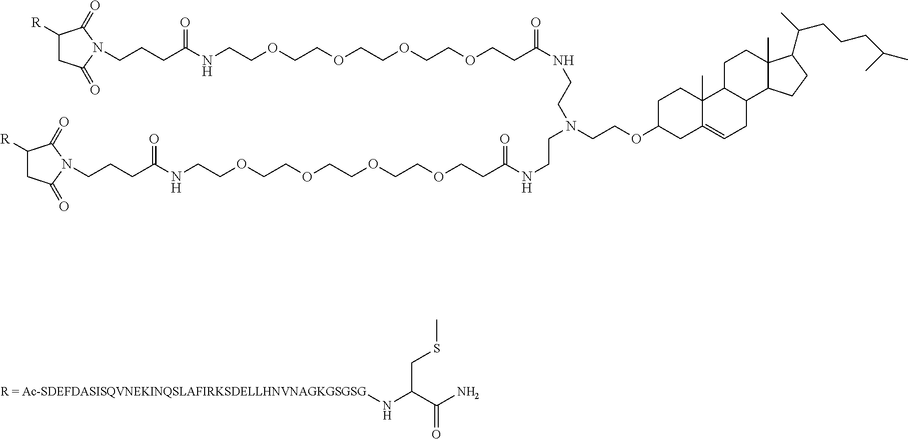

### 2.6 Exemplary Dimeric Fusion Inhibitor of Nipah Virus (NiV)

The sequence of the HRN-binding peptide is the same as for the exemplary fusion inhibitor of HPIV3, and corresponds to SEQ ID NO: 100, with the addition of the C-terminal sequence Gly-Ser-Gly-Ser-Gly-Cys:

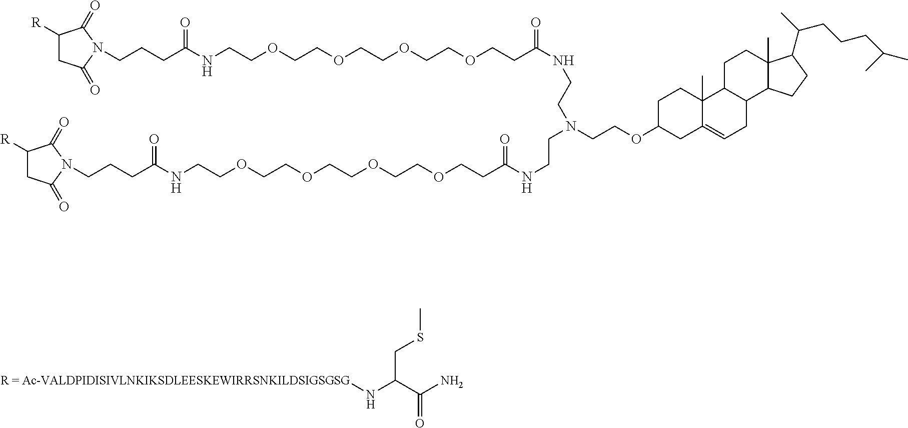

### 2.7 Exemplary Dimeric Fusion Inhibitor of Hendra Virus (HeV)

The sequence of the HRN-binding peptide is the same as for the exemplary fusion inhibitor of HPIV3, and corresponds to SEQ ID NO: 100, with the addition of the C-terminal sequence Gly-Ser-Gly-Ser-Gly-Cys:

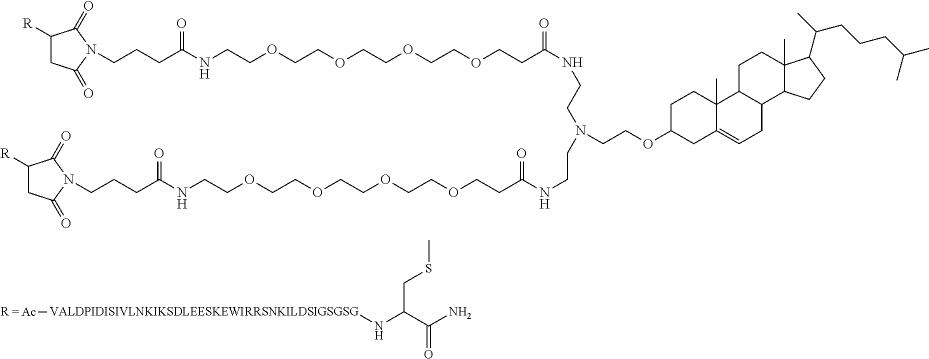

### 2.8 Exemplary Dimeric Fusion Inhibitor of Influenza A Virus

The sequence of the HRN-binding peptide corresponds to SEQ ID NO: 83, with the addition of the C-terminal sequence Gly-Ser-Gly-Ser-Gly-Cys:

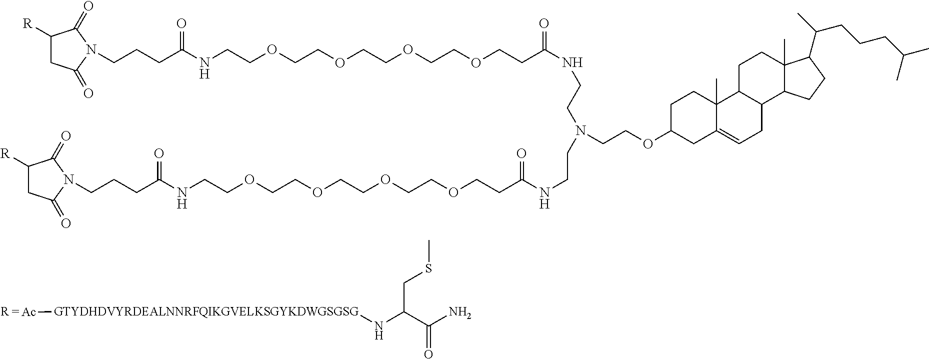

### 2.9 Exemplary Dimeric Fusion Inhibitor of Newcastle Disease Virus

The sequence of the HRN-binding peptide corresponds to SEQ ID NO: 26, with the addition of the C-terminal sequence Gly-Ser-Gly-Ser-Gly-Cys:

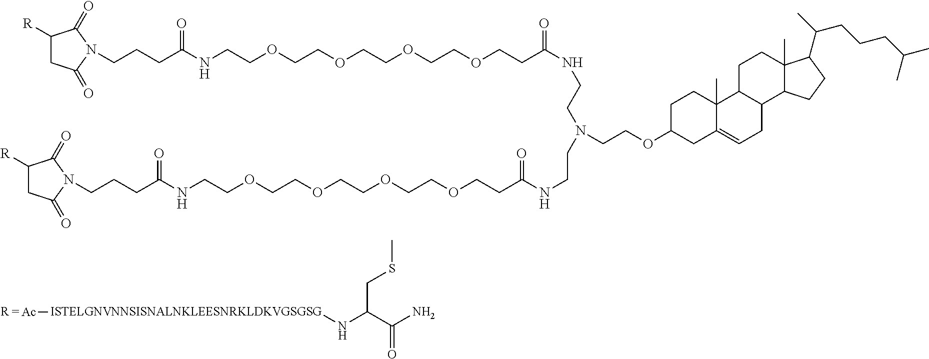

### 2.10 Exemplary Dimeric Fusion Inhibitor of Measles Virus

The sequence of the HRN-binding peptide corresponds to peptide T-265 in Lambert et al. (6) (SEQ ID NO: 121), with the addition of the C-terminal sequence Gly-Ser-Gly-Ser-Gly-Cys:

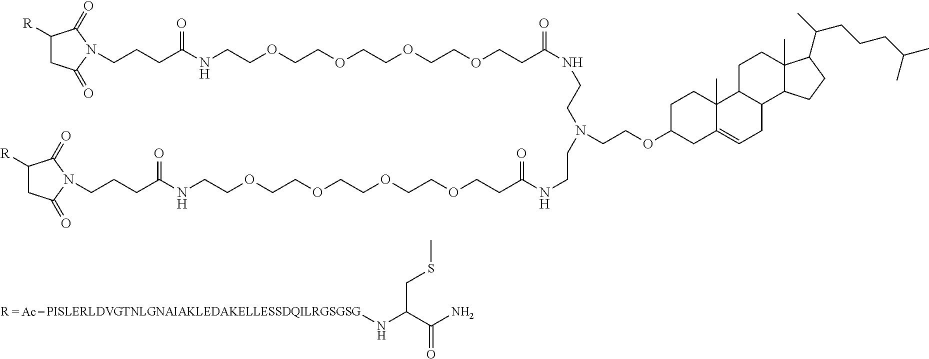

### 2.11 Exemplary Dimeric Fusion Inhibitor of Mumps Virus

The sequence of the HRN-binding peptide corresponds to SEQ ID NO: 22, with the addition of the C-terminal sequence Gly-Ser-Gly-Ser-Gly-Cys:

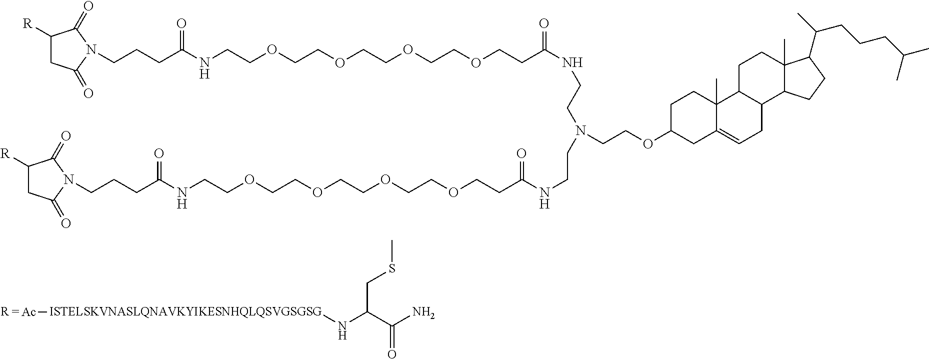

### 2.12 Exemplary Dimeric Fusion Inhibitor of Sendai Virus

The sequence of the HRN-binding peptide corresponds to SEQ ID NO: 20, with the addition of the C-terminal sequence Gly-Ser-Gly-Ser-Gly-Cys:

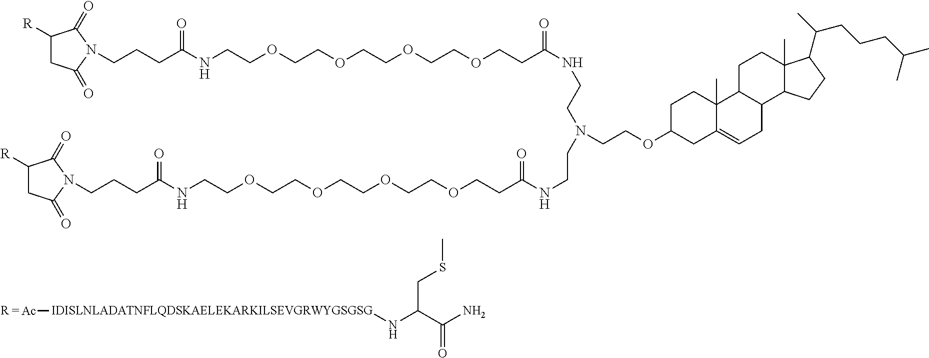

### 2.13 Exemplary Dimeric Fusion Inhibitor of Ebola Virus

The sequence of the HRN-binding peptide corresponds to SEQ ID NO: 53:

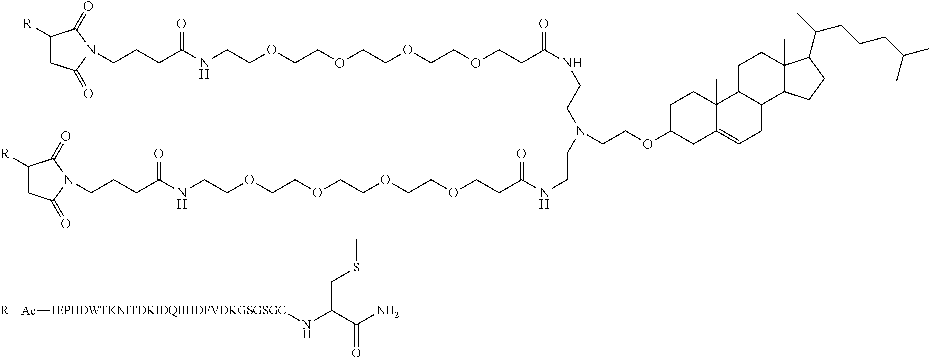

### 2.14 Exemplary Dimeric Fusion Inhibitor of Marburg Virus

The sequence of the HRN-binding peptide corresponds to SEQ ID NO: 34, with the addition of the C-terminal sequence Gly-Ser-Gly-Ser-Gly-Cys:

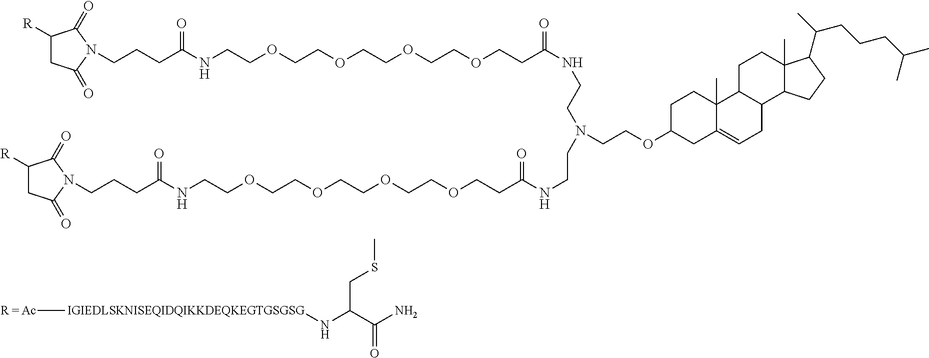

### 2.15 Exemplary Dimeric Fusion Inhibitor of SARS Virus

The sequence of the HRN-binding peptide corresponds to the sequence of peptide P6 (GDISGINASVVNIQKEIDRLNEVAKNL, SEQ ID NO: 122) in Liu et al. (8), with the addition of the C-terminal sequence Gly-Ser-Gly-Ser-Gly-Cys:

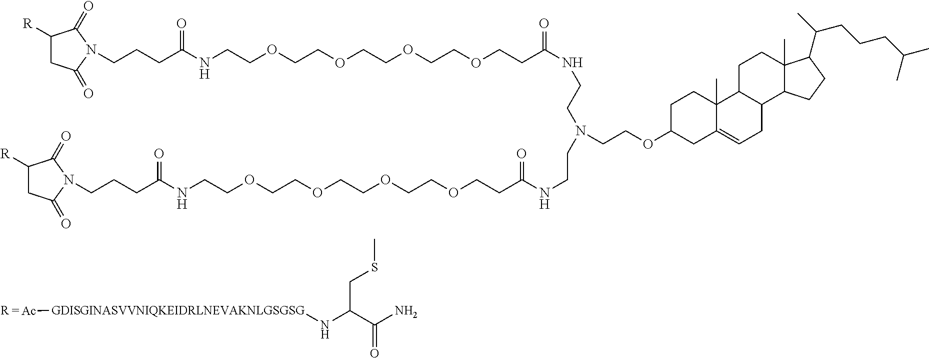

### 2.16 Exemplary Dimeric Fusion Inhibitor of Dengue Virus Type 2 (DV2)

The sequence of the membrane-proximal region (MPR) derived peptide corresponds to the sequence of peptide DV2⁴¹⁹⁻⁴⁴⁰ (AWDFGSLGGVFTSIGKALHQVF, SEQ ID NO: 138) in Schmidt et al. (18), with the addition of the C-terminal sequence Gly-Ser-Gly-Ser-Gly-Cys. It is important to note that DV2⁴¹⁹⁻⁴⁴⁰ has very weak antiviral activity, despite its ability to bind the soluble fusogenic protein E of the dengue virus. This is because DV2⁴¹⁹⁻⁴⁴⁰ lacks amino acids 441-447 which are necessary for membrane association. Accordingly, peptide DV2⁴¹⁹⁻⁴⁴⁷ has the same affinity of DV2⁴¹⁹⁻⁴⁴⁰ for soluble E, but is a potent inhibitor of viral infectivity (18). In the inhibitor claimed in the present invention, the cholesterol group substitutes for the natural membrane-associating sequence 441-447.

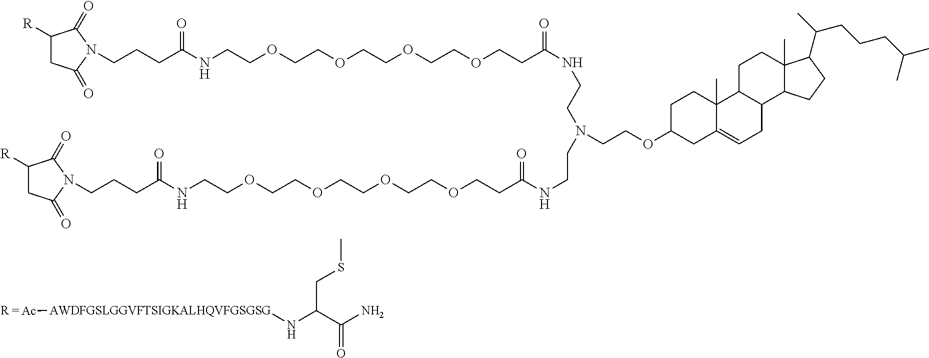

### 2.17 Exemplary Dimeric Fusion Inhibitor of Dengue Virus Type 1 (DV1)

The sequence of the MPR derived peptide corresponds to the sequence of the E protein of DV1 corresponding to the region of DV2 spanned by peptide DV2⁴¹⁹⁻⁴⁴⁰ (AWDFGSIGGVFTSVGKLIHQIF, SEQ ID NO: 137) in Schmidt et al. (18), with the addition of the C-terminal sequence Gly-Ser-Gly-Ser-Gly-Cys:

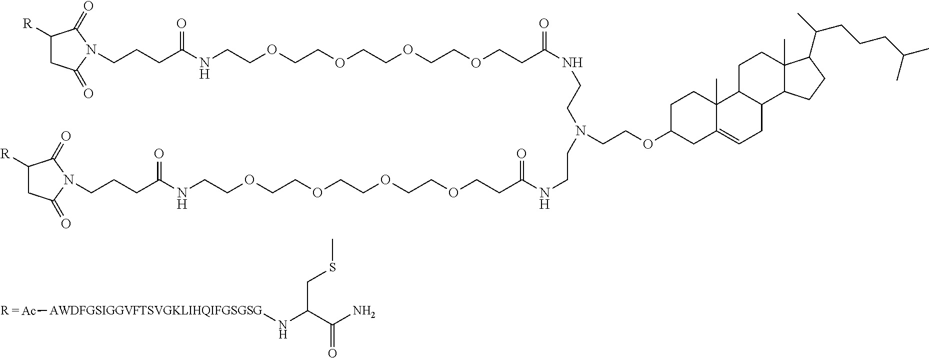

### 2.18 Exemplary Dimeric Fusion Inhibitor of Dengue Virus Type 3 (DV3)

The sequence of the MPR derived peptide corresponds to the sequence of the E protein of DV3 corresponding to the region of DV2 spanned by peptide DV2⁴¹⁹⁻⁴⁴⁰ (AWDFGSVGGVLNSLGKMVHQIF, SEQ ID NO: 139) in Schmidt et al. (18), with the addition of the C-terminal sequence Gly-Ser-Gly-Ser-Gly-Cys:

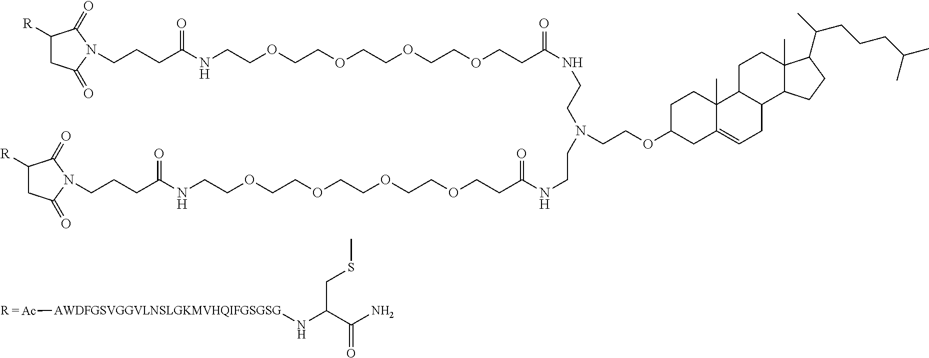

### 2.19 Exemplary Dimeric Fusion Inhibitor of Dengue Virus Type 4 (DV4)

The sequence of the MPR derived peptide corresponds to the sequence of the E protein of DV4 corresponding to the region of DV2 spanned by peptide DV2⁴¹⁹⁻⁴⁴⁰ (AWDFGSVGGLFTSLGKAVHQVF, SEQ ID NO: 140) in Schmidt et al. (18), with the addition of the C-terminal sequence Gly-Ser-Gly-Ser-Gly-Cys:

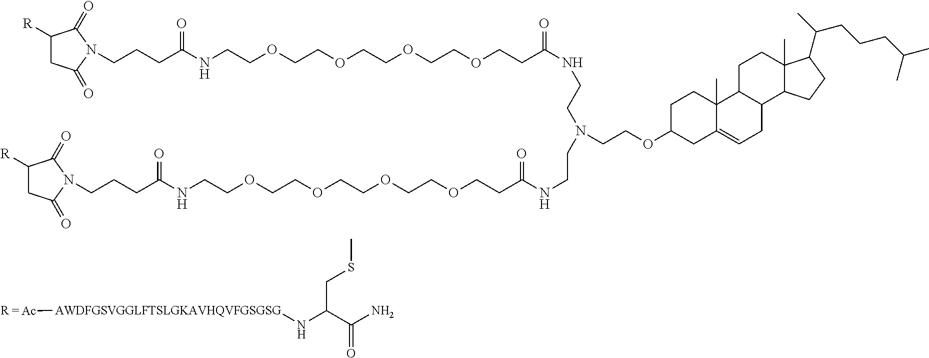

### 2.20 Exemplary Dimeric Fusion Inhibitor of West Nile Virus

The sequence of the MPR derived peptide corresponds to the sequence of the E protein of WNV corresponding to the region of Dengue virus spanned by peptide DV2⁴¹⁹⁻⁴⁴⁰ (AWDFGSVGGVFTSVGKAVHQVF, SEQ ID NO: 141) in Schmidt et al. (18), with the addition of the C-terminal sequence Gly-Ser-Gly-Ser-Gly-Cys:

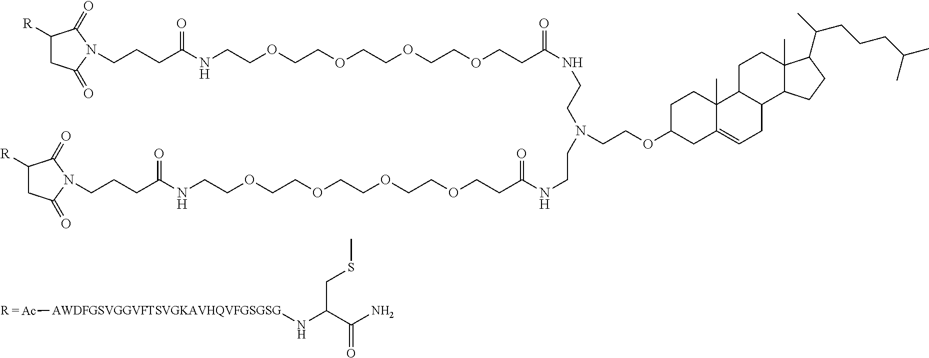

### 2.21 Exemplary Dimeric Fusion Inhibitor of Junin Virus

The sequence of the HRN-binding peptide corresponds to HRC region (amino acids 384-413, SYLNISDFRNDWILESDFLISEMLSKEYSD, SEQ ID NO: 123) of the envelope glycoprotein GP-C of Junin virus as described in (20), with the addition of the C-terminal sequence Gly-Ser-Gly-Ser-Gly-Cys:

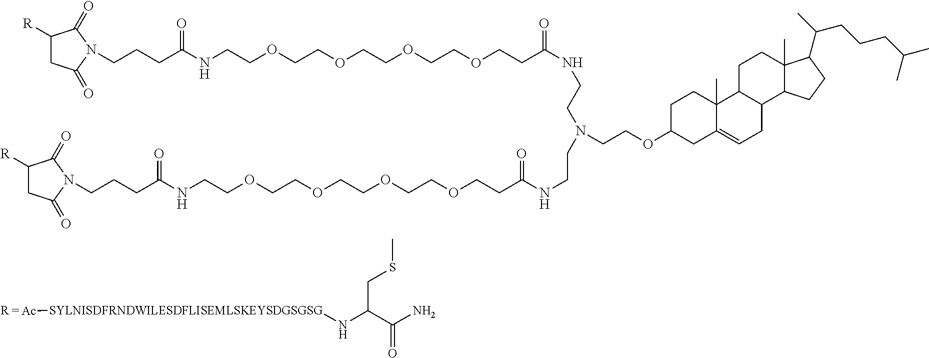

### 2.22 Exemplary Dimeric Fusion Inhibitor of Machupo Virus

The sequence of the HRN-binding peptide corresponds to HRC region (amino acids 384-413, SYLNISEFRNDWILESDHLISEMLSKEYAE, SEQ ID NO: 124) of the envelope glycoprotein GP-C of Machupo virus as described in (20), with the addition of the C-terminal sequence Gly-Ser-Gly-Ser-Gly-Cys:

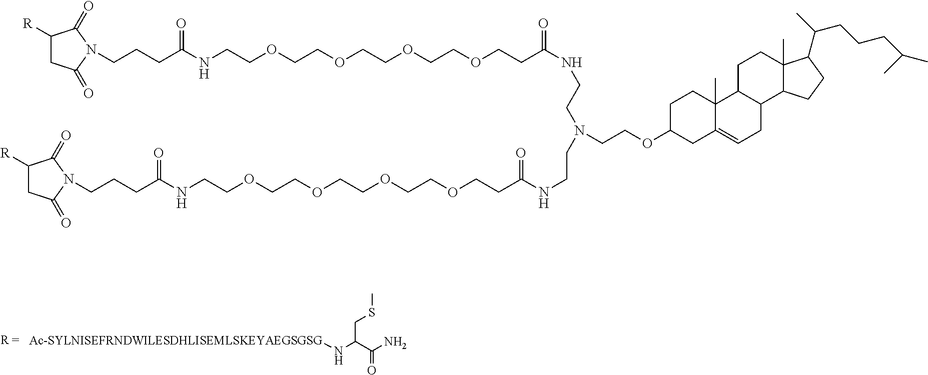

### 2.23 Exemplary Dimeric Fusion Inhibitor of Guanarito Virus

The sequence of the HRN-binding peptide corresponds to HRC region (amino acids 384-413, SYLNESDFRNEWILESDHLISEMLSKEYQD, SEQ ID NO: 125) of the envelope glycoprotein GP-C of Guanarito virus as described in (20), with the addition of the C-terminal sequence Gly-Ser-Gly-Ser-Gly-Cys:

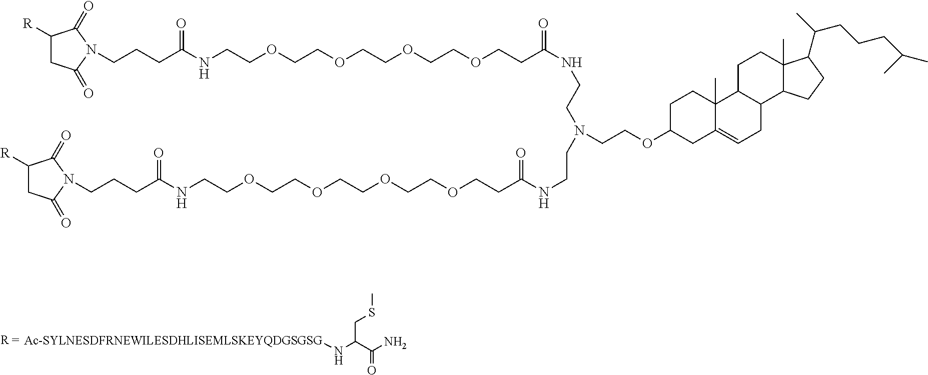

### 2.24 Exemplary Dimeric Fusion Inhibitor of Lassa Virus

The sequence of the HRN-binding peptide corresponds to HRC region (amino acids 384-413, SYLNETHFSDDIEQQADNMITEMLQKEYME, SEQ ID NO: 126) of the envelope glycoprotein GP-C of Lassa virus as described in (20), with the addition of the C-terminal sequence Gly-Ser-Gly-Ser-Gly-Cys:

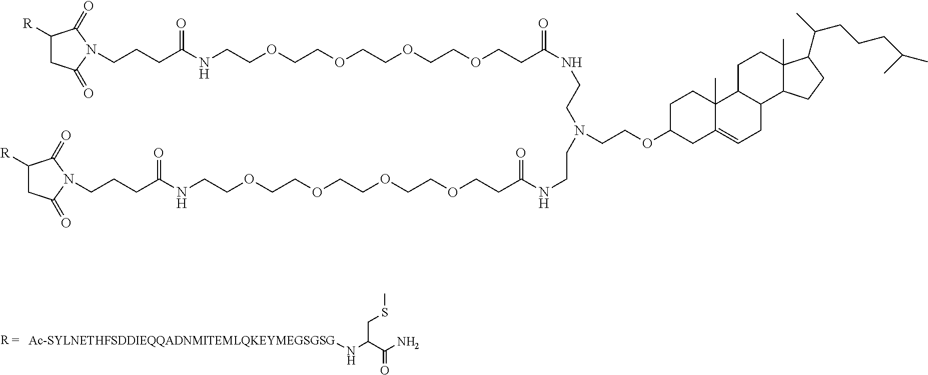

### 3. Exemplary Synthesis of Dimeric Fusion Inhibitors

### 3.1 Example

### Synthesis of [Mal-PEG₄]₂-Chol (11)

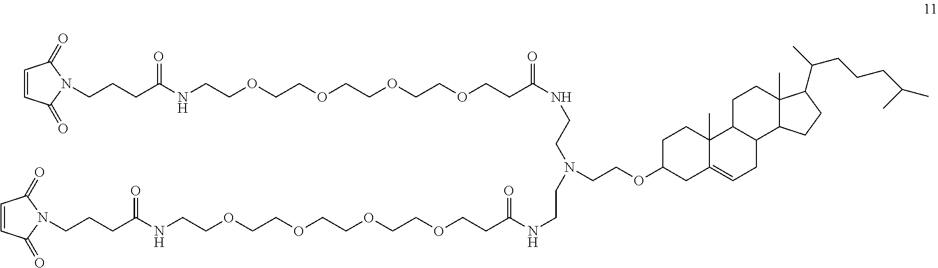 [SMILES: CC(C)CCCC(C)C1CCC2C3CC=C4CC(OCCN(CCNC(=O)CCOCCOCCOCCOCCNC(=O)CCCN5C(=O)C=CC5=O)CCNC(=O)CCOCCOCCOCCOCCNC(=O)CCCN5C(=O)C=CC5=O)CCC4(C)C3CCC12C]

### 1. Synthesis of (1)

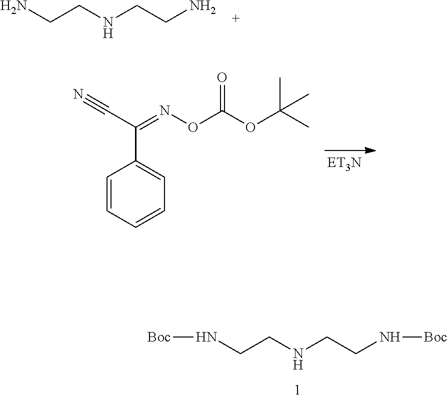 [SMILES: NCCNCCN ; CC(C)(C)OC(=O)O/N=C(/C#N)c1ccccc1 ; CC(C)(C)OC(=O)NCCNCCNC(=O)OC(C)(C)C]

To a flask containing bis(2-aminoethyl)amine (1 g, 9.7 mmol) in 50 mL of THF, cooled at 0° C., was added Et₃N (4.06 mL, 29.1 mmol), and then dropwise 2-(tert-Butoxycarbonyloxyimino)-2-phenylacetonitrile. The mixture was stirred for 1 h at 0° C. and then 2 h at room temperature. After evaporation of the solvent in vacuo, the residue was dissolved in CH₂Cl₂, washed with 1N NaOH, dried over Na₂SO₄, filtered and concentrated, to obtain 2.76 g of 1 as a yellow oil (yield, 94%).

### 2. Synthesis of (3)

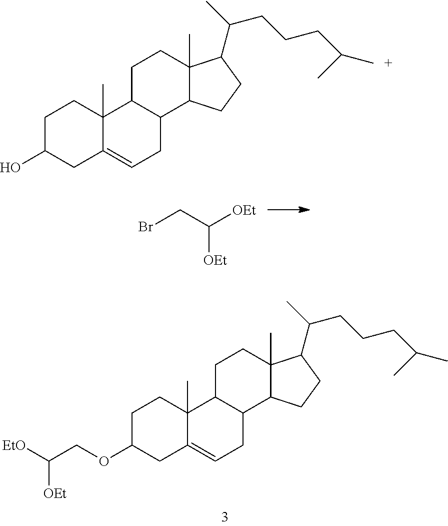

Cholesterol (1 g, 2.6 mmol) was dissolved in 40 mL of THF/DMF (1:1) and 60% sodium hydride (w/w) in mineral oil (0.6 g, 15.5 mmol) was added, followed by stirring for 10 min. 2-bromo-1,1-dimethoxyethane (1.21 mL, 7.8 mmol) was added dropwise, and the mixture was stirred at 90° C. under reflux for 18 h. The mixture was cooled and CH₂CL₂/MeOH (1:1) was added to eliminate excess NaH. After elimination of solvent was eliminated under vacuo, the residue was taken up in EtOAc, washed several times with water, dried over Na₂SO₄, filtered and concentrated. The crude was purified by flash column chromatography (BIOTAGE) on silica gel using a gradient of 2-10% P.E. in EtOAc, to obtain 1.23 g of 3 as a white solid (yield, 94%).

### 3. Synthesis of (4)

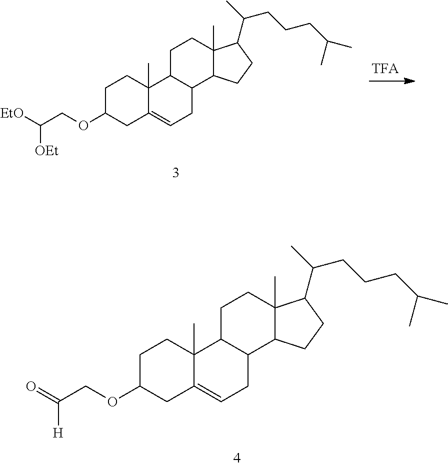 [SMILES: CCOC(COC1CCC2(C)C(=CCC3C2CCC2(C)C(C(C)CCCC(C)C)CCC32)C1)OCC ; CC(C)CCCC(C)C1CCC2C3CC=C4CC(OCC=O)CCC4(C)C3CCC12C]

Trifluoroacetic acid/water (1:1) (2.5 mL, 16.2 mmol) was added to a solution of 3 (0.5 g, 1 mmol) in 10 mL of CH₂Cl₂, and the mixture was stirred at room temperature for 6 h. The mixture was neutralized with 1N NaOH, extracted twice with CH₂Cl₂. dried over Na₂SO₄, filtered and concentrated, to obtain 4 as a white solid, that was used directly in the next step [Literature ref for (4): Bioconj. Chem. 2005, 16(4) 827-836)].

### 4. Synthesis of (5)

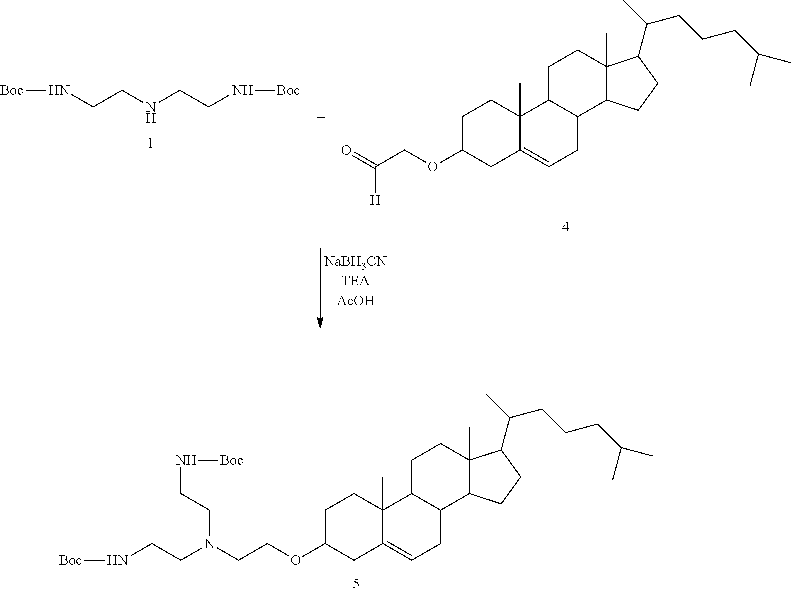 [SMILES: CC(C)(C)OC(=O)NCCNCCNC(=O)OC(C)(C)C ; CC(C)CCCC(C)C1CCC2C3CC=C4CC(OCC=O)CCC4(C)C3CCC12C ; CC(C)CCCC(C)C1CCC2C3CC=C4CC(OCCN(CCNC(=O)OC(C)(C)C)CCNC(=O)OC(C)(C)C)CCC4(C)C3CCC12C]

4 (1.47 mmol) was dissolved in 40 mL of MeOH containing 1 (0.535 g, 1.77 mmol), and Et3N (0.611 mL, 4.41 mmol), AcOH (0.42 mL, 7.35 mmol), and NaBH₃CN (0.185 g, 2.94 mmol) were added in succession. The mixture was stirred for 18 h at room temperature, diluted with EtOAc, washed twice with NaHCO₃ and water, dried over Na₂SO₄, filtered and concentrated. The crude was purified by flash column chromatography (BIOTAGE) on silica gel using a gradient of 0-8% MeOH in CH₂Cl₂, to obtain 0.95 g of 5 as a white solid (yield, 90%).

### 5. Synthesis of (6)

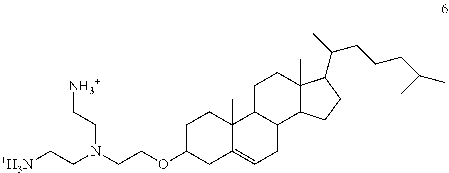 [SMILES: CC(C)CCCC(C)C1CCC2C3CC=C4CC(OCCN(CC[NH3+])CC[NH3+])CCC4(C)C3CCC12C]

Trifluoroacetic acid (3.6 mL, 46.7 mmol) was added to a solution of 5 (1.12 g, 1.57 mmol) in 20 mL of CH₂Cl₂, and the mixture was stirred at room temperature for 4 h. All the volatiles were removed under vacuo and the crude was lyophilized to obtain 6 as a brown solid, that was used directly in the next step.

### 6. Synthesis of (8)

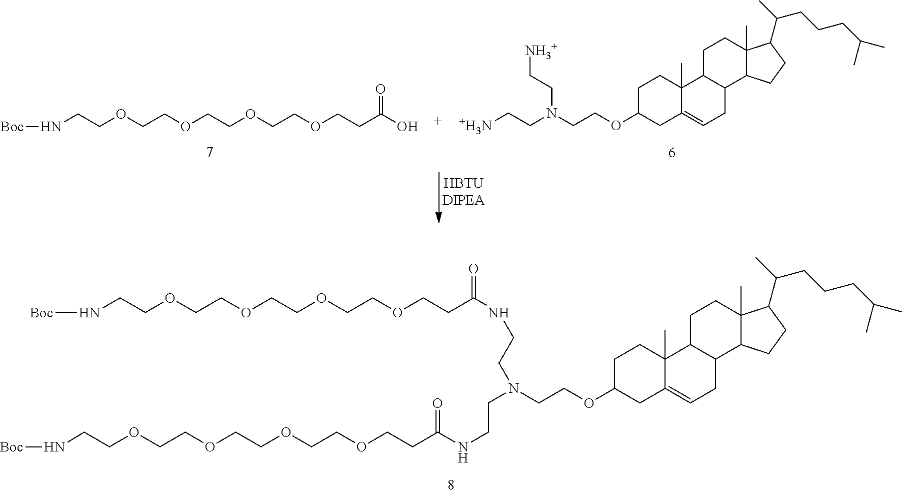 [SMILES: CC(C)(C)OC(=O)NCCOCCOCCOCCOCCC(=O)O ; CC(C)CCCC(C)C1CCC2C3CC=C4CC(OCCN(CC[NH3+])CC[NH3+])CCC4(C)C3CCC12C ; CC(C)CCCC(C)C1CCC2C3CC=C4CC(OCCN(CCNC(=O)CCOCCOCCOCCOCCNC(=O)OC(C)(C)C)CCNC(=O)CCOCCOCCOCCOCCNC(=O)OC(C)(C)C)CCC4(C)C3CCC12C]

N-t-boc-amido-dPEG₄™ acid (7) (1.15 g, 3.14 mmol, Product No 10220, Quanta BioDesign, Ltd.) was dissolved in 47 mL of CH₂Cl₂ together with O-Benzotriazole-N,N,N′,N′-tetramethyl-uronium-hexafluoro-phosphate (HBTU, 1.31 g, 3.45 mmol) and N,N′-diisopropylethylamine (DIPEA, 1.1 mL, 6.28 mmol). The mixture was stirred for 30 min at room temperature until complete dissolution of HBTU. 6 was added and the mixture stirred for 2 h at room temperature. Since some mono-derivatized compound was still present, a new addition was made of 7 (365 mg, 1 mmol), HBTU (402 mg, 1.1 mmol), and DIPEA (0.350 mL, 2 mmol) dissolved in 30 mL of CH₂Cl₂. The mixture was stirred at room temperature for another hour, after which the reaction was complete. After addition of water and CH₂Cl₂, the organic phase was separated, the aqueous phase extracted with CH₂Cl₂, and the combined organic phase was dried over Na₂SO₄, filtered and concentrated. The crude was purified by flash column chromatography (BIOTAGE) on silica gel using as solvent CH₂Cl₂/MeOH/Et₃N (97.5:2:0.5) to obtain 1.88 g of 8 as a yellow oil (yield, 98%).

### 7. Synthesis of (9)

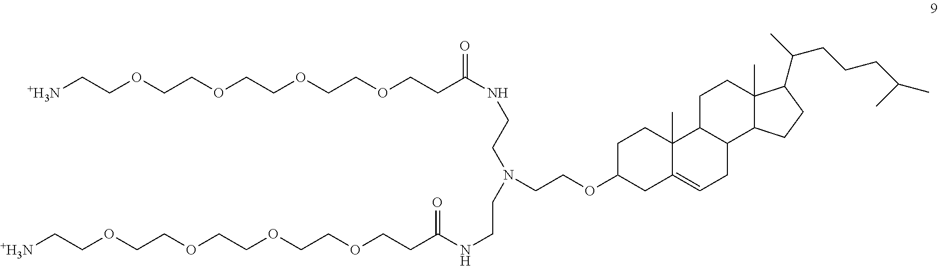 [SMILES: CC(C)CCCC(C)C1CCC2C3CC=C4CC(OCCN(CCNC(=O)CCOCCOCCOCCOCC[NH3+])CCNC(=O)CCOCCOCCOCCOCC[NH3+])CCC4(C)C3CCC12C]

Trifluoroacetic acid (3.6 mL, 46.7 mmol) was added to a solution of 8 (1.88 g, 1.55 mmol) in 30 mL of CH₂Cl₂, and the mixture was stirred at room temperature for 3 h. All the volatiles were removed under vacuo and the crude was lyophilized to obtain 9 as a brown oil, that was used directly in the next step.

### 8. Synthesis of (11)

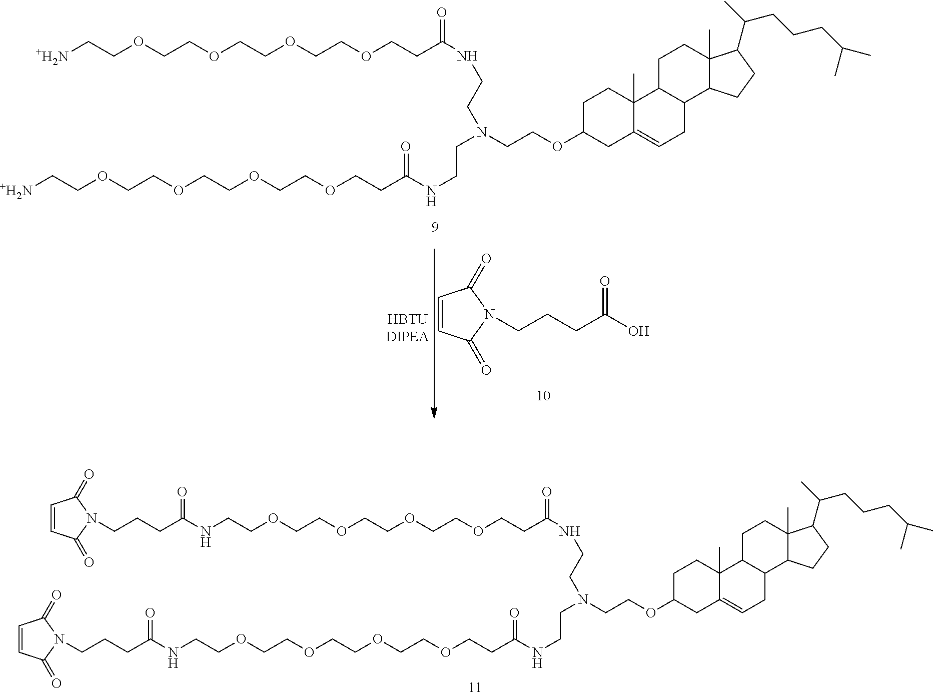 [SMILES: CC(C)CCCC(C)C1CCC2C3CC=C4CC(OCCN(CCNC(=O)CCOCCOCCOCCOCC[NH3+])CCNC(=O)CCOCCOCCOCCOCC[NH3+])CCC4(C)C3CCC12C ; O=C(O)CCCN1C(=O)C=CC1=O ; CC(C)CCCC(C)C1CCC2C3CC=C4CC(OCCN(CCNC(=O)CCOCCOCCOCCOCCNC(=O)CCCN5C(=O)C=CC5=O)CCNC(=O)CCOCCOCCOCCOCCNC(=O)CCCN5C(=O)C=CC5=O)CCC4(C)C3CCC12C]

4-maleimido-butyric acid (10) (0.284 g, 1.55 mmol) was dissolved in 25 mL of CH₂Cl₂ together with HBTU (0.617 g, 1.62 mmol) and DIPEA (0.57 mL, 3.26 mmol). 0.5 mL of DMF was added to help complete dissolution. 9 dissolved in 20 mL of CH₂CL₂ was added, and the mixture was stirred for 2 h at room temperature, after which the reaction was complete. After addition of water and CH₂Cl₂, the organic phase was separated, the aqueous phase extracted with CH₂Cl₂, and the combined organic phase was dried over Na₂SO₄, filtered and concentrated. The crude was purified by flash column chromatography (BIOTAGE) on silica gel using a gradient of 4-15% MeOH in CH₂Cl₂, yielding 0.462 g of 11 as a white solid (yield, 56%).

### 3.2 Example

### Synthesis of [(Ac-VALDPIDISIVLNKAKSDLEESKEWIRRSNGKLDSIGSGSG-C(Mal-PEG₄)]₂—Chol (13)

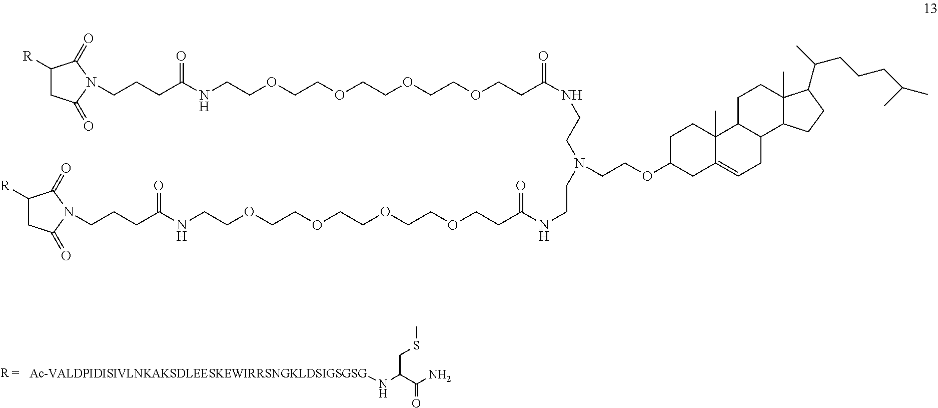

### 1. Synthesis of Ac-VALDPIDISIVLNKAKSDLEESKEWIRRSNGKLDSIGSGSG-C—NH₂(12)

The peptide was synthesized with solid-phase Fmoc chemistry on an APEX396 synthesizer (Advanced Chemtek) using AM-Polysryrene LL resin (100-200 mesh, Novabiochem) derivatized with a modified Rink linker p-[(R,S)-α-[9H-Fluoren-9-yl-methoxyformamido]-2,4-dimethoxybenzyl]-phenoxyacetic acid. The following side chain protecting groups were used: OtBu for Asp and Glu; tBu for Ser; Boc for Lys and Trp; Trt for Asn and Cys. All the amino acids were dissolved at a 0.5 M concentration in a solution of 0.5M HOBt (Hydroxybenzotriazole) in DMF. The acylation reactions were performed for 60 min with 6-fold excess of activated amino acid over the resin free amino groups. The amino acids were activated with equimolar amounts of HATU (2-(1H-benzotriazole-1-yl)-1,1,3,3-tetramethyluronium hexafluorophosphate) and a 2-fold molar excess of DIEA (N,N-diisopropylethylamine). The acetylation reaction was performed at the end of the peptide assembly by reaction with a 10-fold excess of acetic anhydride in DMF.

At the end of the synthesis the dry peptide-resin was treated with the cleavage mixture, 82.5% TFA, 5% phenol, 5% Tioanisole, 5% water, 2.5% EDT for 1.5 h at room temperature.

The resin was filtered and the solution was added to cold methyl-t-butyl ether in order to precipitate the peptide. After centrifugation, the peptide pellets were washed with fresh cold methyl-t-butyl ether to remove the organic scavengers. The process was repeated twice. Final pellets were dried, resuspended in 30% acetonitrile and lyophilized.

PURIFICATION AND ANALYTICAL CHARACTERIZATION. The crude peptide was purified by reverse-phase HPLC using preparative XBridge Prep C18 (50×150 mm, 5 μm) and as eluents (A) 0.1% TFA in water and (B) 0.1% TFA in acetonitrile. The following gradient was used: 33%-33% (5 min)-47% B (20 min)-80% B (3 min), flow rate 80 ml/min at RT, λ=214 nm. The purified peptide was lyophilized and structure and purity was confirmed by analytical UPLC and electrospray mass spectrometry on a SQ Detector Waters platform. Analytical UPLC/MS was performed on a Waters Acquity UPLC BEH300 C4 column (2.1×100 mm, 1.7 μm) with the following gradient of eluent B: 35%-35% (1 min)-55% B (4 min)-80% B (0.5 min), flow rate 0.4 ml/min, T=45° C., λ=214 nm (rt: 3.22 min; MW found: 4543, MW calc: 4543.1 Da; HPLC purity: 95%; yield: 10%).

### 2. Synthesis of (13) [(Ac-VALDPIDISIVLNKAKSDLEESKEWIRRSNGKLDSIGSGSG-C(Mal-PEG₄)]₂-Chol (13)

was synthesized by chemoselective conjugation between the Cys-peptide precursor (12) and the cholesterol derivative (11). 5 mg (3.7 μmol) of (11) dissolved in 0.4 mL of THF (10 mg/mL), were added to 30 mg of (12) (6.6 μmol) dissolved in 1.5 mL of DMSO (conc. 20 mg/mL). The reaction was monitored by UPLC-mass spectrometry on a SQ Detector Waters Platform with a Waters Acquity UPLC BEH300 C4 column (2.1×100 mm, 1.7 μm) using as eluents (A) 0.1% TFA in water and (B) 0.1% TFA in acetonitrile, and the following linear gradient: 30%-30% (B) in 1 min, then—30%-95% (B) in 4 min, flow 0.4 ml/min, T=45° C., λ=214 nm; tr=3.02′. After 60 min the reaction was complete and it was quenched with glacial acetic acid to pH=4, then water was added drop-wise up to the highest amount that did not induce precipitation.

PURIFICATION AND ANALYTICAL CHARACTERIZATION. The resulting cholesterol-peptide product (3) was purified by reverse-phase HPLC on a C4 DELTAPAK Waters cartridge, 300 Å 20×100 mm, 15 μm; Flow: 80 mL/min: Gradient: 30% B isocratic 5 min then linear to 70% B in 20 min; Eluents: (A) 0.2% AcOH in water and (B) 0.2% AcOH in acetonitrile. AcOH was used to obtain the final compound as an acetate salt. Pooling of the cleanest fractions gave 15.4 mg of (13) at ≧95% purity (yield: 51%) (MW: cal., 10426 Da, found, 10421 Da).

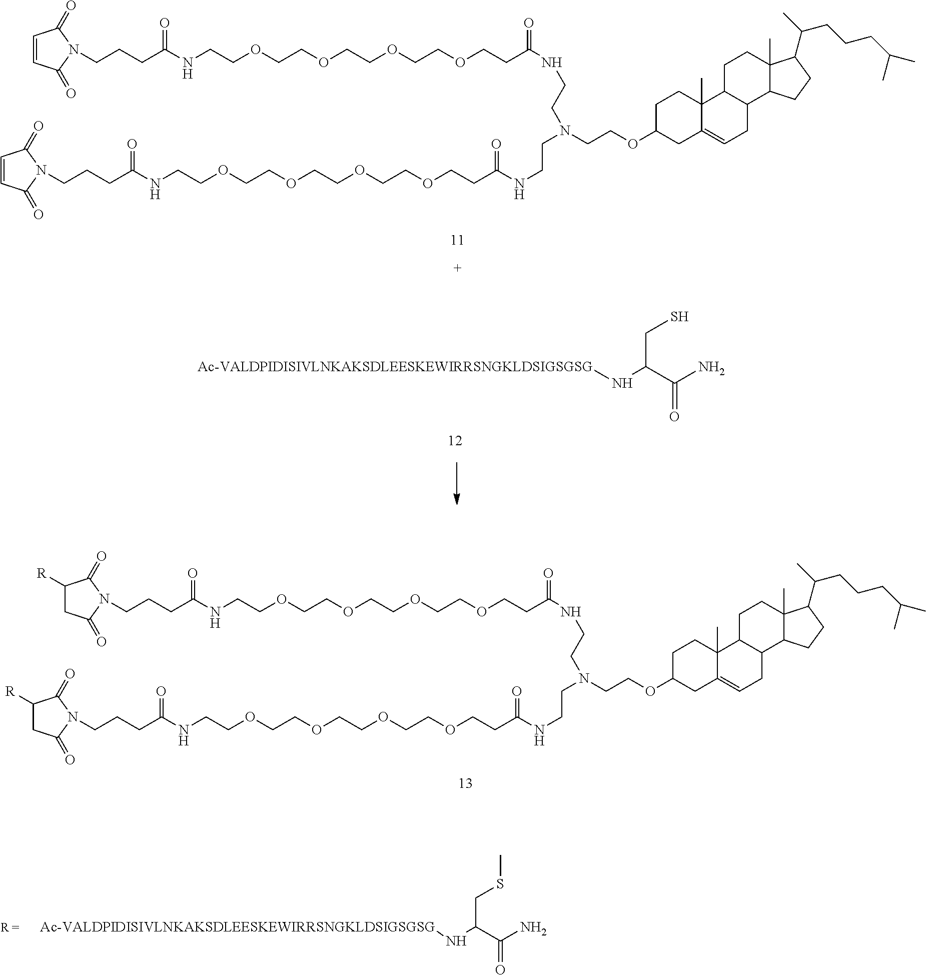

### 3.3 Example

### Synthesis of [(Ac-VALDPIDISIVLNKIKSDLEESKEWIRRSNKILDSIGSGSG-C(Mal-PEG₄)]₂-Chol (15)

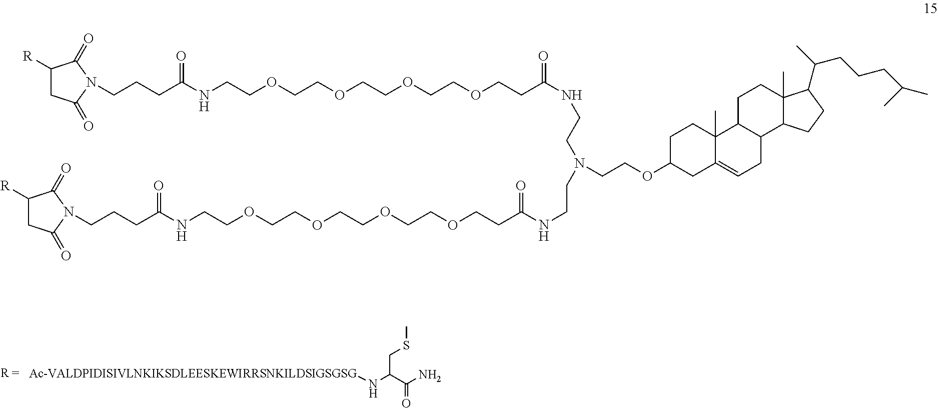

### 1. Synthesis of Ac-VALDPIDISIVLNKIKSDLEESKEWIRRSNKILDSIGSGSG-C—NH₂ (14)

The peptide was synthesized with solid-phase Fmoc chemistry on an APEX396 synthesizer (Advanced Chemtek) using AM-Polysryrene LL resin (100-200 mesh, Novabiochem) derivatized with a modified Rink linker p-[(R,S)-α-[9H-Fluoren-9-yl-methoxyformamido]-2,4-dimethoxybenzyl]-phenoxyacetic acid. The following side chain protecting groups were used: OtBu for Asp and Glu; tBu for Ser; Boc for Lys and Trp; Trt for Asn and Cys. All the amino acids were dissolved at a 0.5 M concentration in a solution of 0.5M HOBt (Hydroxybenzotriazole) in DMF. The acylation reactions were performed for 60 min with 6-fold excess of activated amino acid over the resin free amino groups. The amino acids were activated with equimolar amounts of HATU (2-(1H-benzotriazole-1-yl)-1,1,3,3-tetramethyluronium hexafluorophosphate) and a 2-fold molar excess of DIEA (N,N-diisopropylethylamine). The acetylation reaction was performed at the end of the peptide assembly by reaction with a 10-fold excess of acetic anhydride in DMF.

At the end of the synthesis the dry peptide-resin was treated with the cleavage mixture, 82.5% TFA, 5% phenol, 5% Tioanisole, 5% water, 2.5% EDT for 1.5 h at room temperature.

The resin was filtered and the solution was added to cold methyl-t-butyl ether in order to precipitate the peptide. After centrifugation, the peptide pellets were washed with fresh cold methyl-t-butyl ether to remove the organic scavengers. The process was repeated twice. Final pellets were dried, resuspended in 30% acetonitrile and lyophilized.

PURIFICATION AND ANALYTICAL CHARACTERIZATION. The crude peptide was purified by reverse-phase HPLC using preparative XBridge Prep C18 (50×150 mm, 5 μm) and as eluents (A) 0.1% TFA in water and (B) 0.1% TFA in acetonitrile. The following gradient was used: 33%-33% (5 min)-47% B (20 min)-80% B (3 min), flow rate 80 ml/min at RT, λ=214 nm. The purified peptide was lyophilized and structure and purity was confirmed by analytical UPLC and electrospray mass spectrometry on a SQ Detector Waters platform. Analytical UPLC/MS was performed on a Waters Acquity UPLC BEH300 C4 column (2.1×100 mm, 1.7 μm) with the following gradient of eluent B: 35%-35% (1 min)-55% B (4 min)-80% B (0.5 min), flow rate 0.4 ml/min, T=45° C., λ=214 nm (rt: 3.22 min; MW found: 4639.6, MW calc: 4641.3 Da; HPLC purity: 95%; yield: 10%).

### 2. Synthesis of (15) [(Ac-VALDPIDISIVLNKIKSDLEESKEWIRRSNKILDSIGSGSG-C(Mal-PEG₄)]₂-Chol (15)

was synthesized by chemoselective conjugation between the Cys-peptide precursor (14) and the cholesterol derivative (11). 5 mg (3.7 mop of (11) dissolved in 0.4 mL of THF (10 mg/mL), were added to 30.6 mg of (14) (6.6 μmol) dissolved in 1.5 mL of DMSO (conc. 20 mg/mL). The reaction was monitored by UPLC-mass spectrometry on a SQ Detector Waters Platform with a Waters Acquity UPLC BEH300 C4 column (2.1×100 mm, 1.7 μm) using as eluents (A) 0.1% TFA in water and (B) 0.1% TFA in acetonitrile, and the following linear gradient: 30%-30% (B) in 1 min, then—30%-95% (B) in 4 min, flow 0.4 ml/min, T=45° C., λ=214 nm; tr=3.02′. After 60 min the reaction was complete and it was quenched with glacial acetic acid to pH=4, then water was added drop-wise up to the highest amount that did not induce precipitation.

PURIFICATION AND ANALYTICAL CHARACTERIZATION. The resulting cholesterol-peptide product (15) was purified by reverse-phase HPLC on a C4 DELTAPAK Waters cartridge, 300 Å 20×100 mm, 15 μm; Flow: 80 mL/min: Gradient: 30% B isocratic 5 min then linear to 70% B in 20 min; Eluents: (A) 0.2% AcOH in water and (B) 0.2% AcOH in acetonitrile. AcOH was used to obtain the final compound as an acetate salt. Pooling of the cleanest fractions gave 17.9 mg of (15) at ≧95% purity (yield: 58%) (MW: cal., 10622 Da, found, 10618 Da).

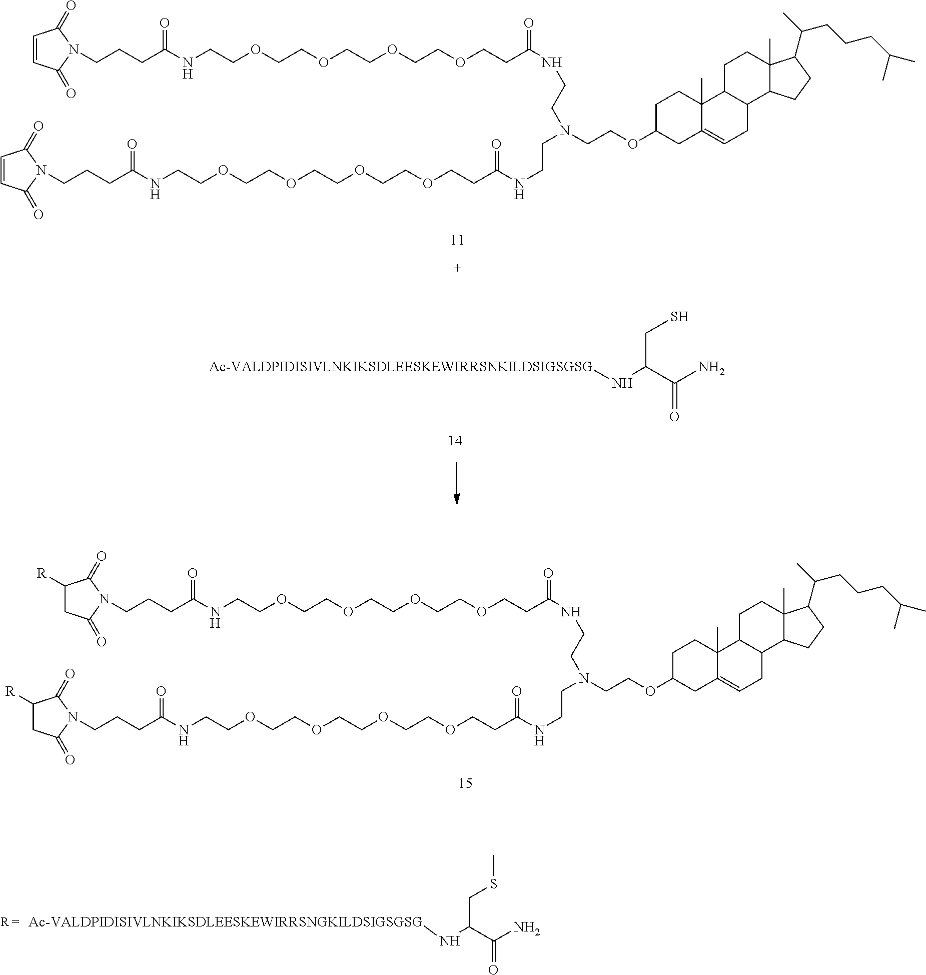

### 3.4 Example

### Synthesis of [(Ac-WMEWDREINNYTSLIHSLIEESQNQQEKNEQELL-GSG-C(Mal-PEG₄)]₂-Chol (17)

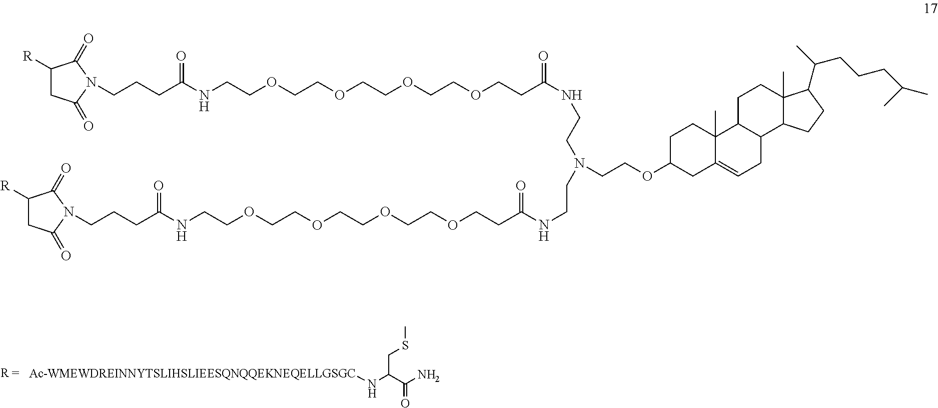

### 1. Synthesis of Ac-WMEWDREINNYTSLIHSLIEESQNQQEKNEQELLGSG-C—NH₂ (16)

Peptide (16) was synthesized with solid-phase Fmoc chemistry with an APEX396 synthesizer (Advanced Chemtek) using AM-Polysryrene LL resin (100-200 mesh, Novabiochem) derivatized with a modified Rink linker p-[(R,S)-α-[9H-Fluoren-9-yl-methoxyformamido]-2,4-dimethoxybenzyl]-phenoxyacetic acid. The following side chain protecting groups were used: OtBu for Asp and Glu; tBu for Ser, Thr and Tyr; Boc for Lys and Trp; Trt for Asn, Cys, His and Gln; Pbf for Arg. All the amino acids were dissolved at a 0.5 M concentration in a solution of 0.5M HOBt (Hydroxybenzotriazole) in DMF. The acylation reactions were performed for 60 min with 6-fold excess of activated amino acid over the resin free amino groups. The amino acids were activated with equimolar amounts of HATU (2-(1H-benzotriazole-1-yl)-1,1,3,3-tetramethyluronium hexafluorophosphate) and a 2-fold molar excess of DIEA (N,N-diisopropylethylamine). The acetylation reaction was performed at the end of the peptide assembly by reaction with a 10-fold excess of acetic anhydride in DMF.

At the end of the synthesis, the dry peptide-resin was treated with the cleavage mixture, 82.5% trifluoroacetic acid (TFA), 5% phenol, 5% thioanisole, 2.5% ethandithiole and 5% water for 1.5 hours at room temperature (0.1 g peptide-resin/1.5 mL mixture). The resin was filtered and the solution was added to cold methyl-t-butyl ether in order to precipitate the peptide. After centrifugation, the peptide pellets were washed with fresh cold methyl-t-butyl ether to remove the organic scavengers. The process was repeated twice. Final pellets were dried, resuspended in H₂O, 20% acetonitrile, 0.1% TFA and lyophilized.

PURIFICATION AND ANALYTICAL CHARACTERIZATION. The crude peptide was purified by reverse-phase HPLC using preparative Waters Reprosil Pure C4 300 Å (50×150 mm, 10 μm) and using as eluents (A) 0.1% TFA in water and (B) 0.1% TFA in acetonitrile. The following gradient of eluent B was used: 35%-35% over 5 min and 35%-43% over 20 min, flow rate 80 mL/min, T=22° C., λ=214 nm (RT 21.8 min). Analytical UPLC was performed on a Waters Acquity UPLC BEH300 C4 column (2.1×100 mm, 1.7 μm) with the following gradient of eluent B: 35%-35% (in 1 min)-50% B (in 4 min)-80% (in 20 sec), flow rate 0.4 mL/min, T=45° C., λ=214 nm (RT=3.26 min). The purified peptide was lyophilized and structure and purity above 97% were confirmed by analytical UPLC and electrospray mass spectrometry on a SQ Detector Waters platform (Mw found: 4593 Da; Mw calc: 4594 Da).

### 2. Synthesis of (17) [(Ac-WMEWDREINNYTSLIHSLIEESQNQQEKNEQELL-GSG-C(Mal-PEG₄)]₂-Chol (17)

was synthesized by chemoselective conjugation between the Cys-peptide precursor (16) and the cholesterol derivative (11). 2.7 mg (2 mol) of (11) dissolved in 0.3 mL of THF (10 mg/mL), were added to 20 mg of (16) (4.3 μmol) dissolved in 1 mL of DMSO (conc. 20 mg/mL), followed by addition of 12.7 μL of DIPEA and 64 μL of a 70 mM aqueous EDTA solution (pH=8). The reaction was monitored by UPLC-mass spectrometry on a SQ Detector Waters Platform with a Waters Acquity UPLC BEH300 C4 column (2.1×100 mm, 1.7 μm) using as eluents (A) 0.1% TFA in water and (B) 0.1% TFA in acetonitrile, and the following linear gradient: 30%-30% (B) in 1 min, then—30%-95% (B) in 4 min, flow 0.4 ml/min, T=45° C., λ=214 nm; tᵣ=3.02′. After 30 min the reaction was complete and it was quenched with trifluoroacetic acid to pH=3, then water was added drop-wise up to the highest amount that did not induce precipitation.

PURIFICATION AND ANALYTICAL CHARACTERIZATION. The resulting cholesterol-peptide product (17) was purified by reverse-phase HPLC on a C4 DELTAPAK Waters cartridge, 300 Å 25×100 mm, 15 μm; Flow: 80 mL/min: Gradient: 30% B isocratic 5 min then linear to 70% B in 20 min; Eluents: (A) 0.2% TFA in water and (B) 0.2% TFA in acetonitrile. Pooling of the cleanest fractions gave 7.2 mg of (17) at ≧95% purity (yield: 35%) (MW: cal., 10528 Da, found, 10525 Da).

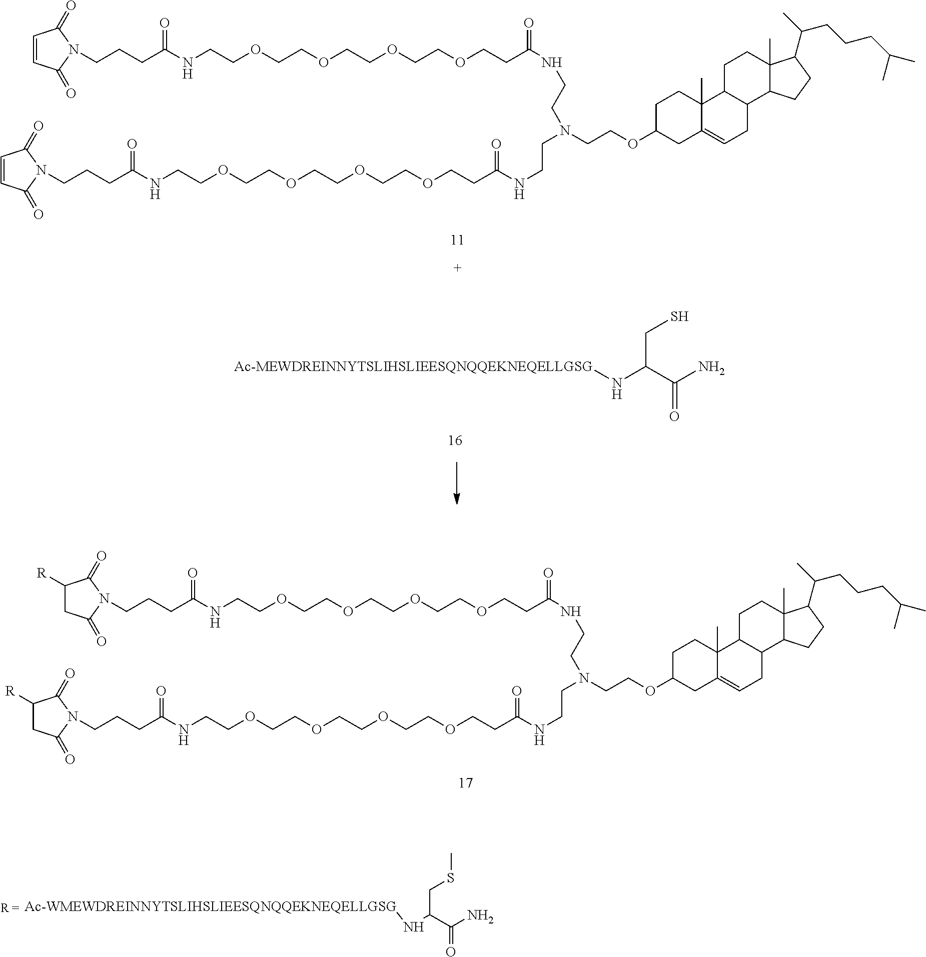

### 4. Exemplary Antiviral Activity of Cholesterol-Derivatized Fusion Inhibitors

### 4.1 Example

### Antiviral Activity of Cholesterol-Derivatized Inhibitors Derived from the Sequence of Human Parainfluenza Virus Type 3 (HPIV3) Against HPIV3

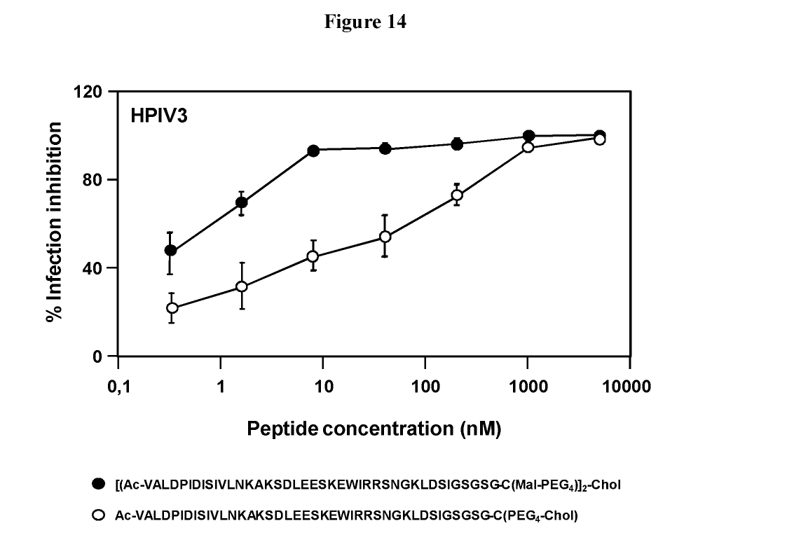

The antiviral activity of a dimeric cholesterol-derivatized inhibitor derived from the sequence of Human Parainfluenza virus type 3 (HPIV3) against HPIV3 has been tested. FIG. 14 shows the antiviral activity against human parainfluenza virus type 3 (HPIV3) of the dimeric Fusion Inhibitor [(Ac-VALDPIDISIVLNKAKSDLEESKEWIRRSNGKLDSIGSGSG-C(Mal-PEG₄)]₂-Chol, in comparison with the corresponding monomeric fusion inhibitor Ac-VALDPIDISIVLNKAKSDLEESKEWIRRSNGKLDSIGSGSG-C(PEG₄-Chol). The plaque reduction assay is performed as described in (13, 15-17).

### 4.2 Example

### Antiviral Activity of Cholesterol-Derivatized Inhibitors Derived from the Sequence of Human Parainfluenza Virus Type 3 (HPIV3) Against Nipah Virus

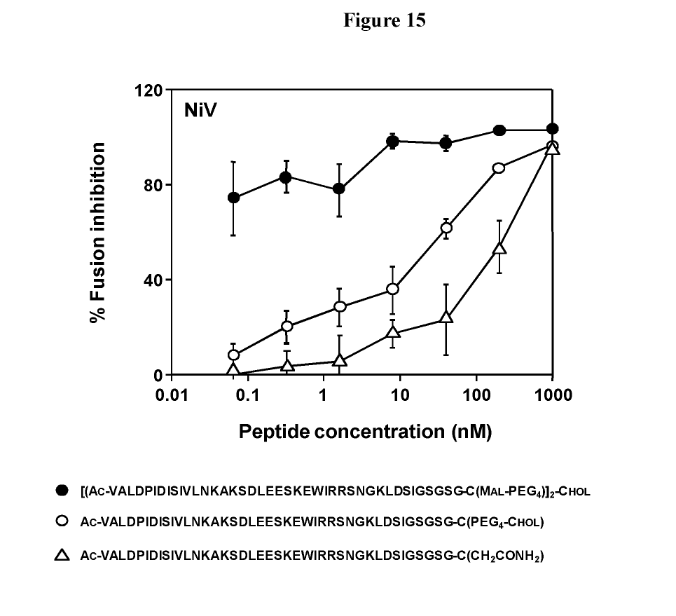

The antiviral activity of a dimeric cholesterol-derivatized inhibitor derived from the sequence of Human Parainfluenza virus type 3 (HPIV3) against Nipah virus (NiV) has been tested. FIG. 15 shows the antiviral activity of the dimeric Fusion Inhibitor [(Ac-VALDPIDISIVLNKAKSDLEESKEWIRRSNGKLDSIGSGSG-C(Mal-PEG₄)]₂-Chol against NiV in a fusion inhibition assay, in comparison with the corresponding monomeric fusion inhibitor Ac-VALDPIDISIVLNKAKSDLEESKEWIRRSNGKLDSIGSGSG-C(PEG₄-Chol), and the control peptide lacking cholesterol Ac-VALDPIDISIVLNKAKSDLEESKEWIRRSNGKLDSIGSGSG-C(CH₂CONH₂), where the cysteine residue is alkylated with iodoacetamide. The assay is performed as described in (14).

### 4.3 Example

### Antiviral Activity of Cholesterol-Derivatized Inhibitors Derived from the Sequence of Human Parainfluenza Virus Type 3 (HPIV3) Against Measles Virus (MV), Edmonton Strain

The antiviral activity of a dimeric cholesterol-derivatized inhibitor derived from the sequence of Human Parainfluenza virus type 3 (HPIV3) was tested in a fusion inhibition assay against the Edmonton strain of Measles Virus (MV): this is the strain used in the measles vaccine.

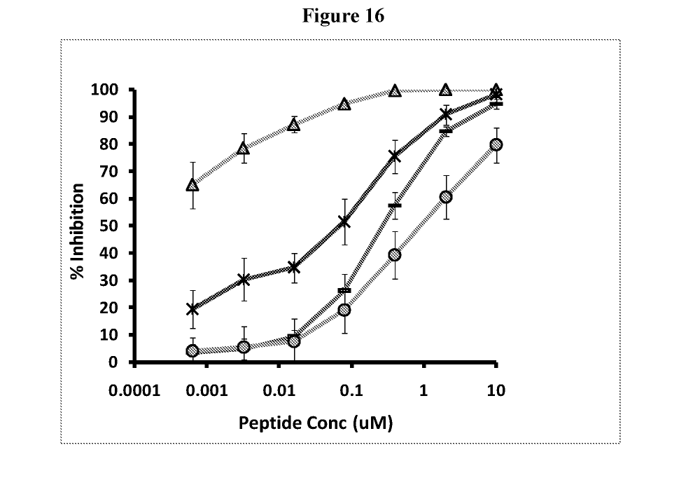

FIG. 16 shows a comparison of the inhibition of MV fusion by the dimeric fusion inhibitor [(Ac-VALDPIDISIVLNKAKSDLEESKEWIRRSNGKLDSI-GSGSG-C(Mal-PEG₄)]₂-Chol (A), by the monomeric fusion inhibitor Ac-VALDPIDISIVLNKAKSDLEESKEWIRRSNGKLDSI-GSGSG-C(PEG₄-Chol) (X), by the dimeric fusion inhibitor with a benzyl group instead of cholesterol [(Ac-VALDPIDISIVLNKAKSDLEESKEWIRRSNGKLDSI-GSGSG-C(Mal-PEG₄)]₂-OBz (−), and by the control monomeric peptide without cholesterol Ac-VALDPIDISIVLNKAKSDLEESKEWIRRSNGKLDSI-GSGSG-C(CH₂CONH₂) (∘).

### 4.4 Example

### Antiviral Activity of Cholesterol-Derivatized Inhibitors Derived FROM the Sequence of Human Parainfluenza Virus Type 3 (HPIV3) Against Measles Virus (MV), Wild-Type Isolate

The antiviral activity of a cholesterol-derivatized inhibitors derived from the sequence of Human Parainfluenza virus type 3 (HPIV3) was tested against a wild-type (WT) strain of Measles Virus (MV).

As apparent from the Table below, the monomeric cholesterol-derivatized inhibitor is 5-fold more potent than the underivatized peptide against the WT strain, while the dimeric cholesterol-derivatized inhibitor is 250-fold more potent than the underivatized peptide.

| | IC₅₀ |
|---|---|
| Compound | WT |
| Ac-VALDPIDISIVLNKAKSDLEESKEWIRRSNGKLDSIGSGSG-C(CH₂CONH₂) | 3824 |
| Ac-VALDPIDISIVLNKAKSDLEESKEWIRRSNGKLDSIGSGSG-C(PEG₄-Chol) | 735 |
| [(Ac-VALDPIDISIVLNKAKSDLEESKEWIRRSNGKLDSIGSGSG-C(Mal-PEG₄)]₂-Chol | 15 |

### 4.5 Example

### Antiviral Activity of Cholesterol-Derivatized Inhibitors Against Human Immunodeficiency Virus (HIV)

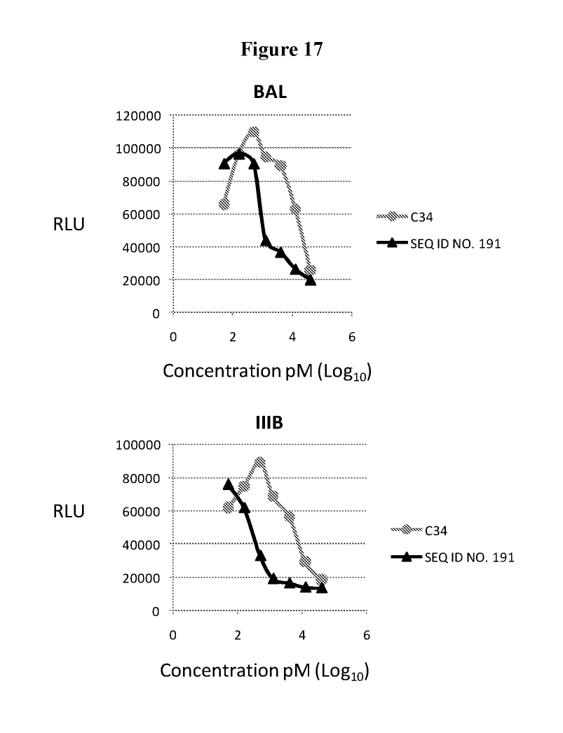

The antiviral activity of a monomeric cholesterol-derivatized inhibitor was tested against Human Immunodeficiency Virus (HIV). The target cells were HIV-LTR-Luc+/GFP+ HeLa-derived TZMBL (10⁵ cells/ml in 0.1 ml/well). The cells were preincubated for 1 h at 37° C. with the peptide(s). The cells were then incubated with CCR5-dependent (R5-Bal) or CXCR4-dependent (LAI/IIIB) HIV-1 strains at a multiplicity of infection (MOI) of 4, corresponding to a 10⁻³ dilution. MOI is the ratio between the number of infectious units and the number of target cells Three doses of both Bal and Lai/IIIB virus corresponding to 10⁻⁵, 10⁻⁴, and 10⁻³ dilutions were tested in the absence of compounds. The lower dilution (10⁻³) was chosen because it gave a higher dynamic range. Quantification of luciferase activity is given in Relative Luciferase Units (RLU). The results are reported as the means of two independent experiments. FIG. 17 shows the antiviral activity against a CCR5-dependent (R5-Bal) and a CXCR4-dependent (Lai/IIIB) strain of HIV-1 of the monomeric fusion inhibitor Ac-SWETWEREIENYTRQIYRILEESQEQQDRNERDLLEGSGC(PEG₄-Chol)-NH₂ (SEQ ID NO. 191) (black, ▴) and of the peptide inhibitor C34 lacking cholesterol (dark grey, ).

### 4.6 Example

### Antiviral Activity of Cholesterol-Derivatized Inhibitors Against Human Immunodeficiency Virus (HIV)

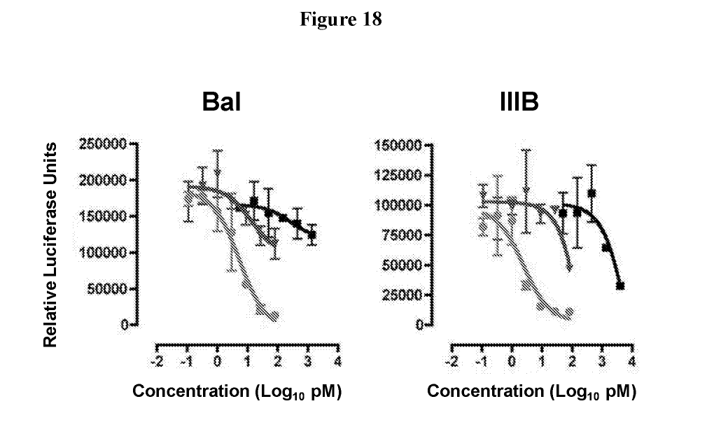

The antiviral activity of a dimeric cholesterol-derivatized inhibitor was tested against Human Immunodeficiency Virus (HIV) as described in Example 4.5. FIG. 18 shows the antiviral activity against a CCR5-dependent (R5-Bal) and a CXCR4-dependent (Lai/IIIB) strain of HIV-1 of the dimeric fusion inhibitor [(Ac-WMEWDREINNYTSLIHSLIEESQNQQEKNEQELL-GSG-C(MAL-PEG₄)]₂-Chol (grey, ), of the monomeric fusion inhibitor Ac-WMEWDREINNYTSLIHSLIEESQNQQEKNEQELL-GSG-C(PEG₄-Chol) (black, ▴), and of the monomeric peptide lacking cholesterol Ac-WMEWDREINNYTSLIHSLIEESQNQQEKNEQELL-GSG-C(CH₂CONH₂) (black, □).

### REFERENCES

1. Dwyer, J. J., K. L. Wilson, D. K. Davison, S. A. Freel, J. E. Seedorff, S. A. Wring, N. A. Tvermoes, T. J. Matthews, M. L. Greenberg, and M. K. Delmedico. 2007. Design of helical, oligomeric HIV-1 fusion inhibitor peptides with potent activity against enfuvirtide-resistant virus. Proc Natl Acad Sci USA 104:12772-7.
2. Eckert, D. M., and P. S. Kim. 2001. Mechanisms of viral membrane fusion and its inhibition. Annu Rev Biochem 70:777-810.
3. Fantini, J., N. Garmy, R. Mahfoud, and N. Yahi. 2002. Lipid rafts: structure, function and role in HIV, Alzheimer's and prion diseases. Expert Rev Mol Med 4:1-22.
4. Ingallinella, P., E. Bianchi, N. A. Ladwa, Y.-J. Wang, R. Hrin, M. Veneziano, F. Bonelli, T. J. Ketas, J. P. Moore, M. D. Miller, and A. Pessi. 2009. Addition of a cholesterol group to an HIV-1 Peptide Fusion Inhibitor dramatically increases its antiviral potency. Proc. Natl. Acad. Sci. U.S.A. 106:5801-5806.
5. Judice, J. K., J. Y. Tom, W. Huang, T. Wrin, J. Vennari, C. J. Petropoulos, and R. S. McDowell. 1997. Inhibition of HIV type 1 infectivity by constrained alpha-helical peptides: implications for the viral fusion mechanism. Proc Natl Acad Sci USA 94:13426-30.
6. Lambert, D. M., S. Barney, A. L. Lambert, K. Guthrie, R. Medinas, D. E. Davis, T. Bucy, J. Erickson, G. Merutka, and S. R. Petteway, Jr. 1996. Peptides from conserved regions of paramyxovirus fusion (F) proteins are potent inhibitors of viral fusion. Proc Natl Acad Sci USA 93:2186-91.
7. Lingwood, D., and K. Simons. 2010. Lipid rafts as a membrane-organizing principle. Science 327:46-50.
8. Liu, I. J., C. L. Kao, S. C. Hsieh, M. T. Wey, L. S. Kan, and W. K. Wang. 2009. Identification of a minimal peptide derived from heptad repeat (HR) 2 of spike protein of SARS-CoV and combination of HR1-derived peptides as fusion inhibitors. Antiviral Res 81:82-7.
9. Matthews, T., M. Salgo, M. Greenberg, J. Chung, R. DeMasi, and D. Bolognesi. 2004. Enfuvirtide: the first therapy to inhibit the entry of HIV-1 into host CD4 lymphocytes. Nat. Rev. Drug Discov. 3:215-25.
10. Oishi, S., S. Ito, H. Nishikawa, K. Watanabe, M. Tanaka, H. Ohno, K. Izumi, Y. Sakagami, E. Kodama, M. Matsuoka, and N. Fujii. 2008. Design of a novel HIV-1 fusion inhibitor that displays a minimal interface for binding affinity. J Med Chem 51:388-91.
11. Ono, A., and E. O. Freed. 2005. Role of lipid rafts in virus replication. Adv Virus Res 64:311-58.
12. Otaka, A., M. Nakamura, D. Nameki, E. Kodama, S. Uchiyama, S, Nakamura, H. Nakano, H. Tamamura, Y. Kobayashi, M. Matsuoka, and N. Fujii. 2002. Remodeling of gp41-C34 peptide leads to highly effective inhibitors of the fusion of HIV-1 with target cells. Angew Chem Int Ed Engl 41:2937-40.
13. Porotto, M., P. Carta, Y. Deng, G. E. Kellogg, M. Whitt, M. Lu, B. A. Mungall, and A. Moscona. 2007. Molecular determinants of antiviral potency of paramyxovirus entry inhibitors. J Virol 81:10567-74.
14. Porotto, M., L. Doctor, P. Carta, M. Formabaio, O. Greengard, G. E. Kellogg, and A. Moscona. 2006. Inhibition of hendra virus fusion. J Virol 80:9837-49.
15. Porotto, M., M. Formabaio, G. E. Kellogg, and A. Moscona. 2007. A second receptor binding site on human parainfluenza virus type 3 hemagglutinin-neuraminidase contributes to activation of the fusion mechanism. J Virol 81:3216-28.
16. Porotto, M., C. Yokoyama, G. Orefice, H.-S. Kim, and A. Moscona. 2009. Kinetic dependence of paramyxovirus entry inhibition. J. Virol. 83:6947-6951.
17. Porotto, M., C. Yokoyama, L. M. Palermo, B. Mungall, M. Aljofan, R. Cortese, A. Pessi, and A. Moscona. 2010. Viral entry inhibitors targeted to the membrane site of action. J. Virol.: JVL00135-10.
18. Schmidt, A. G., P. L. Yang, and S. C. Harrison. 2010. Peptide Inhibitors of Dengue-Virus Entry Target a Late-Stage Fusion Intermediate. PLoS Pathog 6:e1000851.
19. Steger, H. K., and M. J. Root. 2006. Kinetic dependence to HIV-1 entry inhibition. J Biol Chem 281:25813-21.
20. York, J., S. S. Agnihothram, V. Romanowski, and J. H. Nunberg. 2005. Genetic analysis of heptad-repeat regions in the G2 fusion subunit of the Junin arenavirus envelope glycoprotein. Virology 343:267-74.
21. He, Y., Y. Xiao, H. Song, Q. Liang, D. Ju, X. Chen, H. Lu, W. Jing, S. Jiang, and L. Zhang. Design and Evaluation of Sifuvirtide, a Novel HIV-1 Fusion Inhibitor. 2008. J Biol Chem 283:11126-34

1. A multimeric inhibitor of viral fusion comprising:
(i) at least two polypeptides capable of inhibiting fusion of at least one enveloped virus with a cellular membrane, and
(ii) a membrane integrating lipid selected from the group consisting of cholesterol, a sphingolipid, a glycolipid, a glycerophospholipid and membrane integrating derivatives thereof, which is attached to said polypeptides;
or a pharmaceutically acceptable salt thereof.

2. The multimeric inhibitor of claim 1, wherein said at least two polypeptides capable of inhibiting fusion of at least one enveloped virus bind to
(i) a viral coat protein of at least one enveloped virus, or
(ii) a protein which is associated with the cellular membrane and which mediates the entry of an enveloped virus into a cell.

3. The multimeric inhibitor of claim 1, wherein the at least one enveloped virus is selected from the group consisting of orthomyxoviridae, Paramyxoviridae, filoviridae, retroviridae, coronaviridae, bornaviridae, togaviridae, arenaviridae, herpesviridae, hepadnaviridae, flaviviridae, rhabdoviridae.

4. The multimeric inhibitor of claim 1, wherein the at least one enveloped virus is selected from the group consisting of Influenza virus, Parainfluenza virus, Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Herpes simplex virus (HSV), Human herpesvirus (HHV) 6A, Human herpesvirus (HHV) 6B, Cytomegalovirus, Varicella-zoster virus, Chikunguya virus, Hepatitis C virus (HCV), Rabies virus, Dengue virus (DV), West Nile virus, Junin virus, Machupo virus, Guanarito virus, Japanese encephalitis virus, Yellow fever virus, and Lassa virus.

5. The multimeric inhibitor of claim 2, wherein
(i) the viral coat protein is a viral fusogenic protein, preferably a Type I, II, or III viral fusogenic protein, or
(ii) the protein is associated with the cellular membrane and mediates the entry of an enveloped virus selected from the group consisting of Influenza virus, Parainfluenza virus, Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Herpes simplex virus (HSV), Human herpesvirus (HHV) 6A, Human herpesvirus (HHV) 6B, Cytomegalovirus, Varicella-zoster virus, Chikunguya virus, Hepatitis C virus (HCV), Rabies virus, Dengue virus (DV), West Nile virus, Junin virus, Machupo virus, Guanarito virus, Japanese encephalitis virus, Yellow fever virus, and Lassa virus into a cell.

6. The multimeric inhibitor of claim 1, wherein at least one of said peptides that are comprised in said at least two polypeptides is selected from the group consisting of
a) a polypeptide comprising an amino sequence WX₁EWX₂REINX₃YX₄SLIX₅SLIEEX₆QX₇QQX₈KNEX₉X₁₀LX₁₁X₁₂L (SEQ ID NO: 188), wherein
X₁ is selected from M, Nle (norleucine), Q and N, preferably from N or Q, most preferably N;
X₂ is selected from D and E, preferably E;
X₃ is selected from N and K, preferably K;
X₄ is selected from T and I, preferably T;
X₅ is selected from H and Y, preferably Y;
X₆ is selected from S, A, L and Abu (2-aminobutyric acid), preferably S or L, more preferably A;
X₇ is selected from N and K, preferably N;
X₈ is selected from E, e (D-glutamic acid), D, d (D-aspartic acid), preferably E or D, more preferably D;
X₉ is selected from K, k (D-lysine), R, r (D-arginine), preferably K or R, more preferably K;
X₁₀ may not be present or is selected from E, D, A, preferably E or D, more preferably D;
X₁₁ may not be present or is selected from L, K, R, preferably L or K, more preferably L; and
X₁₂ may not be present or is selected from E, e (D-glutamic acid), A, preferably E or e, more preferably E;
b) a polypeptide comprising an amino acid sequence having at least 75% identity to WNEWEREINKYTSLIYSLIEEAQNQQDKNEKDLLEL (SEQ ID NO: 192); or
c) a polypeptide comprising an amino acid sequence SWETWEREIENYTRQIYRILEESQEQQDRNERDLLE (SEQ ID NO: 189);

7. The multimeric inhibitor of claim 1, wherein said at least two polypeptides each comprise a peptide, wherein at least one of said peptides is capable of inhibiting fusion of at least one enveloped virus by binding to
(i) a heptad repeat (HR) domain of a Type I or III viral fusogenic protein of at least one enveloped virus, preferably a heptad repeat 1 (HR1) domain or heptad repeat 2 (HR2) domain of a Type I viral fusogenic protein of at least one enveloped virus, or
(ii) a beta-sheet domain of a Type II viral fusogenic protein of at least one enveloped virus, preferably a beta-sheet domain comprised in domain II of a Type II viral fusogenic protein of at least one enveloped virus.

8. The multimeric inhibitor of viral fusion of claim 7, wherein
(i) the HR1 domain of a Type I viral fusogenic protein is selected from the group consisting of HR1 domains with an amino acid sequence according to SEQ ID NO: 1 to SEQ ID NO: 17, SEQ ID NO: 105, and SEQ ID NO: 144 to SEQ ID NO: 150, or
(ii) the HR2 domain of a Type I viral fusogenic protein with an amino acid sequence according to SEQ ID NO: 151.

9. The multimeric inhibitor of viral fusion of claim 7, wherein at least one of said peptides that are comprised in said at least two polypeptides
(i) has a length of at least ten contiguous amino acids and is from a HR domain of a Type I or III viral fusogenic protein of at least one enveloped virus, preferably a HR1 domain or HR2 domain of a Type I viral fusogenic protein of at least one enveloped virus, or
(ii) has a length of at least ten contiguous amino acids and is from a membrane-proximal region (MPR) of a Type II viral fusogenic protein of at least one enveloped virus.

10. The multimeric inhibitor of claim 9, wherein at least one of said peptides that are comprised in said at least two polypeptides has at least ten contiguous amino acids of SEQ ID NO: 99 or of a derivative thereof, wherein the derivative consists of the following amino acids:
amino acid 1 is selected from Val, Leu, and Tyr;
amino acid 2 is selected from Ala, Ser, Asp, Tyr, and Phe;
amino acid 3 is selected from Leu, Ile, Pro, and Thr;
amino acid 4 is selected from Asp, Leu, and Phe;
amino acid 5 is selected from Pro, Val, and Lys;
amino acid 6 is selected from Ile, Leu, Phe, Val, and Ala;
amino acid 7 is selected from Asp and Glu;
amino acid 8 is selected from Ile and Phe;
amino acid 9 is selected from Ser and Asp;
amino acid 10 is selected from Ile, Gln, Ala, and Ser;
amino acid 11 is selected from Glu, Asn, Ser, Gln, and Val;
amino acid 12 is selected from Leu, Ile, and Asn;
amino acid 13 is selected from Asn, Ala, and Ser;
amino acid 14 is selected from Lys, Ala, Gln, and Ser;
amino acid 15 is selected from Ala, Val, Met, and Ile;
amino acid 16 is selected from Lys and Asn;
amino acid 17 is selected from Ser, Lys, Glu, and Gln;
amino acid 18 is selected from Asp, Ser, and Lys;
amino acid 19 is selected from Leu and Ile;
amino acid 20 is selected from Glu, Ser, Asn, and Gln;
amino acid 21 is selected from Glu, Asp, and Gln;
amino acid 22 is selected from Ser, Ala, and Ile;
amino acid 23 is selected from Lys and Leu;
amino acid 24 is selected from Glu, Gln, Ala, and Asp;
amino acid 25 is selected from Trp, His, Phe, and Tyr;
amino acid 26 is selected from Ile and Leu;
amino acid 27 is selected from Arg, Ala, and Lys;
amino acid 28 is selected from Arg, Gln, Lys, and Glu;
amino acid 29 is selected from Ser, Ala, and Ile;
amino acid 30 is selected from Asn, Asp, and Gln;
amino acid 31 is selected from Gly, Thr, Glu, Lys, Arg, and Gln;
amino acid 32 is selected from Lys, Tyr, Leu, and Ile;
amino acid 33 is Leu;
amino acid 34 is selected from Asp, Ser, and His;
amino acid 35 is selected from Ser, Ala, Asn, and Thr;
amino acid 36 is selected from Ile and Val; and
wherein the derivative may optionally comprise the three additional amino acids Pro, Ser, and Asp between amino acid 6 and amino acid 7.

11. The multimeric inhibitor of claim 10, wherein amino acid 31 of the derivative is Lys and amino acid 32 of the derivative is Ile.

12. The multimeric inhibitor of claim 9, wherein at least one of said peptides that are comprised in said at least two polypeptides has at least ten contiguous amino acids of an amino acid sequence selected from the group consisting of SEQ ID NOs: 106, 107, 108, 109, 110, 111, 112, 113, 114, 115, 116, 117, 118, and 119;
wherein each X recited in the sequences specified by said SEQ ID NOs is individually selected from any amino acid with the proviso that said amino acid sequence has at least 85% sequence identity with SEQ ID NO: 99.

13. The multimeric inhibitor of claim 9, wherein at least one of said peptides that are comprised in said at least two polypeptides has an amino acid sequence selected from the group consisting of SEQ ID NOs: 106, 107, 108, 109, 110, 111, 112, 113, 114, 115, 116, 117, 118, and 119;
wherein each X recited in the sequences specified by said SEQ ID NOs is individually selected from any amino acid with the proviso that the peptide has at least 85% sequence identity with SEQ ID NO: 99.

14. The multimeric inhibitor according to claim 9,
wherein at least one of said peptides that are comprised in said at least two polypeptides has the amino acid sequence V₁XXDXXDISXXL₁₂XXXK₁₆XXLXXS₂₂XXXI₂₆XXS₂₉XKILXXI₃₆ (SEQ ID NO: 110) or a derivative thereof, wherein the derivative comprises at least one of the following amino acids substitutions:
V₁ may be substituted with L, A or I;
L₁₂ may be substituted with I or V;
K₁₆ may be substituted with N or H;
S₂₂ may be substituted with A;
I₂₆ may be substituted with L or V;
S₂₉ may be substituted with A; and/or
I₃₆ may be substituted with V or L;
wherein each X is individually selected from any amino acid with the proviso that the peptide has at least 85% sequence identity with SEQ ID NO: 99.

15. The multimeric inhibitor of claim 9, wherein at least one of said peptides that are comprised in said at least two polypeptides has at least ten contiguous amino acids from a HR2 domain of a Type I viral fusogenic protein of at least one enveloped virus, wherein the amino acid sequence of said domain is selected from the group consisting of SEQ ID NO: 18 to SEQ ID NO: 104, SEQ ID NO: 120 to SEQ ID NO: 127, and a sequence having at least 85% sequence identity thereto.

16. The multimeric inhibitor of claim 9, wherein at least one of said peptides that are comprised in said at least two polypeptides has an amino acid sequence selected from the group consisting of SEQ ID NO: 18 to SEQ ID NO: 104, SEQ ID NO: 120 to SEQ ID NO: 127 and a sequence having at least 85% sequence identity thereto.

17. The multimeric inhibitor of any of claim 9, wherein at least one of said peptides that are comprised in said at least two polypeptides has at least ten contiguous amino acids from a HR1 domain of a Type I viral fusogenic protein of an enveloped virus, wherein the amino acid sequence of said domain is selected from the group consisting of SEQ ID NO: 128 and a sequence having at least 85% sequence identity thereto.

18. The multimeric inhibitor of claim 9, wherein at least one of said peptides that are comprised in said at least two polypeptides has an amino acid sequence selected from the group consisting of SEQ ID NO: 128 and a sequence having at least 85% sequence identity thereto.

19. The multimeric inhibitor of claim 9, wherein at least one of said peptides that are comprised in said at least two polypeptides has at least ten contiguous amino acids from a HR domain of a Type III viral fusogenic protein of an enveloped virus, wherein the amino acid sequence of said domain is selected from the group consisting of SEQ ID NO: 129 to SEQ ID NO: 136 and a sequence having at least 85% sequence identity thereto.

20. The multimeric inhibitor of claim 9, wherein at least one of said peptides that are comprised in said at least two polypeptides has an amino acid sequence selected from the group consisting of SEQ ID NO: 129 to SEQ ID NO: 136 and a sequence having at least 85% sequence identity thereto.

21. The multimeric inhibitor of claim 9, wherein at least one of said peptides that are comprised in said at least two polypeptides has at least ten contiguous amino acids from a membrane-proximal region (MPR) of a Type II viral fusogenic protein of an enveloped virus of SEQ ID NO: 137 or of a derivative thereof, wherein the derivative consists of the following amino acids:
amino acid 1 is Ala,
amino acid 2 is Trp;
amino acid 3 is Asp;
amino acid 4 is Phe;
amino acid 5 is selected from Gly and Ser;
amino acid 6 is Ser;
amino acid 7 is selected from Ile, Leu, Val, and Ala;
amino acid 8 is Gly;
amino acid 9 is Gly;
amino acid 10 is selected from Val, Leu, and Phe;
amino acid 11 is selected from Phe and Leu;
amino acid 12 is selected from Thr and Asn;
amino acid 13 is Ser;
amino acid 14 is selected from Val, Ile, and Leu;
amino acid 15 is Gly;
amino acid 16 is Lys;
amino acid 17 is selected from Leu, Ala, Met, and Gly;
amino acid 18 is selected from Ile, Leu, and Val;
amino acid 19 is His;
amino acid 20 is selected from Gln and Thr;
amino acid 21 is selected from Ile and Val; and
amino acid 22 is Phe.

22. The multimeric inhibitor of claim 9, wherein at least one of said peptides that are comprised in said at least two polypeptides has at least ten contiguous amino acids from a membrane-proximal region (MPR) of a Type II viral fusogenic protein of an enveloped virus, wherein the amino acid sequence of said domain is selected from the group consisting of SEQ ID NO: 137 to SEQ ID NO: 143 and a sequence having at least 85% sequence identity thereto.

23. The multimeric inhibitor of claim 9, wherein at least one of said peptides that are comprised in said at least two polypeptides has an amino acid sequence selected from the group consisting of SEQ ID NO: 137 to SEQ ID NO: 143 and a sequence having at least 85% sequence identity thereto.

24. The multimeric inhibitor of claim 1, wherein at least one of said polypeptides is an antibody or a fragment thereof, and wherein preferably the membrane integrating lipid is attached to an amino acid comprised in a VL; VH; VL; VH1, CH2, or CH3 domain of said antibody or fragment thereof.

25. The multimeric inhibitor of claim 24, wherein the amino acid is located:
(i) N-terminal to the CDR-1 region of the VL domain of said antibody or fragment thereof,
(ii) N-terminal to the CDR-1 region of the VH domain of said antibody or fragment thereof,
(iii) within the CDR-3 region of the VL domain of said antibody or fragment thereof, or
(iv) within the CDR-3 region of the VH domain of said antibody or fragment thereof.

26. The multimeric inhibitor of claim 24, wherein the amino acid is located:
(i) at position 20 or 22 of the VL domain of said antibody or fragment thereof,
(ii) at position 19 or 21 of the VL domain of said antibody or fragment thereof,
(iii) at position 7 or 25 of the VH domain of said antibody or fragment thereof,
(iv) at position 197 of the CL domain of said antibody or fragment thereof,
(v) at position 125 of the CH1 domain of said antibody or fragment thereof,
(vi) at position 248 or 326 of the CH2 domain of said antibody or fragment thereof, or
(vii) at position 415 or 442 of the CH3 domain of said antibody or fragment thereof.

27. The multimeric inhibitor of claim 24, wherein the antibody is a monoclonal antibody selected from the group consisting of MAB F10, MAB CR6261, MAB D5, MAB 2F5, MAB 4E10, MAB VRC01, MAB VRC02, palivizumab, and motavizumab, wherein said monoclonal antibody optionally comprises one or two single amino acid substitutions, deletions, modifications and/or insertions.

28. The multimeric inhibitor of claim 24, wherein the antibody or fragment thereof is an antibody or fragment thereof with a CDR3 domain of the heavy chain which comprises or consists of the sequence:

| | |
|---|---|
| RRGPTTXXXXXXARGPVNAMDV | (SEQ ID NO: 185) |
| or | |
| EGTTGXXXXXXPIGAFAH; | (SEQ ID NO: 186) |

wherein X may be any amino acid and wherein the lipid is covalently bound to one of the amino acids designated as X; and
wherein said sequence according to SEQ ID NO: 185 or 186 optionally comprises one single amino acid substitution, deletion, modification and/or insertion.

29. The multimeric inhibitor of claim 1, wherein the membrane integrating lipid is attached to
(i) the C-terminal region of at least one of said at least two polypeptides, or
(ii) the N-terminal region of at least one of said at least two polypeptides.

30. The multimeric inhibitor of claim 1, wherein the C-terminal amino acid, the N-terminal amino acid, and/or one or more internal amino acids of at least one of said at least two polypeptides is (are) modified.

31. The multimeric inhibitor of claim 30, wherein
(i) the C-terminal amino acid is modified by amidation,
(ii) the N-terminal amino acid comprises a chemical modification selected from the group consisting of one or more L-amino acids and/or D-amino acids, an acyl group, beta-alanine, 9H-fluoren-9-ylmethoxycarbonyl (Fmoc), Benzyloxy-carbonyl, and (t) ert-(B)ut(O)xy(c)arbonyl (Boc), and/or
(iii) at least two amino acids spaced by at least one amino acid apart are connected, preferably by an amide (lactam) bond, a disulfide bond, a thioether bond, or a hydrocarbon bridge between the amino acid side chains.

32. The multimeric inhibitor of claim 1, wherein at least one of said at least two polypeptides further comprises one or more linker amino acid(s) at its C-terminus and/or N-terminus.

33. The multimeric inhibitor of claim 32, wherein the one or more linker amino acid(s) comprise(s) a cysteine at its (their) C-terminus and/or N-terminus.

34. The multimeric inhibitor of claim 32, wherein the linker amino acids are selected from the group consisting of (Gly)ₘ₊₁, (GlySerGly)ₘ, (GlySerGlySerGly)ₘ, (GlyPro)ₘ, (Gly)ₘ₊₁Cys, (GlySerGly)ₘCys, (GlySerGlySerGly)ₘCys and (GlyPro)ₘCys, wherein m is an integer of 1 to 20.

35. The multimeric inhibitor of claim 1, wherein said at least two polypeptides are covalently linked to said membrane integrating lipid via a linker.

36. The multimeric inhibitor of claim 35, wherein said linker comprises a moiety having a structure according to formula (I) 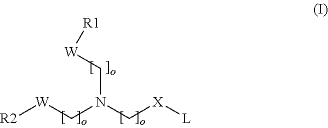
wherein each of R₁ and R₂ is independently selected from the group consisting of:
(i) R₃;
(ii) a structure according to formula (II): 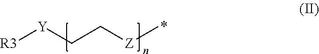
and
(iii) a structure according to formula (III): 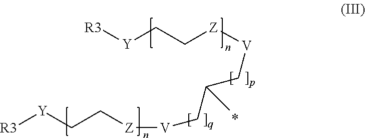
wherein
W is in each instance independently selected from —NH—C(O)—O—, —O—C(O)—NH—, —C(O)—O—, —O—C(O)—, —(CH₂)ₘ—, —NH—C(O)—, —C(O)—NH—, —NH—, and —C(X)— most preferably W is —C(O)—NH—;
V is in each instance independently selected from —(CH₂)ₘ—, —(CH₂)ₘ—C(X)—NH—, —NH—C(X)—(CH₂)ₘ—, —(CH₂)ₘ—NH—C(O)—O—, —O—C(O)—NH—(CH₂)ₘ—, —(CH₂)ₘ—C(O)—O—, —O—C(O)—(CH₂)ₘ—, —NH—C(X)—, —C(X)—NH—, —NH—C(O)—O—, —O—C(O)—NH—, —C(O)—O—, and —O—C(O)—; most preferably V is —CH₂CH₂—C(O)—NH—;
X is in each instance either O, S, or NH;
Y is in each instance independently selected from —C(O)CH₂—, —CH₂C(O)—, —NHCH₂—, —CH₂NH—, —NHC(O)—, —C(O)NH—, —NH—, —CH₂—, —CH₂C(O)NH— and —NHC(O)CH₂—; most preferably Y is —NHCH₂—;
Z is in each instance independently selected from —CH₂—, —NH—, —O—, —CH₂O—, —NHCH₂— and —OCH₂—; most preferably Z is —O—;
R₃ is in each case independently selected from any of said polypeptides, which may be the same or different;
m is in each instance independently selected from an integer of between 0 and 5; preferably between 0 and 3, preferably m is the same in each instance;
n is in each instance independently selected from an integer of between 0 and 40; preferably between 3 and 10, preferably n is the same in each instance;
o is in each case independently selected from an integer of between 0 and 5; preferably 2, preferably o is the same in each instance;
p is in each instance independently selected from an integer of between 0 and 5; preferably between 0 and 3, preferably p is the same in each instance;
q is in each instance independently selected from an integer of between 0 and 5; preferably between 0 and 3; preferably q is the same in each instance and/or preferably q≦p
L is said membrane integrating lipid; and
wherein * marks, where the structures (II-III) are linked to structure (I).

37. The multimeric inhibitor of claim 35, wherein the structure according to formula (III) has a structure according to formula (IV): 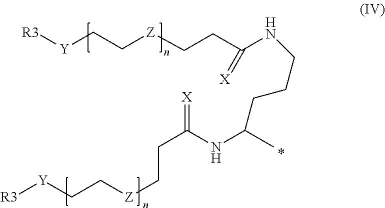
and, preferably o is 2.

38. The multimeric inhibitor of claim 36, wherein said moiety has a structure according to formula (V) 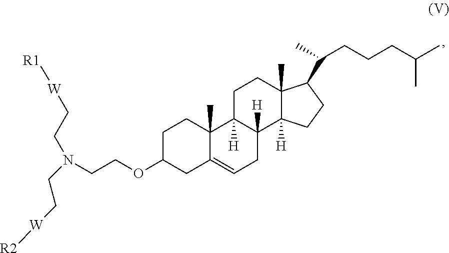
wherein
W is in each instance independently selected from —NH—C(O)—O—, —O—C(O)—NH—, —C(O)—O—, —O—C(O)—, (CH₂)ₘ, —NH—C(O)—, —(O)C—NH—, and —NH—; and
m is an integer of between 0 and 3; preferably 0.

39. The inhibitor of claim 35, wherein said linker comprises the structure NH₂—CH₂—CH₂—O—(CH₂—CH₂—O)ₙ—CH₂—CH₂—COOH, with n=1-35.

40. The inhibitor of claim 39, wherein said linker comprises a structure selected from the group consisting of Cys-(CH₂—CH₂—O)₄-cholesterol, Cys-(CH₂—CH₂—O)₂₄-cholesterol and NH—(CH₂—CH₂—O)₂₄—CO-Cys-(CH₂—CH₂—O)₄-cholesterol.

41. The multimeric inhibitor of claim 35, wherein the membrane integrating lipid or membrane integrating derivative thereof is attached via the linker to the polypeptides through the oxygen moiety at the 3 position of the cholesterol or derivative thereof.

42. A pharmaceutical composition comprising the multimeric inhibitor of claim 1 or a pharmaceutically acceptable salt thereof and a pharmaceutically acceptable excipient.

43. A multimeric inhibitor of claim 1 or a pharmaceutically acceptable salt thereof for the treatment or prevention of infection(s) by (an) enveloped virus(es).

44. The multimeric inhibitor or a pharmaceutically acceptable salt thereof of claim 43, wherein the enveloped virus(es) is (are) selected from the group consisting of Influenza virus, Parainfluenza virus, Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Herpes simplex virus (HSV), Human herpesvirus (HHV) 6A, Human herpesvirus (HHV) 6B, Cytomegalovirus, Varicella-zoster virus, Chikunguya virus, Hepatitis C virus (HCV), Rabies virus, Dengue virus (DV), West Nile virus, Junin virus, Machupo virus, Guanarito virus, Japanese encephalitis virus, Yellow fever virus, and Lassa virus.

45. The multimeric inhibitor of claim 1 or a pharmaceutically acceptable salt thereof for use in treating or preventing infection(s) by (an) enveloped virus(es).

46. The multimeric inhibitor of claim 1, wherein the enveloped virus(es) is (are) selected from the group consisting of Influenza virus, Parainfluenza virus, Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Herpes simplex virus (HSV), Human herpesvirus (HHV) 6A, Human herpesvirus (HHV) 6B, Cytomegalovirus, Varicella-zoster virus, Chikunguya virus, Hepatitis C virus (HCV), Rabies virus, Dengue virus (DV), West Nile virus, Junin virus, Machupo virus, Guanarito virus, Japanese encephalitis virus, Yellow fever virus, and Lassa virus.

47. A method for making a broad-spectrum multimeric inhibitor of viral fusion effective against at least two, preferably three or four, different enveloped viruses, wherein the method comprises the steps of:
(i) generating at least two polypeptides each comprising a peptide as defined in claim 1, and/or wherein at least one of said peptides is a hybrid peptide which is capable of inhibiting fusion of at least two, preferably three or four, different enveloped viruses by binding to a HR1 domain or HR2 domain of a Type I viral fusogenic protein of said enveloped viruses selected from the group consisting of HR domains with an amino acid sequence according to SEQ ID NO: 1 to SEQ ID NO: 17, SEQ ID NO: 105, and SEQ ID NO: 144 to SEQ ID NO: 151, and wherein said hybrid peptide comprises amino acids from HR domains of a Type I viral fusogenic protein of at least two different enveloped viruses; and
(ii) covalently linking a membrane integrating lipid selected from the group consisting of cholesterol, a sphingolipid, a glycolipid, a glycerophospholipid and membrane integrating derivatives thereof to the C-terminal or N-terminal region of said polypeptides.

48. A method for making a broad-spectrum multimeric inhibitor of viral fusion effective against at least two, preferably three or four, different enveloped viruses, wherein the method comprises the steps of:
(i) generating at least two polypeptides each comprising a peptide as defined in claim 1, and/or wherein at least one of said peptides is a hybrid peptide which is capable of inhibiting fusion of at least two, preferably three or four, different enveloped viruses by binding to a beta-sheet domain of a Type II viral fusogenic protein of said enveloped viruses selected from the group consisting of Dengue virus, West Nile virus, Yellow fever virus, and Japanese encephalitis virus, and wherein said hybrid peptide comprises amino acids from membrane-proximal regions (MPRs) of a Type II viral fusogenic protein of at least two different enveloped viruses selected from the group consisting of MPRs with an amino acid sequence according to SEQ ID NO: 137 to SEQ ID NO: 143; and
(ii) covalently linking a membrane integrating lipid selected from the group consisting of cholesterol, a sphingolipid, a glycolipid, a glycerophospholipid and membrane integrating derivatives thereof to the C-terminal region of said polypeptides.

49. A method for making a broad-spectrum multimeric inhibitor of viral fusion effective against at least two, preferably three or four, different enveloped viruses, wherein the method comprises the steps of:
(i) generating at least two polypeptides each comprising a peptide as defined in claim 1, and/or wherein at least one of said peptides is a hybrid peptide which is capable of inhibiting fusion of at least two, preferably three or four, different enveloped viruses by binding to a HR domain of a Type III viral fusogenic protein of said enveloped viruses selected from the group consisting of Herpes simplex virus (HSV), Human herpesvirus 6A; Human herpesvirus 6B, and Cytomegalovirus, and wherein said hybrid peptide comprises amino acids from HR domains of a Type III viral fusogenic protein of at least two different enveloped viruses selected from the group consisting of HR domains with an amino acid sequence according to SEQ ID NO: 129 to SEQ ID NO: 136; and
(ii) covalently linking a membrane integrating lipid selected from the group consisting of cholesterol, a sphingolipid, a glycolipid, a glycerophospholipid and membrane integrating derivatives thereof to the C-terminal or N-terminal region of said polypeptides.

50. A method for making a broad-spectrum multimeric inhibitor of viral fusion effective against at least two, preferably three or four, different enveloped viruses, wherein the method comprises the steps of:
(i) generating at least two polypeptides each comprising a peptide as defined in claim 1, and/or wherein at least one of said peptides is a hybrid peptide which is capable of inhibiting fusion of at least two, preferably three or four, different enveloped viruses by binding to a HR1 domain or HR2 domain of a Type I viral fusogenic protein of said enveloped viruses selected from the group consisting of Influenza virus, Parainfluenza virus, Sendai virus, Measles virus, Newcastle disease virus, Mumps virus, Respiratory syncytical virus (RSV), human metapneumovirus (hMPV), Hendra virus (HeV), Nipah virus (NiV), Ebola virus (EBOV), Marburg virus, Human immunodeficiency virus (HIV), Severe acute respiratory syndrome (SARS) virus, Rabies virus, Junin virus, Machupo virus, Guanarito virus, and Lassa virus, and wherein said hybrid peptide comprises amino acids from HR domains of a Type I viral fusogenic protein of at least two different enveloped viruses selected from the group consisting of HR domains with an amino acid sequence according to SEQ ID NO: 18 to SEQ ID NO: 34, SEQ ID NO: 50 to SEQ ID NO: 54, SEQ ID NO: 83 to SEQ ID NO: 99, SEQ ID NO: 102 to SEQ ID NO: 104 and SEQ ID NO: 120 to SEQ ID NO: 128; and
(ii) covalently linking a membrane integrating lipid selected from the group consisting of cholesterol, a sphingolipid, a glycolipid, a glycerophospholipid and membrane integrating derivatives thereof to the C-terminal or N-terminal region of said polypeptides.


SEQUENCE LISTING (192 sequences)

SEQ ID NO: 1 - protein, 35 aa, Human parainfluenza virus 3
LKEAIRDTNKAVQSVQSSIGNLIVAIKSVQDYVNK

SEQ ID NO: 2 - protein, 34 aa, Sendai virus (Strain Harris)
IKESMTKTHKSVELLQNAVGEQILALKTLQDFVN

SEQ ID NO: 3 - protein, 34 aa, Sendai virus (Strain Fusihimi); identical to SEQ ID NO: 2

SEQ ID NO: 4 - protein, 34 aa, Human parainfluenza virus 1 (Strain C39)
IKDSIIKTHNSVELIQRGIGEQIIALKTLQDFVN

SEQ ID NO: 5 - protein, 34 aa, Measles virus (Strain Yamagta)
LRASLETTNQAIEAIRQTGQEMILAVQGVQDYIN

SEQ ID NO: 6 - protein, 34 aa, Measles virus (Strain Alk-C)
LRASLETTNQAIEAIRQAGQEMILAVQGVQDYIN

SEQ ID NO: 7 - protein, 34 aa, Newcastle disease virus (Strain Queensland)
LKESIAATNEAVHEVTNGLSQLAVAVGKMQQFVN

SEQ ID NO: 8 - protein, 34 aa, Newcastle disease virus (Strain D26/76); identical to SEQ ID NO: 7

SEQ ID NO: 9 - protein, 34 aa, Mumps virus (Strain SBL)
MKNSIQATNRAVFEVKEGTQQLAIAVQAIQDHIN

SEQ ID NO: 10 - protein, 35 aa, Human parainfluenza virus 2
LASSIQSTNKAVSDVIDASRTIATAVQAIQDHING

SEQ ID NO: 11 - protein, 46 aa, Human respiratory syncytial virus
VLHLEGEVNKIKSALLSTNKAVVSLSNGVSVLTSKVLDLKNYIDKQ

SEQ ID NO: 12 - protein, 46 aa, Human respiratory syncytial virus (Subgroup B)
VLHLEGEVNKIKNALLSTNKAVVSLSNGVSVLTSKVLDLKNYINNR

SEQ ID NO: 13 - protein, 42 aa, Hendra virus
AMKNADNINKLKSSIESTNEAVVKLQETAEKTVYVLTALQDY

SEQ ID NO: 14 - protein, 42 aa, Ebola-like virus
LICGLRQLANETTQALQLFLRATTELRTFSILNRKAIDFLLQ

SEQ ID NO: 15 - protein, 42 aa, Ebola virus (Strain Reston)
LICGLRQLANETTQALQLFLRATTELRTYSLLNRKAIDFLLQ

SEQ ID NO: 16 - protein, 42 aa, Ebola virus (Strain Sudan)
LVCGLRQLANETTQALQLFLRATTELRTYTILNRKAIDFLLR

SEQ ID NO: 17 - protein, 41 aa, Marburg virus (Strain Lake Victoria)
LVCRLRRLANQTAKSLELLLRVTTEERTFSLINRHAIDFLL

SEQ ID NO: 18 - protein, 35 aa, Human parainfluenza virus 3
IDISIELNKAKSDLEESKEWIRRSNQKLDSIGNWH

SEQ ID NO: 19 - protein, 35 aa, Human parainfluenza virus 1
VDISLNLASATNFLEESKIELMKAKAIISAVGGWH

SEQ ID NO: 20 - protein, 35 aa, Sendai virus (Strain Z)
IDISLNLADATNFLQDSKAELEKARKILSEVGRWY

SEQ ID NO: 21 - protein, 35 aa, Sendai virus (Strain Harris)
VDISLNLADATNFLQDSKAELEKARKILSEVGRWY

SEQ ID NO: 22 - protein, 29 aa, Mumps virus (Strain SBL)
ISTELSKVNASLQNAVKYIKESNHQLQSV

SEQ ID NO: 23 - protein, 29 aa, Newcastle disease virus
ISTELGNVNNSISNALDKLEESNSKLDKV

SEQ ID NO: 24 - protein, 29 aa, Measles virus (Strain Alk-C)
VGTNLGNAIAKLEDAKELLESSDQILRSM

SEQ ID NO: 25 - protein, 29 aa, Measles virus (Strain Yamagata)
VGTSLGSAIAKLEDAKELLESSDQILRSM

SEQ ID NO: 26 - protein, 29 aa, Newcastle Disease virus
ISTELGNVNNSISNALNKLEESNRKLDKV

SEQ ID NO: 27 - protein, 36 aa, Human respiratory syncytial virus
FDASISQVNEKINQSLAFIRKSDELLHNVNAGKSTT

SEQ ID NO: 28 - protein, 34 aa, Human respiratory syncytial virus (Subgroup B)
FDASISQVNEKINQSLAFIRRSDELLHNVNTGKS

SEQ ID NO: 29 - protein, 33 aa, Hendra virus
KVDISSQISSMNQSLQQSKDYIKEAQKILDTVN

SEQ ID NO: 30 - protein, 33 aa, Nipa virus
KVDISSQISSMNQSLQQSKDYIKEAQRLLDTVN

SEQ ID NO: 31 - protein, 24 aa, Ebola virus (Strain Zaire)
IEPHDWTKNITDKIDQIIHDFVDK

SEQ ID NO: 32 - protein, 23 aa, Ebola virus (Strain Sudan)
IEPHDWTKNITDKINQIIHDFID

SEQ ID NO: 33 - protein, 23 aa, Ebola virus (Strain Reston)
IEPHDWTKNITDEINQIKHDFID

SEQ ID NO: 34 - protein, 26 aa, Marburg virus (Strain Lake Victoria)
IGIEDLSKNISEQIDQIKKDEQKEGT

SEQ ID NO: 35 - protein, 36 aa, Artificial Sequence
  Derivative of HPIV3 HR2 domain
VALDPIDISIVLNKAKSDLEESKEWIRRSNKILDSI

SEQ ID NO: 36 - protein, 36 aa, Artificial Sequence
  Derivative of HPIV3 HR2 domain
VALDPIDISIVLNKAKSDLEESKEWIRRSNRLLDSI

SEQ ID NO: 37 - protein, 36 aa, Artificial Sequence
  Derivative of HPIV3 HR2 domain
VALDPIDISIVLNKAKSDLEESKEWIRESNKILDSI

SEQ ID NO: 38 - protein, 36 aa, Artificial Sequence
  Derivative of HPIV3 HR2 domain
VALDPIDISIVLNKAKSDLEESKEWIRESNRLLDSI

SEQ ID NO: 39 - protein, 31 aa, Artificial Sequence
  Derivative of HPIV3 HR2 domain
IDISIVLNKAKSDLEESKEWIRRSNGKLDSI

SEQ ID NO: 40 - protein, 36 aa, Artificial Sequence
  Derivative of HPIV3 HR2 domain
VALDPIDISEVLNKAKSDLEESKEWIRRSNGKLDSI

SEQ ID NO: 41 - protein, 36 aa, Artificial Sequence
  Derivative of HPIV3 HR2 domain
VALDPIDISIVLNKMKSDLEESKEWIRRSNGKLDSI

SEQ ID NO: 42 - protein, 36 aa, Artificial Sequence
  Derivative of HPIV3 HR2 domain
VALDPIDISIVLNKIKSDLEESKEWIRRSNGKLDSI

SEQ ID NO: 43 - protein, 36 aa, Artificial Sequence
  Derivative of HPIV3 HR2 domain
VALDPIDISIVLNKAKSDLEESKEWIRRSNGILDSI

SEQ ID NO: 44 - protein, 36 aa, Artificial Sequence
  Derivative of HPIV3 HR2 domain
VALDPIDISIVLNKAKSDLEESKEWIRESNGKLDSI

SEQ ID NO: 45 - protein, 36 aa, Artificial Sequence
  Derivative of HPIV3 HR2 domain
VALDPIDISIVLNKAKSDLEESKEWIRKSNGKLDSI

SEQ ID NO: 46 - protein, 36 aa, Artificial Sequence
  Derivative of HPIV3 HR2 domain
VALDPIDISIVLNKAKSELEESKEWIRRSNGKLDSI

SEQ ID NO: 47 - protein, 36 aa, Artificial Sequence
  Derivative of HPIV3 HR2 domain
VALDPIDISIVLNKAKSXLEESKEWIRRSNGKLDSI

SEQ ID NO: 48 - protein, 36 aa, Artificial Sequence
  Derivative of HPIV3 HR2 domain
VALDPIDISIVLNKAKSDLEESKEWIRRSNKILESI

SEQ ID NO: 49 - protein, 42 aa, Artificial Sequence
  HPIV3 HR2 domain with C-terminally attached linker
VALDPIDISIVLNKAKSDLEESKEWIRRSNGKLDSIGSGSGC

SEQ ID NO: 50 - protein, 24 aa, Artificial Sequence
  Derivative of Ebola virus HR2 domain
IEPHDWTKNITEKIDQIIHDFVDK

SEQ ID NO: 51 - protein, 24 aa, Artificial Sequence
  Derivative of Ebola virus HR2 domain
IEPHDWTKNITDKIDEIIHDFVDK

SEQ ID NO: 52 - protein, 24 aa, Artificial Sequence
  Derivative of Ebola virus HR2 domain
IEPHDWTKNITDKIEQIIKDFVDK

SEQ ID NO: 53 - protein, 30 aa, Artificial Sequence
  Derivative of Ebola virus HR2 domain
IEPHDWTKNITDKIDQIIHDFVDKGSGSGC

SEQ ID NO: 54 - protein, 32 aa, Human respiratory syncytial virus
SDEFDASISQVNEKINQSLAFIRKSDELLHNV

SEQ ID NO: 55 - protein, 32 aa, Artificial Sequence
  Chimere of HPV3 and RSV HR2 domain sequence
SALDPIDISIELNKAKSDLEESKEWIRRSNGK

SEQ ID NO: 56 - protein, 32 aa, Artificial Sequence
  Chimere of HPV3 and RSV HR2 domain sequence
VDLDPIDISIELNKAKSDLEESKEWIRRSNGK

SEQ ID NO: 57 - protein, 32 aa, Artificial Sequence
  Chimere of HPV3 and RSV HR2 domain sequence
VAEDPIDISIELNKAKSDLEESKEWIRRSNGK

SEQ ID NO: 58 - protein, 32 aa, Artificial Sequence
  Chimere of HPV3 and RSV HR2 domain sequence
VALFPIDISIELNKAKSDLEESKEWIRRSNGK

SEQ ID NO: 59 - protein, 32 aa, Artificial Sequence
  Chimere of HPV3 and RSV HR2 domain sequence
VALDDIDISIELNKAKSDLEESKEWIRRSNGK

SEQ ID NO: 60 - protein, 32 aa, Artificial Sequence
  Chimere of HPV3 and RSV HR2 domain sequence
VALDPADISIELNKAKSDLEESKEWIRRSNGK

SEQ ID NO: 61 - protein, 32 aa, Artificial Sequence
  Chimere of HPV3 and RSV HR2 domain sequence
VALDPISISIELNKAKSDLEESKEWIRRSNGK

SEQ ID NO: 62 - protein, 32 aa, Artificial Sequence
  Chimere of HPV3 and RSV HR2 domain sequence
VALDPIDISQELNKAKSDLEESKEWIRRSNGK

SEQ ID NO: 63 - protein, 32 aa, Artificial Sequence
  Chimere of HPV3 and RSV HR2 domain sequence
VALDPIDISIVLNKAKSDLEESKEWIRRSNGK

SEQ ID NO: 64 - protein, 32 aa, Artificial Sequence
  Chimere of HPV3 and RSV HR2 domain sequence
VALDPIDISIENNKAKSDLEESKEWIRRSNGK

SEQ ID NO: 65 - protein, 32 aa, Artificial Sequence
  Chimere of HPV3 and RSV HR2 domain sequence
VALDPIDISIELEKAKSDLEESKEWIRRSNGK

SEQ ID NO: 66 - protein, 32 aa, Artificial Sequence; identical to SEQ ID NO: 65
  Chimere of HPV3 and RSV HR2 domain sequence

SEQ ID NO: 67 - protein, 32 aa, Artificial Sequence
  Chimere of HPV3 and RSV HR2 domain sequence
VALDPIDISIELNKANSDLEESKEWIRRSNGK

SEQ ID NO: 68 - protein, 32 aa, Artificial Sequence
  Chimere of HPV3 and RSV HR2 domain sequence
VALDPIDISIELNKAKQDLEESKEWIRRSNGK

SEQ ID NO: 69 - protein, 32 aa, Artificial Sequence
  Chimere of HPV3 and RSV HR2 domain sequence
VALDPIDISIELNKAKSSLEESKEWIRRSNGK

SEQ ID NO: 70 - protein, 32 aa, Artificial Sequence
  Chimere of HPV3 and RSV HR2 domain sequence
VALDPIDISIELNKAKSDLAESKEWIRRSNGK

SEQ ID NO: 71 - protein, 32 aa, Artificial Sequence
  Chimere of HPV3 and RSV HR2 domain sequence
VALDPIDISIELNKAKSDLEFSKEWIRRSNGK

SEQ ID NO: 72 - protein, 32 aa, Artificial Sequence
  Chimere of HPV3 and RSV HR2 domain sequence
VALDPIDISIELNKAKSDLEEIKEWIRRSNGK

SEQ ID NO: 73 - protein, 32 aa, Artificial Sequence
  Chimere of HPV3 and RSV HR2 domain sequence
VALDPIDISIELNKAKSDLEESREWIRRSNGK

SEQ ID NO: 74 - protein, 32 aa, Artificial Sequence
  Chimere of HPV3 and RSV HR2 domain sequence
VALDPIDISIELNKAKSDLEESKKWIRRSNGK

SEQ ID NO: 75 - protein, 32 aa, Artificial Sequence
  Chimere of HPV3 and RSV HR2 domain sequence
VALDPIDISIELNKAKSDLEESKESIRRSNGK

SEQ ID NO: 76 - protein, 32 aa, Artificial Sequence
  Chimere of HPV3 and RSV HR2 domain sequence
VALDPIDISIELNKAKSDLEESKEWDRRSNGK

SEQ ID NO: 77 - protein, 32 aa, Artificial Sequence
  Chimere of HPV3 and RSV HR2 domain sequence
VALDPIDISIELNKAKSDLEESKEWIERSNGK

SEQ ID NO: 78 - protein, 32 aa, Artificial Sequence
  Chimere of HPV3 and RSV HR2 domain sequence
VALDPIDISIELNKAKSDLEESKEWIRLSNGK

SEQ ID NO: 79 - protein, 32 aa, Artificial Sequence
  Chimere of HPV3 and RSV HR2 domain sequence
VALDPIDISIELNKAKSDLEESKEWIRRLNGK

SEQ ID NO: 80 - protein, 32 aa, Artificial Sequence
  Chimere of HPV3 and RSV HR2 domain sequence
VALDPIDISIELNKAKSDLEESKEWIRRSHGK

SEQ ID NO: 81 - protein, 32 aa, Artificial Sequence
  Chimere of HPV3 and RSV HR2 domain sequence
VALDPIDISIELNKAKSDLEESKEWIRRSNNK

SEQ ID NO: 82 - protein, 32 aa, Artificial Sequence
  Chimere of HPV3 and RSV HR2 domain sequence
VALDPIDISIELNKAKSDLEESKEWIRRSNGV

SEQ ID NO: 83 - protein, 31 aa, Influenza A virus
GTYDHDVYRDEALNNRFQIKGVELKSGYKDW

SEQ ID NO: 84 - protein, 28 aa, Influenza A virus
GTFNAGEFSLPTFDSLNITAASLNDDGL

SEQ ID NO: 85 - protein, 28 aa, Influenza A virus
GTYDHTEYAEESKLKRQEIDGIKLKSED

SEQ ID NO: 86 - protein, 28 aa, Influenza A virus
GTYDHKEFEEESKINRQEIEGVKLDSSG

SEQ ID NO: 87 - protein, 28 aa, Influenza A virus
NTYDHSTYREEAMQNRVKIDPVKLSSGY

SEQ ID NO: 88 - protein, 28 aa, Influenza A virus
NTYDHSQYREEALLNRLNINSVKLSSGY

SEQ ID NO: 89 - protein, 28 aa, Influenza A virus
GTYDHDVYRDEALNNRFQIKGVELKSGY

SEQ ID NO: 90 - protein, 28 aa, Influenza A virus
GTYDHDIYRDEAINNRFQIQGVKLIQGY

SEQ ID NO: 91 - protein, 28 aa, Influenza A virus
GTYDYPKYEEESKLNRNEIKGVKLSSMG

SEQ ID NO: 92 - protein, 28 aa, Influenza A virus
GTYDYPQYSEEARLNREEISGVKLESMG

SEQ ID NO: 93 - protein, 28 aa, Influenza A Virus
GTYDYPKYSEESKLNREEIDGVKLESMG

SEQ ID NO: 94 - protein, 27 aa, Influenza A virus
GTYDHDVYRDEALNNRFQIKGVELKSG

SEQ ID NO: 95 - protein, 21 aa, Influenza A virus
GTYDHDVYRDEALNNRFQIKG

SEQ ID NO: 96 - protein, 36 aa, RSV
SDEFDASISQVNEKINQSLAFIRKSDELLHNVNAGK

SEQ ID NO: 97 - protein, 39 aa, RSV
YDPLVFPSDEFDASISQVNEKINQSLAFIRKSDELLHNV

SEQ ID NO: 98 - protein, 36 aa, HPIV3
VALDPIDISIELNKAKSDLEESKEWIRRSNQKLDSI

SEQ ID NO: 99 - protein, 36 aa, HPIV3
VALDPIDISIELNKAKSDLEESKEWIRRSNGKLDSI

SEQ ID NO: 100 - protein, 36 aa, HPIV3/HeV
VALDPIDISIVLNKIKSDLEESKEWIRRSNKILDSI

SEQ ID NO: 101 - protein, 42 aa, HPIV3/HeV
VALDPIDISIVLNKIKSDLEESKEWIRRSNKILDSIGSGSGC

SEQ ID NO: 102 - protein, 36 aa, SV5
LSIDPLDISQNLAAVNKSLSDALQHLAQSDTYLSAI

SEQ ID NO: 103 - protein, 36 aa, HeV
VYTDKVDISSQISSMNQSLQQSKDYIKEAQKILDTV

SEQ ID NO: 104 - protein, 36 aa, NiV
VFTDKVDISSQISSMNQSLQQSKDYIKEAQRLLDTV

SEQ ID NO: 105 - protein, 37 aa, Influenza A virus
ADLKSTQAAIDQINGKLNRLIGKTNEKFHQIEKEFSE

SEQ ID NO: 106 - protein, 36 aa, artificial
  preferred optimized consensus sequence of inhibitor peptide
XXXXXXXXXXXXXXXXXXXXXXXXXXXXXXKIXXXX

SEQ ID NO: 107 - protein, 36 aa, artificial
  preferred optimized consensus sequence of inhibitor peptide
XXXXXXXXXXVXXXXXXXXXXXXXXXXXXXKIXXXX

SEQ ID NO: 108 - protein, 36 aa, artificial
  preferred optimized consensus sequence of inhibitor peptide
XXXXXXXXXXVXXXIXXXXXXXXXXXXXXXKIXXXX

SEQ ID NO: 109 - protein, 36 aa, artificial
  preferred optimized consensus sequence of inhibitor peptide
XXXDXXDISXVXXXXXXXLXXXXXXXXXXXKILXXX

SEQ ID NO: 110 - protein, 36 aa, artificial
  preferred optimized consensus sequence of inhibitor peptide
VXXDXXDISXXLXXXKXXLXXSXXXIXXSXKILXXI

SEQ ID NO: 111 - protein, 36 aa, artificial
  preferred optimized consensus sequence of inhibitor peptide
VXXDXXDISXVLXXIKXXLXXSXXXIXXSXKILXXI

SEQ ID NO: 112 - protein, 42 aa, artificial
  preferred optimized consensus sequence of inhibitor peptide
VXXDXXDISXVLXXIKXXLXXSXXXIXXSXKILXXIGSGSGC

SEQ ID NO: 113 - protein, 36 aa, artificial
  preferred optimized consensus sequence of inhibitor peptide
XXXXXXXXXXXXXXXXXXXXXXXXXXXXXXGKXXXX

SEQ ID NO: 114 - protein, 36 aa, artificial
  preferred optimized consensus sequence of inhibitor peptide
XXXXXXXXXXVXXXXXXXXXXXXXXXXXXXGKXXXX

SEQ ID NO: 115 - protein, 36 aa, artificial
  preferred optimized consensus sequence of inhibitor peptide
XXXXXXXXXXVXXXIXXXXXXXXXXXXXXXGKXXXX

SEQ ID NO: 116 - protein, 36 aa, artificial
  preferred optimized consensus sequence of inhibitor peptide
XXXDXXDISXVXXXXXXXLXXXXXXXXXXXGKLXXX

SEQ ID NO: 117 - protein, 36 aa, artificial
  preferred optimized consensus sequence of inhibitor peptide
VXXDXXDISXXLXXXKXXLXXSXXXIXXSXGKLXXI

SEQ ID NO: 118 - protein, 36 aa, artificial
  preferred optimized consensus sequence of inhibitor peptide
VXXDXXDISXVLXXIKXXLXXSXXXIXXSXGKLXXI

SEQ ID NO: 119 - protein, 42 aa, artificial
  preferred optimized consensus sequence of inhibitor peptide
VXXDXXDISXVLXXAKXXLXXSXXXIXXSXGKLXXIGSGSGC

SEQ ID NO: 120 - protein, 34 aa, Human immunodeficiency virus (HIV)
WMEWDREINNYTSLIHSLIEESQNQQEKNEQELL

SEQ ID NO: 121 - protein, 35 aa, Measles virus
PISLERLDVGTNLGNAIAKLEDAKELLESSDQILR

SEQ ID NO: 122 - protein, 27 aa, SARS Virus
GDISGINASVVNIQKEIDRLNEVAKNL

SEQ ID NO: 123 - protein, 30 aa, Junin virus
SYLNISDFRNDWILESDFLISEMLSKEYSD

SEQ ID NO: 124 - protein, 30 aa, Machupo virus
SYLNISEFRNDWILESDHLISEMLSKEYAE

SEQ ID NO: 125 - protein, 30 aa, Guanarito virus
SYLNESDFRNEWILESDHLISEMLSKEYQD

SEQ ID NO: 126 - protein, 30 aa, Lassa virus
SYLNETHFSDDIEQQADNMITEMLQKEYME

SEQ ID NO: 127 - protein, 32 aa, human metapneumovirus (hMPV)
FNVALDQVFENIENSQALVDQSNRILSSAEKG

SEQ ID NO: 128 - protein, 48 aa, human metapneumovirus (hMPV)
AKTIRLESEVTAIKNALKKTNEAVSTLGNGVRVLATAVRELKDFVSKN

SEQ ID NO: 129 - protein, 36 aa, Human herpesvirus (HHV) 6A, 6B
SPDELSRANVFDLENILREYNSYKSALYTIEAKIAT

SEQ ID NO: 130 - protein, 21 aa, Human herpesvirus (HHV) 6A, 6B
INTTESLTNYEKRVTRFYEPP

SEQ ID NO: 131 - protein, 26 aa, Human herpesvirus (HHV) 6A, 6B
ATFVDETLNDVDEVEALLLKFNNLGI

SEQ ID NO: 132 - protein, 29 aa, Cytomegalovirus
NVFDLEEIMREFNSYKQRVKYVEDKVVDP

SEQ ID NO: 133 - protein, 21 aa, Cytomegalovirus
NQVDLTETLERYQQRLNTYAL

SEQ ID NO: 134 - protein, 23 aa, herpes simplex virus 1 (HSV-1)
DYTEVQRRNQLHDLRFADIDTVI

SEQ ID NO: 135 - protein, 22 aa, herpes simplex virus 1 (HSV-1)
ARLQLLEARLQHLVAEILEREQ

SEQ ID NO: 136 - protein, 25 aa, herpes simplex virus 1 (HSV-1)
SDVAAATNADLRTALARADHQKTLF

SEQ ID NO: 137 - protein, 22 aa, Dengue virus 1 (DV1)
AWDFGSIGGVFTSVGKLIHQIF

SEQ ID NO: 138 - protein, 22 aa, Dengue virus 2 (DV2)
AWDFGSLGGVFTSIGKALHQVF

SEQ ID NO: 139 - protein, 22 aa, Dengue virus 3 (DV3)
AWDFGSVGGVLNSLGKMVHQIF

SEQ ID NO: 140 - protein, 22 aa, Dengue virus 4 (DV4)
AWDFGSVGGLFTSLGKAVHQVF

SEQ ID NO: 141 - protein, 22 aa, West Nile virus
AWDFGSVGGVFTSVGKAVHQVF

SEQ ID NO: 142 - protein, 22 aa, Yellow fever virus
AWDFSSAGGFFTSVGKGIHTVF

SEQ ID NO: 143 - protein, 22 aa, Japanese encephalitis virus
AWDFGSIGGVFNSIGKAVHQVF

SEQ ID NO: 144 - protein, 46 aa, Human immunodeficiency virus (Strain HXB2)
TLTVQARQLLSGIVQQQNNLLRAIEAQQHLLQLTVWGIKQLQARIL

SEQ ID NO: 145 - protein, 75 aa, Measles virus
AGVVLAGAALGVATAAQITAGIALHQSMLNSQAIDNLRASLETTNQAIEAIRQSGQEMILAVQGVQDYINNELIP

SEQ ID NO: 146 - protein, 29 aa, Junin virus
QVNLMGQTINALISDNLLMKNKIRELMSV

SEQ ID NO: 147 - protein, 29 aa, Guanarito virus
AVNMLTHSINSLISDNLLMRNKLREILKV

SEQ ID NO: 148 - protein, 29 aa, Machupo virus
EINFLSQTVNALISDNLLMKNKIRELMSV

SEQ ID NO: 149 - protein, 29 aa, Lassa virus
SIQLINKAVNALINDQLIMKNHLRDIMGI

SEQ ID NO: 150 - protein, 55 aa, human metapneumovirus (hMPV)
AKTIRLESEVTAIKNALKKTNEAVSTLGNGVRVLATAVRELKDFVSKNLTRAINK

SEQ ID NO: 151 - protein, 40 aa, human metapneumovirus (hMPV)
PVKFPEDQFNVALDQVFENIENSQALVDQSNRILSSAEKG

SEQ ID NO: 152 - protein, 208 aa, Mus musculus
DIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKS

SEQ ID NO: 153 - protein, 217 aa, Mus musculus
QVQLVQSGAEVRKPGASVKVSCKASGDTFSSYAISWVRQAPGQGLEWMGGIIPIFGTANYAQAFQGRVTITANESTSTAYMELSSLRSEDTAIYYCARDNPTLLGSDYWGAGTLVTVSSASTKGPSVFPLAPCSRSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 154 - protein, 208 aa, Artificial Sequence
  for lipid conjugation
DIQMTQSPSTLSASIGDRVCITCRASEGIYHWLAWYQQKPGKAPKLLIYKASSLASGAPSRFSGSGSGTDFTLTISSLQPDDFATYYCQQYSNYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKS

SEQ ID NO: 155 - protein, 208 aa, Artificial Sequence
  for lipid conjugation
DIQMTQSPSTLSASIGDRVTICCRASEGIYHWLAWYQQKPGKAPKLLIYKASSLASGAPSRFSGSGSGTDFTLTISSLQPDDFATYYCQQYSNYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKS

SEQ ID NO: 156 - protein, 213 aa, Mus musculus
ALQLTQSPSSLSASVGDRITITCRASQGVTSALAWYRQKPGSPPQLLIYDASSLESGVPSRFSGSGSGTEFTLTISTLRPEDFATYYCQQLHFYPHTFGGGTRVDVRRTVAAPSVFIFPPSDEQKSGTASVVCLLNNFYPREAKVQWKVDNALQSGNSQESVTEQDSKDSTYSLSSTLTLSKADYEKHKVYECEVTHQGLSSPVTKSFNRGEC

SEQ ID NO: 157 - protein, 237 aa, Mus musculus
RITLKESGPPLVKPTQTLTLTCSFSGFSLSDFGVGVGWIRQPPGKALEWLAIIYSDDDKRYSPSLNTRLTITKDTSKNQVVLVMTRVSPVDTATYFCAHRRGPTTLFGVPIARGPVNAMDVWGQGITVTISSTSTKGPSVFPLAPSSKSTSGGTAALGCLVKDYFPEPVTVSWNSGALTSGVHTFPAVLQSSGLYSLSSVVTVPSSSLGTQTYTCNVNHKPSNTKVDKRVEPKSCDK

SEQ ID NO: 158 - protein, 213 aa, Artificial Sequence
  for lipid conjugation
ALQLTQSPSSLSASVGDRICITCRASQGVTSALAWYRQKPGSPPQLLIYDASSLESGVPSRFSGSGSGTEFTLTISTLRPEDFATYYCQQLHFYPHTFGGGTRVDVRRTVAAPSVFIFPPSDEQKSGTASVVCLLNNFYPREAKVQWKVDNALQSGNSQESVTEQDSKDSTYSLSSTLTLSKADYEKHKVYECEVTHQGLSSPVTKSFNRGEC

SEQ ID NO: 159 - protein, 213 aa, Artificial Sequence
  for lipid conjugation
ALQLTQSPSSLSASVGDRITICCRASQGVTSALAWYRQKPGSPPQLLIYDASSLESGVPSRFSGSGSGTEFTLTISTLRPEDFATYYCQQLHFYPHTFGGGTRVDVRRTVAAPSVFIFPPSDEQKSGTASVVCLLNNFYPREAKVQWKVDNALQSGNSQESVTEQDSKDSTYSLSSTLTLSKADYEKHKVYECEVTHQGLSSPVTKSFNRGEC

SEQ ID NO: 160 - protein, 237 aa, Artificial Sequence
  non-functional mutant
RITLKESGPPLVKPTQTLTLTCSFSGFSLSDFGVGVGWIRQPPGKALEWLAIIYSDDDKRYSPSLNTRLTITKDTSKNQVVLVMTRVSPVDTATYFCAHRRGPTTSSGVPIARGPVNAMDVWGQGITVTISSTSTKGPSVFPLAPSSKSTSGGTAALGCLVKDYFPEPVTVSWNSGALTSGVHTFPAVLQSSGLYSLSSVVTVPSSSLGTQTYTCNVNHKPSNTKVDKRVEPKSCDK

SEQ ID NO: 161 - protein, 215 aa, Mus musculus
EIVLTQSPGTQSLSPGERATLSCRASQSVGNNKLAWYQQRPGQAPRLLIYGASSRPSGVADRFSGSGSGTDFTLTISRLEPEDFAVYYCQQYGQSLSTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 228 aa, Mus musculus
QVQLVQSGAEVKRPGSSVTVSCKASGGSFSTYALSWVRQAPGRGLEWMGGVIPLLTITNYAPRFQGRITITADRSTSTAYLELNSLRPEDTAVYYCAREGTTGWGWLGKPIGAFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPK

SEQ ID NO: 163 - protein, 215 aa, Artificial Sequence
  for lipid conjugation
EIVLTQSPGTQSLSPGERACLSCRASQSVGNNKLAWYQQRPGQAPRLLIYGASSRPSGVADRFSGSGSGTDFTLTISRLEPEDFAVYYCQQYGQSLSTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 215 aa, Artificial Sequence
  for lipid conjugation
EIVLTQSPGTQSLSPGERATLCCRASQSVGNNKLAWYQQRPGQAPRLLIYGASSRPSGVADRFSGSGSGTDFTLTISRLEPEDFAVYYCQQYGQSLSTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 210 aa, Homo sapiens [sequence omitted]

SEQ ID NO: 166 - protein, 225 aa, Homo sapiens [sequence omitted]

SEQ ID NO: 167 - protein, 210 aa, Artificial Sequence
  for lipid conjugation
EIVLTQSPGTLSLSPGETACISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 168 - protein, 210 aa, Artificial Sequence
  for lipid conjugation
EIVLTQSPGTLSLSPGETAIICCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 169 - protein, 103 aa, Homo sapiens
EIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIK

SEQ ID NO: 170 - protein, 121 aa, Homo sapiens
QVQLVQSGGQMKKPGESMRISCQASGYEFIDCTLNWVRLAPGRRPEWMGWLKPRGGAVNYARPLQGRVTMTRDVYSDTAFLELRSLTADDTAVYYCTRGKNCDYNWDFEHWGRGTPVTVSS

SEQ ID NO: 171 - protein, 103 aa, Artificial Sequence
  for lipid conjugation
EIVLTQSPGTLSLSPGETACISCRTSQYGSLAWYQQRPGQAPRLVIYSGSTRAAGIPDRFSGSRWGPDYNLTISNLESGDFGVYYCQQYEFFGQGTKVQVDIK

SEQ ID NO: 172 - protein, 103 aa, Artificial Sequence
  for lipid conjugation
EIVLTQSPGTLSLSPGETAIICCRTSQYGSLAWYQQRPGQAPRLVIYSGSTRAAGIPDRFSGSRWGPDYNLTISNLESGDFGVYYCQQYEFFGQGTKVQVDIK

SEQ ID NO: 173 - protein, 221 aa, Mus musculus
QSVLTQPPSVSAAPGQKVTISCSGSSSNIGNDYVSWYQQLPGTAPKLLIYDNNKRPSGIPDRFSGSKSGTSATLGITGLQTGDEANYYCATWDRRPTAYVVFGGGTKLTVLGAAAGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 174 - protein, 226 aa, Mus musculus
EVQLVESGAEVKKPGSSVKVSCKASGGPFRSYAISWVRQAPGQGPEWMGGIIPIFGTTKYAPKFQGRVTITADDFAGTVYMELSSLRSEDTAMYYCAKHMGYQVRETMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 175 - protein, 221 aa, Artificial Sequence
  for lipid conjugation
QSVLTQPPSVSAAPGQKVCISCSGSSSNIGNDYVSWYQQLPGTAPKLLIYDNNKRPSGIPDRFSGSKSGTSATLGITGLQTGDEANYYCATWDRRPTAYVVFGGGTKLTVLGAAAGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 176 - protein, 221 aa, Artificial Sequence
  for lipid conjugation
QSVLTQPPSVSAAPGQKVTICCSGSSSNIGNDYVSWYQQLPGTAPKLLIYDNNKRPSGIPDRFSGSKSGTSATLGITGLQTGDEANYYCATWDRRPTAYVVFGGGTKLTVLGAAAGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 177 - protein, 213 aa, Mus musculus
DIQMTQSPSTLSASVGDRVTITCKCQLSVGYMHWYQQKPGKAPKLLIYDTSKLASGVPSRFSGSGSGTAFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 227 aa, Mus musculus
QVTLRESGPALVKPTQTLTLTCTFSGFSLSTSGMSVGWIRQPPGKALEWLADIWWDDKKDYNPSLKSRLTISKDTSANQVVLKVTNMDPADTATYYCARSMITNWYFDVWGAGTTVTVSSASTKGPSVFPLAPSSAAAAGGTAALGCLVKDYFPEPVTVSWNSGALTSGVHTFPAVLQSSGLYSLSSVVTVPSSSLGTQTYICNVNHKPSNTKVDKRVEPKSCDKTH

SEQ ID NO: 179 - protein, 213 aa, Artificial Sequence
  for lipid conjugation
DIQMTQSPSTLSASVGDRVCITCKCQLSVGYMHWYQQKPGKAPKLLIYDTSKLASGVPSRFSGSGSGTAFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - protein, 213 aa, Artificial Sequence
  for lipid conjugation
DIQMTQSPSTLSASVGDRVTICCKCQLSVGYMHWYQQKPGKAPKLLIYDTSKLASGVPSRFSGSGSGTAFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 181 - protein, 213 aa, Mus musculus
DIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 182 - protein, 225 aa, Mus musculus
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 183 - protein, 213 aa, Artificial Sequence
  for lipid conjugation
DIQMTQSPSTLSASVGDRVCITCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 184 - protein, 213 aa, Artificial Sequence
  for lipid conjugation
DIQMTQSPSTLSASVGDRVTICCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 185 - protein, 22 aa, Mus musculus
  MISC_FEATURE (7)..(12): Xaa can be any naturally occurring amino acid
RRGPTTXXXXXXARGPVNAMDV

SEQ ID NO: 186 - protein, 18 aa, Mus musculus
  MISC_FEATURE (6)..(11): Xaa can be any naturally occurring amino acid
EGTTGXXXXXXPIGAFAH

SEQ ID NO: 187 - protein, 36 aa, HPIV3
VALDPIDISIVLNKAKSDLEESKEWIRRSNGKLDSI

SEQ ID NO: 188 - protein, 36 aa, Artificial Sequence
  for lipid conjugation
WXEWXREINXYXSLIXSLIEEXQXQQXKNEXXLXXL

SEQ ID NO: 189 - protein, 36 aa, Artificial Sequence
  a peptide derived from HIV trans-membrane glycoprotein gp41 for lipid conjugation
SWETWEREIENYTRQIYRILEESQEQQDRNERDLLE

SEQ ID NO: 190 - protein, 40 aa, Artificial Sequence
  for lipid conjugation
WQEWEREINKYISLIYSLIEEAQNQQXKNEXALLXLGSGC

SEQ ID NO: 191 - protein, 40 aa, Artificial Sequence
  for lipid conjugation with linker amino acids
SWETWEREIENYTRQIYRILEESQEQQDRNERDLLEGSGC

SEQ ID NO: 192 - protein, 36 aa, Artificial Sequence
  for lipid conjugation
WNEWEREINKYTSLIYSLIEEAQNQQDKNEKDLLEL